SEQUENCE LISTING (32 sequences)

SEQ ID NO: 1 - protein, 10 aa, artificial sequence
  TAT nuclear localization signal peptide
YGRKRRQRRR

SEQ ID NO: 2 - DNA, 21 bp, artificial sequence
  Vitravene (fomivirsen)
gcgtttgctcttcttcttgcg

SEQ ID NO: 3 - DNA, 20 bp, artificial sequence
  oligonucleotide- ISIS 102453
cccctcgctctccgctcccg

SEQ ID NO: 4 - DNA, 20 bp, artificial sequence
  oligonucleotide- ISIS 112043
gtgnttggctgagttnanaa

SEQ ID NO: 5 - DNA, 20 bp, artificial sequence
  oligonucleotide-- ISIS 112043
aganatttattcagcgtnan

SEQ ID NO: 6 - DNA, 20 bp, artificial sequence
  oligonucleotide-- ISIS 105990
agnaaaagatnaatnngtta

SEQ ID NO: 7 - DNA, 20 bp, artificial sequence
  oligonucleotide-- ISIS 105990
tttgntgtaattnanantga

SEQ ID NO: 8 - DNA, 20 bp, artificial sequence
  oligonucleotide-- ISIS 101528
ngngnaccgcccggcngnnc

SEQ ID NO: 9 - DNA, 20 bp, artificial sequence
  oligonucleotide-- ISIS 101528
aggangccgccatcttnnnn

SEQ ID NO: 10 - DNA, 20 bp, artificial sequence
  oligonucleotide-- ISIS 21329
gtttngcaaaatcactnggg

SEQ ID NO: 11 - DNA, 20 bp, artificial sequence
  oligonucleotide-- ISIS 21329
gggtttcgcaaaatcantng

SEQ ID NO: 12 - DNA, 18 bp, artificial sequence
  oligonucleotide-- ISIS 25237
gnnnattgctggacatgn

SEQ ID NO: 13 - DNA, 18 bp, artificial sequence
  oligonucleotide-- ISIS 25962
gtntgactttagcatntn

SEQ ID NO: 14 - DNA, 18 bp, artificial sequence
  oligonucleotide-- ISIS 25962
ggtatcttttcttctgtt

SEQ ID NO: 15 - DNA, 18 bp, artificial sequence
  oligonucleotide-- ISIS 29714
caagttgctgcctgggaa

SEQ ID NO: 16 - DNA, 18 bp, artificial sequence
  oligonucleotide-- ISIS 29714
nngacgtcttgtggatga

SEQ ID NO: 17 - DNA, 18 bp, artificial sequence
  oligonucleotide-- ISIS 29176
agtntactgctcggnnat

SEQ ID NO: 18 - DNA, 18 bp, artificial sequence
  oligonucleotide-- ISIS 29176
ntaggccccaccagtnta

SEQ ID NO: 19 - DNA, 18 bp, artificial sequence
  oligonucleotide-- ISIS 28030
atttcctgggatgtgngg

SEQ ID NO: 20 - DNA, 20 bp, artificial sequence
  oligonucleotide--  ISIS 2302
gcccaagctggcatccgtca

SEQ ID NO: 21 - DNA, 20 bp, artificial sequence
  oligonucleotide--  3521/CGP, 64128A (Isis, Novartis)
gttctcgctggtgagtttca

SEQ ID NO: 22 - DNA, 20 bp, artificial sequence
  oligonucleotide--  5132/CPG, 64128A (Isis, Novartis)
tcccgcctgtgacatgcatt

SEQ ID NO: 23 - DNA, 20 bp, artificial sequence
  oligonucleotide-- ISIS 2503
tccgtcatcgctcctcaggg

SEQ ID NO: 24 - DNA, 18 bp, artificial sequence
  oligonucleotide-- G3139 Genta
tctcccagcgtgcgccat

SEQ ID NO: 25 - DNA, 14 bp, artificial sequence
  oligonucleotide-- LR3280 Lynx
aacgttgagggcat

SEQ ID NO: 26 - DNA, 24 bp, artificial sequence
  Oligonucleotide--LR3001-Lynx
tatgctgtgccggggtcttcgggc

SEQ ID NO: 27 - DNA, 18 bp, artificial sequence
  oligonucleotide-- LR4437-Lynx
ggaccctcctccggagcc

SEQ ID NO: 28 - DNA, 20 bp, artificial sequence
  oligonucleotide-- GEM-132 (Hybridon)
uggggcttaccttgcgaaca

SEQ ID NO: 29 - DNA, 21 bp, artificial sequence
  Oligonucleotide-- GEM-92 (Hybridon)
ucgcacccatctctctccuuc

SEQ ID NO: 30 - DNA, 18 bp, ARTIFICIAL SEQUENCE
  Oligonucleotide-- GEM-231 (hybridon)
gcgugcctcctcacuggc

SEQ ID NO: 31 - DNA, 20 bp, artificial sequence
  oligonucleotide GPI-2A- Novopharm
ggttcttttggtccttgtct

SEQ ID NO: 32 - DNA, 21 bp, artificial sequence
  oligonucleotide- ISIS 13312
gcgtttgctcttcttcttgcg

(19) United States
(12) Patent Application Publication
(10) Pub. No.: US 2006/0074034 A1
(43) Pub. Date: Apr. 6, 2006

(54) Cobalamin mediated delivery of nucleic acids, analogs and derivatives thereof

(76) Inventors: Douglas A. Collins, Rochester, MN (US); Franklyn G. Prendergast, Rochester, MN (US); Matthew R. Callstrom, Rochester, MN (US)

Correspondence Address: Sherry M. Knowles;King & Spalding, 45th Floor, 191 Peachtree Street, Atlanta, GA, 30303, US

(21) Appl. No.: 10/246,300

(22) Filed: Sep. 17, 2002

Related U.S. Application Data
(60) Provisional application No. 60/322,861, filed on Sep. 17, 2001.
(60) Provisional application No. 60/410,627, filed on Sep. 13, 2002.

(51) Int. Cl. A61K 48/00; A61K 31/714; C07H 21/04; C07H 23/00

(52) U.S. Cl. 514/44; 514/52; 536/23.1; 536/26.44

(57) ABSTRACT

This invention is in the area of cobalamin-mediated delivery of nucleic acids and analogs and derivatives thereof to a host to affect gene expression.

This application claims priority to U.S. Ser. No. 60/322,861, filed on Sep. 17, 2001 and an additional U.S. provisional application filed on Sep. 13, 2002.

### FIELD OF THE INVENTION

This invention is in the area of cobalamin-mediated delivery of nucleic acids and analogs and derivatives thereof to a host to affect gene expression.

### BACKGROUND OF THE INVENTION

The goal of gene therapy is to treat disorders resulting from genetic defects, or to enhance health generally. Gene therapy can act to modulate the expression or inhibition of expression of a target protein that mediates a disorder (FIG. 1). The modulation can take place at the level of translation or transcription via an antisense or stabilized antisense sequence or an antisense mimic such as a peptide nucleic acid (PNA), mopholinonucleic acid (MNA), locked nucleic acid (LNA), pseudocyclic oligonucleobase (PCO), or 2′-O,4′-C-ethylene bridged nucleic acid (ENA).

Gene therapy can also include, for example, the insertion or deletion of a gene to cause gene expression or inhibition of expression via genetic engineering of cells. The engineered cell can be a foreign cell that is implanted or otherwise administered to the host organism (heterologous gene therapy), or can be a cell of the host (autologous gene therapy).

Gene therapy can be used to create a new cellular function by introducing a particular gene of interest that expresses a protein not currently expressed by the cell. In one embodiment, a nucleic acid sequence encoding a cytocide can be delivered to cancer cells. When the nucleic acid sequence is expressed, the cancer cell dies.

In another embodiment, gene therapy can be accomplished by administering a transcriptional factor, receptor/ligand complex or other protein or protein analog or stabilized variant that turns on or off gene expression.

Extracellular genetic material can be introduced into cells using a variety of techniques. Deoxyribonucleic acid (DNA) or ribonucleic acid (RNA) can be introduced into a cell by co-precipitation with calcium phosphate, electroporation or using liposomes. Nucleic acids are also known to be internalized by cells without external assistance albeit at a very small amount due in part to the hydrophilic character of the nucleic acid and the hydrophobic character of cellular membranes. Viral vectors are routinely used to introduce nucleic acid sequences into cells; however, the safety of viral vectors is a concern because of the possibility of side effects and random mutations in the vector generating a fully active virus. Further, these techniques do not adequately address the problems of targeting the nucleic acid sequences to the cells or tissues of interest.

I. Antisense Oligonucleotides

Antisense oligonucleotides are short, synthetic strands of DNA (or analogs) that are complimentary, or antisense, to a target sequence (DNA or RNA) and are designed to halt a biological event, such as transcription, translation or splicing. Antisense is a powerful tool for the molecular biologist. The first antisense drug (Isis's Fomivirsen) recently received FDA approval for the treatment of CMV (cytomegalovirus). The antisense field is experiencing an explosion of interest now that the concept of the inhibition of gene expression by antisense sequences is being increasingly confirmed. See www.trilink.biotech.com/Technical_Information/Antisense%20primer.html.

One of the challenges of antisense therapy is to stabilize the oligonucleotide to increase bioavailablility and half life while maintaining strong hybridization with the target sequence and ease of manufacture. One of the most simple and straightforward modifications that can be made to an oligonucleotide is to replace a non-bridging oxygen on the phosphate backbone with sulfur, producing a phosphorothioate linkage. The ability of this modification to retard nuclease degradation of oligonucleotides is long known (Matzura and Eckstein, 1968). It was later learned that this modification is also a substrate for RNaseH (Stein et al., 1988; Furdon et al., 1989). These properties, combined with the relative ease of synthesis have led to the ascendancy of this form of stabilization in the antisense drug. However, the road has not been easy. Despite the issues, most of the compounds progressing through clinical trials at this time are phosphorothioates.

Second generation oligonucleotide constructs are available commercially and the less complex ones are not much more expensive than phosphorothioate oligonucleotides at the smaller scales. In fact, most of them include some phosphorothioate linkages, and many are still completely modified with phosphorothioates. A common design is to have nuclease resistant arms (such as 2′-O-methyl (OMe) nucleosides) that surround a phosphorothioate modified deoxyribose core that retains the RNase H activity of the oligonucleotide. Oligonucleotides that contain mixtures of stablization chemistries are called chimeric oligonucleotides. Chimeric oligonucleotides containing 2′-OMe arms were used to help understand the underlying principles of the RNase H mechanism. The most significant enhancements offered by this class of compound are a general reduction in toxicity, increased hybrid stability and increased nuclease stability.

An alternative means to stablize an antisense oligonucleotide is to use one or more 2′-fluoro-nucleosides in the antisense sequence. See generally U.S. Pat. Nos. 5,686,242 and 5,670,633 to Isis Pharmaceuticals.

II. Peptide Nucleic Acids (PNA)

PNA is a nucleic acid analog with an achiral polyamide backbone consisting of N-(2-aminoethyl)glycine units (FIG. 2). The purine or pyrimidine bases are linked to each unit via a methylene carbonyl linker (1-3) to target the complementary nucleic acid. PNA binds to complementary RNA or DNA in a parallel or antiparallel orientation following the Watson-Crick base-pairing rules. The uncharged nature of the PNA oligomers enhances the stability of the hybrid PNA/DNA (RNA) duplexes as compared to the natural homoduplexes. The non-natural character of the PNA makes PNA oligomers highly resistant to protease and nuclease attacks. These properties of PNA oligomers suggest that they could serve as efficient antisense or antigene reagents. Indeed, peptide nucleic acids have been applied to block protein expression on the transcriptional and translational level, and microinjected PNA oligomers demonstrate a strong antisense effect in intact cells. However, contrary to the “normal” nucleic acid analogs, PNA oligomers are not efficiently delivered into the cytoplasm of the cell, and until recently this has hindered the application of PNA oligomers as antisense reagents. See www.bioscience.org/1999/v4/d/soomets/fulltext.htm; and Frontiers in Bioscience, 4, d782-786 (Nov. 1, 1999) for details on recent achievements on PNA antisense application, especially these concerned with whole cell or tissue delivery of the PNA. See also Nielsen, P. E., Egholm. M., Berg, R. H. & Buchardt, O. (1993) Peptide nucleic acids (PNA). DNA analogues with a polyamide backbone. In “Antisense Research and Application” Crook, S. & Lebleu, B. (eds.) CRC Press, Boca Raton, pp 363-373.

PNA is both biologically and chemically stable and readily available by automated synthesis ((Hyrup, B., Egholm, M., Rolland, M., Nielsen, P. E., Berg, R. H. & Buchardt, O. (1993) Modification of the binding affinity of peptide nucleic acids (PNA). PNA with extended backbones consisting of 2-Aminoethyl—Alanine or 3-Aminopropylglycine units. J. Chem. Soc. Chem. Commun. 518-519); Demidov, V., Frank-Kamenetskii, M. D., Egholm, M., Buchardt, O. & Nielsen, P. E. (1993). Sequence selective double strand DNA cleavage by PNA targeting using nuclease S1. Nucleic Acids Res. 21, 2103-2107). These properties have made PNA a very good lead for developing antisense gene therapeutic drugs, and in vitro studies have further substantiated the antisense potential (Nielsen, P. E. “Peptide Nucleic Acid (PNA) A model structure for the primordial genetic material” Origins of Life 1993, 23, 323-327; Egholm, M., Behrens, C., Christensen, L., Berg, R. H., Nielsen, P. E. & Buchardt, O. “Peptide nucleic acids containing adenine or guanine recognize thymine and cytosine in complementary DNA sequences” J. Chem. Soc. Chem. Commun. 1993, 800-801; Kim, S. K., Nielsen, P. E., Egholm, M., Buchardt, O., Berg, R. H. & Nordén, B. “Right-handed triplex formed between peptide nucleic acid PNA-T₈ and poly(dA) shown by linear and circular dichroism spectroscopy” J. Amer. Chem. Soc. 1993, 115, 6477-6481; Egholm, M., Buchardt, O., Christensen, L., Behrens, C., Freier, S. M., Driver, D. A., Berg, R. H., Kim, S. K., Nordén, B. & Nielsen, P. E. “PNA hybridizes to complementary oligonucleotides obeying the Watson-Crick hydrogen bonding rules” Nature 1993, 365, 556-568; Buchardt, O., Egholm, M., Berg, R. & Nielsen, P. E. “Peptide Nucleic Acids (PNA) and their potential applications in medicine and biotechnology” Trends Biotechnology, 1993, 11, 384-386).

PNAs appear to be the very useful for a number of special applications. When targeted against rare all-purine sequences, PNAs can block translation anywhere in a mRNA by forming a double-clamp structure. With such rare RNA sequences one segment of the PNA binds to the target sequence by Watson/Crick bonds and the other segment of the PNA binds to major-groove sites of the resulting PNA/RNA duplex. Probably because of their very flexible backbone structure, PNAs also readily form triple helix structures with rare duplex DNA sequences comprising mostly purines in one strand and mostly pyrimidines in the other strand. Lastly, under low salt conditions in cell-free systems PNAs have been shown to achieve sequence-specific invasion of duplex DNA sequences, resulting in inhibition of transcription of the invaded duplex.

PNAs targeted to the AUG-initiation region of mRNA are very potent and specific inhibitors of translation. Unlike phosphorothioate oligonucleotides, RNase H does not play a role in the antisense mechanism of PNAs.

PNAs have been targeted to the RNA template of reverse transcriptase of HIV and have been shown to efficiently block elongation by the enzyme in vitro (Koppelhus et al. 1997 Nucleic Acid Res. 25:2167-2173; Lee et al. 1998, Biochemistry 37:900-910). PNAs targeted to the TAR sequence of HIV have been shown to inhibit HIV expression (Mayhood, T. et al. 2000 Biochemistry 26:11532-9). Yang et al. blocked HIV-1 replication using PNAs targeted to the 3′ end of the HIV-1 gag-pol transframe region (Yang et al. 2000 J. Virol. 74:4621-33). Peptide-PNA conjugates containing HIV Rev peptides have been reported to bind HIV RRE IIB RNA (Kumagai, I. et al. 2000 Bioorg. Med. Chem. Lett. 4:377-379). A DNA-PNA hybrid molecule was found to be recognized by NF-kappaB p52 suggesting that DNA-PNA hybrids may be useful for the decoy approach in gene therapy (Mischiati, P. F. et al. 1999 4:33114-22).

Good and Nielsen have recently published studies (Good, L. & P. E. Nielsen: Antisense inhibition of gene expression in bacteria by PNA targeted to mRNA. Nat Biotechnol 1998, 16, 355-358; and Good, L. & P. E. Nielsen: Inhibition of translation and bacterial growth by peptide nucleic acid targeted to ribosomal RNA. Proc Nat Acad Sci USA 1998, 95, 2073-2076) showing that PNA can inhibit reporter gene expression in E. coli, probably through an antisense mechanism. The inhibition was shown to be specific and concentration dependent, and was more efficient in antibiotic permeable bacteria than in the wild type strain.

Some studies have shown anti-gene activity of PNAs in the cells showing its potential as possible regulator of gene expression. Vickers et al (Vickers, T. A., M. C. Griffith, K. Ramasamy, L. M. Risen & S. M. Freier: Inhibition of NF-kappa B specific transcriptional activation by PNA strand invasion. Nucleic Acids Res 1995, 23, 3003-3008) studied the ability of 15-mer PNA to specifically block interaction of the transcription factor NF-kB with its binding site in the IL2-Rα promoter. Complete inhibition of transcription was shown when the cells were transfected with an IL2-Rα plasmid pre-incubated with PNA, while the treatment of cells with PNA after the transfection failed to modulate the transcription.

Boffa et al (Boffa, L. C., E. M. Carpaneto, M. R. Mariani, M. Louissaint & V. G. Allfrey: Contrasting effects of PNA invasion of the chimeric DMMYC gene on transcription of its myc and PVT domains. Oncology Res 1997, 9, 41-51) reported that the 18-mer PNA complementary to the poly-CAG triplet area (Boffa, L. C., E. M. Carpaneto & V. G. Allfrey: Isolation of active genes containing CAG repeats by DNA strand invasion by a peptide nucleic acid. Proc Nat Acad Sci USA 1995, 92, 1901-1905) of the androgen receptor and a TATA binding protein has a specific anti-gene activity in permeabilized prostatic cancer cells (Boffa, L. C., P. L. Morris, E. M. Carpaneto, M. Louissaint & V. G. Allfrey: Invasion of the CAG triplet repeats by a complementary peptide nucleic acid inhibits transcription of the androgen receptor and TATA-binding protein genes and correlates with refolding of an active nucleosome containing a unique AR gene sequence. J Biol Chem 1996, 271, 13228-13233). Furthermore, the same authors showed that 17-mer PNA complementary to the sense strand of the second myc exon inhibits transcription in permeabilized COLO320-DM cells.

Although PNAs have several characteristics required for a good antisense molecule, they suffer from poor membrane penetrability. Therefore, the initial antisense experiments using PNA relied on microinjection and cell permeabilization techniques.

In addition, while PNAs have dramatically improved properties relative to S-DNAs, PNAs do have some limitations. An 18 subunit length is the longest commercially available and many sequences are difficult to make, probably because the extreme flexibility of the acyclic backbone of PNAs allows undue intrastrand interactions. Most PNAs also have limited aqueous solubility, which can present difficulties in their routine use. PNAs also provide less than ideal sequence specificity, probably because their very high RNA binding affinities result in significant binding to short sequences in a variety of cellular mRNAs.

Wittung et al have shown, using an liposomal model system for the plasma membrane, that PNAs do not readily diffuse through a membrane barrier (Wittung, P., J. Kajanus, K. Edwards, G. Haaima, P. E. Nielsen, B. Nordén & B. G. Malmström: Phospholipid membrane permeability of peptide nucleic acid [corrected and republished with original paging, article originally printed in FEBS Lett 1995 May 22; 365(1):27-9] FEBS Letters 1995, 375, 27-29). Furthermore, Bonham et al showed that incubating CV-1 cells with FITC-labeled PNA resulted only in cytoplasmic vesicular staining (Bonham, M. A., S. Brown, A. L. Boyd, P. H. Brown, D. A. Bruckenstein, J. C. Hanvey, S. A. Thomson, A. Pipe, F. Hassman, J. E. Bisi, B. C. Froehler, M. D. Matteucci, R. W. Wagner, S. A. Noble & L. E. Babiss: An assessment of the antisense properties of RNase H-competent and steric-blocking oligomers. Nucleic Acids Res 1995, 23, 1197-1203). Indeed, the neuron is the only cell type to date that has been shown to efficiently internalize unmodified PNAs, demonstrating the need for an effective transporter for these molecules for other tissues. To this end, several potential delivery systems have been developed.

However, Liposomal delivery that is often used for transfection with oligonucleotides has not been successfully used for PNA transport.

U.S. Pat. No. 5,736,392 to Hawley-Nelson et al. discloses the stabilization of the PNA complex with cationic lipids.

An interesting solution to the PNA uptake problem was proposed by Uhlmann et al. They showed that a synthetic PNA-DNA chimeric molecule was internalized by cells as efficiently as normal oligos (Uhlmann, E., D. W. Will, G. Breipohl, D. Langner & A. Ryte: Synthesis and properties of PNA/DNA chimeras. Angew. Chem. Int. Ed. Engl. 1996, 35, 2793-2797) and, furthermore, in contrast to PNAs, were able to activate RNAse H (Uhlmann, E., A. Peyman & D. W. Will: Antisense: Chemical modifications. In: Encyclopedia of Cancer. Eds: Bertino, J. R., Academic Press, San Diego, 1997, Vol. 1, 64-81).

Recent studies by several groups have shown that coupling of PNA to different carriers will improve their uptake into cells. Among these, “cellular uptake peptides,” fatty acids or DNA, especially several peptide sequences have been shown to be able to carry PNA oligomers across the cell membranes. Vector peptide-PNA conjugates have been shown to cross the neuron membrane and suppress targeted mRNA (Aldrian-Herrada, G. et al. (1998) Nucleic Acid Res. 26:4920). Biotinylated PNA linked to a conjugate of steptavidin and the OX26 murine monoclonal antibody to the rat transferrin receptor have been reported to cross the rat blood-brain barrier in vivo (Pardridge, W. et al. 1995 PNAS 92:5592-5596). Chinnery, P. F. et al. attached the presequence peptide of the nuclear-encoded human cytochrome c oxidase (COX) subunit VIII to biotinylated PNA which was successfully imported into isolated mitochondria in vitro (Chinnery, P. F. et al. 1999 Gene Ther. 6:1919-28). Delivery of the biotinylated peptide-PNA to mitochondria in intact cells was confirmed by confocal microscopy.

A short hydrophobic peptide with the sequence biotinyl-FLFL coupled to a PNA trimer has been shown to internalize into human erythrocytes and Namalwa cells (Scarfi, S., A. Gasparini, G. Damonte & U. Benatti: Synthesis, uptake, and intracellular metabolism of a hydrophobic tetrapeptide-peptide nucleic acid (PNA)-biotin molecule. Biochem Biophys Res Commun 1997, 236, 323-326). However, no data on transport of longer PNAs has been shown. Basu and Wickström (Synthesis and characterization of a peptide nucleic acid conjugated to a D-peptide analog of insulin-like growth factor 1 for increased cellular uptake. Bioconjugate Chem 1997, 8, 481-488) showed that PNA conjugated to an all-D-amino acid insulin-like growth factor 1 (IGF1) mimicking peptide was specifically taken up by cells expressing the IGF1 receptor, although no antisense activity was described.

In recent years, some peptides that translocate over the plasma membrane in an energy and endocytotic independent manner, have been designed and synthesized. An extensively studied sequence, derived from the third helix of the Antennapedia homeodomain (Derossi, D., S. Calvet, A. Trembleau, A. Brunissen, G. Chassaing & A. Prochiantz: Cell internalization of the third helix of the Antennapedia homeodomain is receptor-independent. J Biol Chem 1996, 128, 18188-18193), is called penetratin (for review see Derossi, D., G. Chassaing & A. Prochiantz: Trojan peptides: the penetratin system for intracellular delivery. Trends Cell 1998, 8, 84-87). Penetratin or penetratin analogs have been used by Pooga, M., U. Soomets, M. Hällbrink, A. Valkna, K. Saar, K. Rezaei, U. Kahl, J. X. Hao, X. J. Xu, Z. Wiesenfeld-Hallin, T. Hökfelt, T. Bartfai & Ü. Langel: Cell penetrating PNA constructs regulate galanin receptor levels and modify pain transmission in vivo. Nat Biotechnol 1998, 16, 857-861 and others (Aldrian-Herrada, G., M. G. Desarménien, H. Orcel, L. Boissin-Agasse, J. Méry, J. Brugidou & A. Rabié: A peptide nucleic acid (PNA) is more rapidly internalized in cultured neurons when coupled to a retro-inverso delivery peptide. The antisense activity depresses the target mRNA and protein in magnocellular oxytocin neurons. Nucleic Acids Res 1998, 26, 4910-4916; and Simmons, C. G., A. E. Pitts, L. D. Mayfield, J. W. Shay & D. R. Corey: Synthesis and membrane permeability of PNA-peptide conjugates. Bioorg Med Chem Lett 1997, 7, 3001-3006) to transport PNAs over the plasma membrane of cells in culture. Moreover, we used the chimeric peptide transportan as an alternative transport peptide, showing that penetratin is not the only transport peptide that can mediate PNA transport (Pooga, M., U. Soomets, M. Hällbrink, A. Valkna, K. Saar, K. Rezaei, U. Kahl, J. X. Hao, X. J. Xu, Z. Wiesenfeld-Hallin, T. Hökfelt, T. Bartfai & Ü. Langel: Cell penetrating PNA constructs regulate galanin receptor levels and modify pain transmission in vivo. Nat Biotechnol 1998, 16, 857-861). The conjugation of a transporter peptide to PNA greatly improved uptake in neurons (Aldrian-Herrada, G., M. G. Desarménien, H. Orcel, L. Boissin-Agasse, J. Méry, J. Brugidou & A. Rabié: A peptide nucleic acid (PNA) is more rapidly internalized in cultured neurons when coupled to a retro-inverso delivery peptide. The antisense activity depresses the target mRNA and protein in magnocellular oxytocin neurons. Nucleic Acids Res 1998, 26, 4910-4916) and was necessary for any significant uptake in Bowes (Pooga, M., U. Soomets, M. Hällbrink, A. Valkna, K. Saar, K. Rezaei, U. Kahl, J. X. Hao, X. J. Xu, Z. Wiesenfeld-Hallin, T. Hökfelt, T. Bartfai & Ü. Langel: Cell penetrating PNA constructs regulate galanin receptor levels and modify pain transmission in vivo. Nat Biotechnol 1998, 16, 857-861) and DU-145 cells (Simmons, C. G., A. E. Pitts, L. D. Mayfield, J. W. Shay & D. R. Corey: Synthesis and membrane permeability of PNA-peptide conjugates. Bioorg Med Chem Lett 1997, 7, 3001-3006).

In their study (Aldrian-Herrada, G., M. G. Desarménien, H. Orcel, L. Boissin-Agasse, J. Méry, J. Brugidou & A. Rabié: A peptide nucleic acid (PNA) is more rapidly internalized in cultured neurons when coupled to a retro-inverso delivery peptide. The antisense activity depresses the target mRNA and protein in magnocellular oxytocin neurons. Nucleic Acids Res 1998, 26, 4910-4916) Aldrian-Herrada and co-workers synthesized antisense PNA against the starting codon region of prepro-oxytocin mRNA. Treatment of cultured magnocellular oxytocin neurons with antisense PNA or vector peptide-PNA conjugate resulted in reduced immunohistochemical signal for prepro-oxytocin and reduced amount of oxytocin mRNA in a dose- and time-dependent manner. The mechanism behind the decrease of mRNA levels is not clear yet, but the authors suggest that the PNA-induced RNA degradation could occur in RNaseH independent metabolic pathways.

For some examples of other works, see Nielsen, P. E., M. Egholm, R. H. Berg & O. Buchardt: Sequence-selective recognition of DNA by strand displacement with a thymine-substituted polyamide. Science 254, 1497-1500 (1991); Egholm, M., O. Buchart, P. E. Nielsen & R. H. Berg: Peptide nucleic acids (PNA). Oligonucleotide analogues with an achiral peptide backbone. J Am Chem Soc 114, 1895-1897 (1992); Nielsen, P. E., M. Egholm & O. Buchardt: Peptide nucleic acid (PNA). A DNA mimic with a peptide backbone. Bioconjugate Chemistry 5, 3-7 (1994); Egholm, M., P. E. Nielsen, O. Buchardt & R. H. Berg: Recognition of guanine and adenine in DNA by cytosine and thymine containing peptide nucleic acid. J Am Chem Soc 114, 9677-9678 (1992); Egholm, M., O. Buchardt, L. Christensen, C. Behrens, S. M. Freier, D. A. Driver, R. H. Berg, S. K. Kim, B. Nordén & P. E. Nielsen: PNA hybridizes to complementary oligonucleotides obeying the Watson-Crick hydrogen-bonding rules [see comments]. Nature 365, 566-568 (1993); Wittung, P., P. E. Nielsen, O. Buchardt, M. Egholm & B. Nordén: DNA-like double helix formed by peptide nucleic acid. Nature 368, 561-563 (1994); Brown, S. C., S. A. Thomson, J. M. Veal & D. G. Davis: NMR solution structure of a peptide nucleic acid complexed with RNA. Science 265, 777-780 (1994); Demidov, V. V., V. N. Potaman, M. D. Frank-Kamenetskii, M. Egholm, O. Buchard, S. H. Sönnichsen & P. E. Nielsen: Stability of peptide nucleic acids in human serum and cellular extracts. Biochemical Pharmacology 48, 1310-1313 (1994); Peffer, N. J., J. C. Hanvey, J. E. Bisi, S. A. Thomson, C. F. Hassman, S. A. Noble & L. E. Babiss: Strand-invasion of duplex DNA by peptide nucleic acid oligomers. Proc Nat Acad Sci USA 90, 10648-10652 (1993).

Other viruses that have been inhibited using PNAs include bovine papillomavirus E2 (Kurg, R. et al. 2000 66:39-50). The PNA bound to double stranded DNA and prevented the E2 protein from binding to its DNA binding site thereby interfering with the E2 initiation of DNA replication in vivo.

PNAs have also been the subject matter of several patents. For example, U.S. Pat. No. 6,015,887 to Teng discloses methods and compositions for chiral peptide nucleic acids.

U.S. Pat. No. 6,046,307 to Shay et al. discloses PNAs that inhibit telomerase activity in mammalian cells.

U.S. Pat. No. 5,789,573 to Baker et al. disclose compositions and methods for inhibiting the translation of capped targeted mRNA using PNAs.

U.S. Pat. No. 6,165,720 disclose labeling of the PNA complex.

WO 99/20643 filed by Mayo Foundation for Medical Education and Research describes PNA oligomers that cross a biological barrier and engender a biological response.

III. Morpholino Nucleic Acids (MNA)

Morpholino antisense oligonucleotides (oligos) are so named because they are assembled from morpholino subunits, each of which contains one of the four genetic bases (adenine, cytosine, guanine, and thymine) linked to a 6-membered morpholine ring. Eighteen to twenty-five subunits of these four subunit types are joined in a specific order by non-ionic phosphorodiamidate intersubunit linkages to give a morpholino oligo. FIG. 2 shows a short segment of a morpholino oligo, comprising two subunits joined by an intersubunit linkage. These morpholino oligos, with their 6-membered morpholine backbone moieties joined by non-ionic linkages, afford substantially better antisense properties than do RNA, DNA, and their analogs having 5-membered ribose or deoxyribose backbone moieties joined by ionic linkages (see wwwgene-tools.com/Morpholinos/body_morpholinos.HTML).

Morpholinos, devised by Summerton in 1985, constitute a radical redesign of natural nucleic acids, with the potential advantages of low cost starting materials and inexpensive assembly. Like PNAs, morpholinos are completely resistant to nucleases and they appear to be free of most or all of the non-antisense effects that plague S-DNAs. In contrast to PNAs, most morpholinos exhibit excellent aqueous solubility. Morpholinos also have much higher RNA binding affinities than do S-DNAs, though not as high as PNAs. Probably as a result of their substantial RNA binding affinities, long morpholinos (25-mers) provide predictable targeting and very high efficacy. Most notable, morpholinos provide good sequence specificity. The same factors that underlie their exceptional sequence specificity also render them unsuitable for targeting point mutations.

U.S. Pat. No. 6,153,737 to Manoharan et al. is directed to derivatized oligonucleotides wherein the linked nucleosides are functionalized with peptides, proteins, water soluble vitamins or lipid soluble vitamins. This disclosure was directed towards antisense therapeutics by modification of oligonucleotides with a peptide or protein sequence that aids in the selective entry of the complex into the nuclear envelope. Similarly, water-soluble and lipid-soluble vitamins can be used to assist in the transfer of the anti-sense therapeutic or diagnostic agent across cellular membranes.

IV. LNAs, PCOs, and ENAs.

LNA is a novel class of DNA analogue that possesses some features that make it a prime candidate for improving nucleic acid properties. The LNA monomers are bi-cyclic compounds structurally similar to RNA-monomers. LNA share most of the chemical properties of DNA and RNA, it is water-soluble, can be separated by gel electrophoreses, ethanol precipitated etc (Tetrahedron, 54, 3607-3630 (1998)). However, introduction of LNA monomers into either DNA or RNA oligos results in high thermal stability of duplexes with complementary DNA or RNA, while, at the same time obeying the Watson-Crick base-pairing rules. This high thermal stability of the duplexes formed with LNA oligomers together with the finding that primers containing 3′ located LNA(s) are substrates for enzymatic extensions, e.g. the PCR reaction, is used in the present invention to significantly increase the specificity of detection of variant nucleic acids in the in vitro assays described in the application. The amplification processes of individual alleles occur highly discriminative (cross reactions are not visible) and several reactions may take place in the same vessel. See for example U.S. Pat. No. 6,316,198.

Pseudo-cyclic oligonucleobases (PCOs) can also be used in the regulator (see U.S. Pat. No. 6,383,752). PCOs contain two oligonucleotide segments attached through their 3′-3′ or 5′-5′ ends. One of the segments (the “functional segment”) of the PCO has some functionality (e.g., an antisense oligonucleotide complementary to a target mRNA). Another segment (the “protective segment”) is complementary to the 3′- or 5′-terminal end of the functional segment (depending on the end through which it is attached to the functional segment). As a result of complementarity between the functional and protective segment segments, PCOs form intramolecular pseudo-cyclic structures in the absence of the target nucleic acids (e.g., RNA). PCOs are more stable than conventional antisense oligonucleotides because of the presence of 3′-3′ or 5′-5′ linkages and the formation of intramolecular pseudo-cyclic structures. Pharmacokinetic, tissue distribution, and stability studies in mice suggest that PCOs have higher in vivo stability than and, pharmacokinetic and tissue distribution profiles similar to, those of PS-oligonucleotides in general, but rapid elimination from selected tissues. When a fluorophore and quencher molecules are appropriately linked to the PCOs of the present invention, the molecule will fluoresce when it is in the linear configuration, but the fluorescence is quenched in the cyclic conformation.

2′-O,4′-C-Ethylene bridged nucleic acids (ENA) are described, for example in Morita K, Hasegawa C, Kaneko M, Tsutsumi S, Sone J, Ishikawa T, Imanishi T, Koizumui M; 2′-O,4′-C-ethylene bridged nucleic acids (ENA): highly nuclease resistant and thermodynamically stable olionucleotides for antisense drugs. Bioorg Med Chem Lett 2002 Jan. 7; 12(1):73-6; and Synthesis of 2′-O-[2-[N,N-dimethylamino)oxy]ethyl] modified nucleosides and oligonucleotides. Prakash T P, Kawasaki A M, Fraser A S, Vasquez G, Monoharan M. J Org Chem 2002 Jan. 25; 67 (2):357-69.

IV. Delivery of Materials with Vitamin B₁₂

The targeting of small molecules to specific tissues and cells has been accomplished using vitamins. Vitamin B₁₂ has been conjugated to many different molecules.

U.S. Pat. Nos. 5,739,313; 6,004,533; 6,096,290, 6,211,355 and PCT Publication WO 97/18231 listing Collins and Hogenkamp as inventors disclose radionuclide labeling of vitamin B₁₂ through the propionamide moieties on naturally occurring vitamin B₁₂. The inventors converted the propionamide moieties at the b-, d-, and e-positions of the corrole ring to monocarboxylic acids, through a mild hydrolysis, and separated the carboxylic acids by column chromatography. The inventors then attached a bifunctional linking moiety to the carboxylate function through an amide linkage, and a chelating agent to the linking moiety again through an amide linkage. The chelating moiety is used to attach a radionuclide to the vitamin that can be used for therapeutic or diagnostic purposes.

Collins, et al. in WO 01/28595 (PCT/US00/10098) disclose a series of novel cobalamin conjugates that are linked via a protein linker to a detectable group, which are useful in the imaging of tumors.

Collins, et al. in WO 01/28592 (PCT/US00/10097) disclose a series of novel cobalamin conjugates that are linked directly or by a linker to a residue of a chemotherapeutic agents, which are useful in the treatment of abnormal cellular proliferation.

Collins, et al. in WO 00/62808 (PCT/US00/10100) disclose a series of novel cobalamin conjugates that are linked directly or by a linker to a residue of a molecule comprising B-10 or Gd-157, which are useful in the treatment of abnormal cellular proliferation.

PCT Publication WO 98/08859 listing Grissom et al as inventors discloses conjugates containing a bioactive agent and an organocobalt complex in which the bioactive agent is covalently bound directly or indirectly, via a spacer, to the cobalt atom. The bioactive agent is released from the bioconjugate by the cleavage of the weak covalent bond between the bioactive agent and the cobalt atom as a result of normal displacement by cellular nucleophiles or enzymatic action, or by application of an external signal (e.g., light, photoexcitation, ultrasound, or the presence of a magnetic field).

U.S. Pat. No. 5,428,023 to Russell-Jones et al. discloses a vitamin B₁₂ conjugate for delivering oral hormone formulations. Russell-Jones teaches that the vitamin B₁₂ conjugate must be capable of binding in vivo to intrinsic factor, enabling uptake and transport of the complex from the intestinal lumen of a vertebrate host to the systemic circulation of the host. The hormones are attached to the vitamin B₁₂ through a hydrolyzed propionamide linkage on the vitamin. The patent states that the method is useful for orally administering hormones, bioactive peptides, therapeutic agents, antigens, and haptens, and lists as therapeutic agents neomycin, salbutamol cloridine, pyrimethamine, penicillin G, methicillin, carbenicillin, pethidine, xylazine, ketamine hydrochloride, mephanesin and iron dextran. U.S. Pat. No. 5,548,064 to Russell-Jones et al. discloses a vitamin B₁₂ conjugate for delivering erythropoietin and granulocyte-colony stimulating factor, using the same approach as the '023 patent.

PCT Publication WO 94/27641 to Russell-Jones et al discloses vitamin B₁₂ linked through a polymer to various active agents wherein the conjugate is capable of binding to intrinsic factor for systemic delivery. In particular, the document discloses the attachment of various polymeric linkers to the propionamide positions of the vitamin B₁₂ molecule, and the attachment of various bioactive agents to the polymeric linker. Exemplary bioactive agents include hormones, bioactive peptides and polypeptides, antitumor agents, antibiotics, antipyretics, analgesics, antiinflammatories, and haemostatic agents. Exemplary polymers include carbohydrates and branched chain amino acid polymers. The linkers used in WO 94/27641 are polymeric (each having a molecular weight of about 5000 or greater). Importantly, the linkers are described as exhibiting a mixture of molecular weights, due to the polymerization process by which they are made. See in particular, page 11, lines 25-26 wherein it is stated that the polymer used in that invention is of uncertain size and/or structure.

PCT Publication WO 99/65930 to Russell-Jones et al. discloses the attachment of various agents to the 5′-OH position on the vitamin B₁₂ ribose ring. The publication indicates that the system can be used to attach polymers, nanoparticles, therapeutic agents, proteins, and peptides to the vitamin.

U.S. Pat. No. 5,574,018 to Habberfield et al. discloses conjugates of vitamin B₁₂ in which a therapeutically useful protein is attached to the primary hydroxyl site of the ribose moiety. The patent lists erythropoietin, granulocyte-colony stimulating factor and human intrinsic factor as therapeutically useful proteins, and indicates that the conjugates are particularly well adapted for oral administration.

U.S. Pat. No. 5,840,880 to Morgan, Jr. et al. discloses vitamin B₁₂ conjugates to which are linked receptor modulating agents, which affect receptor trafficking pathways that govern the cellular uptake and metabolism of vitamin B₁₂. The receptor modulating agents are linked to the vitamin at the b-, d-, or e-position.

Other patent filings which describe uses of Vitamin B₁₂ include U.S. Pat. No. 3,936,440 to Nath (Method of Labeling Complex Metal Chelates with Radioactive Metal Isotopes); U.S. Pat. No. 4,209,614 to Bernstein et al., (Vitamin B₁₂ Derivatives Suitable for Radiolabeling); U.S. Pat. No. 4,279,859 (Simultaneous Radioassay of Folate and Vitamin B₁₂); U.S. Pat. No. 4,283,342 to Yollees (Anticancer Agents and Methods of Manufacture); U.S. Pat. No. 4,301,140 to Frank et al (Radiopharmaceutical Method for Monitoring Kidneys); U.S. Pat. No. 4,465,775 to Houts (Vitamin B₁₂ and labeled Derivatives for Such Assay); U.S. Pat. No. 5,308,606 to Wilson et al (Method of Treating and/or Diagnosing Soft Tissue Tumors); U.S. Pat. No. 5,405,839 (Vitamin B₁₂ Derivative, Preparation Process Thereof, and Use Thereof); U.S. Pat. No. 5,449,720 to Russell-Jones et al., (Amplification of the Vitamin B₁₂ Uptake System Using Polymers); U.S. Pat. No. 5,589,463 to Russell Jones (Oral Delivery of Biologically Active Substances Bound to Vitamin B₁₂); U.S. Pat. No. 5,608,060 to Axworthy et al (Biotinidase-Resistant Biotin-DOTA Conjugates); U.S. Pat. No. 5,807,832 to Russell-Jones et al (Oral Delivery of Biologically Active Substances Bound to Vitamin B₁₂); U.S. Pat. No. 5,869,465 to Morgan et al (Method of Receptor Modulation and Uses Therefor); U.S. Pat. No. 5,869,466 to Russell-Jones et al (vitamin B₁₂ Mediated Oral Delivery systems for GCSF). See also Ruma Banerjee, Chemistry and Biochemistry of B₁₂ John Wiley & Sons, Inc. (11999), and in particular Part II, Section 15 of that book, entitled “Diagnostics and Therapeutic Analogues of Cobalamin,” by H. P. C. Hogenkamp, Douglas A. Collins, Charles B. Grissom, and Frederick G. West.

Vitamins have also been used to target the deliver of specific compounds to specific cells or tissues. U.S. Pat. No. 6,093,701 to Wolff et al. discloses methods for covalently attaching compounds to genes. The '701 patent generally discloses that vitamins can be used to target the invention, but vitamin B₁₂ is not specifically disclosed.

U.S. Pat. No. 6,056,973 to Allen et al. discloses a kit for preparing liposomes containing nucleic acids and having vitamin B₁₂ as a targeting ligand attached to the liposomes.

U.S. Pat. No. 6,395,492 to Isis Pharmaceuticals, Inc. describes compounds that comprise a plurality of linked nucleosides wherein at least one of the nucleosides is functionalized at the 2′-position with a substituent such as, for example, a steroid molecule, a reporter moleclue, a non-aromatice lipophilic molecule, a reporter enzyme, a peptide, a protein, a water soluble vitamin, a lipid soluble vitamin, an RNA cleaving complex, a metal chelator, a porphyrin, an alkylator, a hybrid photonuclease/intercalator, a pyrene, or an aryl azide photocrosslinking agent. Alternatively, a method for enhancing the binding affinity and/or stability of an antisense oligonucleotide comprising functionalizing the oligonucleotide generally with a steroid, reporter molecule, a non-aromatice lipophilic molecule, a reporter enzyme, a peptide or water soluble or lipid soluble vitamin.

EP 0 804 456 B1 describes a peptide nucleic acid that contains a plurality of amino groups which each have a tethered nucleobase, and a conjugate bound to the PNA that can be a terpene, an aromatic lipophilic molecule, a phospholipid, a cell receptor binding molecule, a crosslinking agent, a water soluble vitamin, a lipid soluble vitamin, an RNA/DNA cleaving complex, a porphyrin, or a polymeric compound. Because of the adverse side-effects and poor cellular targeting of virus based gene delivery systems, there exists a need in the art for non-viral based gene delivery systems with increased safety and targeting efficiency.

Therefore, it is an object of the present invention to provide a non-viral based gene delivery system.

It is yet another object of the present invention to provide a means and composition to deliver nucleic acids, analogs and derivatives (including antisense or stabilized antisense sequences) to desired locations to affect cell processes, including but not limited to gene transcription or translation.

It is another goal to increase the efficacy of gene therapy by increasing the efficiency of delivery of materials that affect gene translation or transcription.

It is another object of the present invention to provide an improved method and composition for delivering nucleic acids and nucleic acid substitutes to specific cells and tissues.

It is still another object of the present invention to provide a method and compositin for treating diseases by administering a nucleic acid sequence targeted to specific nucleic acid sequences.

### SUMMARY OF THE INVENTION

It has been discovered that an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA can be effectively delivered to cells by conjugation to a ligand for the transcobalamin receptor or intrinsic factor receptor. In one embodiment, a nucleic acid can be delivered that is antisense to a target sequence. Nucleic acids can be delivered that can be incorporated into the host genome. Alternatively, nucleic acids, analogs or mimics thereof with any alternative known function can be used in this invention. Nucleic acids can be stabilized in any known manner to decrease digestion by nucleases and thus increasing bioavailability or half life, or to otherwise enhance the desirable properties of the sequence. Nonlimiting examples of stabilized antisense sequences include PNA, MNA, LNA, PCO, ENA (also referred to as stabilized mimics) and any other published, known or developed nucleic acid mimic. Any of these can be conjugated to the ligand by known means to create new compositions of matter. For the purposes of this specification, the term “nucleic acid” nucleic acid” is intended to include all of these embodiments, unless otherwise indicated.

The method of the present invention can be used to systemically deliver nucleic acids to treat diseases by inhibiting the expression of specific genes or by introducing nucleic acids that encode for a specific protein or fragment of a protein. Complexation to a ligand for the transcobalamin receptor or intrinsic factor receptor with the nucleic acids must be sufficiently stable in vivo to prevent significant uncoupling of the nucleic acids extracellularly prior to internalization by the target cell. However, the complex is cleavable under appropriate conditions within or at the cell so that the nucleic acids are released in functional, hybridizable form. For example, the complex can be labile in the acidic and enzyme rich environment of lysosomes. A non-covalent bond based on electrostatic attraction between the binding agent and the oligonucleotide provides extracellular stability and is releasable under intracellular conditions. Covalent ligand binding increases the stability of the gene-mediating complex.

Nonlimiting examples of diseases that can be treated using antisense oligonucleotides include cancer and viral diseases such as infections caused by HIV, hepatitis (including hepatitis B, hepatitis C and hepatitis D), herpes (including herpes simplex virus and type 6 herpes), TB, Epstein-Barr Virus, malaria, influenza virus (A, B and C), parainfluenza virus (serotypes 1-4), mumps virus, adenoviruses, reoviruses, respiratory syncytial virus, rhinoviruses, polioviruses, coxsackie-viruses, echoviruses, enteroviruses, gastroenteritis viruses, rubeola virues, rubella virus, molluseum contagiosum virus, human parvovirus B19, cytomegalovirus, human papillomavirus, varicella zoster (including chickenpox and herpes zoster), arenaviruses, filoviruses, etc. In one preferred embodiment, antisense oligonucleotides that bind to viral mRNAs are conjugated to vitamin B₁₂ or a ligand of a transcobalamin receptor or intrinsic factor receptor.

In another embodiment, antisense oligonucleotides or stabilized mimics that bind to viral DNA or RNA, and in particular mRNA, are conjugated to vitamin B₁₂ or a ligand of a transcobalamin receptor or intrinsic factor receptor.

In yet another embodiment, antisense oligonucleotides or stabilized mimics that bind to the DNA or RNA, and in particular mRNA, of oncogenes are conjugated to vitamin B₁₂ or a ligand of a transcobalamin receptor or intrinsic factor receptor.

In one embodiment the antisense oligonucleotide is a peptide nucleic acid (PNA) or a morpholino nucleic acid (MNA).

In another embodiment, an antisense oligonucleotide or stabilized mimic is attached to vitamin B₁₂ or a ligand of a transcobalamin receptor or intrinsic factor receptor.

In other embodiments, the oligonucleotide can be a stabilized oligonucleotide used in antisense therapy, or a stabilized mimic molecule that has catalytic activity, such as a ribozyme. Ribozymes are advantageous because they specifically cleave and, thus, destroy the targeted RNA sequence. Ribozymes are described in U.S. Pat. No. 4,987,071.

In yet other embodiments, an optionally stabilized nucleic acid or nucleic acid analogue which encodes for a peptide, protein or other biological modifier is delivered by conjugation to one or more cobalamin moieties as described below to accomplish gene therapy.

In another embodiment, a “nonsense” sequence, sometimes also referred to as an aptamer is delivered. The aptamer, for example be a ligand for a naturally occurring compound such as a peptide, protein, glycoprotein, saccaride, carbohydrate, hormone, enzyme, receptor, transcriptional factor, lipid, or other biological mediator.

In yet a further embodiment an antisense mimic, including but not limited to PNA, MNA, LNA, PCO or ENA is delivered.

In another embodiment the nucleic acid conjugate or antisense oligonucleotide or stabilized mimic of the present invention can contain a carrier compound represented by formula I: 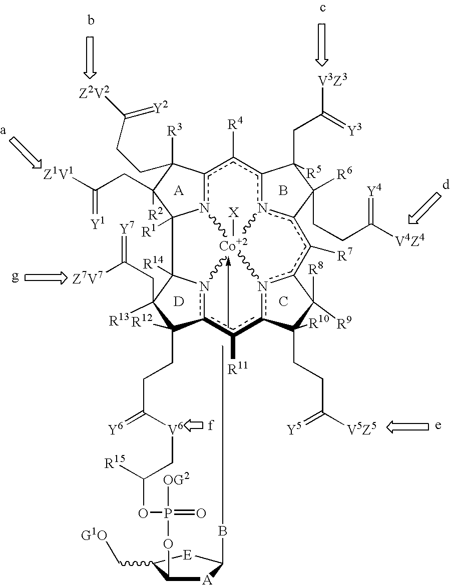
or its enantiomer, diastereomer, salt or prodrug thereof, wherein:
(i) X is hydrogen, cyano, amino, amido, hydroxyl, adenosyl L-T, alkyl, alkenyl, alkynyl, cylcoalkyl, aryl, aralkyl, heterocycle, heteroaryl or alkylheteroaryl;
(ii) B is a divalent heterocycle wherein the radical positions can be within the ring or a substituent to the ring such that at least one radical is on a heteroatom to form a dative bond with cobalt, optionally substituted by L-T;
(iii) A is O, S, NJ¹, CR¹⁰⁰R¹⁰¹ or C(R¹⁰⁰)V⁸Z⁸;
(iv) E is O or S;
(v) G¹ and G² are independently hydrogen, alkyl, acyl, silyl, phosphate, or L-T;
(vi) Y¹, Y², Y³, Y⁴, Y⁵, Y⁶ and Y⁷ independently are O, S or NJ²;
(vii) V¹, V², V³, V⁴, V⁵, V⁶, V⁷ and V⁸ independently are O, S or NJ³; CR¹⁰²R¹⁰³, or a direct bond;
(viii) Z¹, Z², Z³, Z⁴, Z⁵, Z⁷ and Z⁸ independently are R¹⁰⁴ or L-T;
(ix) each L is independently a direct bond or the residue of a multivalent moiety that does not significantly impair the ability of the compound to bind transcobalamin II;
(x) each T is independently an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA;
(xi) at least one of Z¹, Z², Z³, Z⁴, Z⁵, Z⁷, Z⁸, A, B, G¹, and G² comprises an a nucleic acid sequence useful in antisense technology, a peptide nucleic acid or morpholino nucleic acid;
(xii) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xiii) R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(xiv) R¹³ and R¹⁴ optionally can come together to form a pi bond; and
(xv) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

In another embodiment, a nucleic acid sequence, stabilized mimic complementary to a viral gene is conjugated to vitamin B₁₂, a ligand of a transcobalamin receptor or intrinsic factor receptor or carrier of the present invention is provided.

In still another embodiment, a antisense oligonucleotide, in particular a protein nucleic acid (PNA) or a morpholino nucleic acid (MNA), specific for viral mRNA or viral DNA is conjugated to vitamin B₁₂, a ligand of a transcobalamin receptor or intrinsic factor receptor or carrier of the present invention. In a preferred embodiment, the antisense oligonucleotide conjugated to vitamin B₁₂, a ligand of a transcobalamin receptor or intrinsic factor receptor or carrier of the present invention binds to double stranded DNA.

In yet another embodiment, a PNA-DNA, PNA-RNA, MNA-DNA or MNA-RNA chimera conjugated to vitamin B₁₂, a ligand of a transcobalamin receptor or intrinsic factor receptor or carrier of the present invention inhibits the translation of viral mRNA.

In another embodiment, a PNA-DNA, PNA-RNA, MNA-DNA or MNA-RNA chimera conjugated to vitamin B₁₂, a ligand of a transcobalamin receptor or intrinsic factor receptor or carrier of the present invention inhibits the transcription of viral DNA. It is understood that Stabilized mimic chimeras can comprise any combination of PNA-DNA, PNA-RNA, MNA-DNA or MNA-RNA or multiples thereof.

In still another embodiment, a stabilized mimic conjugated to vitamin B₁₂, a ligand of a transcobalamin receptor or intrinsic factor receptor or carrier of the present invention inhibits the translation of an oncogenic mRNA.

In another embodiment, a stabilized mimic conjugated to vitamin B₁₂, a ligand of a transcobalamin receptor or intrinsic factor receptor or carrier of the present invention inhibits the transcription of an oncogene.

In a preferred embodiment, the stabilized mimic conjugated to vitamin B₁₂, a ligand of a transcobalamin receptor or intrinsic factor receptor or carrier of the present invention contains a nuclear targeting peptide.

In another embodiment, a PNA, MNA, PNA chimera or MNA chimera conjugated to vitamin B₁₂, a ligand of a transcobalamin receptor or intrinsic factor receptor or carrier of the present invention binds to a transcription factor.

In another embodiment, a method for targeting antisense oligonucleotides to specific cells or tissues is provided comprising conjugating an antisense oligonucleotide to vitamin B₁₂, a ligand of a transcobalamin receptor or intrinsic factor receptor or carrier of the present invention and administering the conjugate to a host, preferably a mammal, more preferably a human in need thereof.

In another embodiment, a method for targeting stabilized mimics to specific cells or tissues is provided comprising conjugating a stabilized mimic, respectively, to vitamin B₁₂, a ligand of a transcobalamin receptor or intrinsic factor receptor or carrier of the present invention, and administering the conjugate to a host, preferably a mammal, more preferably a human in need thereof.

In another embodiment, a method for increasing the uptake of stabilized mimics by cells is provided comprising conjugating a stabilized mimic, respectively, to vitamin B₁₂, a ligand of a transcobalamin receptor or intrinsic factor receptor or carrier of the present invention and administering the conjugate to a host, preferably a mammal, more preferably a human in need thereof.

### BRIEF DESCRIPTION OF THE FIGURES

FIG. 1 is a non-limiting illustration of the usage of antisense nucleotide sequences, including stabilized mimics for the treatment of disease. Antisense therapy effects abnormal cellular proliferation and viral replication prior to translation, unlike most traditional medicines which only effect disease progression post-translation.

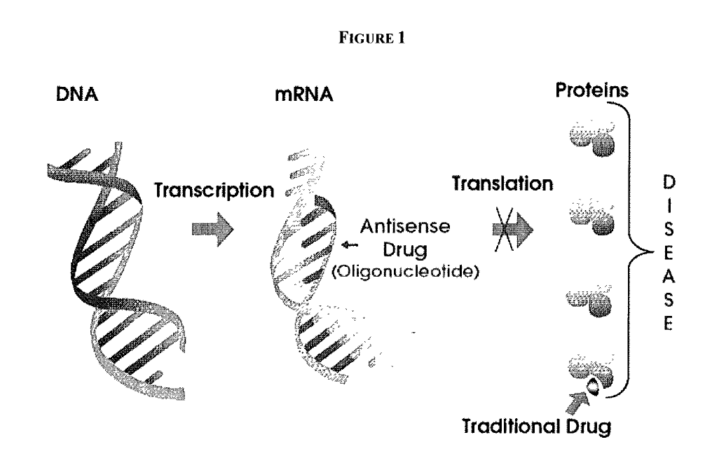

FIG. 2 are some non-limiting examples of peptide nucleic acids and morpholino nucleic acids with respect to RNA (wherein R is OH) or DNA (wherein R is H). B can be any purine or pyrimidine base.

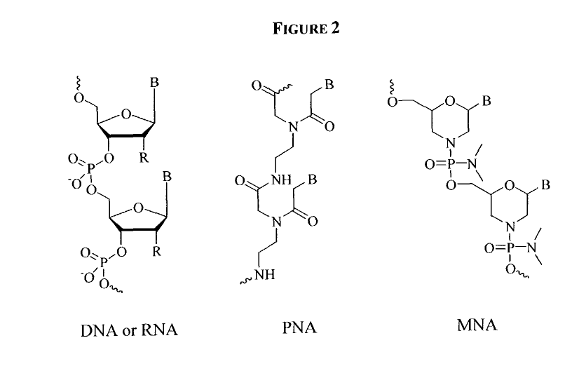

### DETAILED DESCRIPTION OF THE INVENTION

The present invention can be utilized to deliver an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA to various kinds of organisms or cells, preferably mammals, more preferably humans, in need thereof by suitably selecting an appropriate sequence, or any combination thereof and conjugating the sequence to a ligand for the transcobalamin II receptor or a ligand for the intrinsic factor-cobalamin receptor.

In preferred embodiments of the present invention, the ligand is vitamin B₁₂ or a derivative thereof selected from 5′-adenosylcobalamin, methylcobalamin, hydroxycobalamin, cyanocobalamin or any derivative thereof, as described herein. In another preferred embodiment, the nucleic acid, analogue, aptamer, antisense or antisense mimic is conjugated to the cobalamin moiety through at least one of Z¹, Z², Z³, Z⁴ or Z⁵. In a more preferred embodiment, the T moiety is conjugated though the “b” carboxamide of vitamin B₁₂ (“Z²”). In another embodiment, the moiety is conjugated to more than one transcobalamin receptor ligand. In other embodiments of the present invention, nucleic acids can be conjugated to a complex of transcobalamin II-vitamin B₁₂ or intrinsic factor-vitamin B₁₂. The present invention can be used to treat diseases by delivering to cells expressing transcobalamin II receptors or intrinsic factor receptors nucleic acid, peptide nucleic acid, mopholinonucleic acid, locked nucleic acid, pseudocyclic oligonucleobase, or 2′-O,4′-C-ethylene bridged nucleic acid sequences, or any combination thereof, that regulate the expression of specific genes or encode for specific proteins or fragments of proteins.

The nucleic acid can be RNA, DNA, stabilized mimics of short (less than 15, 20, or 25 nucleotides) intermediate (between 20 or 25 and 100 nucleotides) or long chain length (greater than 100 nucleotides), as desired, doubly or singly stranded. In a preferred embodiment the nucleic acid sequence can be an antisense RNA, an antisense oligonucleotide, or antisense stabilized mimic of 20 or 25 nucleotides or less.

The method of targeting nucleic acid sequences of the present invention can be used to systemically deliver nucleic acid sequences to treat diseases, for example, viral infections, cancer and other abnormal cellular proliferative diseases. In one preferred embodiment, an antisense nucleic acid sequence that binds to oncogenic or viral mRNA can be conjugated to vitamin B₁₂, a ligand of a transcobalamin receptor or intrinsic factor receptor or carrier of the present invention.

In a preferred embodiment, the vitamin B₁₂, a ligand of a transcobalamin receptor or intrinsic factor receptor or carrier of the present invention is conjugated to a nucleic acid, peptide nucleic acid, mopholinonucleic acid, locked nucleic acid, pseudocyclic oligonucleobase, or 2′-O,4′-C-ethylene bridged nucleic acid sequence, in addition to a nuclear localization sequence, such as TAT, a nuclear localization signal peptide of the sequence Tyr-Gly-Arg-Lys-Arg-Arg-Gln-Arg-Arg-Arg (Sequence No. 1).

In another embodiment of the invention, the vitamin B₁₂, a ligand of a transcobalamin receptor or intrinsic factor receptor or carrier of the present invention is conjugated to a nucleic acid, peptide nucleic acid, mopholinonucleic acid, locked nucleic acid, pseudocyclic oligonucleobase, or 2′-O,4′-C-ethylene bridged nucleic acid, in addition to a nuclear localization sequence can be optionally bound to transcobalamin protein (including, but not limited to intrinsic factor, transcobalamin I, transcobalamin II and transcobalamin III).

In anther preferred embodiment, an antisense nucleic acid sequence that binds to oncogenic or viral DNA can be conjugated to vitamin B₁₂, a ligand of a transcobalamin receptor or intrinsic factor receptor or carrier of the present invention.

In another embodiment, antisense oligonucleotides that bind to the mRNA of oncogenes can be conjugated to vitamin B₁₂, a ligand of a transcobalamin receptor or intrinsic factor receptor or carrier of the present invention.

In still another embodiment, antisense oligonucleotides that bind to the DNA of oncogenes can be conjugated to vitamin B₁₂, a ligand of a transcobalamin receptor or intrinsic factor receptor or carrier of the present invention.

In one embodiment, the antisense oligonucleotide is a stabilized mimic.

In yet another embodiment, a PNA-DNA, PNA-RNA, MNA-DNA or MNA-RNA chimera inhibits the translation of viral mRNA.

In another embodiment, a PNA-DNA, PNA-RNA, MNA-DNA or MNA-RNA chimera inhibits the transcription of viral DNA.

In still another embodiment, a stabilized mimic inhibits the translation of an oncogenic mRNA.

In another embodiment, a stabilized mimic inhibits the transcription of an oncogene.

In another embodiment, a PNA, MNA, PNA chimera or MNA chimera binds to a transcription factor.

In one embodiment, the stabilized mimic is bound to a carrier compound of the present invention. In a particular embodiment, the carrier compound is vitamin B₁₂.

In another embodiment, a method for targeting antisense oligonucleotides to specific cells or tissues is provided comprising conjugating an antisense oligonucleotide to vitamin B₁₂, a ligand of a transcobalamin receptor or intrinsic factor receptor, optionally conjugated to a nuclear localization sequence, optionally conjugated to a transcobalamin protein, and administering the nucleic acid conjugate to a host, preferably a mammal, more preferably a human in need thereof.

In another embodiment, a method for targeting stabilized mimics to specific cells or tissues is provided comprising conjugating a stabilized mimic, respectively, to vitamin B₁₂, a ligand of a transcobalamin receptor or intrinsic factor receptor, optionally conjugated to a nuclear localization sequence, optionally conjugated to a transcobalamin protein, and administering the conjugate to a host, preferably a mammal, more preferably a human in need thereof.

In another embodiment, a method for increasing the up take of stabilized mimics by cells is provided comprising conjugating a stabilized mimic, respectively, to vitamin B₁₂, a ligand of a transcobalamin receptor or intrinsic factor receptor, optionally conjugated to a nuclear localization sequence, optionally conjugated to a transcobalamin protein, and administering the conjugate to a host, preferably a mammal, more preferably a human in need thereof.

In another embodiment, a method for the delivery of a nucleic acid, and in particular a stabilized mimic, sequence to a cell or tissue is provided wherein, a nucleic acid, stabilized mimic sequence that binds to viral or oncogenic mRNA is conjugated to vitamin B₁₂, a ligand of a transcobalamin receptor or intrinsic factor receptor, optionally conjugated to a nuclear localization sequence, optionally conjugated to a transcobalamin protein, is administered to a host in need thereof.

In another embodiment, a method for delivery a nucleic acid, and in particular a stabilized mimic, sequence to a cell or tissue is provided wherein, an antisense nucleic acid sequence specific for an oncogene is conjugated to vitamin B₁₂, a ligand of a transcobalamin receptor or intrinsic factor receptor, optionally conjugated to a nuclear localization sequence, optionally conjugated to a transcobalamin protein, is administered to a host in need thereof.

In another embodiment of the present invention, a peptide nucleic acid conjugated to vitamin B₁₂, a ligand of a transcobalamin receptor or intrinsic factor receptor, optionally conjugated to a nuclear localization sequence, optionally conjugated to a transcobalamin protein, is disclosed wherein the peptide nucleic acid prevents the translation of viral mRNA is provided.

In another embodiment of the present invention, a morpholino nucleic acid conjugated to vitamin B₁₂, a ligand of a transcobalamin receptor or intrinsic factor receptor, optionally conjugated to a nuclear localization sequence, optionally conjugated to a transcobalamin protein, is disclosed wherein the morpholino nucleic acid prevents the translation of viral mRNA is provided.

In another embodiment, the present invention provides a soluble molecular complex comprising a single-stranded antisense oligonucleotide that hybridizes to an RNA in a target cell, said antisense oligonucleotide complexed with a carrier comprised of a ligand for the transcobalamin II receptor or intrinsic factor receptor, optionally conjugated to a nuclear localization sequence, optionally conjugated to a transcobalamin protein, and a polycation.

In one embodiment, the agent and carrier are administered in a slow release formulation such as an implant, bolus, microparticle, microsphere, nanoparticle or nanosphere. Nonlimiting examples of sustained release compositions include semi-permeable polymer matrices in the form of shaped articles, e.g. films, microcapsules or microspheres. Sustained release matrices include, for example, polylactides (U.S. Pat. No. 3,773,919), copolymers of L-glutamic acid and γ-ethyl-L-glutamate (Sidman et al., Biopolymers 22:547-556, 1983) or poly-D-(−)-3-hydroxybutyric acid (EP 133,988). Sustained release compositions also include one or more liposomally entrapped compounds of formula I. Such compositions are prepared by methods known per se, e.g. as taught by Epstein et al. Proc. Natl. Acad. Sci. USA 82:3688-3692, 1985. Ordinarily, the liposomes are of the small (200-800 Å) unilamellar type in which the lipid content is greater than about 30 mol % cholesterol, the selected proportion being adjusted for the optimal therapy.

A number of sustained-release implants are known in the art. Most implants are “matrix” type and comprise an active compound dispersed in a matrix of a carrier material. The carrier material may be either porous or non-porous, solid or semi-solid and permeable or impermeable to the active compound. Matrix devices are typically biodegradable, i.e. they slowly erode after administration. Alternatively, matrix devices may be nondegradable and rely on diffusion of the active compound through the walls or pores of the matrix. Matrix devices are preferred for the applications contemplated herein.

Thus, in one embodiment the invention provides a surgical implant for localized delivery of an active agent comprising the cobalamin conjugate of the present invention and a biodegradable binder. The implant preferably is capable of releasing and delivering the cobalamin conjugate to substantially all of an area of clear margin that results from a surgical resection and is also preferably capable of releasing the cobalamin conjugate at a substantially constant rate. In another embodiment the invention provides a method of delivering an imaging agent to an area of clear margin following a surgical resection comprising (i) providing an implant comprising a TC- or IF-binding agent linked to an imaging agent and a biodegradable binder; and (ii) placing the implant into a void created by surgical resection.

The surgical implant can exhibit a variety of forms. In one embodiment the implant is a bolus, comprising a viscous and deformable material capable of being shaped and sized before or during implantation to complement a void created by a surgical resection and sufficiently deformable upon implantation to contact substantially all of an area of clear margin. The surgical implant can also comprising a plurality of capsules that can be poured into the void created by a surgical resection. These capsules will contain the cobalamin conjugate and a suitable binder. Because they are flowable, they can be poured into the void created by a surgical lumpectomy and thereby contact substantially all of the areas of clear margin.

Many suitable compositions for the implant are known and can be used in practicing the invention. Such compositions are described in, for example, Chasin et al. Biodegradable Polymers as Drug Delivery Systems, Marcel Dekker Inc., NY, ISBN 0-8247-8344-1. Preferable compositions are pharmaceutically acceptable, biodegradable and meet the particular release profile characteristics that are required to achieve the administration regime involved.

The implant typically comprises a base composition that acts as a matrix to contain and hold the contents of the implant together. The base composition can, in turn, comprise one or more constituents. Examples of base compositions include polymers and copolymers of anhydrides or thioester, lactic acid, glycolic acid, dioxonane, trimethylene carbonate, β-caprolactone, phosphazene and glyceryl monostearate.

In one embodiment the base composition for the matrix comprises a polyanhydride, which can be synthesized via the dehydration of diacid molecules by melt condensation. Degradation times can be adjusted from days to years according to the hydrophobicity of the monomer selected. The materials degrade primarily by surface erosion and possess excellent in vivo compatibility. In one embodiment the polyanhydride is formed from sebasic acid and hexadecandioic acid (poly(SA-HDA anhydride). Wafer-like implants using this base composition have been approved for use in brain cancer, as Giadel®, by Guilford Pharmaceuticals.

The implant optionally can comprise erosion and biodegradation enhancers that facilitate the erosion of the matrix, the dissolution of the core composition or the uptake of the core composition via metabolic processes. Particularly suitable erosion and biodegradation enhancers are biodegradable in biological fluids and biocompatible. Hydrophilic constituents are typical, because they are capable of enhancing the erosion of the implant in the presence of biological fluids. For example, K. Juni et al., Chem. Pharm. Bull., 33, 1609 (1985) disclose that the release rate of bleomycin from polylactic acid microspheres is greatly enhanced by incorporating fatty acid esters into the microspheres. Other exemplary hydrophilic constituents are described, for example, in Wade & Weller, Handbook of pharmaceutical Excipients (London: Pharmaceutical Press; Washington D.C.: American Pharmaceutical Ass'n 1995) and include the polyethylene glycols (“PEGs”), propylene glycol (“PG”), glycerin and sorbitol.

Surfactants further enhance the erosion of the matrix and the release of the drug. Surfactants are generally capable of increasing the wettability and the solubility of the base composition in biological fluids and thereby causing the disintegration and erosion of the implant. Surfactants can also help to break down the core composition matrix when, for example, the method of forming the dosage form has reduced the solubility of any of the constituents. Surfactants can also improve the uptake of the dosage forms into the bloodstream. Suitable surfactants include, for example, glyceryl based surfactants such as glyceryl monooleate and glyceryl monolaurate, poloxamers such as Pluronic F127 and polysorbates such as polyoxyethylene sorbitan monooleate (“Tween 80”).

The implant could also include components that retard the rate at which the implant erodes or biodegrades (erosion and/or biodegradation retardants). Hydrophobic constituents are a particularly suitable class of components for retarding the rate at which the outer layer biodegrades. Suitable hydrophobic constituents are described, for example, in the Handbook of Pharmaceutical Excipients, the disclosure from which being hereby incorporated by reference. Exemplary hydrophobic constituents include peanut oil, olive oil and castor oil.

Any proportions or types of constituents can be chosen that effectively achieve a desired release profile and thereby carry out the prescribed administration regime. The most desirable base compositions generally release the drug substantially continuously and biodegrade completely shortly after substantially all of the drug has been effectively released. The amount of drug included in the dosage forms is determined by the total amount of the drug to be administered and the rate at which the drug is to be delivered. The total amount of the drug to be delivered is determined according to clinical requirements and in keeping with the considerations that typically inform drug dosage determinations in other contexts. The surgical implant also can contain one or more other drugs having therapeutic efficacy in the intended applications, such as an antibiotic, an analgesic or an anesthetic.

I. Carrier Compound

In one embodiment, the carrier is any ligand that will bind effectively to a vitamin B₁₂ transport protein (i.e. transcobalamin I, II or III or intrinsic factor) and which when appropriately linked to a nucleic acid sequence useful in antisense technology, peptide nucleic acid, mopholinonucleic acid, locked nucleic acid, pseudocyclic oligonucleobase, or 2′-O,4′-C-ethylene bridged nucleic acid, and optionally bound to a transport protein, will fit into a transcobalamin receptor. Methods for the assessment of whether a moiety binds the TC receptor are known and include those described by Pathare et al., Bioconjugate Chem. 1996, 7, 217-232; and Pathare, et al., Bioconjugate Chem. 8, 161-172. An assay that assesses binding to a mixture of transcobalamin I and II receptors is found in Chaiken, et al, Anal. Biochem. 1992, 201, 197. An unsaturated Vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204. See also Fairbanks, V. F. Mayo Clinical Proc. 83, Vol 58, 203-204. The ligand preferably displays a binding affinity to transcobalamin of at least 50% of the binding affinity displayed by vitamin B₁₂, more preferably at least 75% and even more preferably at least 90%.

In another embodiment, the conjugate construct of the present invention can include a carrier molecule selected from the group consisting of, but not limited to cyanocobalamin, adenosylcobalamin or hydroxycobalamin. Adenosylcobalamin is a vitamin B₁₂ coenzyme in which the sixth coordination position of the cobalt atom is linked covalently to the 5′-carbon of 5′-deoxyadenosine. Hydroxycobalamin is a vitamin B₁₂ coenzyme in which the sixth coordination position of the cobalt atom is linked covalently to a hydroxyl. In another preferred embodiment, the nucleic acid, analogue, aptamer, antisense or antisense mimic is conjugated to the cobalamin moiety through at least one of Z¹, Z², Z³, Z⁴ or Z⁵. In a more preferred embodiment, the T moiety is conjugated though the “b” carboxamide of vitamin B₁₂ (“Z²”). In another embodiment, the moiety is conjugated to more than one transcobalamin receptor ligand.

In an embodiment the conjugate of the present invention can contain a carrier molecule based molecule represented by formula I: 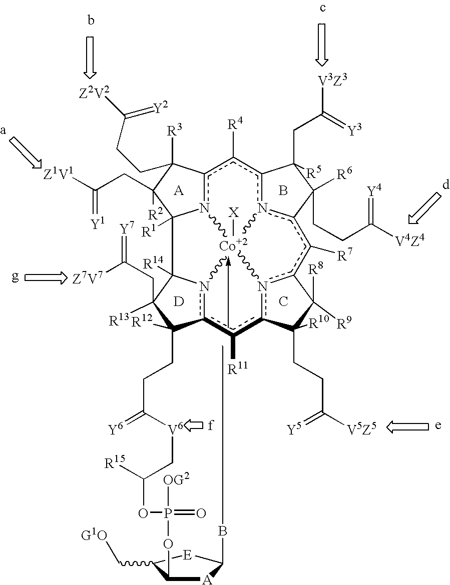
or its enantiomer, diastereomer, salt or prodrug thereof, wherein:
(i) X is hydrogen, cyano, amino, amido, hydroxyl, adenosyl L-T, alkyl, alkenyl, alkynyl, cylcoalkyl, aryl, aralkyl, heterocycle, or heteroaryl, or alkylheteroaryl;
(ii) B is a divalent heterocycle wherein the radical positions can be within the ring or a substituent to the ring such that at least one radical is on a heteroatom to form a dative bond with cobalt, optionally substituted by L-T;
(iii) A is O, S, NJ¹, CR¹⁰⁰R¹⁰¹ or C(R¹⁰⁰)V⁸Z⁸;
(iv) E is O or S;
(v) G¹ and G² are independently hydrogen, alkyl, acyl, silyl, phosphate or L-T;
(vi) Y¹, Y², Y³, Y⁴, Y⁵, Y⁶ and Y⁷ independently are O, S or NJ²;
(vii) V¹, V², V³, V⁴, V⁵, V⁶, V⁷ and V⁸ independently are O, S or NJ³; CR¹⁰²R¹⁰³ or a direct bond;
(viii) Z¹, Z², Z³, Z⁴, Z⁵, Z⁷ and Z⁸ independently are R¹⁰⁴ or L-T;
(ix) each L is independently a direct bond or the residue of a multivalent moiety that does not significantly impair the ability of the compound to bind transcobalamin II;
(x) each T is independently an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA;
(xi) at least one of Z¹, Z², Z³, Z⁴, Z⁵, Z⁷, Z⁸, A, B, G¹, and G² contains a T
(xii) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xiii) R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(xiv) R¹³ and R¹⁴ optionally can come together to form a pi bond; and
(xv) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

In another embodiment, the conjugate of the present invention can contain a carrier molecule based molecule represented by formula I, and further comprises one or more T that is independently an imaging agent and/or another therapeutic agent.

In a particular embodiment, a protein nucleic acid (PNA) or morpholino nucleic acid (MNA) specific for viral mRNA or viral DNA is conjugated to vitamin B₁₂, a ligand of a transcobalamin receptor or intrinsic factor receptor, optionally conjugated to a nuclear localization sequence, optionally conjugated to a transcobalamin protein.

In a particular embodiment, a protein nucleic acid (PNA) or morpholino nucleic acid (MNA) specific for bacterial mRNA or viral DNA is conjugated to vitamin B₁₂, a ligand of a transcobalamin receptor or intrinsic factor receptor, optionally conjugated to a nuclear localization sequence, optionally conjugated to a transcobalamin protein.

In another particular embodiment, a protein nucleic acid (PNA) or morpholino nucleic acid (MNA) specific for oncogenic mRNA or oncogenic DNA is conjugated to vitamin B₁₂, a ligand of a transcobalamin receptor or intrinsic factor receptor, optionally conjugated to a nuclear localization sequence, optionally conjugated to a transcobalamin protein.

In a particular embodiment, a protein nucleic acid (PNA) or morpholino nucleic acid (MNA) specific for mRNA or DNA that is abnormally proliferating is conjugated to vitamin B₁₂, a ligand of a transcobalamin receptor or intrinsic factor receptor, optionally conjugated to a nuclear localization sequence, optionally conjugated to a transcobalamin protein.

In yet another preferred embodiment, the stabilized mimic binds to double stranded DNA.

In naturally occurring vitamin B₁₂, there is an α-D-5,6-dimethylbenzimidazolyl ribose 3′-phosphate that is bound through the phosphate to the B₁₂ moiety and coordinated to the cobalt ion. In a modified vitamin B₁₂ TC- or IF-binding agent, the M-sugar component is likewise in an α-D configuration, although other configurations (i.e. α-L, β-D and β-L) are possible.

One of the biologically active forms of vitamin B₁₂ has a 5′-deoxyadenosyl moiety in the X position. Coenzyme B₁₂ catalysis occurs via the detachment and reattachment of the methylene radical at the 5′-deoxy position of the vitamin.

In one particular embodiment the linker is a polyamine such as spermine or spermidine.

Because vitamin B₁₂ is preferentially taken up in or near sites of excess proliferation, the TC- or IF-binding agent/active agent of the present invention provides a delivery system capable of targeting abnormal cellular proliferation, infection or viruses and selectively targeting a greater proportion of such a site in relation to healthy cells. A wide range of analogs and derivatives are capable of attaining these properties, as reflected by the above referenced chemical structure and variables.

The TC- or IF-binding agent can be modified in any manner that does not interfere with its fundamental ability to bind a transcobalamin transport protein and thereafter bind the TC receptor. In one embodiment, however, each variable on the vitamin B₁₂ structure independently either (i) retains its natural vitamin B₁₂ structure, (ii) imparts an imaging agent and/or “T” sequence to the cobalamin conjugate, (iii) renders the cobalamin conjugate more water soluble or more stable, (iv) increases the bioavailability of the carrier; (v) increases or at least does not decrease the binding affinity of the carrier for the TC-binding or IF-binding protein over vitamin B₁₂, or (vi) imparts another characteristic that is desired for pharmaceutical or diagnostic performance.

The “T” can be linked to the TC-binding or IF-binding moiety through a number of positions, including any of the V-Z moieties, the X moiety, the M moiety, the K moiety and/or the G¹ moiety, though as mentioned above at least one of Z¹, Z², Z³, Z⁴, Z⁵, Z⁷, Z⁸, M and G¹ moieties comprises a “T”. In one embodiment a nucleic acid sequence useful in antisense technology, a peptide nucleic acid, mopholinonucleic acid, locked nucleic acid, pseudocyclic oligonucleobase, or 2′-O,4′-C-ethylene bridged nucleic acid is linked to the TC- or IF-binding agent through Z², Z⁴, and/or Z⁵ (i.e. one or more of Z², Z⁴ and Z⁵ is L-T and T is an imaging agent). In a more particular embodiment a “T” sequence is linked to the TC- or IF-binding agent through the Z² moiety (i.e. Z² is L-T and T is an imaging agent). In each of the foregoing embodiments, the Z moiety or moieties not containing a “T” preferably retain its natural vitamin B₁₂ configuration, in which VZ is NH₂. Alternatively, the Z moieties not containing a “T” may comprise a secondary or tertiary amino analog of NH₂ substituted by one or two of J¹.

In any Z¹, Z², Z³, Z⁴, Z⁵, Z⁶, Z⁷, Z¹, X, M or G¹ moieties through which a “T” sequence is linked, it will be understood that such moiety may comprise more than sequence, for example, each T can independently comprise the residue of one or more nucleic acid sequence useful in antisense technology, a peptide nucleic acid, mopholinonucleic acid, locked nucleic acid, pseudocyclic oligonucleobase, or 2′-O,4′-C-ethylene bridged nucleic acid bound to L through one or more chelating moieties. More specifically, in a series of embodiments, each T can comprise 1, 2, 3, 4, 5, 6, 7, 8, 9 or 10 sequences bound through one or more chelating moieties.

R¹, R², R³, R⁴, R⁵, R⁶, R⁷, R⁸, R⁹, R¹⁰, R¹¹, R¹² and R¹³ independently represent moieties that do not interfere with binding between the compound and the transcobalamin transport protein or receptor. Vitamin B₁₂ can be modified through these moieties to modulate physical properties of the molecule, such as water solubility, stability or λₘₐₓ Preferred groups for enhancing water solubility include heteroalkyl, amino, C₁₋₆ alkylamino, C₁₋₆ alcohol, C₁₋₆ carboxylic acid and SO₃⁻.

In another embodiment, one, some or all of R¹, R², R³, R⁴, R⁵, R⁶, R⁷, R⁸, R⁹, R¹⁰, R¹¹, R¹² and R¹³ independently assume their natural roles in vitamin B₁₂. Thus, one, some or all of R¹, R², R⁴, R⁵, R⁸, R⁹, R¹¹, R¹² and R¹⁵ are independently methyl in one embodiment and one, some or all of R³, R⁶, R⁷, R¹⁰, R¹³ and R¹⁴ are independently hydrogen.

In another embodiment, one, some or all of Y¹, Y², Y³, Y⁴, Y⁵, Y⁶ and Y⁷ assume their natural roles in vitamin B₁₂ and are O. Similarly, in another embodiment V⁶ assumes its natural role in vitamin B₁₂ and is NH or a primary amine analog thereof substituted by J¹.

In still another embodiment, position X assumes its natural role in vitamin B₁₂, i.e. as cyano, hydroxyl, methyl or 5′-deoxyadenosyl, most preferably 5′-deoxyadenosyl.

In another embodiment M is the radical of a purine or pyrimidine base. In another embodiment M is the radical of adenosine, guanine, cytosine, uridine or thymine.

In still another embodiment M is the radical of 5,6-dimethylbenzimidazole.

In still another embodiment K is CH(OH).

In yet another embodiment E is O.

In another embodiment G¹ is OH.

In still another embodiment, all constituents of the conjugate assume their natural roles in vitamin B₁₂, except for the moieties through which “T” is linked. The “T” is preferably linked to the vitamin B₁₂ structure through Z², Z⁴ and/or Z⁵ and even more preferably through the Z² moieties.

II. Linkers

As noted above, L is the residue of a linker molecule that conjugates “T” to the TC ligand. The structure of the linker from which L is derived (in any one of the Z¹, Z², Z³, Z⁴, Z⁵, Z⁶, Z⁷, X, M or G¹ moieties) is not crucial, provided it does not significantly impair the ability of the conjugate to bind to the transcobalamin or IF transport protein or receptor. L is preferably any multivalent molecule (divalent or greater) that does not significantly impair the ability of the TC carrier to bind to the transcobalamin transport protein or receptor. The ability of vitamin B₁₂ or any other TC-binding carrier to bind to the transcobalamin transport protein or receptor is “significantly impaired” when attaching a linking moiety to the B₁₂ or TC-binding carrier lessens the affinity of the vitamin B₁₂ or the TC-binding carrier for the transcobalamin transport protein to which the vitamin B₁₂ or TC-binding carrier is most readily bound by 50% or more. The unsaturated vitamin B₁₂ binding capacity (UBBC) assay described by D. A. Collins and H. P. C. Hogenkamp in J. Nuclear Medicine, 1997, 38, 717-723 can be used to compare the relative affinities of ligands for this receptor.

In one embodiment the linker is of precise molecular weight and does not posses a molecular weight distribution. In one embodiment, the linker has a molecular weight less than about 2,500, 2,000, 1900, 1800, 1,500, 1,000 or 500 or 250.

A particularly preferred linker is one having multiple sites for conjugation to one or more imaging agents, wherein the linker has a unimodal molecular weight. Recombinant protein production techniques can be employed to obtain poly(amino acid) linkers of substantially constant molecular weight.

In one embodiment the linker is an amino acid or a polymer or peptide formed from a plurality of amino acids. The polymer or peptide can be derived from one or more amino acids. The amino acid, poly(amino acid) or peptide can link T to V through the carboxy terminus or the amino terminus. The amino acid residue, peptide residue or poly(amino acid) residue can conveniently be linked to V and T through an amide (e.g. —N(R)C(—O)— or —C(═O)N(R)—), ester (e.g. —OC(═O)— or —C(═O)O—), ether (e.g. —O—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C_{1-C}₁₄) alkyl.

Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right, but are meant to also include the opposite flow. Particularly suitable peptides and poly(amino acids) comprise from 2 to about 20 amino acids, from 2 to about 15 amino acids or from 2 to about 12 amino acids.

One exemplary poly(amino acid) is poly-L-lysine ((—NHCH((CH₂)₄—NH₂)CO—)ₘ-Q, wherein Q is H, (C₁-C₁₄)alkyl or a suitable carboxy protecting group and m is from 2 to about 20, from about 5 to about 15 or from about 8 to about 11. The polylysine offers multiple primary amine sites to which active agents can be readily attached. Alternatively, the linkers can be formed with multiple cysteines, to provide free thiols or multiple glutamates or aspartates, to provide free carboxyls for conjugation using suitable carbodiimides. Similarly the linker can contain multiple histidines or tyrosines for conjugation. Other exemplary poly(amino acid) linkers are poly-L-glutamic acid, poly-L-aspartic acid, poly-L-histidine, poly-L-ornithine, poly-L-serine, poly-L-threonine, poly-L-tyrosine, poly-L-lysine-L-phenylalanine or poly-L-lysine-L-tyrosine. When the linker is derived from a poly(amino acid) other than polylysine, the linker is, in a series of embodiments, prepared from 2 to about 30 amino acids, 5 to about 20 amino acids or 8 to about 15 amino acids.

In another particular embodiment L is a polyamine residue (having at least three amino moieties) of the following chemical structure: NR′ (alkylene-NR′)ₙalkyleneNR′, wherein n is from 1 to 20, the carbon length of alkylene can vary within the n units and each R′ is independently hydrogen, lower alkyl or T. N is preferably from 1 to 10. Moreover, L preferably has a backbone along its longest length of no more than 100, 75, 50, 40, 30, 20 or 15 atoms. Exemplary polyamines from which L can be derived include spermine (H₂N(CH₂)₃NH(CH₂)₄NH(CH₂)₃NH₂), spermidine (H₂N(CH₂)₃NH(CH₂)₄NH₂), deca-methylene tetraamine and pentamethylene hexamine. These linkers are a definite size and thus provide consistent and predictable targeting by the cobalamin conjugate, in addition to multiple binding sites for the imaging agent.

In another embodiment L is a diamine represented by the formula NH₂(CH₂)ₙNH₂, in which x is 2-20 and preferably 2-12. Thus, the linker can be prepared from 1,6-diaminohexane, 1,5-diaminopentane, 1,4-diaminobutane and 1,3-diaminopropane.

Other suitable linkers are formed from the covalent linkage of various water soluble molecules with amino acids, peptides, poly(amino acids), polyamines, polyoxyalkylenes, polyanhydrides, polyesters, polyamides, polyglycolides and diamines. Suitable water soluble molecules include, for example, polyethylene glycol and dicarboxylic monosaccharides such as glucaric acid, galactaric acid and xylaric acid.

Other suitable linkers include those represented by the formula HO(O)C(CH₂)ₙC(O)OH, in which x is 2-20 and preferably 2-12. Thus, the linker can be prepared from succinic acid, glutaric acid, adipic acid, suberic acid, sebacic acid, azelaic acid or maleic acid. Still other suitable linkers comprise carboxylic acid derivatives that yield an amide upon reaction with an amine. Such reactive groups include, by way of example, carboxylic acid halides such as acid chlorides and bromides; carboxylic acid anhydrides such as acetic anhydrides and trifluoroacetic anhydrides; esters such as p-nitrophenyl esters and N-hydroxysuccinimide esters; and imidazolides. Techniques for using such linkers are described in detail in Bodanszky, Principles of Peptide Synthesis, Springer Verlag, Berlin, 1984.

In one embodiment, the linker is modified to facilitate its conjugation either to V or T. Suitable molecules for modifying the linker include: disuccinimidyl suberate (DSS), bis(sulfosuccinimidyl) suberate (BSS), ethylene glycolbis(succinimidylsuccinate) (EGS), ethylene glycolbis(sulfosuccinimidyl-succinate) (Sulfo-EGS), p-aminophenylacetic acid, dithio-bis-(succinimidyl-propionate) (DSP), 3,3′-dithiobis-(sulfosuccinimidylpropionate) (DTSSP), disuccinimidyl tartarate (DST), disulfosuccinimidyl tartarate (Sulfo-DST), bis(2-(succinimidooxycarbonyloxy)-ethylene)sulfone (BSOCOES), bis(2-(sulfosuccinimidooxy-carbonyloxy)ethylene)sulfone (Sulfo-BSOCOES), dimethyl adipimidate.2HCl (DMA), dimethyl pimelimidate.2HCl (DMP) and dimethyl suberimidate.2HCl (DMS).

(A) Biodegradable Linkers

Various degradable linkers can be used to link the TC-binding or IF-binding moiety to the active agent. The desired linkers can degrade under biological conditions such as by enzymatic cleavage or by systemic pH or temperature. Alternatively, these linkers can be induced to degrade by external manipulation such as changes in pH, temperature, ultrasound, magnetic field, radiation (i.e. UV radiation) or light.

U.S. Pat. No. 5,639,885 entitled “Redox amino acids and peptides containing them;” U.S. Pat. No. 5,637,601 entitled “Anticholinergic compounds, compositions and methods of treatment;” U.S. Pat. No. 5,624,894 entitled “Brain-enhanced delivery of neuroactive peptides by sequential metabolism;” U.S. Pat. No. 5,618,826 entitled “Anticholinergic compounds, compositions and methods of treatment;” U.S. Pat. No. 5,618,803 entitled “Targeted drug delivery via phosphonate derivatives;” U.S. Pat. No. 5,610,188 entitled “Anticholinergic compounds, compositions and methods of treatment;” U.S. Pat. No. 5,525,727 entitled “Brain-specific drug delivery;” U.S. Pat. No. 5,418,244 entitled “Anticholinergic compounds, compositions and methods of treatment;” U.S. Pat. No. 5,413,996 entitled “Targeted drug delivery via phosphonate derivatives;” U.S. Pat. No. 5,389,623 entitled “Redox carriers for brain-specific drug delivery;” U.S. Pat. No. 5,296,483 entitled “Brain-specific analogues of centrally acting amines;” U.S. Pat. No. 5,258,388 entitled “Anticholinergic compounds, compositions and methods of treatment;” U.S. Pat. No. 5,231,089 entitled “Method of improving oral bioavailability of carbamazepine;” U.S. Pat. No. 5,223,528 entitled “Anticholinergic compounds, compositions and methods of treatment;” U.S. Pat. No. 5,187,158 Brain-specific drug delivery;” U.S. Pat. No. 5,177,064 entitled “Targeted drug delivery via phosphonate derivatives;” U.S. Pat. No. 5,155,227 entitled “Compounds for site-enhanced delivery of radionuclides;” U.S. Pat. No. 5,136,038 entitled “Radiopharmaceuticals and chelating agents useful in their preparation;” U.S. Pat. No. 5,087,618 entitled “Redox carriers for brain-specific drug delivery;” U.S. Pat. No. 5,079,366 entitled “Quaternary pyridinium salts;” U.S. Pat. No. 5,053,215 entitled “NMR-assayable ligand-labeled trifluorothymidine containing composition and method for diagnosis of HSV infection;” U.S. Pat. No. 5,024,998 entitled “Pharmaceutical formulations for parenteral use;” U.S. Pat. No. 5,017,618 entitled “Labile derivatives of ketone analogs of 3-substituted-1-alkylamino-2-propanols and their use as beta-adrenergic blockers;” U.S. Pat. No. 5,017,566 entitled “Redox systems for brain-targeted drug delivery;” U.S. Pat. No. 5,008,257 entitled “Brain-specific drug delivery;” U.S. Pat. No. 5,002,935 entitled “Improvements in redox systems for brain-targeted drug delivery;” U.S. Pat. No. 4,983,586 entitled “Pharmaceutical formulations for parenteral use;” U.S. Pat. No. 4,963,688 entitled “Compounds for site-enhanced delivery of radionuclides and uses thereof;” U.S. Pat. No. 4,963,682 entitled “Novel radiopharmaceuticals and chelating agents useful in their preparation;” U.S. Pat. No. 4,933,438 entitled “Brain-specific analogues of centrally acting amines;” U.S. Pat. No. 4,900,837 entitled “Brain-specific drug delivery of steroid sex hormones cleaved from pyridinium carboxylates and dihydro-pyridine carboxylate precursors;” U.S. Pat. No. 4,892,737 entitled “Composition and method for enhancing permeability of topical drugs;” U.S. Pat. No. 4,888,427 entitled “Amino acids containing dihydropyridine ring systems for site-specific delivery of peptides to the brain;” U.S. Pat. No. 4,880,921 entitled “Brain-specific drug delivery;” 35. U.S. Pat. No. 4,863,911 entitled “Method for treating male sexual dysfunction;” U.S. Pat. No. 4,829,070 entitled “Novel redox carriers for brain-specific drug delivery;” U.S. Pat. No. 4,824,850 entitled “Brain-specific drug delivery;” U.S. Pat. No. 4,801,597 entitled “Certain inositol-nicotinate ester derivatives and polyionic complexes therefore useful for treating diabetes meuitus, hyperlipidemia and lactic acidosis;” U.S. Pat. No. 4,771,059 entitled “Brain-specific analogues of centrally acting amines;” U.S. Pat. No. 4,727,079 entitled “Brain-specific dopaminergic activity involving dihydropyridine carboxamides, dihydroquinoline and isoquinoline carboxamides;” U.S. Pat. No. 4,540,564 entitled “Brain-specific drug delivery;” and U.S. Pat. No. 4,479,932 entitled “Brain-specific drug delivery” to Nicholas S. Bodor, et al., disclose several biodegradable linkers that target the brain. For example, a lipoidal form of dihydropyridine pyridinium salt redox carrier, DHC, linked to a centrally acting drug which can be reduced and biooxidized to pass through the blood brain barrier. The dihydropyridine nucleus readily and easily penetrates the blood brain barrier in increased concentrations; furthermore, the in vivo oxidation of the dihydropyridine moiety to the ionic pyridinium salts thereby prevents its elimination from the brain, while elimination from the general circulation is accelerated, resulting in a prolongedly sustained brain-specific drug activity. This dihydropyridine can be incorporated into the linkers set forth above for biodegradation.

Additionally U.S. Pat. No. 4,622,218 entitled “Testicular-specific drug delivery,” discloses linkers that can specifically deliver drugs to the testes in much the same manner and which can be used in the linkers of the present invention. The lipoidal form [D—DHC] of a dihydropyridine pyridinium salt redox carrier, e.g. 1,4-dihydrotrigonelline, penetrates the blood-testis barrier. Oxidation of the dihydropyridine carrier moiety in vivo to the ionic pyridinium salt type drug/carrier entity [D—QC]⁺ prevents elimination thereof from the testes, while elimination from the general circulation is accelerated, resulting in significant and prolongedly sustained testicular-specific drug activity.

Margerum, et al. in U.S. Pat. No. 5,976,493 discloses the use of polychelant compounds which are degradable in vivo to release excretable fragments for diagnostic imaging which also are suitable in the linkers of the present invention. These compounds contain a linker moiety which is metabolically cleavable to release macrocyclic monochelant fragments, wherein the macrocyclic skeleton preferably has 9 to 25 ring members and a biotolerable polymer, preferably a substantially monodisperse polymer. Other suitable linkers are disclosed, for example, in Krejcarek et al. (Biochemical and Biophysical Research Communications 77: 581 (1977)) (mixed anhydrides), Hnatowich et al. (Science 220: 613 (1983))(cyclic anhydrides), U.S. Pat. No. 5,637,684 to Cook, et al. (Phosphoramidate and phosphorothioamidate oligomeric compounds).

Other suitable biodegradable polymers from which the linker can be formed are the polyanhydrides and polyorthoesters, which take advantage of labile backbone linkages (see: Domb et al. Macromolecules, 22, 3200, 1989; and Heller et al. Biodegradable Polymers as Drug Delivery Systems, Dekker, NY: 1990). Other linker materials include hydrogels, such as the PEG-oligoglycolyl-acrylates disclosed in U.S. Pat. No. 5,626,863 to Hubbell et al. Other biodegradable linkers are formed from oligoglycolic acid is a poly(a-hydroxy acid), polylactic acid, polycaprolactone, polyorthoesters, polyanhydrides and polypeptides.

Nonlimiting examples of U.S. patents that describe controlled release formulations suitable for use as linking agents are: U.S. Pat. No. 5,356,630 to Laurencin et al. (Delivery System for Controlled Release of Bioactive Factors); U.S. Pat. No. 5,797,898 to Santini, Jr. et al. (Microchip Drug Delivery Devices); U.S. Pat. No. 5,874,064 to Edwards et al. (Aerodynamically Light Particles for Pulmonary Drug Delivery); U.S. Pat. No. 5,548,035 to Kim et al. (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al (Drug Delivery Porphyrin Composition and Methods); U.S. Pat. No. 5,741,329 to Agrawal et al (Methods of Controlling the pH in the Vicinity of Biodegradable Implants); U.S. Pat. No. 5,820,883 to Tice et al (Methods for Delivering Bioactive Agents into and Through the Mucosally-Associated Lymphoid Tissues and Controlling Their Release);U.S. Pat. No. 5,955,068 to Gouin et al. (Biodegradable polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et al (Continuous Release Polymeric Implant Carriers); U.S. Pat. No. 6,060,582 to Hubbell et al (Photopolymerizable Biodegradable Hydrogels as Tissue Contacting Materials and Controlled Release Carriers); U.S. Pat. No. 6,113,943 to Okada et al (Sustained-Release Preparation Capable of Releasing a Physiologically Active Substance); and PCT Publication No. WO 99/59548 to Oh et al (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Improved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No. 5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Implantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 Ionically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-co-anhydride) for biological application; U.S. Pat. No. 4,948,587 Ultrasound enhancement of transbuccal drug delivery; U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, bioerodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

Nonmetallic radioisotopes can conveniently be linked to the vitamin B₁₂ structure through a residue of a peptide having the following formula: 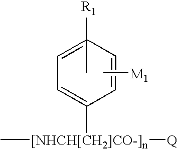
wherein each M is independently a non-metallic radionuclide; each R is independently (C₁-C₁₄)alkyl, (C₂-C₁₄)alkenyl, (C₂-C₁₄)alkynyl, (C₁-C₁₄)alkoxy, hydroxy, cyano, nitro, halo, trifluoromethyl, N(Rₐ)(R_{b}), (C₁-C₁₄)alkanoyl, (C₂-C₁₄)alkanoyloxy, (C₆-C₁₀)aryl or (C₃-C₈)cycloalkyl wherein Rₐ and R_{b} are each independently H or (C₁-C₁₄)alkyl; P; Q is H, (C₁-C₁₄)alkyl or a suitable carboxy protecting group; n is 2 to about 20; I is 1-5, j is 0-4 and I+j is ≦5; or a pharmaceutically acceptable salt thereof. Specifically, i can be 1, j can be 0, M can be a positron emitter such as Fluorine-18, Bromine-76, Iodine-124 or a gamma emitter such as Iodine-123 or Iodine-131 and n can be about 6 to about 12.

The above discussion has demonstrated how the various variables associated with the cobalamin conjugates of the present invention can be independently varied to more particularly define specific classes of cobalamin conjugates encompassed by this invention. It is to be understood that the modification of one variable can be made independently of the modification of any other variable. Moreover, any number of embodiments can be defined by modifying two or more of the variables in such embodiments. A few of such embodiments are described below for purposes of exemplification.

Subembodiment 1: X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, mono-, di- or tri-phosphate or L-T; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z¹, Z², Z¹, Z⁴, Z⁵, Z⁷ and Z⁸, M or G¹ comprises a “T”; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 2: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z¹, Z², Z³, Z⁴, Z⁵, Z⁷ and Z⁸, M or G¹ comprises a “T”; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 3: X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, mono-, di- or tri-phosphate or L-T; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 4: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z¹, Z², Z³, Z⁴, Z⁵, Z⁷, Z⁸, M and G¹ comprises a radionuclide; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 5: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V¹, V⁶, V¹ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z², Z⁴ or Z⁵ comprises a “T”, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 6: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V¹, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z², Z⁴ or Z⁵ comprises a “T”, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 7: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z¹, Z², Z³, Z⁴, Z⁵, Z⁷, Z⁸, M and G¹ comprises a “T”; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 8: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V¹, V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z², Z⁴ or Z⁵ comprises a “T”, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 9: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z², Z⁴ or Z⁵ comprises a “T”, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 10: X is 5′-deoxyadenosyl; M, K, E and G¹ retain their natural vitamin B₁₂ configuration; Y¹, Y², Y³, Y⁴, Y⁵₃ Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z²_{, Z}³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises an optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA; at least one of Z², Z⁴ or Z⁵ comprises a “T”, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiments 11-20: Any one of subembodiments 1-10, wherein the linker has a substantially constant molecular weight.

Subembodiments 21-30: Any one of subembodiments 1-10, wherein the linker is a polyamine of the following chemical structure: NR′(alkylene-NR′)ₙalkyleneNR′, wherein n is from 1 to 20, the carbon length of alkylene can vary within the n units and each R′ is independently hydrogen, lower alkyl or T.

Subembodiments 31-40: Any one of subembodiments 1-10, wherein the linker is spermine, spermidine, decamethylene tetraamine or pentamethylene hexamine.

III. Definitions for the Invention

The following definitions and term construction are intended, unless otherwise indicated. Specific and preferred values listed below for radicals, substituents and ranges, are for illustration only; they do not exclude other defined values or other values within defined ranges for the radicals and substituents. Explanations of terms below if not used elsewhere in the text are considered embodiments of the invention.

A wavy line in the chemical structures herein indicates either a dative or covalent bond such that there are three covalent Co—N bonds and one dative Co—N bond, wherein, in the case of the dative bond, the valance of nitrogen is completed either with a double bond with an adjacent ring carbon or with a hydrogen.

A dotted line in the chemical structures herein indicates either a double or single bond such that the double bond does not over-extend the valence of the element (i.e. to give pentavalent carbons) and, in the case of a single bond, the valence is completed with hydrogen.

Halo is fluoro, chloro, bromo or iodo.

Alkyl, alkoxy, alkenyl, alkynyl, etc. denote both straight and branched groups; but reference to an individual radical such as “propyl” embraces only the straight chain radical, while a branched chain isomer such as “isopropyl” being specifically referred to embraces only the branched radical.

The term heterocycle or heterocyclic, as used herein except where noted represents a stable 5- to 7-membered monocyclic or stable 8- to 11-membered bicyclic heterocyclic ring which is either saturated or unsaturated, and which consists of carbon atoms and from one to three heteroatoms selected from the group consisting of N, O and S; and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom that results in the creation of a stable structure.

The term alkyl, as used herein, unless otherwise specified, refers to a saturated straight, branched, or cyclic, hydrocarbon of C1 to C10, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Moieties with which the alkyl group can be substituted are selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term lower alkyl, as used herein, and unless otherwise specified, refers to a C1 to C4 saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The terms alkenyl and alkynyl refer to alkyl moieties wherein at least one saturated C—C bond is replaced by a double or triple bond. Thus, (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl. Similarly, (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “alkylene” refers to a saturated, straight chain, divalent alkyl radical of the formula —(CH2)n-, wherein n can be 1, 2, 3, 4, 5, 6, 7, 8, 9 or 10.

As used herein, with exceptions as noted, “aryl” is intended to mean any stable monocyclic, bicyclic or tricyclic carbon ring of up to 7 members in each ring, wherein at least one ring is aromatic. Examples of aryl ring systems include phenyl, naphthyl, tetrahydronaphthyl and biphenyl. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term purine or pyrimidine base includes, but is not limited to, adenine, N6-alkylpurines, N6-acylpurines (wherein acyl is C(O)(alkyl, aryl, alkylaryl, or arylalkyl), N6-benzylpurine, N6-halopurine, N6-vinylpurine, N6-acetylenic purine, N6-acyl purine, N6-hydroxyalkyl purine, N6-thioalkyl purine, N2-alkylpurines, N2-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C5-alkylpyrimidines, C5-benzylpyrimidines, C5-halopyrimidines, C5-vinylpyrimidine, C5-acetylenic pyrimidine, C5-acyl pyrimidine, C5-hydroxyalkyl purine, C5-amidopyrimidine, C5-cyanopyrimidine, C5-nitropyrimidine, C5-aminopyrimidine, N2-alkylpurines, N2-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limmted to, guanine, adenine, hypoxanthine, 2,6-diaminopurine and 6-chloropurine. Functional oxygen and nitrogen groups on the base can be protected as necessary or desired. Suitable protecting groups are well known to those skilled in the art, and include trimethylsilyl, dimethylhexylsilyl, t-butyldimethylsilyl and t-butyldiphenylsilyl, trityl, alkyl groups, and acyl groups such as acetyl and propionyl, methanesulfonyl, and p-toluenesulfonyl.

The term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C1 to C4 alkyl or C1 to C4 alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The term heteroaryl or heteroaromatic, as used herein, refers to an aromatic moiety that includes at least one sulfur, oxygen, nitrogen or phosphorus in the aromatic ring. The term heterocyclic refers to a nonaromatic cyclic group wherein there is at least one heteroatom, such as oxygen, sulfur, nitrogen or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N6-alkylpurines, N6-benzylpurine, N6-halopurine, N6-vinypurine, N6-acetylenic purine, N6-acyl purine, N6-hydroxyalkyl purine, N6-thioalkyl purine, thymine, cytosine, 6-azapyrimidine, 2-mercaptopyrmidine, uracil, N5-alkylpyrimidines, N5-benzylpyrimidines, N5-halopyrimidines, N5-vinylpyrimidine, N5-acetylenic pyrimidine, N5-acyl pyrimidine, N5-hydroxyalkyl purine, and N6-thioalkyl purine, and isoxazolyl. The heteroaromatic and heterocyclic moieties can be optionally substituted as described above for aryl, including substituted with one or more substituent selected from halogen, haloalkyl, alkyl, alkoxy, hydroxy, carboxyl derivatives, amido, amino, alkylamino, dialkylamino. The heteroaromatic can be partially or totally hydrogenated as desired. As a nonlimiting example, dihydropyridine can be used in place of pyridine. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenesulfonyl.

The term aralkyl, as used herein, and unless otherwise specified, refers to an aryl group as defined above linked to the molecule through an alkyl group as defined above. The term alkaryl, as used herein, and unless otherwise specified, refers to an alkyl group as defined above linked to the molecule through an aryl group as defined above.

The term alkoxy, as used herein, and unless otherwise specified, refers to a moiety of the structure —O-alkyl, wherein alkyl is as defined above.

The term amino, as used herein, refers to a moiety represented by the structure —NR₂, and includes primary amines, and secondary, and tertiary amines substituted by alkyl (i.e. alkylamino). Thus, R₂ may represent two hydrogens, two alkyl moieties or one hydrogen and one alkyl moiety.

The term amido, as used herein, refers to a moiety represented by the structure —C(O)NR₂, wherein R₂ is as defined for amino.

As used herein, “adenosyl” is an adenosine radical attached to the 6-position of cobalamin via the 5′ position of adenosine.

As used herein, an “amino acid” is a natural amino acid residue (e.g. Ala, Arg, Asn, Asp, Cys, Glu, Gln, Gly, His, Hyl, Hyp, Ile, Leu Lys, Met, Phe, Pro, Ser, Thr, Trp, Tyr, and Val) in D or L form, or an unnatural amino acid (e.g. phosphoserine; phosphothreonine; phosphotyrosine; hydroxyproline; gamma-carboxyglutamate; hippuric acid; octahydro-indole-2-carboxylic acid; statine; 1,2,3,4,-tetrahydmisoquinoline-3-carboxylic acid; penicillamine; ornithine; cituline; α-methyl-alanine; para-banzoylphenylalanine; phenyl-glycine; propargylglycine; sarcosine; and tert-butylglycine) residue having one or more open valences. Other unnatural amino acids include those represented by the formula NH₂(CH₂)_{y}COOH, wherein y=2-20, and preferably 2-12, and include the aminoalkanoic acids such as 1-amino caproic acid (H₂N—(CH₂)₅—COOH).

The term also comprises natural and unnatural amino acids bearing amino protecting groups such as acetyl, acyl, trifluoroacetyl, and benzyloxycarbonyl), as well as natural and unnatural amino acids protected at carboxy with protecting groups such as a C₁-C₆ alkyl, phenyl or benzyl ester and amide. Other suitable amino and carboxy protecting groups are known to those skilled in the art. See for example, T. W. Greene, Protecting Groups in Organic Synthesis; Wiley: New York, 1981; D. Voet, Biochemistry, Wiley: New York, 1990; L. Stryer, Biochemistry, (3rd Ed), W.H. Freeman and Co.: New York, 1975; J. March, Advanced Organic Chemistry, Reactions, Mechanisms and Structure, (2nd Ed.), McGraw Hill: New York, 1977; F. Carey and R. Sundberg, Advanced Organic Chemistry, Part B: Reactions and Synthesis, (2nd Ed.), Plenum: New York, 1977; and references cited therein.

According to the invention, the amino or carboxy protecting group can also comprise a non-metallic radionuclide (e.g. Fluorine-18, Iodine-123 or Iodine-124).

As used herein, a “peptide” is a sequence of 2 to 25 amino acids (e.g. as defined hereinabove) or peptidic residues having one or more open valences. The sequence may be linear or cyclic. For example, a cyclic peptide can be prepared or may result from the formation of disulfide bridges between two cysteine residues in a sequence. A peptide can be linked through the carboxy terminus, the amino terminus or through any other convenient point of attachment, such as, for example, through the sulfur of a cysteine. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

As used herein, “adenosyl” is an adenosine radical in which any synthetically feasible atom or groups of atoms have been removed, thereby providing an open valence. Synthetically feasible atoms that may be removed include the hydrogen atom of the hydroxy group at the 5′ position. Accordingly, adenosyl can conveniently be attached to the 6-position of a compound of formula I via the 5′ position of adenosyl.

The term “aptamer” as used herein is a random, nonencoding nucleic acid sequence having a desirable action on a target. A desirable action includes, but is not limited to, binding of the target, catalytically changing the target, reacting with the target in a way which modifies/alters the target or the functional activity of the target, covalently attaching to the target as in a suicide inhibitor, or facilitating the reaction between the target and another molecule. In the preferred embodiment, the action is specific binding affinity for a target molecule, such target molecule being a three dimensional chemical structure other than a polynucleotide that binds to the aptamer through a mechanism which predominantly depends on Watson/Crick base pairing or triple helix binding, wherein the aptamer is not a nucleic acid having the known physiological function of being bound by the target molecule. In one embodiment of the invention, the aptamers are identified using the SELEX methodology. Aptamers includes nucleic acids that are identified from a candidate mixture of nucleic acids, wherein the aptamer being a ligand of a given target by the method comprising a) contacting the candidate mixture with the target, wherein nucleic acids having an increased affinity to the target relative to the candidate mixture may be partitioned from the remainder of the candidate mixture; b) partitioning the increased affinity nucleic acids from the remainder of the candidate mixture; and c) amplifying the increased affinity nucleic acids to yield a ligand-enriched mixture of nucleic acids. As used herein aptamer or aptamers denotes both singular and plural sequences of nucleic acids which are capable of binding to a protein or other molecule, and thereby disturbing the protein's or other molecule's function.

A “complementary DNA,” or “cDNA” gene includes DNA synthesized by reverse transcription of RNA.

The term “derivative” includes the “chemical derivatives” of the molecule. As used herein, a molecule is said to be a “chemical derivative” of another molecule when it contains additional chemical moietics not normally a part of the molecule. Such moiety may improve the molecule's solubility, absorption, biological half-life, etc. The moieties may alternatively decrease the toxicity of the molecule, eliminate or attenuate any undesirable side-effect of the molecule, etc. Examples of moieties capable of mediating such effects are disclosed in Remington's Pharmaceutical Sciences (1980) and will be apparent to those of ordinary skill in the art. Derivatives/modifications should be selected so that the modified nucleic acid may be harmless to the patient/mammal.

The term “modified nucleotides” includes nucleotides, polynucleotides and oligonucleotides with modified or substituted sugar groups and the like.

The term “oncogene” means a gene that induces cancer or other uncontrolled cell proliferation including a mutated or activated proto-oncogene that is associated with the development and proliferation of tumor cells. Exemplary oncogenes include but are not limited to neu, src, abl, lck, fyn, phl-abl, H-ras, N-ras, K-ras, myc and mos.

The term “naturally occurring nucleotides” includes deoxyribonucleotides and ribonucleotides.

The term “oligonucleotide linkages” includes natural phosphate linkages as well as synthetic oligonucleotide linkages such as phosphorthioate, phophorodithioate, phosphoroselenoate, phosphorodeselenoate, phosphoranilo-thioate, phosphoraniladate, phospboroamidate, and the like, as either described herein or otherwise known.

The terms “oligonucleotide” or “primer” include naturally occurring, and modified nucleotides linked together by naturally occurring, and non-naturally occurring oligonucleotide linkages. Oligonucleotides are a polynucleotide subset with 200 or fewer bases in length. Preferably, oligonucleotides are about 10 to about 6 bases in length and most preferably from about 12 to 20 to about 40 bases in length. Oligonucleotides are usually single stranded, e.g., for probes, although oligonucleotides may be double stranded. Oligonucleotides can be either sense or antisense oligonucleotides.

Generally, “operably linked” means that the DNA sequences being linked are contiguous and, in the case of a secretory leader, contiguous and in reading phase. However, enhancers do not have to be contiguous. Linking is accomplished (either wild-type) by ligation at convenient restriction sites. If such sites do not exist, synthetic oligonucleotide adaptors or linkers may be used in accord with conventional practice, or any other technique suitable for linking pieces of DNA, such as seeing by overlap extension (SOE).

The term “nucleic acid/nucleic acid” includes polyribonucleotide or polydeoxyribonucleotide, which may be unmodified RNA or DNA or modified RNA or DNA. “Nucleic acids” include, without limitations, single- and double-stranded DNA, DNA that is a mixture of single- and double-stranded regions, single- and double-stranded RNA, and RNA that is a mixture of single- and double-strand regions, hybrid molecules including DNA and RNA that may be single-stranded or, more typically, double-stranded or a mixture of single- and double-stranded regions. In addition, “nucleic acid” refers to triple stranded region of RNA or DNA or both RNA and DNA. The term nucleic acid also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA. Thus, nucleic acid” embraces chemically, enzymatically or metabolically modified forms of polynucleotides as typically found in nature, as well as chemical forms of DNA and RNA, and DNA and RNA characteristic of viruses and cells. “Nucleic acid” also embraces relatively short polynucleotides, often referred to as oligonucleotides.

The term “polypeptide” refers to any peptide or protein having two or more amino acids joined to each other by peptide bonds or by modified peptide bonds, i.e., peptide isosteres. “Polypeptide” refers to both short chains, commonly referred to as peptides, oligopeptides or oligomers and to longer chains, generally referred to as proteins. Polypeptides may contain amino acids other than the 20 gene-encoded amino acids.

Promoter as used herein is a DNA sequence generally described as the 5′ region of a gene located proximal to the start codon. The transcription of an adjacent gene(s) is initialed at the promoter region. If a promoter is an inducible promoter, than the rate of transcription increases in response to an inducing agent. In contrast, the rate of transcription is not regulated by an inducing agent if the promoter is a constitutive promoter. In one embodiment, it is preferred that the promoter is tissue-specific, that is, it is induced to selectively express in a specific tissue. Also, tissue-specific enhancer elements may be employed. Additionally, such promoters may include tissue- and cell-specific promoters of an organism.

The term “variant” or “variation” refers to a nucleic acid or polypeptide that differs in sequence from a reference nucleic acid or polypeptide respectively, but retains essential properties. A typical variant of a nucleic acid differs in nucleotide sequence from another, reference nucleic acid. Changes in the nucleotide sequence of the variant may or may not alter the amino acid sequence of a polypeptide encoded by the reference nucleic acid. Nucleotide changes may result in amino acid substitutions, additions, deletions, fusions, and truncations in the polypeptide encoded by the identified sequence. A typical variant of a polypeptide differs in amino acid sequence from another, reference polypeptide. Generally, differences are limited so that the sequences of the reference polypeptide and the variant are substantially similar, and, and in many regions, have identity. Similar identity means at least 60% sequence homology. Variant and reference polypeptides may differ in amino acid sequence by one or more substitutions, additions, or deletions in any combinations. A substituted or inserted amino acid residue may or may not be encoded by the genetic code, e.g., a D-amino acid or one other than an alpha amino acid, such as 3-amino propionic acid, or taurine. A variant of a polynucleotide or polypeptide may be naturally occurring such as an allelic variant or a mutation, or it may be a variant that is not known to occur naturally. Non-naturally occurring variant of polynucleotides and polypeptide may be made by mutagenesis techniques or by direct synthesis.

A variant of a reference polypeptide is a polypeptide that has at least about 80%, preferably at least about 90%, but less than 100%, contiguous amino acid sequence homology or identity to the amino acid sequence corresponding to the reference polypeptide.

A polypeptide of the invention, and thus the DNA encoding the polypeptide, may include amino acid residues not present in the polypeptide, e.g., amino acid substitutions, and amino and/or carboxy termini, or internal deletions or insertions, of amino acid residues relative the reference polypeptide, Variant polypeptides of the invention may include polypeptides having at least one D-amino acid, as well as moieties other amino acid residues that correspond to the reference polypeptide, such as amino acid residues that form a part of the fusion protein nucleic acid molecules or targeting moieties such as antibodies or fragments thereof.

“Antisense nucleic acids” refers to nucleotide sequences, including natural nucleotide sequences, sequences derived from phosphorothioate nucleic acids, sequences derived from PNA, MNA, LNA, PCO, ENA or other stablized antisense sequences (also referred to as stabilized mimics), which are not templates for synthesis but yet interact with complementary sequences in other nucleic acid molecules thereby causing function of those molecules to be affected. Antisense sequences may hybridize with and inactivate mRNA or DNA, thus inhibiting and/or preventing transcription, translation and/or splicing of the target gene. An alternative antisense approach is the use of ribozymes that catalyze RNA cleavage and inhibit the translation of RNA into protein. Aptamers are synthetic chains of nucleotides that bind directly to target proteins, inhibiting their activity and can be considered to be antisense compounds. Antisense therapy is considered to be a form of gene therapy because it is modulation of gene function for therapeutic purposes. However, these oligonucleotides differ from standard gene therapies because they cannot give rise to proteins but can only block the expression of existing genes. Several antisense approaches use gene therapy technologies, e.g., ribozymes and antisense RNA using vectors.

This relationship between an antisense compound such as an oligonucleotide and its complementary nucleic acid target, to which it hybridizes, is commonly referred to as “antisense.” “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent (e.g., viral). In the present invention, the targets include, but are not limited to, are nucleic acid sequences that modulate the expression of viral genes, oncogenes or cell cycle regulatory genes. The targeting process also includes determination of a site or sites within the nucleic acid sequence for the antisense interaction to occur such that modulation of gene expression will result.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon in eukaryotes is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, event though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from the genes of interest. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TEA, respectively). The terms “start codon region,” “AUG region” and “translation initiation codon region” refer to a portion of such a mRNA or gene that encompasses from about 25 to about 50 contiguous nucleotides in either direction (i.e., 5′ or 3′) from a translation initiation codon. This region is a preferred target region. Similarly, the terms “stop codon region” and “translation termination codon region” refer to a portion of such a mRNA or gene that encompasses from about 25 to about 50 contiguous nucleotides in either direction (i.e., 5′ or 3′) from a translation termination codon. This region is a preferred target region. The open reading frame (ORF) or “coding region,” which is known in the art to refer to the region between the translation initiation codon and the translation termination codon, is also a region which may be target, effectively. Other preferred target regions include the 5′ untranslated region (5′UTR), known in the art to refer to the portion of an mRNA in the 5′ direction from the translation initiation codon, and thus including nucleotide between the 5′ cap site and the translation initiation codon of an mRNA or corresponding nucleotides on the gene and the 3′ untranslated region (3′UTR), known in the art to refer to the portion of an mRNA in the 3′ direction from the translation termination codon and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′5′ triphosphate linkage. The 5′ cap region of a mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a pre-mRNA transcript to yield one or more mature mRNA. The remaining (and therefore translated) regions are known as “exons” and are spliced together to form a continuous mRNA sequence, mRNA splice sites, i.e., exon—exon or intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Targeting particular exons in alternatively spliced mRNAs may also be preferred. It has also been found that introns can also be effective, and therefore preferred, target regions for antisense compounds targeted, for example, to DNA or pre-mRNA.

Once the target site or sites have been identified, oligonucleotides are chosen which are sufficiently complementary to the target, i.e., hybridize sufficiently well and with sufficient specificity, to give the desired modulation.

“Hybridization” in the context of this invention, means hydrogen bonding, also known as Watson-Crick base pairing, between complementary bases, usually on opposite nucleic acid strands or two regions of a nucleic acid strand. Guanine and cytosine are examples of complementary bases that are known to form three hydrogen bonds between them. Adenine and thymine are examples of complementary bases that form two hydrogen bonds between them.

“Specifically hybridizable” and “complementary” are terms that are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vitro assays or therapeutic treatment and, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

The overall effect of interference with mRNA function is modulation of expression of the proteins of interest. In the context of this invention “modulation” means either inhibition or stimulation; i.e., either a decrease or increase in expression. This modulation can be measured in ways which are routine in the art, for example by Northern blot assay of mRNA expression, or reverse transcriptase PCR, as taught in the examples of the instant application or by Western blot or ELISA assay of protein expression, or by an immunoprecipitation assay of protein expression. Effects on cell proliferation or tumor cell growth can also be measured, as taught in the examples of the instant application. Inhibition is presently preferred.

The nucleic acid conjugates of this invention can be used in diagnostics, therapeutics, prophylaxis, and as research reagents and in kits.

Specific examples of preferred antisense compounds useful in this invention include oligonucleotides containing modified backbones or non-natural internucleoside linkages.

“Chimeric” oligonucleotides or “chimeras,” in the context of this invention, are oligonucleotides which contain two or more chemically distinct regions, such as different sugars and/or backbone chemistries within the same compound to impart different properties, each made up of at least one nucleotide, such as chimeraplast, composed of both DNA and RNA designed to specifically bind to the target DNA sequence and create a mismatched base-pair. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids. Ribozymes are not comprehended by the present invention.

The compounds of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acid conjugates of the invention and prodrugs of such nucleic acid conjugates. “Pharmaceutically acceptable salts” are physiologically and pharmaceutically acceptable salts of the nucleic acids of the invention: i.e., salts that retain the desired biological activity of the parent compound and do not impart undesired toxicological effects thereto (see, for example, Bergs et al, “Pharmaceutical Salts,” J. of Pharma Sci., 1977, 66, 1-19).

The term host, as used herein, refers to a unicellular or multicellular organism in which the infectious agent can replicate, including cell lines and animals, and preferably a human. Alternatively, the host can be carrying a part of the infectious agent's genome, whose replication or function can be altered by the compounds of the present invention. The term host specifically refers to infected cells, cells transfected with all or part of the infectious agent's genome and animals, in particular, primates (including chimpanzees) and humans. In most animal applications of the present invention, the host is a human patient. Veterinary applications, in certain indications, however, are clearly anticipated by the present invention (such as chimpanzees).

IV. Pharmaceutically Acceptable Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The term “pharmaceutically acceptable salt or prodrug” is used throughout the specification to describe any pharmaceutically acceptable form (such as an ester, mono-, di- or tri-phosphate ester, salt of an ester or a related group) of a TC- or IF-binding carrier, which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess activity against infectious disease or are metabolized to a compound that exhibits such activity

V. Antigene and Antisense Therapies

In principle, two general strategies can be adapted to design gene therapy drugs. Oligonucleotides or analogs thereof such as stablized mimics can be designed to recognize and hybridize to complementary sequences of a particular gene whereby they interefere with the transcription of that particluar gene (antigene therapy). Alternatively, oligonucleotides or analogs thereof such as stablized mimics can be designed to recognize and hybridize to complementary sequences in mRNA and thereby inhibit its translation (antisense therapy).

Antisense oligonucleotides or analogs thereof such as stablized mimics having sequence specificity for a sequence near an initiation region of the mRNA molecule can prevent translation by forming a complex that interferes with the formation of an initiation complex at that site. If a particular mRNA molecule has multiple initiation sites, then an antisense oligomer having sequence specificity for a sequence at or near an upstream initiation site can be used to direct translation initiation to a downstream site.

Antisense oligonucleotides or analogs thereof such as stablized mimics having sequence specificity for a portion of the coding sequence of an mRNA molecule can prevent translation by forming a complex that interferes with elongation.

Antisense oligonucleotides or analogs thereof such as stablized mimics having sequence specificity for a sequence near a stop codon can reduce polypeptide synthesis by forming a complex that interferes with the termination process. Interfering with the termination process can prevent the disassociation of the ribosome, polypeptide complex from the mRNA molecule, resulting in an accumulation of such complexes along the mRNA molecule. In this case, the newly synthesized polypeptide would not be released from the ribosome and therefore would be unable to perform its biological function.

Although not limited to any particular mode of action, oligonucleotides or analogs thereof such as stablized mimics useful for antigene therapy can interact in a sequence specific manner with the template strand of a nucleic acid molecule and prevent transcription of that template. This interaction within a cell can result in a complex that prevents transcription by steric hindrance. For example, a complex can reduce the production of RNA by interfering with an RNA polymerase. The template strand of nucleic acid can be a DNA or RNA molecule. Oligonucleotides or analogs thereof such as stablized mimics can exhibit sequence specificity for the template strand of a host DNA molecule that can be transcribed into an mRNA molecule, or a viral RNA molecule that can be transcribed into DNA by reverse transcriptase. In addition, oligonucleotides or analogs thereof such as stablized mimics can exhibit sequence specificity for at least a portion of a regulatory, intron, or exon region of a template strand of nucleic acid.

Any oligonucleotide can be conjugated to vitamin B₁₂, ligands of transcobalamin receptors, ligands of intrinsic factor receptors or carriers of the present invention, to assist in the delivery of the antisense into the cell, preferably in a cell specific manner. Viable antisense oligonucleotides include the following:

Vitravene (fomivirsen): approved by the FDA in 1998. Vitravene, a 21-base long oligonucleotide (GCGTTTGCTCTTCTTCTTGCG), is used for treating cytomegalovirus (CMV)-induced retinitis, an opportunistic infection of the eye that destroys the retina and results in blindness, from Isis Pharmaceuticals Inc. partnered with Novartis. Currently, Vitravene is the only antisense drug to win FDA approval, the solitary commercial achievement of an idea reaching back to 1978, when Stephenson and Zamecnik showed an antisense oligonucleotide inhibited replication of Rous sarcoma virus in vitro. (Proc. Natl. Acad. Sci. USA. 1978 January; 75(1):285-8.).

ISIS 2503 is a potent, selective antisense inhibitor of Ha-ras gene expression in Phase I/II trials developed by Isis Pharmaceuticals, Inc. ISIS 2503 is an effective antitumor agent against tumor types that express mutant Ha-ras, mutant Ki-ras and tumor types that express normal ras genotype, by binding to the Ha-ras gene to inhibit the production of the Ha-ras protein. ISIS 2503 has displayed antitumor activity against a wide range of human cancer cell lines and in human tumor types, including bladder, breast, colon adenocarcinomas and, most sensitively non-small lung carcinoma.

ISIS 3521 is an antisense anticancer compound being developed by Isis Pharmaceuticals, Inc. that is a potent, selective inhibitor of protein kinase C-alpha (PKC-α) expression in Phase III trials. PKC is a family of closely-related signal transduction proteins that regulate information flow in and out of cells and modulate cellular responses to environmental stimuli. The PKC family plays a role in normal cell function, and is also involved in abnormal cell growth. ISIS 3521 is an antisense compound that binds to a mRNA sequence specific to PKC-A and thus selectively inhibits production of this protein without inhibiting production of other proteins in the PKC family, thereby selectively inhibiting a single family member that may play a role in disease while allowing other members of the family to continue to perform normal cellular functions

ISIS 5132 is an antisense inhibitor of C-raf kinase being developed by Isis Pharmaceuticals, Inc. that plays a role in signal processes that regulate cell growth and proliferation and part of the raf kinase family that is thought to play an important role in the development of some solid tumors in Phase I/II trials. In addition, raf has been shown to facilitate the ras protein function, an oncogene known to be involved in the initiation and progression of some human tumors. Thus, novel cancer therapies directed against raf kinase are useful in the treatment of ras-dependent tumors. Activated raf has also been detected in a variety of human cancers including small-cell lung carcinoma and breast cancer, and it has been reported that 60% of all lung carcinoma cells express unusually high levels of normal C-raf mRNA and protein.

ISIS 104838 is an antisense being developed by Isis Pharmaceuticals, Inc. that inhibits TNF-α, which plays a role in rheumatoid arthritis and Crohn's Disease.

ISIS 102453 is an antisense being developed by ISIS Pharmaceuticals, Ins. The antisense molecule modulates the expression of human β-catenin, which plays a role in a number of proliferation disorders including cancer. One antisense sequence that has been identified is a 20 mer phosphorothioate consisting of 5′-CCCCTCGCTCTCCGCTCCCG-3′, and is directed towards nucleotides 39-58 of the 5′ UTR. This, and other potential antisense sequences, are described in WO 0100872.

ISIS 14803 is a 20-base phosphorothioate oligodeoxynucleotide antisense inhibitor complementary to HCV RNA sequences adjacent to the polyprotein initiation codon of HCV, in Phase II trials. The largest sequence is highly conserved among independent HCV isolates. Upon binding to the complementary target sequence, the oligonucleotide inhibits expression of HCV proteins required for HCV replication. Specific inhibition of HCV core protein expression has been demonstrated in biochemical and cell culture assays. ISIS 14803 treatment also inhibits expression of an HCV-luciferase reporter gene in livers of mine infected which recombinant vaccinia virus expressing the reporter construct.

ISIS 112043 is an chimeric phosphorotioate antisense directed to the 3′UTR of human heterogenous nuclear and ongogenesis. One molecule is directed to nucleotides 1137-1156 of the human cDNA sequence and contains the sequence 5′-GTGC*TTGGCTGAGTTC*AC*AA-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution. Alternatively, an antisense molecule containing the sequences 5′-AGAC*ATTTATTCAGCGTC*AC*-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, directed towards nucleotides 1376-1395 of the 3′-UTR, has been developed. These molecules, and other potential antisense sequences, are described in U.S. Pat. No. 6,165,789 and have been developed by ISIS Pharmaceutical, Inc.

ISIS 105990 is a antisense directed to the 5′-UTR mRNA of human peroxisome proliferator activated receptor (PPARγ). This protein plays a role in infection, inflammation, and tumor formation. One potential sequence is a 20 mer chimeric phosphorothioate directed towards nucleotides 17-36 of the 5′-UTR, and has the sequence 5′-AGC*AAAAGATC*AATC*C*GTTA-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution. An alternative antisense molecule has been described having sequence 5′-TTTGC*TGTAATTC*AC*AC*TGA-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, directed to nucleotides 53-72 of the 5′-UTR. These, and other potential antisense sequences are described in WO 0153311 and U.S. Pat. No. 6,159,734.

ISIS 101528 is an antisense being developed by ISIS Pharmaceuticals, Inc. that inhibits Jun N-terminal kinase kinase-2 (JNKK2) expression. One identified antisense molecule sequence, which is directed towards fragment 26-45 nt of the 5′-UTR is a 20 mer chimeric phosphorothioate oligonucleotide whose sequence is 5′-C*GC*GC*ACCGCCCGGCC*GC*C*C-3′, wherein ten central nucleotides are 2′-deoxynucleotides, the underline indicates 2′-O-(2-methyloxyethyl)nucleotide modification and a * indicates a 2′-O-(2-methoxyethyl)-5-methylcytidine substitution. An alternate antisense sequence directed towards the 60-79 nt start codon region of the mRNA is 5′-AGGAC*GCCGCCATCTTC*C*C*C*-3′, wherein the ten central nucleotides are 2′-deoxynucleotides, the underline indicates 2′-O-(2-methyloxyethyl)nucleotide modification and a * indicates a 2″-O-(2-methoxyethyl)nucleotide modification and a * indicates a 2′-O-(2-methoxyethyl)-5-methylcytidine substitution. This protein may play a role in a number of proliferation disorders, including cancer and inflammation related conditions. These antisense molecules have been described in WO 0100646.

ISIS 21329 is a chimeric phosphorothioate antisense molecule that modulates the expression of G-A-S1 mRNA levels, and plays a role in developmental disorders. One potential antisense molecule contains sequence 5′-GTTTC*GCAAAATCACTC*GGG-3′, wherein the ten central nucleotides are 2′-deoxynucleotides the underline indicates 2′-O-(2-methyloxyethyl)nucleotide modification and a * indicates a 2′-O-(2-methoxyethyl)-5-methylcytidine substitution, and is directed to nucleotides 1374-1393 of the 3′UTR. An alternative sequence containing 5′-GGGTTTCGCAAAATCAC*ATC*G-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and directed to nucleotides 1376-1395 of the 3′UTR, has also been described. These antisense molecules, as well as other alternative sequences, have been described in WO 0100861 and are being developed by ISIS Pharmaceuticals, Inc.

ISIS 25237 is a chimeric phosphorothioate antisense molecule that modulates the expression of human integrin B₃ mRNA. The antisense molecule contains the sequence 5′-GC*C*C*ATTGCTGGACATGC*-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and is directed to nucleotides 1798-1815 in the coding region. Potential uses for this antisense include the treatment of proliferation disorders such as cancer, vascular stenosis and restenosis, and bone resorption disorders. This antisense molecule, as well as other potential antisense sequences, is further described in WO 0100645 and is being developed by ISIS Pharmaceutical, Inc.

ISIS 25962 is a chimeric phosphorothioate antisense molecule that modulates the expression of G-α-i3, inhibiting asenylyl cyclase, mediating dopamine, thyrotropin-releasing hormone and somatostatin transduction pathways. One antisense molecule contains sequence 5′-GTC*TGACTTTAGCATC*TC*-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and is directed to nucleotides 1280-1297 of the coding region. An alternative antisense molecule contains sequence 5′-GGTATCTTTTCTTCTGTT-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and is directed to nucleotides 973-980 of the coding region. Areas of useful treatment include hyper-proliferation disorders. These molecules as well as other antisense molecules, are being developed by ISIS Pharmaceuticals, Inc. and have been described in WO 0100651.

ISIS 29714 is a phosphorothioate antisense molecule that modulates the expression of the human transcription factor Ets, and may be useful in the treatment of proliferation disorders such as cancer. One identified sequence includes 5′-CAAGTTGCTGCCTGGGAA-3′, and target nucleotides 1063-1080. An alternative chimeric phosphorothioate sequence includes 5′-C*C*GACGTCTTGTGGATGA-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and is directed to nucleotides 1577-1594 of the coding region These molecules, as well as others, are being developed by ISIS Pharmaceuticals, Inc. and are further described in WO 0100647.

ISIS 29176 is a chimeric phosphorothioate antisence molecule targeted to the nucleic acid encoding the serine/theonine kinase AKT3 (protein kinase B γ), which plays a role in hyper proliferation disorders, infections, and inflammation. One antisense molecule sequence is 5′-AGTC*TACTGCTCGGC*C*AT-3′, wherein the ten central nucleotides are 2′-deoxynucleotides, the underline indicates 2′-O-(2-methyloxyethyl)nucleotide modification and a * indicates a 2′-O-(2-methoxyethyl)-5 -methylcytidine substitution, and is directed to nucleotides 980-997. An alternate sequence is 5-C*TAGGCCCCACCAGTC*TA-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and is directed to nucleotides 992-1009 of the coding region. These and other antisense molecules are being developed by ISIS Pharmaceutical, Inc. and are further described in U.S. Pat. No. 6,187,586.

ISIS 28030 is a chimeric phosphorothioate antisense molecule directed to the mRNA of phosphatidylinositol 3 kinase isoform p85α (PI3 Kp85, also known as GRB1 or PIK3RI). The antisense molecule contains the sequence 5′-ATTTCCTGGGATGTGC*GG-3′, wherein the ten central nucleotides are 2′-deoxynucleotides, the underline indicates 2′-O-(2-methyloxyethyl)nucleotide modification and a * indicates a 2′-O-(2-methoxyethyl)-5-methylcytidine substitution, and is directed to nucleotides 1455-1472 of the coding region. This antisense molecule may be useful as a treatment for cancer and diabetic disorders. It has been developed by ISIS Pharmaceuticals, Inc. and is further described, along with other potential sequences, in WO 0100881.

EPI-2110 is an antisense designed to control overexpression in the lung of the A1 adenosine receptor, a key event in initiating asthma developed by EpiGenesis.

Resten-NG is an antisense that inhibits c-myc gene expression to prevent restenosis after an angioplasty by binding the mRNA AUG start site for translation, developed by AVI BioPharma, Inc. Uniquely, Resten-NG's backbone is neutral.

CYP3A4 is an antisense that targets a set of liver enzymes that metabolize drugs, such as caffeine, Viagra and nicotine, to slow drug metabolism to prolong bioavailability, developed by AVI BioPharma, Inc.

PAN-346 is an antisense that inhibits aspartyl (asparaginyl)-B hydroxylase (AAH), which localizes in the invasive periphery of brain tumors and plays a role in cell motility and invasiveness by inhibiting proteins that trigger programmed cell death, developed by Panacea Pharmaceuticals, Inc.

GEM231 is an antisense that targets the overexpressed cancer gene, the regulatory subunit of protein kinase A, developed by Hybridon in Phase II trials.

GEM-92 is an antisense that targets the overexpressed HIV or HBV gene, developed by Hybridon in Phase II trials.

HGTV43 is an antisense that targets the overexpressed HIV or HBV gene, developed by Enzo Biochem in Phase II trials.

TABLE 1
A LIST OF ANTICANCER ANTISENSE MOLECULES

| | | Proposed Mechanism of | |
|---|---|---|---|
| Drug Name | Target | Action | Cancers |
| Genasense | Bcl-2 | Inhibits Bcl-2 | Chronic |
| (G3139) | | | Lymphocytic |
| (Genta, Inc) | | | Leukemia (CLL); |
| | | | Myeloma; |
| | | | Melanoma |
| Affinitac | PKCα | Inhibits PKCα | Lung |
| (LY900003) | | | |
| (Eli Lilly & Co.) | | | |
| MG98 | DNA | By blocking DNA | Head and neck and |
| | methyltransferase | methyltransferase, this | bladder cancer |
| | | antisense molecule | |
| | | increases the activity of | |
| | | good genes called tumor | |
| | | suppressor genes and also | |
| | | reduces cell division. | |
| ISIS 2503 | Ras | Through inhibition of Ras | Breast, lung, |
| | | protein, ISIS 2503 stops | pancreatic, and |
| | | tumor cell growth. | colon |
| | | | cancer |
| ISIS 5132 | Raf | Through inhibition of Raf | Various tumors, |
| | | protein, ISIS 5132 stops | lymphoma |
| | | tumor cell growth. | |
| OGX-011/ | Clusterin | Clusterin is a survival | Prostate cancer, |
| ISIS 112989 | | protein that makes tumor | potential for renal, |
| | | cells resistant to | bladder, lung, and |
| | | chemotherapy, radiation | ovarian cancer |
| | | therapy, and hormonal | |
| | | therapy. OGX-011 will | |
| | | inhibit this protein and | |
| | | make the cells sensitive | |
| | | again. | |
| AP 12009 | TGF-b2 | TGF-b2 suppresses the | Brain tumors |
| | | immune system, stimulates | |
| | | tumor cell growth and | |
| | | migration, and stimulates | |
| | | the development of new | |
| | | blood vessels for the | |
| | | tumor. AP 12009 inhibits | |
| | | TGF-b2 production. | |
| GEM 231 | Protein Kinase A | By blocking the action of | Various cancers |
| | | protein kinase A, this | |
| | | antisense molecule blocks | |
| | | cell division. | |
| IGF- | Insulin-Like | Induces death in the tumor | Astrocytoma |
| 1R/AS/ODN | Growth Factor | cells and stimulates the | |
| | | immune system to | |
| | | eradicate the tumor. | |

TABLE 2

| COMPANY | TRADE NAME | DISEASE | TARGET |
|---|---|---|---|
| Antisense Pharma | AP 12009 | Brain tumors | TGF-beta |
| AVI BioPharma | Resten NG | Restenosis | c-myc |
| | Oncomyc NG | cancer | c-myc |
| | AVI 4126 | cancer | c-myc |
| | AVI 4557 | cancer | cytochrome P450 |
| | AVI 4014 | inflammatory disease | NF-kB |
| | Neubiotics | bacterial disease | 16S ribosome |
| | CYP3A4 | liver enzymes | cytochrome P450 |
| Corgentech | E2F Decoy | neointimal hyperplasia | E2F |
| Cytogenix | general antisense | | |
| | therapies | | |
| Enzo Therapeutics | HGTV43 | HIV | rev/tat |
| Epigenesis | EPI-2010 (Durason) | asthma | adenosine A1 |
| | | | receptor |
| Gemini | 2-5a antisense | cancer | telomerase |
| Technologies | | | |
| Genta | Genasense | cancer | Bcl-2 |
| Hybridon | GEM231 | Solid tumors | PKA-RI-α |
| | GEM92 | HIV | gag |
| Inex | INX 3280 | lymphoma, solid | c-myc |
| Pharmaceutical | | tumors | |
| | INX 3001 | CML | c-myb |
| ISIS | ISIS 2302 | ulcerative colitis, | ICAM-1 |
| Pharmaceutical | | psoriasis | |
| | ISIS 2302 | Chrohn's disease | ICAM-1 |
| | ISIS 14803 | Hepatitis C | polyprotein |
| | | | initiation codon |
| | ISIS 3521 | solid tumors | PKC-α |
| | ISIS 5132 | solid tumors | C-raf Kinase |
| | ISIS 2503 | cancer | Ha-ras |
| | Vitravene | CMV retinitis | immediate early |
| | (formivirsen, ISIS | | region 2 |
| | 2922) | | |
| | ISIS 104838 | autoimmune disease, | TNF-α |
| | | inflammatory disease, | |
| | | psoriasis | |
| | ISIS 113715 | diabetes | PTP-1B |
| | ISIS 13650 | diabetic retinopathy, | C-raf kinase |
| | | macular degeneration | |
| | ISIS 107248 | MS, inflammatory | VLA-4 |
| | | disease | |
| | ISIS 102453 | cancer | Beta-catenin |
| | ISIS 112043 | cancer | hnRNP A1 |
| | ISIS 105990 | infection, | PPARγ |
| | | inflammation, cancer | |
| | ISIS 101528 | cancer, inflammation | JNKK2 |
| | ISIS 21329 | developmental | G-α-S1 |
| | | disorders | |
| | ISIS 25237 | cancer, stenosis, | human integrin B3 |
| | | restenosis | |
| | ISIS 25962 | hyper-proliferation | G-α-i3 |
| | | disorders | |
| | ISIS 29714 | cancer | Ets |
| | ISIS 29176 | proliferation disorders, | AKT3 |
| | | inflammation, cancer | |
| | ISIS 28030 | cancer, diabetic | PI3Kp85 |
| | | disorders | |
| | OGX-011 | cancer, prostate | clusterin |
| | ISIS 23722 | cancer | survivin |
| Lorus Therapeutics | GTI 2501 | cancer | R1 ribonucleotide |
| | | | reductase mRNA |
| | GTI 2040 | cancer | R2 ribonucleotide |
| | | | reductase mRNA |
| Lynx Therapeutics | LR3001 | CML | c-myb |
| | LR3280 | restenosis | c-myc |
| | LR4437 | cancer | IGF-IR |
| Methylgene | MG98 | head and neck | DNA |
| | | carcinoma, renal cell | methyltransferase |
| | | carcinoma | |
| NeoPharm | LE-AON | cancer | c-raf |
| Novapharm | GPI-2A | HIV | gag |
| Panacea | PAN-346 | cancer | aspartyl |
| Pharmaceuticals | | | (aparaginyl)-B |
| | | | hydroxylase |
| | | | (AAH) |
| Pantheco | PNAbiotics | infectious diseases | |
| Systemix (Novartis) | rev M10polAS | HIV | pol |

(A) Phosphorothioate Antisense Oligonucleotide

Any phosphorothioate antisense oligonucleotide can be conjugated to vitamin B₁₂, ligands of transcobalamin receptors, ligands of intrinsic factor receptors or carriers of the present invention, to assist in the delivery of the antisense into the cell, preferably in a cell specific manner. Viable phosphorothioate antisense oligonucleotides include the following:

ISIS 2302 topical is a phosphorothioate oligodexoynucleotide is an intercellular adhesion molecule-1 (ICAM-1) inhibitor in Phase II studies for the treatment of Psoriasis and ulceratice colitis (UC), such as Crohn's Disease.

ISIS 14803 is a 20-base phosphorothioate oligodeoxynucleotide antisense inhibitor complementary to HCV RNA sequences adjacent to the polyprotein initiation codon of HCV, in Phase II trials. The target sequence is highly conserved among independent HCV isolates. Upon binding to the complementary target sequence, the oligonucleotide inhibits expression of HCV proteins required for HCV replication. Specific inhibition of HCV core protein expression has been demonstrated in biochemical and cell culture assays. ISIS 14803 treatment also inhibits expression of an HCV-luciferase reporter gene in livers of mice infected with recombinant vaccinia virus expressing the reporter construct.

In particular, antisense nucleotides which can be conjugated to the carriers of the present invention are distinguished in Table 7.

TABLE 3

| | | |
|---|---|---|
| Name and | | |
| Sponsor | Sequence | Target/Disease |

| | | |
|---|---|---|
| Fomivirsen | GCGTTTGCTCTTCTT CTTGCG | IE-2/CMV Retinitis |
| (Isis) | | |
| 2302 | GCCCAAGCTGGCATCCGTCA | 3′-UTR/ICAM-1, |
| (Isis) | | Crohn's Disease, |
| | | Psoriasis, |
| | | Rheumatoid |
| | | Arthritis, Ulcerative |
| | | Colitis, Renal |
| | | Allograft |
| 3521/CPG, | GTTCTCGCTGGTGAGTTTCA | 3′-UTR/PKC-a, |
| 64128A (Isis/ | | Ovarian Cancer |
| Novartis) | | |
| 5132/CPG, | TCCCGCCTGTGACATGCATT | c-RAF kinase, |
| 69846A (Isis/ | | Breast, prostrate, |
| Novartis) | | colon, brain, |
| | | ovarian cancer |
| 2503 (Isis) | TCCGTCATCGCTCCTCAGGG | Ha-ras oncogene |
| | | variety of solid |
| | | tumors |
| G3139 | TCTCCCAGCGTGCGCCAT | bcl-2, Proto |
| (Genta) | | oncogene, Non- |
| | | Hodgkin's, |
| | | Lymphoma, |
| | | Prostrate, Breast |
| LR3280 | AACGTTGAGGGCAT | c-myc/proto |
| (Lynx) | | oncogene, Stent |
| | | Restenosis |
| LR3001 | TATGCTGTGCCGGGG | c-myb, Proto |
| (Lynx) | TCTTCGGGC | oncogene, Chronic |
| | | Myeloid, Leukemia |
| LR4437 | GGACCCTCCTCCGGA GCC | IGF-IR, Ex-vitro |
| (Lynx) | | tumor cells |
| GEM-132 | UGGCGCTTACCTTGC GAACA | Intron-exon, |
| (Hybridon) | | UL36/27, CMV- |
| | | retinitis |
| GEM-92 | UCGCACCCATCTCTC TCCUUC | Gag/HIV-1, AIDS |
| (Hybridon) | | |
| GEM-231 | GCGUGCCTCCTCACU GGC | pka-1, Refractory |
| (Hybridon) | | Solid Tumors |
| GPI-2A | G(ps)GTTC(ps)TTTTG(ps) | Gag/HIV-1, AIDS |
| (Novopharm) | G(ps)TCC(ps)TTG(ps)TC | |
| | (ps)T | |
| 13312 (Isis) | GC(ps)GTTTGC(ps)TC(ps) | IE-2, CMV retinitis |
| | TTC(ps)TTC(ps)TTGCG | |

Note: The underlined bases in GEM-132, GEM-92, and GEM-231 are 2′OMe sugar modifications.
In GPI-2A, there are seven PS linkages represented by (ps) and the rest of the oligo is a phosphodiester.
In 13312, the underlined bases are 2′-O(CH₂)₂OCH₃ sugar modifications and all U and C residues are 5-methyl substituted.
Cited from: Sanghvi, Y. S. et al. in Manuals of Antisense Methodology. Eds., Hartmann, G., and Endres, S., Kluwer Academic Publisher, 1998, In Press.

B. Antisense Mimics

Antisense mimics resemble antisense in function and are comprised of non-nucleic acids that hybridize to nucleic acids or that otherwise interrupt the hybridization of nucleic acids or perform any other function of an antisense sequence. Nonlimiting examples include peptide nucleic acids (PNA), mopholinonucleic acids (MNA), locked nucleic acids (LNA), pseudocyclic oligonucleobases (PCO), and 2′-O,4′-C-ethylene bridged nucleic acids (ENA).

PNAs and MNAs

Any peptide nucleic acid, mopholinonucleic acid, locked nucleic acid, pseudocyclic oligonucleobase, or 2′-O,4′-C-ethylene bridged nucleic acid can be conjugated to vitamin B₁₂, ligands of transcobalamin receptors, ligands of intrinsic factor receptors or carriers of the present invention, to assist in the delivery of the antisense into the cell, preferably in a cell specific manner.

Peptide nucleic acids, mopholinonucleic acids, locked nucleic acids, pseudocyclic oligonucleobases, or 2′-O,4′-C-ethylene bridged nucleic acids capable of binding to vitamin B₁₂, ligands of the transcobalamin receptor, ligands of the intrinsic factor receptor, or carriers of the present invention, are analogues of DNA in which the backbone is a pseudopeptide or pseudomorpholino, respectively, rather than a sugar. The PNAs and MNAs mimic the behavior of DNA and bind complementary nucleic acid strands. The neutral backbone of PNA and MNA results in stronger binding and greater specificity than normally achieved.

The efficient and sequence specific binding to RNA or DNA combined with very high biological stability has made PNAs and MNAs extremely attractive leads for the development of gene therapeutic antisense drugs, particularly as anti-infectives (against bacterial, viral and fungal diseases) and anti-proliferatives (against cancer and other abnormal proliferative diseases such as psoriasis). It has also been shown by in vitro experiments that PNAs and MNAs may be used to control gene expression both negatively (inhibition) and positively (activation). In particular, recent results have demonstrated convincing antisense gene repression in E. coli (3), and using PNA-peptide conjugates to facilitate PNA uptake in eukaryoyic cells (4), antisense inhibition of gene expression in nerve cells and even in rat brain has been reported (Nielsen, P. E. “Peptide nucleic acids (PNA): Potential antiviral agents” Antiviral News 1993, 1, 37-39; Cherny, D. Y., Belotserkovskii, B. P., Frank-Kamenetskii, M. D., Egholm, M., Buchardt, O., Berg, R. H. & Nielsen, P. E. “DNA unwinding upon strand displacement of binding of PNA to double stranded DNA” Proc. Natl. Acad. Sci. USA. 1993, 90, 1667-1670). Thus the applications of PNA are still expanding and merely for this reason further chemical improvement and studies of PNA properties are warranted. (For recent reviews on PNA see Nielsen, P. E., Egholm. M., Berg, R. H. & Buchardt, O. “Peptide Nucleic Acids (PNA). Potential antisense and anti-gene agents” Anti Cancer Drug Design. 1993, 8, 53-63; Nielsen, P. E., Egholm. M., Berg, R. H. & Buchardt, O. “Peptide nucleic acids (PNA). DNA analogues with a polyamide backbone” In “Antisense Research and Application” Crook, S. & Lebleu, B. (eds.). CRC Press, Boca Raton, 1993, pp 363-373). In addition to their application as antisense and antigene agents, the unique chemical, physical and biological properties of PNA and MNA have been exploited to produce powerful biomolecular tools, molecular probes and biosensors. See also Peptide Nucleic Acids: Protocols and Applications Interresidue hydrogen bonding in a peptide nucleic acid-RNA heteroduplex Proceedings of the National Academy of Sciences Volume 93, Number O₂; Pages: 649-653; Improved PCR Amplification of VNTR Locus D1S80 Using PNA (Peptide Nucleic Acid Daniel B. Demers et al. Use of PNA (peptide nucleic acid) for Mycobacteria DNA purification prior to diagnostic PCR. Michael Naesby, et al. Chapter Abstracts: Molecular Biology Current Innovations and Future Trends Par Horizon Scientific Press Chapter Abstracts Application of peptide nucleic acid in cancer therapy. Anti-Cancer Drugs. 8: 113-118. Gene Cloning and Analysis: Current Innovations. Horizon Scientific Press Book Reviews Molecular Modelling of an Artificial Self-Pairing Peptide Nucleic Acid (PNA) Andrea Sommer, Ralf Lyssy, Michael Krug, Christian R. Noe et al Nucleic acid-based genetic screening methodology Maria Dulay. Peptide nucleic acid pre-gel hybridization: An alternative to Southern hybridization Proceedings of the National Academy of Sciences Volume 93, Number 25; Pages: 14670-14675; Heather Perry-O'Keefe, Xian-Wei Yao, James M. Coull, Martin Fuchs. Peptide Nucleic Acid Oligomers from Dts-Protected Monomers Knud J. Jensen, Eduard Bardaj, Fernando Albericio, James M. Coull, and George Barany Gene Chemistry: Functionally and Conformationally Intact Fluorescent Plasmid DNA; Zelphati O, Liang X, Hobart P, Felgner P L. 1999. Human Gene Therapy, Volume 10, pages 15-24.

In particular, the peptide nucleic acids which can be conjugated to the carriers of the present invention are distinguished in U.S. Pat. No. 5,864,010 entitled Peptide Nucleic Acid Combinatorial Libraries and Improved Methods of Synthesis, developed by ISIS Pharmaceuticals, which is hereby incorporated by reference. In addition, the peptide nucleic acids which can be conjugated to the carriers of the present invention are distinguished in U.S. Pat. No. 5,986,053 entitled Peptide nucleic acids complexes of two peptide nucleic acid strands and one nucleic acid strand.

In addition, Atlas Index disclosed the following Peptide Nucleic Acid, which can complex to DNA from Betts et al. NH₂—P(*C*T*C*T*T*C*T*T*C-HIS-GLY-SER-SER-GLY-HIS-C*T*T*C*T*T*C*T*C)—COOH/5′-D(GP*AP*AP*GP*AP*AP*GP*AP*G)-3′ (PNA001), which has been crystallized.

PNA oligomers can be obtained from PerSeptive Biosystems (Framingham, NIA, USA) or from authorized suppliers. Alternatively, PNA oligomers can be synthesized manually from PNA monomers obtained from PerSeptive Biosystems as described elsewhere (Norton J. C., Bioorg. Med. Chem. 3:437-445 (1995) and Cory D. R., Trends in Biotech. 15:224-229 (1997)). PNA oligomers can be any length providing they contain at least two PNA monomers. Thus, PNA oligomers can range in size from dinucleotides to entire genes or more. PNA oligomers also can have any sequence. For example, a PNA oligomer can have sequence specificity for any nucleic acid sequence that encodes a polypeptide or regulates the expression of a polypeptide. With the current advances in recombinant nucleic acid and nucleic acid sequencing technology, countless nucleic acid sequences are not only known but also readily available from sequence databases such as GenebanV.

In addition, PNA oligomers can be either modified or unmodified. Possible types of modification can include, but are not limited to, modifications with acridine, protein, backbone chemistries, DNA, peptide, bis-PNA, biotin, and fluorescein.

Peptide-based nucleic acid surrogates can also be used as a stabilized mimic for antisense technology. One class of peptide-based nucleic acid surrogates include αPNAs; the nucleobases of αPNAs are attached along one face of a peptide α-helix backbone to give a hydrid molecule capable of base-pairing to complementary ssDNA or ssRNA targets. See, for example, Garner, P.; Yoo, J. U. “Peptide-Based Nucleic Acid Surrogates Incorporating Ser[CH₂]-Gly Subunits” Tetrahedron Lett. 1993, 34, 1275-1278; Garner, P.; Dey, S.; Huang, Y.; Zhang, X. “Modular Nucleic Acid Surrogates. Solid Phase Synthesis of α-Helical Peptide Nucleic Acids (αPNAs)” Org. Lett. 1999, 1, 403-405; Garner, P.; Dey, S.; Huang, Y. “α-Helical Peptide Nucleic Acids (□PNAs): A New Paradigm for DNA-Binding Molecules” J. Am. Chem. Soc. 2000, 122, 2405-2406; U.S. Pat. No. 5,731,416 (Mar. 24, 1998).

PNAs can also include mismatch PNA oligomers. A mismatch PNA oligomer can be any PNA oligomer, including a sense or antisense PNA oligomer, having a sequence that contains at least one base pair mismatch with respect to a target sequence, such as those disclosed in PCT publication No. WO 99/20643.

(B) PNAs and MNAs Useful in Pain Therapies

The Mayo Clinic has developed peptide nucleic acids that can pass the blood-brain barrier to target neurotensin, a protein found in the brain involved in pain perception and lowering body temperature, thereby blocking neurotensins' ability to lower body temperature and reduced its ability to block the sensation of pain See, for example, PCT Publication No. WO 99/20643; “Peptide Nucleic Acids Targeted to the Neurotensin Receptor and Administered i.p. Cross the Blood-brain Barrier and Specifically Reduce Gene Expression”, B. M. Tyler et al., Proceedings of the National Academy of Sciences, 96:7053-7058, 1999. Similar peptide nucleic acids are being developed to inhibit dopamine, morphine and various other pain receptors.

(C) PNAs and MNAs Useful in Antimicrobial Therapies

Today the majority of prescribed antibiotics are naturally occurring substances or modifications hereof and resistance towards antibiotics has developed, as many bacteria have been very successful in generating antibiotic-inactivating enzymes. Many multi-resistant bacteria are able to combat antibiotics, as they are capable of producing several enzymes, which inactivate the antibiotics very efficiently. Other resistant bacteria have developed transport systems, which pump the antibiotic out of the bacteria. The best known example of resistance is the lactamase enzyme, which degrades penicillin. Other enzymes are able to modify antibiotics e.g. chloramphenicol and streptomycin.

Due to the evolutionary selection pressure, bacteria have been very efficient in developing resistance to antibiotics and also developed systems thereby they are able to pass on the resistance to other bacteria. The bacteria acquire the resistant genes encoding the various enzymes from other bacteria via different forms of gene transfer (plasmid transfer). Via such mechanisms bacteria can obtain multiresistance against various antibiotics very rapidly. In addition to the plasmid containing multi-resistance, bacteria will over time evolve new enzymes (mutation of naturally occurring or acquired bacterial enzymes) as rapidly as new antibiotics are developed by the pharmaceutical industry.

In contrast, PNAs and MNAs are a complete new chemical entity not found in nature and no microbe has inherent abilities to combat Stabilized mimics. Furthermore, no known enzymes are able to degrade it. It is therefore highly unlikely that any microbe will readily be able to produce enzymes capable of cleaving Stabilized mimic. Furthermore, as highly conserved microbial genome sequences can be selected as target sequences for the Stabilized mimic drug, it will be very “costly” for the microbe to create mutations in the target sequence. It is therefore unlikely that such mechanisms of resistance will develop. PNAs and MNAs are therefore perfect candidates for a novel anti-microbial class of drugs.

Various viable PNA-peptide antimicrobials are disclosed in “Bactericidal antisense effects of peptide-PNA conjugates” Nature Biotechnology April 2001, 19, 360-364.

In particular, antisense peptide nucleic acid (PNA) can be used to control cell growth, gene expression and growth phenotypes in the bacterium Escherichia coli. PNAs targeted to the RNA components of the ribosome can inhibit translation and cell growth, and PNAs targeted to mRNA can specifically limit gene expression, with gene and sequence specificity. For in vitro experiments, efficient inhibition is observed when using PNA concentrations in the nanomolar range, and for in vivo experiments the concentrations required are in the micromolar range. A mutant strain of E. coli that is more permeable to antibiotics is more susceptible to antisense PNAs than wild type cells.

Liam Good and Peter E. Nielsen (Curr. Issues Mol. Biol. 1999, 1(2), 111-116) disclose the effect of peptide nucleic acids (PNA) in Escherichia coli. They found that antisense peptide nucleic acid (PNA) can be used to control cell growth, gene expression and growth phenotypes in the bacteria Escherichia coli. PNAs targeted to the RNA components of the ribosome can inhibit translation and cell growth, and PNAs targeted to mRNA can limit gene expression with gene and sequence specificity. In an E. coli cell extract, efficient inhibition was observed when using PNA concentrations in the nanomolar range, whereas micromolar concentrations are required for inhibition in growing cells. A mutant strain of E. coli that is more permeable to antibiotics also is more susceptible to antisense PNAs than the wild type. Specificity of the antisense towards selective inhibition was shown via the effects of an anti-β-galactosidase PNA in comparison to control PNAs.

Therefore, in particular, these PNAs, conjugated to vitamin B₁₂, a ligand of a transcobalamin receptor, a ligand of an intrinsic factor receptor, or a carrier compound of the present invention can be used to increase the cellular uptake of these antisense PNAs to obtain effective antimicrobial agents.

In a presentation at the 23rd International Symposium on Chromatography in October 2000 entitled “Determination of Peptide Nucleic Acid (PNA) based anti-bacterials in biological matrices by on-line extraction and microbore multi-column HPLC” a method was disclosed for pharmacokinetic screening of modified PNA compounds with antibacterial effects against Gram negative bacteria. The method comprised an extraction and quantitative determination of the concentration of the modified PNA in plasma, peritoneal fluid and tissue homogenates. The biological samples were extracted on-line on an Oasis 2.1×20 mm column (Waters) and separated on two (in-line) analytical colums, either 1) C18 protein/peptide column (Vydac), 2×150 mm+Eclipse XDC-C8 (Zorbax) 2.1×50 mm (for plasma and peritoneal fluid), or 2) C8 (Vydac), 2×150 mm+218TM C18 (Vydac), 1×50 mm (for tissue homogenates). After an ultrafast wash, elution was carried out using a mobile phase of 0.1% TFA in water/acetonitrile with a gradient elution at 0.4 ml/min. The HPLC system consisted of Waters Alliance with PDA detection. Data acquisition was carried out using Millennium32. The LOQ was 80-100 ng/ml plasma or peritoneal fluid and 100 ng/g tissue.

Additional disclosure on viable PNAs which can be conjugated to obtain viable antimicrobial agents have been described by Liam Good, Satish Kumar Awasthi, Rikard Dryselius, Ola Larsson and Peter E. Nielsen “Bacterial anitsense effects of peptide-PNA conjugates” Nature Biotechnology, 2001, 19, 360-364; Good, L., Sandberg, R., Larson, O., Nielsen, P. E. and Wahlestedt, C. “Antisense PNA effects in Eschericia coli are limited by the outer membrane LPS layer” Microbiology, 2000, 146, 2665-2670; Wahlestedt, C., Salmi, P., Good, L., Kela, J., Johnsson, T., Hökfelt, T., Broberger, C., Porreca, F., Lai, J., Ren, K., Ossipov, M., Koshkin, A., Jakobsen, N., Skouv, J., Oerum, H., Havesteen-Jacobsen, M. and Wengel, J. “Locked Nucleic Acid: A novel and non-toxic oligonucleotide component for antisense studies” Proc. Natl. Acad. Sci, USA, 2000, 97, 5633-5638; Good, L. and P. E. Nielsen “Inhibition of translation and bacterial growth by peptide nucleic acid targeted to ribosomal RNA” Proc Natl Acad Sci USA, 1998, 95, 2073-2076; Good, L. and P. E. Nielsen “Antisense inhibition of gene expression in bacteria by PNA targeted to mRNA” Nature Biotechnol., 1998, 16, 355-358; and Good, L., Intine, R. V. A., and Nazar, R. N. “Interdependence in the processing of ribosomal RNAs in Schizosaccharomyces pombe” J Mol. Biol., 1998, 273, 782-788.

(D) PNAs and MNAs Useful in Viral Therapies

Reticulose: Advanced Viral Research Corp. (OTC Bulletin Board: ADVR) of Hallandale, Fla. has developed Reticulose, a peptide nucleic acid preparation that, based on previous history, has been shown to be effective against a number of viral diseases. The laboratory investigations of Dr. Hirschman, published in the Journal Of Investigative Medicine (Vol. 44) August 1996, showed that Reticulose inhibits the replication of HIV and stimulates the immune system to produce immuno-modulators (Gamma Interferon, IL1, & I16). Currently, Reticulose is in a double blind clinical AIDS trial assessing the efficacy of the drug in human patients with AIDS.

(E) PNAs and MNAs Useful in Diabetes and Cardiovascular Therapies

Isis/Panenthco has reported a peptide nucleic acid sequence useful in the treatment of diabetes and cardiovascular therapies.

(F) PNAs and MNAs Useful in Cancer Therapies

With rapidly increasing understanding of the molecular mechanisms underlying malignant transformation of human cells, not least the discoveries of a multitude of genetic mutations that are associated with uncontrolled cell growth (1), a range of gene targets are emerging as obvious candidates for gene therapeutic approaches to cancer (2,3).

Restoration of the activity of tumor suppressor gene proteins, such as p53 or pRB, lends itself to gene therapy, whereas the down regulation of oncogene products and other transforming proteins are typical goals for antisense therapy.

In vivo systems for site-directed incorporation of non-natural amino acids into proteins. The use of site-directed mutagenesis to replace amino acids at any chosen position in a protein, coupled with the development of novel analytical procedures, has greatly advanced our understanding of biological structure-function relationships in recent years. During the same period it has also become clear that although most functional requirements can be fulfilled with the standard set of twenty amino acids, this is not always so. Perhaps the most obvious example of this in nature is selenocysteine, which has become known as the 21^{st} amino acid, while another example of the use of a modified amino acid in nature is formyl-methionine. Both selenocysteine and formyl-methionine are also examples of context-dependent deciphering of the genetic code and it is this flexibility in the code which is essential during the incorporation of other non-canonical amino acids.

It has been anticipated that expanding the range of available amino acids for translation will allow biochemists to “tailor the structure of an amino acid to address a specific structure-function relation.” This has long been possible through the use of amino acid analogues that mimic their natural counterparts during protein synthesis. However, this approach has had comparatively little impact as it does not allow site-specific replacements to be made. This problem has been partially solved by the development of a technique that allows the site-specific incorporation of novel, non-natural amino acids into proteins in vitro while overcoming restrictions of protein size associated with chemical peptide synthesis. The more critical aspects of this system and its numerous applications have recently been extensively reviewed. The major advantage of this system is that it allows the incorporation of a wide range of non-natural amino acids at any codon amenable to mutagenesis to TAG. The overwhelming disadvantage is that it is strictly an in vitro technique and thus suffers limitations of both scale and scope of target proteins. The availability of a comparable in vivo system for the site-directed incorporation of non-natural amino acids would overcome these drawbacks.

Recent efforts to develop an in vivo system have focused on the engineering of aminoacyl-tRNA synthetases (AARSs) and tRNAs with a view to the development of a novel aminoacylation pathway which utilizes a non-natural amino acid and a functionally redundant tRNA independently of the normal cellular translational machinery.

(G) PNAs and MNAs as Screening Tool

Protocols for using PNAs to examine the action of enzymes that interact with DNA have been described. PNAs complementary to the RNA template of telomerase can be used to inhibit addition of telomeric repeats. PNAs can also be used to probe substrate recognition by helicases. These protocols and the results obtained through their use support the conclusion that PNAs have important advantages for studying enzymatic activity.

C. Carlsson, M. T. Dulay, R. N. Zare, J. Noolandi, B. Nordén, P. N. Nielsen, J. Zielenski, L. Tsui, and M. Jonsson. “Screening for genetic mutations,” Nature 380, (1996) was able to use peptide nucleic acids (PNAs) to detect single-base substitution in sample DNA. Because peptide nucleic acids are a fully synthetic DNA-recognizing ligand with neutral peptide-like backbones that are structurally homomorphous to the deoxyribose phosphate backbone of DNA, and purine- and pyrimidine-based nucleobases (i.e., adenine, cytosine, thymine and guanine). The neutrality of the PNA backbone results in stronger binding of PNA to DNA as compared to DNA-DNA binding. Using the mutations associated with cystic fibrosis (CF) as a model system, Carlsson et al. have demonstrated that PNA can distinguish normal and mutant sequences in the CF gene.

(H) PNAs and MNAs as Probes

PNAs and MNAs have applications in hybridization based DNA detection methods such as PCR, in situ hybridization and DNA biosensors, and for use as a diagnostic probe for detecting genetic mutations, as well as mismatch analysis. In particular, the PNAs and MNAs conjugated to the vitamin B₁₂, ligands of the transcobalmin receptor, ligands of the intrinsic factor receptor, or carriers of the present invention, can also be labeled, for example with a detectable agent, such as a fluorescent marker, to provide detection of the hybridized complex.

For example, the PNAs and MNAs conjugated to the vitamin B₁₂, ligands of the transcobalmin receptor, ligands of the intrinsic factor receptor, or carriers of the present invention, can be labeled with biotin, digoxigenin, flourescent dyes, thiazole orange (see, for example, Svanvik et al., Analytical Biochemistry: 281:26-35, 2000); or reporter enzymes. The PNAs and MNAs conjugated to the vitamin B₁₂, ligands of the transcobalmin receptor, ligands of the intrinsic factor receptor, or carriers of the present invention, can be probes for hybridization experiments such as DNA arrays, Northern blots, Southern blots, FISH, detection of single point mutations, or DNA mapping.

In situ hybridization based techniques are gaining increasing importance in a wide variety of areas such as molecular biology, microbiology, histochemistry, cytogenetics, cytochemistry and others. Over the latest years the use of PNA probes for in situ hybridization in these areas have been explored by several groups. These investigations consistently show that PNA probes are superior to traditional oligonucleotide probes. The unique physico-chemical properties of PNA probes translate into unique behaviour in in situ hybridization that enable design of sensitive, robust and user-friendly protecols.

In recent years, the use of PNA-array technologies for the analysis of nucleic acids has taken a leap forward, with much more still to come. Apart from miniaturisation and other technical advances, also the chemistry of the production of such arrays is a focus of attention. Because of the unique features of PNA-DNA interaction, the use of arrayed PNA oligomers could be superior for such purposes in many respects.

Non-labeled PNA “blocker” probes can be used to prevent mismatch hybridization of labeled probes to non-target sequences. The use of PNA blockers significantly decreases unwanted hybridization without a corresponding decrease in the sensitivity of detection of complementary targets. Furthermore, PNA probes and blockers provided higher signal to noise ratios than corresponding probes and blockers made of DNA. As a result, following PCR amplification, it is possible to detect a single base mutation in the K-ras gene at levels of only 1.5 copies per 100 copies of wild type DNA.

The detection of single nucleotide polymorphisms in DNA can also be achieved using allele-specific, mass-labeled, PNA hybridization probes, and analysis by matrix-assisted laser desorption/ionization (MALDI) time-of-flight (TOF) mass spectrometry (MS). MALDI-TOF MS detection of the PNA probes produces composite mass spectra containing peaks of distinct masses corresponding to each allele present, resulting in a mass spectral “fingerprint” for each DNA sample. PNA oligomers offer unique advantages in their use as allele-specific hybridization probes and for their detection by MALDI-TOF MS. The hybridization characteristics of PNA-DNA duplexes are highly dependent on both base content and sequence. For example, one can analyze single nucleotide polymorphisms contained in exon 4 of the human tyrosinase gene.

Biosensor devices, based on the conversion of nucleic acid recognition reactions into useful electrical signals, offer considerable promise for DNA diagnostics. The unique hybridization properties of solution-phase PNA can be extrapolated onto transducer surfaces in connection with the design of remarkably specific DNA biosensors.

PNA-assisted rare cleavage (PARC) is based on the general Achilles' heel cleavage strategy. The PARC technique makes it possible to convert usual restriction enzymes into infrequent genome cutters. In this method, a very stable and sequence-specific complex is formed between double-stranded genomic DNA and a cationic pyrimidine bis-PNA. Then the sample is treated with a DNA methyltransferase (methylase), the bis-PNA is removed from the DNA and the sample is treated with a restriction enzyme. The restriction enzyme recognizes the same sites as the methylase did and thus cannot cleave them. The only exceptions are very few non-methylated sites, which were protected against methylation by the bis-PNA overlapping the methylation sites. These rare sites become accessible for enzymatic recognition after PNA is removed. As a result, the restriction enzyme cuts the genomic DNA into a small number of fragments with lengths from several hundreds kbp to several Mbp. A pool of numerous combinations of various bis-PNAs with different methylation/restriction enzymatic pairs generates a new class of genome rare cutters. These cutters cover the range of recognition specificities, where very few, if any, cutters are now available. Biomolecular tools of that kind may find applications for processing chromosomes.

The sequence-specific isolation and purification of intact double-stranded DNA (dsDNA) by oligonucleotide/PNA-assisted affinity capture (OPAC), i.e. the OPAC assay, is based on selective tagging of a DNA duplex by biotinylated oligodeoxyribonucleotide (ODN) through formation of a so-called PD-loop. The PD-loop is assembled with the aid of a pair of PNA “openers” that allow sequence-specific targeting with a Watson-Crick complementary ODN probe in the exposed region of the dsDNA. The protocol involves three steps. First, two cationic bis-PNAs locally pry the DNA duplex apart at a predetermined site. Then, the exposed DNA single strand is targeted by a complementary biotinylated ODN to selectively form a stable PD-loop complex. Finally, the capture of dsDNA is performed using streptavidin covered magnetic beads. This OPAC procedure has many advantages in the isolation of highly purified native DNA over other affinity capture and amplification techniques.

The use of affinity tagged PNA capture probes offers an efficient means for the purification of nucleic acids by hybridization. Two different approaches are described. A sequence specific method and a generic method. The sequence specific method requires sequence information on the target and synthesis of a dedicated PNA. It can be used to selectively purify the nucleic acid containing the target from non-related nucleic acids and other cellular components. The generic method uses a “universal” triplex forming PNA and requires no sequence information on the target. It can be used in the bulk purification of large nucleic acids.

An efficient, PCR based method for the selective amplification of DNA target sequences that differs by a single base pair is described. The method utilizes the high affinity and specificity of PNA for their complementary nucleic acids and that PNA cannot function as primers for DNA polymerases.

An effective approach using a peptide nucleic acid (PNA) ‘clamp’ to directly and essentially irreversibly modify plasmid DNA, without affecting either its supercoiled conformation or its ability to be efficiently transcribed has also been described. As an example, we demonstrate the generation of a highly fluorescent preparation of plasmid DNA by hybridizing fluorescently labeled PNA to the plasmid. Fluorescent plasmid prepared in this way is neither functionally nor conformationally altered. The PNA clamp binding is sequencespecific, saturable, extremely stable, and does not influence the nucleic acid intracellular distribution. This method can be utilized to study the biodistribution of conformationally intact plasmid DNA in living cells after cationic lipid mediated transfection. A Rhodaminelabeled fluorescent plasmid expressing green fluorescent protein (GFP) enables simultaneous colocalization of both plasmid and expressed protein in living cells and in realtime.

A series of dyes, called carbocyanine dyes, are being studied in terms of their relationship to PNA-DNA hybrids. These dyes, due to a sensitivity to electric potential, were initially used by molecular biologists to investigate changes in electric potential across the plasma membrane of nerve cells. These dyes are multi-ring aromatic compounds and absorb intensely in the visible range, resulting in bright blue-greenish colors for their solutions.

Carbocyanine dyes do not have to be physically attached to a PNA strand to be useful in the laboratory. Rather, they preferentially bind to PNA-DNA hybrids in solution, creating a color change. To be more specific, multiple dye molecules will bind to a PNA-DNA hybrid in the minor groove of the double helix. An increase in wavelength turns a blue dye solution purple when exposed to a PNA-DNA hybrid. This color change mechanism forms the basis for a simple method of detecting PNA-DNA hybrids visually.

The mechanism by which this change occurs is believed to be an interaction between the electrons in the aromatic rings in successive dye molecules that results in an increase in the wavelength of the emitted photons.

The localization of trinucleotide repeat sequences in myotonic dystrophy cells was discovered using a single fluorochrome-labeled PNA probe (BioTechniques 1998 March; 24(3), 472-476). A labeled peptide nucleic acid (PNA) antisense probe was used to study the spatial distribution of triplet repeats (CTG) in human myotonic dystrophy (DM) cells by high-resolution fluorescence in situ hybridization (FISH). It was found that transcripts containing triplet repeats were present as a number of discrete foci in the DM nuclei. Greater numbers of foci were visible with the PNA probe than a comparable DNA probe. The PNA probe was also used to visualize the triplet expansion within the DM gene located on the chromosome as a reference, it was estimated there were between 15-230 RNA molecules in each focus observed in DM nuclei.

In another example, PNAs conjugated to flourescent markers can be combined with a cationic conjugated polymer. The light-harvesting properties of the cationic conjugated polymers can be used to sensitize the emission of a dye on a PNA sequence for the purpose of homogeneous, “real-time”, highly sensitive DNA detection in which signal transduction is controlled by hybridization of the neutral PNA probe and the negative DNA target. See, for example, Gaylord et al Proc. Natl. Acad. Sci. USA, Vol. 99, Issue 17, 10954-10957.

LNAs

LNA is a novel class of DNA analogues that possess some features that make it a prime candidate for improving nucleic acid properties. The LNA monomers are bi-cyclic compounds structurally similar to RNA-monomers. LNA share most of the chemical proprties of DNA and RNA, it is water-soluble, can be separated by gel electrophoreses, ethanol precipitated etc (Tetrahedron, 54, 3607-3630 (1998)). However, introduction of LNA monomers into either DNA or RNA oligos results in high thermal stability of duplexes with complementary DNA or RNA, while, at the same time obeying the Watson-Crick base-pairing rules. This high thermal stability of the duplexes formed with LNA oligomers together with the finding that primers containing 3′ located LNA(s) are substrates for enzymatic extensions, e.g. the PCR reaction, is used in the present invention to significantly increase the specificity of detection of variant nucleic acids in the in vitro assays described in the application. The amplification processes of individual alleles occur highly discriminative (cross reactions are not visible) and several reactions may take place in the same vessel. See for example U.S. Pat. No. 6,316,198.

Certain conformational restriction has been applied in recent years to oligonucleotides in the search for analogues displaying improved hybridisation properties compared to unmodified (2′-deoxy)oligonucleotide-s. For instance, there have been reported bicyclo[3.3.0]nucleosides with an additional C-3′, c-5′-ethano-bridge (see e.g., M. Tarkoy et al., Helv. Chim. Acta, 1993, 76, 481); bicarbocyclo[3.1.0]nucleosides with an additional C-1′, C-6′- OR C-6′, C-4′ methano bridge (see e.g., K.-H. Altmann et al., Tetrahedron Lett., 1994, 35, 2331); bicyclo[3.3.0]- and [4.3.0] nucleosides containing an additional C-2′, C-3′-dioxalane ring synthesised as a dimer with an unmodified nucleoside where the additional ring is part of the internucleoside linkage replacing a natural phosphordiester linkage (see e.g., R. J. Jones et al., J. Am. Chem. Soc., 1993, 115, 9816); dimers containing a bicyclo[3.1.0] nucleoside with a C-2′, C-3′-mthano bridge as part of amide- and sulfonamide type internucleoside linkages (see e.g., C. G. Yannopoulus et al., Synlett, 1997, 378); bicyclo[3.3.0] glucose-derived nucleoside analogue incorporated in the middle of a trimer through formacetal internucleoside linkages (see e.g., C. G. Yannopoulus et al., Synlett, 1997, 378); tricyclo-DNA in which two five membered rings and one three membered ring constitute the backbone (see R. Steffens & C. J. Leumann, J. Am. Chem. Soc, 1997, 199, 11548-49); 1,5-Anhydrohexitol nucleic acids (see Aerschot et al., Angew. Chem. Int. Ed. Engl. 1995, 34(129 1338-39); and bicyclic[4.3.0]- and [3.3.0] nucleosides with additional C-2′, C-3′-connected six and five-membered ring; (see e.g., P. Nielsen et al., XII International Roundtable: Nucleosides, Nucleotides and Their Biological Applications, La Jolla, Calif., Sep. 15-19, 1996, Poster PPI 43). However, oligonucleotides comprising these analogues form in most cases less stable duplexes with complementary nucleic acids compared to the unmodified oligonucleotides.

Locked Nucleic Acids (LNA) have been described (see International Patent Application WO 99/14226; P. Nielsen et al., J. Chem. Soc., Perkin Trans. 1, 1997, 3423; P. Nielsen et al., Chem. Commun., 1997, 9, 825; N. K. Christensen et al., J. Am. Chem. Soc., 1998, 8, 2219-2222; and S. Obika et al., Bioorg. Med. Chem. Lett., 1999, 515). Incorporation of LNA monomers containing a 2′-0,4′-C-methylene bridge into an oligonucleotide sequence led to an improvement in the hybridisation stability of the modified oligonucleotide (see above and e.g., S. K. Singh et al., Chem. Commun., 1998, 455). Olignucleotides comprising the 2′-0,4′-C-methylene bridge (LNA) monomers and also the corresponding 2′-thio-LNA (thio-LNA), 2′-HN-LNA (amino-LNA), and 2′N-(R)-LNA (amino-R-LNA) analogue, form duplexes with complementary DNA and RNA with very favorable thermal stabilities.

The LNA modified antisense oligonucleotide may comprise antisense oligonucleotides specific to any tumour suppressor genes such as TP53, RB1, P16, oncogenes such as RAS and MYC or DNA repair genes such as MSH2 and MLH1 involved in the establishment and growth of a tumour. It may also be targeted against genes which are involved in tumour angiogenesis and metastasis such as for example the genes MMP-1 and MMP-2 which bg to the MMP family of matrix metalloproteinases that degrade connective tissue. Also, the LNA modified oligonucleotides may be directed against genes encoding multidrug transporter proteins such as the genes MDR-1 and MDR-2. Overexpression of such genes leads to multidrug resistance which is a major limitation to the success of current chemotherapy. Also, the LNA modified oligonucleotide may be directed against genes involved in the signal transduction pathway regulating cell growth such as cyclin dependent kinases.

Table 4 below lists a number of genes involved in the establishment, growth, invasion and metastasis of tumors and genes involved in the development of resistance to chemotherapeutic drugs that are particularly interesting as antisense targets. It should be understood that many of the genes listed in Table I are representatives of a larger gene family, the other members of which also constitute potentially important antisense targets, e.g., ADAMTS-1 is a member of the ADAMs gene family that encode cellular disintegrins and metalloproteinases, MMP-1 is a member of the matrix metalloproteinases (MMPs) gene family that encode zinc-dependent endoproteinases, etc.

LNA modified oligonucleotides may be used to modulate the expression of genes involed in inflammatory diseases. Below are lists of some genes such CD markers, adhesion molecules, chemokines and chemokine receptors, and interleukins and their receptors: CD markers CD1a-d CD30 CD61 CD91 CD121 CD2 CD31 CD62E CDw92 CD122 CD3 CD32 CD62L CD93 CDw123 CD4 CD33 CD62P CD94 CD124 CD5 CD34 CD63 CD95 CDw125 CD6 CD35 CD64 CD96 CD126 CD7 CD36 CD65 CD97 CD127 CD8 CD37 CD66a-e CD98 CDw128 CD9 CD38 CD67 CD99 CD129 CD10 CD39 CD68 CD100 CD130 CD11a CD40 CD69 CD101 CDw131 CD11b CD41 CD70 CD102 CD132 CD11c CD42a-d CD71 CD103 CD133 CDw12 CD43 CD72 CD104 CD134 CD13 CD44 CD73 CD105 CD14 CD45 CD74 CD106 CD15 CD46 CDw75 CD107a,b CD16 CD47 CDw76 CDw08 CDw17 CD48 CD77 CD109 CD18 CD49a-f CDw78 CD110 CD19 CD50 CD79a,b CD111 CD20 CD51 CD80 CD 112 CD21 CD52 CD81 CD 113 CD22 CD53 CD82 CD 114 CD23 CD54 CD83 CD115 CD24 CD55 CDw84 CD116 CD25 CD56 CD85 CD117 CD26 CD57 CD86 CD118 CD27 CD58 CD87 CD119 CD28 CD59 CD88 CD120a,b CD29 CDw60 CD89 CD30 CD90; Adhesion molecules: L-selectin TCRγ/δBB-1; Integrin α7; Integrin α6; P-selectin; CD28; N-cadherin; Integrin α8; Integrin β5; E-selectin; LFA-3; E-cadherin; P-Integrinα; V Integrin α; V HNK-1; PECAM-1; cadherin; Integrin P2; Integrin P6; Sialyl-VCAM-1; Integrin β1; Integrinα; L Integrin α; V Lewis X; CD15; ICAM-2; Integrin α1; Integrin αM; Integrin β7; LFA-2; ICAM-3; Integrin α2; IntegrinoαX; Integrinoα; IEL; CD22; Leukosialin; Integrin α3 Integrin α3; Integrin α4; ICAM-1; HCAM; Integrin α4; IntegrinoαV; Integrinβ8; N-CAM; CD45RO; Integrin α5; Integrin αlib; Integrin αV; NG-CAM; CD5; Integrin α6; Integrinα4 TCRα/β; HPCA-2; Chemokines and Chemokine receptors: C—X—C hemokine chemokines C—C chemokines C chemokines eceptors IL-8 MCAF/MCP-1 ABCD-1 Lymphotactin CCR1 NAP-2 MIP-1.alpha.,.beta. LMC CCR2 GRO/MGSA RANTES AMAC-1 CCR3.gamma.IP-10I-309 NCC-4 CCR4 ENA-78 CCF18 LKN-1 CCR5 SDF-1 SLC STCP-1 CCR6 I-TAC TARC TECK CCR7 LIX PARC EST CCR8 SCYB9 LARC MDC CXCR1 B cell-EB1 Eotaxin CXCR2 attracting chemokine 1 HCC-1 CXCR3 HCC-4 CXCR5 CX.sub.3CR; Interleukins and their receptors: G-CSF IL-2 R.alpha. IL-8-IL-16 TGF-.beta.1 G-CSF R IL-2 R.beta. IL-9 IL-17 TGF-.beta.1,2 GM-CSF IL-2 R.gamma.IL-9 R IL-18 TGF-.beta.2 IFN-.gamma. IL-3 IL-10 PDGF TGF-.beta.3 IGF-I IL-3 R.alpha. IL-10 R PDGF A Chain TGF-.beta.5 IGF-I R IL-4 IL-11 PDGF-AA LAP TGF-.beta.1 IGF-II IL-4 R IL-11 R PDGF-AB Latent TGF-.beta.1 IL-1.alpha. IL-5 IL-12 PDGF B Chain TGF-.beta. bpl IL-1.beta.IL-5 R.alpha. IL-12 p40 PDGF-BB TGF-.beta R¹¹ IL-1 R¹ IL-6 IL-12 p70 PDGF R.alpha. TGF-.beta. RIII IL-1 R¹¹ IL-6 R IL-13 PDGF R.beta. IL-Ir.alpha. IL-7 IL-13 R.alpha. TGF-.alpha. IL-2 IL-7 R IL-15 TGF-.beta.

It should be appreciated that an indicated gene means the gene and all currently known variants thereof, including the different mRNA transcripts that the gene and its variants can give rise to, and any further gene variants which may be elucidated. In general, however, such variants will have significant homology (sequence identify) to a sequence of a table above, i.e. a variant will have at least about 70 percent homology (sequence identity) to a sequence of the above tables 2-5, more typically at least about 75, 80, 85, 90, 95, 97, 98 or 99 homology (sequence identity) to a sequence of the above tables 2-5. Homology of a variant can be determined by any of a number of standard techniques such as a BLAST program. Sequences for the genes listed in can be found in GenBank (http://www.ncbi.nlm.nih.gov/). The gene sequences may be genomic, cDNA or mRNA sequences.

Incorporation of LNA monomers into a standard DNA or RNA oilgonuclotide will increase its resistance towards nucleases (endonucleases and exonucleases), the extent of which will depend on the number of LNA monomers used and their position in the oligonucleotide. Nuclease resistance of LNA-modified oligonucleotides can be further enhanced by providing nuclease-resistant internucleosidic linkages. Many such linkages are known in the art, e.g., phosphorothioate: Zon and Geiser, Anti-Cancer Drug Design, 6:539-568 (1991); U.S. Pat. Nos. 5,151,510; 5,166,387; and 5,183,885; phosphorodithioates: Marshall et al., Science, 259:1564-1570 (1993); Caruthers and Nielsen, International Patent Application PCT/US89/02293; phosphoramidates, e.g., —O.sub.2P(.dbd.O)(NR), where R may be hydrogen or C1-C-3 alkyl; Jager et al., Biochemistry, 27:7237-7246 (1988); Froehler et al., International application PCT/US90/03138; peptide nucleic acids: Neilsen et al, Anti-Cancer Drug Design, 8:53-63 (1993), International application PCT/EP92/01220; methylphosphonates: U.S. Pat. Nos. 4,507,433; 4,469,863; and 4,757,055; and P-chiral linkages of various types, especially phosphorothioates, Stec et al., European patent application 506,242 (1992) and Lesnikowski, Bioorganic Chemistry, 21:127-155 (1993). Additional nuclease linkages include phosphoroselenoate, phosphorodiselenoate, alkylphosphotriester such as methyl- and ethylphosphotriester, carbonate such as carboxymethyl ester, carbamate, morpholino carbamate, 3′thioformacetal, silyl such as dialkyl (C₁-C₆)— or diphenylsilyl, sulfamate ester, and the like. Such linkages and methods for introducing them into oligonucleotides are described in many references, e.g. reviewed generally by Peyman and Ulmann, Chemical Reviews 90:543-584 (1990); Milligan et al., J. Med. Chem., 36:1923-1937 (1993); Matteucci et al., International application PCT/US91/06855. Resistance to nuclease digestion may also be achieved by modifying the internucleotide linkage at both the 5′ and 3′ termini with phosphoroamidites according to the procedure of Dagle et al., Nucl. Acids Res. 18, 4751-4757 (1990).

Where triplex formation is desired, there are constraints on the selection of target sequences. Generally, third stand association via Hoogsteen type of binding is most stable along homopyrimidine-homopurine tracks in a double stranded target. Usually, base triplets form in T-A*T or C-G*C motifs (where “-” indicates Watson-Crick pairing and “*” indicates Hoogsteen type of binding); however, other motifs are also possible. For example, Hoogsteen base pairing permits parallel and antiparallel orientations between the third stand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments, e.g., Roberts et al., Proc. Natl. Acad. Sci., 88:9397-9401 (1991); Roberts et al., Science, 58:1463-1466 (1992); Distefano et al, Proc. Natl. Acad. Sci., 90:1179-1183 (1993); Mergny et al., Biochemistry, 30:9791-9798 (1992); Cheng et al., J. Am. Chem. Soc., 114:4465-4474 (1992); Beal and Dervan, Nucleic Acids Research, 20:2773-2776 (1992); Beal and Dervan, J. Am. Chem. Soc., 114:4976-4982; Giovannangeli et al, Proc. Natl. Acad. Sci., 89:8631-8635 (1992); Moser and Dervan, Science, 238:645-650 (1987); McShan et al, J. Biol. Chem., 267:5712-5721 (1992); Yoon et al., Proc Natl. Acad, Sci., 89:3840-3844 (1992); and Blume et al., Nucleic Acids Research, 20:1777-1784 (1992).

The length of the oligonucleotide moieties is sufficiently large to ensure that specific binding will take place only at the desired target polynucleotide and not at other fortuitous sites, as explained in many references, e.g., Rosenberg et al., International application PCT/US92/05305; or Szostak et al, Meth. Enzymol, 68:419-429 (1970). The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, whether modifications to enhance binding or specificity are present, whether duplex or triplex binding is desired, and the like. Usually, antisense compounds of the invention have lengths in the range of about 12 to 40 nucleotides. More preferably 30 nucleotides; and most preferably, they have lengths in the range of about 12 to 20 nucleotides.

PCOs and ENAs

Pseudo-cyclic oligonucleobases (PCOs) can also be used in the regulator (see for example U.S. Pat. No. 6,383,752). PCOs contain two oligonucleotide segments attached through their 3′-3′ or 5′-5′ ends. One of the segments (the “functional segment”) of the PCO has some functionality (e.g., an antisense oligonucleotide complementary to a target mRNA). Another segment (the “protective segment”) is complementary to the 3′- or 5′-terminal end of the functional segment depending on the end through which it is attached to the functional segment). As a result of complementarity between the functional and protective segment segments, PCOs form intramolecular pseudo-cyclic structures in the absence of the target nucleic acids (e.g., RNA). PCOs are more stable than conventional antisense oligonucleotides because of the presence of 3′-3′ or 5′-5′ linkages and the formation of intramolecular pseudo-cyclic structures. Pharmacokinetic, tissue distribution, and stability studies in mice suggest that PCOs have higher in vivo stability than and, pharmacokinetic and tissue distribution profiles similar to, those of PS-oligonucleotides in general, but rapid elimination from selected tissues. When a fluorophore and quencher molecules are appropriately linked to the PCOs of the present invention, the molecule will fluoresce when it is in the linear configuration, but the fluorescense is quenched in the cyclic conformation.

2′-O,4′-C-ethylene bridged nucleic acids (ENAs) are another class of stabilized antisense mimics. See, for example, Morita K, hasegawa C, Kaneko M, Tsutsumi S, Sone J, Ishikawa T, Imanishi T, Koizumui M; 2′-O,4′-C-ethylene bridged nucleic acids (ENA): highly nuclease resistant and thermodynamically stable olionucleotides for antisense drugs. Bioorg Med Chem Lett 2002 Jan. 7; 12(1):73-6; Synthesis of 2′-O-[2-[N,N-dimethylamino)oxy]ethyl] modified nucleosides and oligonucleotides. Prakash T P, Kawasaki A M, Fraser A S, Vasquez G, Monoharan M. J Org Chem 2002 Jan. 25;67(2):357-69.

VI. Delivery of Aptamers

In one embodiment, aptamers can be effectively delivered to cells by conjugation to a ligand for the transcobalamin receptor or intrinsic factor receptortranscobalamin receptor.

Aptamers are nonencoding single-stranded nucleic acid (DNA or RNA) that have the property of binding specifically to a desired target compound or molecule, and which have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. The SELEX method involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising segments of randomized sequences, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity aptamers to the target molecule.

Aptamers possess a number of features that can render them useful as therapeutic agents. They can be made as relatively small (8 kDa to 15 kDa) synthetic compounds and can be selected to possess high affinity and specificity for target molecules (equilibrium dissociation constants ranging from 0.05-10 nM). Aptamers embody both the affinity properties of monoclonal antibodies and single chain antibodies (scFv's) and the manufacturing ease similar to that of a small peptide. Initial studies demonstrated the in vitro use of aptamers for studying protein function, and more recent studies have confirmed the utility of these compounds for studying in vivo protein function (Floege et al, Am J Pathol 154:169-179 (1999), Ostendorf et al, J Clin Invest 104:913-923, (1999)). In addition, animal studies to date have shown that aptamers and compounds of similar composition are well tolerated, exhibit low or no immunogenicity, and are thus suitable for repeated administration as therapeutic compounds (Floege et al, Am J Pathol 154:169-179 (1999), Ostendorf et al, J Clin Invest 104:913-923 (1999), Griffin et al, Blood 81:3271-3276 (1993), Hicke et al, J Clin Invest 106:923-928 (2000)).

Aptamers (also sometimes referred to as nucleic acid ligands) and methods for their production and use, are described, for example, in the following U.S. patents. Any of the nucleic acid ligands described in the patents listed below or other patents, or any nucleic acid ligands described in publications as well as other desired nucleic acid ligands used in medical therapy can be modulated or regulated according to the present invention: U.S. Pat. No. 6,387,635, entitled 2′-fluoropyrimidine anti-calf intestinal phosphatase nucleic acid ligands; U.S. Pat. No. 6,387,620, entitled Transcription-free selex; U.S. Pat. No. 6,379,900, entitled Compositions and methods of use of 8-nitroguanine; U.S. Pat. No. 6,376,474, entitled Systematic evolution of ligands by exponential enrichment: tissue SELEX; U.S. Pat. No. 6,376,190, entitled Modified SELEX processes without purified protein; U.S. Pat. No. 6,355,787, entitled Purine nucleoside modifications by palladium catalyzed methods and compounds produced; U.S. Pat. No. 6,355,431, entitled Detection of nucleic acid amplification reactions using bead arrays; U.S. Pat. No. 6,346,611, entitled High affinity TGFβ nucleic acid ligands and inhibitors; U.S. Pat. No. 6,344,321, entitled Nucleic acid ligands which bind to hepatocyte growth factor/scatter factor (HGF/SF) or its receptor c-met; U.S. Pat. No. 6,344,318, entitled Methods of producing nucleic acid ligands; U.S. Pat. No. 6,331,398, entitled Nucleic acid ligands; U.S. Pat. No. 6,331,394, entitled Nucleic acid ligands to integrins; U.S. Pat. No. 6,329,145, entitled Determining non-nucleic acid molecule binding to target by competition with nucleic acid ligand; U.S. Pat. No. 6,306,598, entitled Nucleic acid-coupled colorimetric analyte detectors; U.S. Pat. No. 6,303,316, entitled Organic semiconductor recognition complex and system; U.S. Pat. No. 6,300,074, entitled Systematic evolution of ligands by exponential enrichment: Chemi-SELEX; U.S. Pat. No. 6,291,184, entitled Systematic evolution of ligands by exponential enrichment: photoselection of nucleic acid ligands and solution selex; U.S. Pat. No. 6,287,765, entitled Methods for detecting and identifying single molecules; U.S. Pat. No. 6,280,943, entitled 2′-fluoropyrimidine anti-calf intestinal phosphatase nucleic acid ligands; U.S. Pat. No. 6,280,932, entitled High affinity nucleic acid ligands to lectins; U.S. Pat. No. 6,264,825, entitled Binding acceleration techniques for the detection of analytes; U.S. Pat. No. 6,261,783, entitled Homogeneous detection of a target through nucleic acid ligand-ligand beacon interaction; U.S. Pat. No. 6,261,774, entitled Truncation selex method; U.S. Pat. No. 6,242,246, entitled Nucleic acid ligand diagnostic Biochip; U.S. Pat. No. 6,232,071, entitled Tenascin-C nucleic acid ligands; U.S. Pat. No. 6,229,002, entitled Platelet derived growth factor (PDGF) nucleic acid ligand complexes; U.S. Pat. No. 6,225,063, entitled RNA channels in biological membranes; U.S. Pat. No. 6,207,816, entitled High affinity oligonucleotide ligands to growth factors; U.S. Pat. No. 6,207,388, entitled Compositions, methods, kits and apparatus for determining the presence or absence of target molecules; U.S. Pat. No. 6,184,364, entitled High affinity nucleic acid ligands containing modified nucleotides; U.S. Pat. No. 6,183,967, entitled Nucleic acid ligand inhibitors to DNA polymerases; U.S. Pat. No. 6,180,348, entitled Method of isolating target specific oligonucleotide ligands; U.S. Pat. No. 6,177,557, entitled High affinity ligands of basic fibroblast growth factor and thrombin; U.S. Pat. No. 6,177,555, entitled Homogeneous detection of a target through nucleic acid ligand-ligand beacon interaction; U.S. Pat. No. 6,171,795, entitled Nucleic acid ligands to CD40 ligand; U.S. Pat. No. 6,168,778, entitled Vascular endothelial growth factor (VEGF) Nucleic Acid Ligand Complexes; U.S. Pat. No. 6,147,204, entitled Nucleic acid ligand complexes; U.S. Pat. No. 6,140,490, entitled High affinity nucleic acid ligands of complement system proteins; U.S. Pat. No. 6,127,119, entitled Nucleic acid ligands of tissue target; U.S. Pat. No. 6,124,449, entitled High affinity TGFβ nucleic acid ligands and inhibitors; U.S. Pat. No. 6,114,120, entitled Systematic evolution of ligands by exponential enrichment: tissue selex; U.S. Pat. No. 6,110,900, entitled Nucleic acid ligands; U.S. Pat. No. 6,083,696, entitled Systematic evolution of ligands exponential enrichment: blended selex; U.S. Pat. No. 6,080,585, entitled Methods for discovering ligands; U.S. Pat. No. 6,051,698, entitled Vascular endothelial growth factor (VEGF) nucleic acid ligand complexes; U.S. Pat. No. 6,048,698, entitled Parallel SELEX; U.S. Pat. No. 6,030,776, entitled Parallel SELEX; U.S. Pat. No. 6,028,186, entitled High affinity nucleic acid ligands of cytokines; U.S. Pat. No. 6,022,691, entitled Determination of oligonucleotides for therapeutics, diagnostics and research reagents; U.S. Pat. No. 6,020,483, entitled Nucleoside modifications by palladium catalyzed methods; U.S. Pat. No. 6,020,130, entitled Nucleic acid ligands that bind to and inhibit DNA polymerases; U.S. Pat. No. 6,013,443, entitled Systematic evolution of ligands by exponential enrichment: tissue SELEX; U.S. Pat. No. 6,011,020, entitled Nucleic acid ligand complexes; U.S. Pat. No. 6,001,988, entitled High affinity nucleic acid ligands to lectins; U.S. Pat. No. 6,001,577, entitled Systematic evolution of ligands by exponential enrichment: photoselection of nucleic acid ligands and solution selex; U.S. Pat. No. 6,001,570</netacgi/nph-Parser?Sect 1=PTO2& Sect2=HITOFF&p=2&u=/netahtml/search-bool.html&r=57&f=G&1=50&col=AND&d=pall&s1=‘5,475,096’&OS=>, entitled Compositions, methods, kits and apparatus for determining the presence or absence of target molecules; U.S. Pat. No. 5,998,142, entitled Systematic evolution of ligands by exponential enrichment: chemi-SELEX; U.S. Pat. No. 5,989,823, entitled Homogeneous detection of a target through nucleic acid ligand-ligand beacon interaction; U.S. Pat. No. 5,972,599, entitled High affinity nucleic acid ligands of cytokines; U.S. Pat. No. 5,962,219, entitled Systematic evolution of ligands by exponential enrichment: chemi-selex; U.S. Pat. No. 5,958,691, entitled High affinity nucleic acid ligands containing modified nucleotides; U.S. Pat. No. 5,874,557, entitled Nucleic acid ligand inhibitors to DNA polymerases; U.S. Pat. No. 5,874,218, entitled Method for detecting a target compound in a substance using a nucleic acid ligand; U.S. Pat. No. 5,871,924, entitled Method for the production of ligands capable of facilitating aminoacyl-RNA synthesis; U.S. Pat. No. 5,869,641, entitled High affinity nucleic acid ligands of CD4; U.S. Pat. No. 5,864,026, entitled Systematic evolution of ligands by exponential enrichment: tissue selex; U.S. Pat. No. 5,861,254, entitled Flow cell SELEX; U.S. Pat. No. 5,859,228, entitled Vascular endothelial growth factor (VEGF) nucleic acid ligand complexes; U.S. Pat. No. 5,858,660, entitled Parallel selex; U.S. Pat. No. 5,853,984, entitled Use of nucleic acid ligands in flow cytometry; U.S. Pat. No. 5,849,890, entitled High affinity oligonucleotide ligands to chorionic gonadotropin hormone and related glycoprotein hormones; U.S. Pat. No. 5,849,479, entitled High-affinity oligonucleotide ligands to vascular endothelial growth factor (VEGF); U.S. Pat. No. 5,846,713, entitled High affinity HKGF nucleic acid ligands and inhibitors; U.S. Pat. No. 5,843,653, entitled Method for detecting a target molecule in a sample using a nucleic acid ligand; U.S. Pat. No. 5,837,834, entitled High affinity HKGF nucleic acid ligands and inhibitors; U.S. Pat. No. 5,837,456, entitled High affinity oligonucleotide ligands to chorionic gonadotropin hormone and related glycoprotein hormones; U.S. Pat. No. 5,834,199, entitled Methods of identifying transition metal complexes that selectively cleave regulatory elements of mRNA and uses thereof; U.S. Pat. No. 5,817,785, entitled Methods of producing nucleic acid ligands; U.S. Pat. No. 5,811,533, entitled High-affinity oligonucleotide ligands to vascular endothelial growth factor (VEGF); U.S. Pat. No. 5,795,721, entitled High affinity nucleic acid ligands of ICP4; U.S. Pat. No. 5,789,163, entitled Enzyme linked oligonucleotide assays (ELONAS); U.S. Pat. No. 5,789,160, entitled Parallel selex; U.S. Pat. No. 5,789,157, entitled Systematic evolution of ligands by exponential enrichment: tissue selex; U.S. Pat. No. 5,786,462, entitled High affinity ssDNA ligands of HIV-1 reverse transcriptase; U.S. Pat. No. 5,780,228, entitled High affinity nucleic acid ligands to lectins U.S. Pat. No. 5,773,598, entitled Systematic evolution of ligands by exponential enrichment: chimeric selex; U.S. Pat. No. 5,766,853, entitled Method for identification of high affinity nucleic acid ligands to selectins; U.S. Pat. No. 5,763,595, entitled Systematic evolution of ligands by exponential enrichment: Chemi-SELEX; U.S. Pat. No. 5,763,566, entitled Systematic evolution of ligands by exponential enrichment: tissue SELEX; U.S. Pat. No. 5,763,177, entitled Systematic evolution of ligands by exponential enrichment: photoselection of nucleic acid ligands and solution selex; U.S. Pat. No. 5,763,173, entitled Nucleic acid ligand inhibitors to DNA polymerases; U.S. Pat. No. 5,756,287, entitled High affinity HIV integrase inhibitors; U.S. Pat. No. 5,750,342, entitled Nucleic acid ligands of tissue target; U.S. Pat. No. 5,734,034, entitled Nucleic acid ligand inhibitors of human neutrophil elastase; U.S. Pat. No. 5,731,424, entitled High affinity TGFβ nucleic acid ligands and inhibitors; U.S. Pat. No. 5,731,144, entitled High affinity TGFβ nucleic acid ligands; U.S. Pat. No. 5,726,017, entitled High affinity HIV-1 gag nucleic acid ligands; U.S. Pat. No. 5,723,594, entitled High affinity PDGF nucleic acid ligands; U.S. Pat. No. 5,723,592, entitled Parallel selex; U.S. Pat. No. 5,723,289, entitled Parallel selex; U.S. Pat. No. 5,712,375, entitled Systematic evolution of ligands by exponential enrichment: tissue selex; U.S. Pat. No. 5,707,796, entitled Method for selecting nucleic acids on the basis of structure; U.S. Pat. No. 5,705,337, entitled Systematic evolution of ligands by exponential enrichment: chemi-SELEX; U.S. Pat. No. 5,696,249, entitled Nucleic acid ligands; U.S. Pat. No. 5,693,502, entitled Nucleic acid ligand inhibitors to DNA polymerases; U.S. Pat. No. 5,688,935, entitled Nucleic acid ligands of tissue target; U.S. Pat. No. 5,686,592, entitled High-affinity oligonucleotide ligands to immunoglobulin E (IgE); U.S. Pat. No. 5,686,242, entitled Determination of oligonucleotides for therapeutics, diagnostics and research reagents; U.S. Pat. No. 5,683,867, entitled Systematic evolution of ligands by exponential enrichment: blended SELEX; U.S. Pat. No. 5,674,685, entitled High affinity PDGF nucleic acid ligands; U.S. Pat. No. 5,670,637, entitled Nucleic acid ligands; U.S. Pat. No. 5,668,264, entitled High affinity PDGF nucleic acid ligands; U.S. Pat. No. 5,663,064, entitled Ribozymes with RNA protein binding site; U.S. Pat. No. 5,660,985, entitled High affinity nucleic acid ligands containing modified nucleotides; U.S. Pat. No. 5,654,151, entitled High affinity HIV Nucleocapsid nucleic acid ligands; U.S. Pat. No. 5,650,275, entitled Target detection method using spectroscopically detectable nucleic acid ligands; U.S. Pat. No. 5,648,214, entitled High-affinity oligonucleotide ligands to the tachykinin substance P; U.S. Pat. No. 5,641,629, entitled Spectroscopically detectable nucleic acid ligands; U.S. Pat. No. 5,639,868, entitled High-affinity RNA ligands for basic fibroblast growth factor; U.S. Pat. No. 5,637,682, entitled High-affinity oligonucleotide ligands to the tachykinin substance P; U.S. Pat. No. 5,637,461, entitled Ligands of HIV-1 TAT protein; U.S. Pat. No. 5,637,459, entitled Systematic evolution of ligands by exponential enrichment: chimeric selex; U.S. Pat. No. 5,635,615, entitled High affinity HIV nucleocapsid nucleic acid ligands; U.S. Pat. No. 5,629,155, entitled High-affinity oligonucleotide ligands to immunoglobulin E (IgE); U.S. Pat. No. 5,622,828, entitled High-affinity oligonucleotide ligands to secretory phospholipase A2 (sPLA.sub.2); U.S. Pat. No. 5,595,877, entitled Methods of producing nucleic acid ligands; U.S. Pat. No. 5,587,468, entitled High affinity nucleic acid ligands to HIV integrase; U.S. Pat. No. 5,580,737, entitled High-affinity nucleic acid ligands that discriminate between theophylline and caffeine; U.S. Pat. No. 5,567,588, entitled Systematic evolution of ligands by exponential enrichment: Solution SELEX; U.S. Pat. No. 5,543,293, entitled DNA ligands of thrombin; U.S. Pat. No. 5,527,894, entitled Ligands of HIV-1 tat protein; U.S. Pat. No. 5,475,096, entitled Nucleic acid ligands; U.S. Pat. No. 5,866,334, entitled Determination and identification of active compounds in a compound library; U.S. Pat. No. 5,864,026, entitled Systematic evolution of ligands by exponential enrichment: tissue selex; U.S. Pat. No. 5,861,254, entitled Flow cell SELEX; U.S. Pat. No. 5,859,228, entitled Vascular endothelial growth factor (VEGF) nucleic acid ligand complexes; U.S. Pat. No. 5,858,660, entitled Parallel selex; U.S. Pat. No. 5,853,984, entitled Use of nucleic acid ligands in flow cytometry; U.S. Pat. No. 5,849,890, entitled High affinity oligonucleotide ligands to chorionic gonadotropin hormone and related glycoprotein hormones; U.S. Pat. No. 5,849,479, entitled High-affinity oligonucleotide ligands to vascular endothelial growth factor (VEGF); U.S. Pat. No. 5,846,713, entitled High affinity HKGF nucleic acid ligands and inhibitors; U.S. Pat. No. 5,843,732, entitled Method and apparatus for determining consensus secondary structures for nucleic acid sequences; U.S. Pat. No. 5,843,653, entitled Method for detecting a target molecule in a sample using a nucleic acid ligand; U.S. Pat. No. 5,840,867, entitled Aptamer analogs specific for biomolecules; U.S. Pat. No. 5,840,580, entitled Phenotypic characterization of the hematopoietic stem cell; U.S. Pat. No. 5,837,838, entitled Bax inhibitor proteins; U.S. Pat. No. 5,837,834, entitled High affinity HKGF nucleic acid ligands and inhibitors; U.S. Pat. No. 5,837,456, entitled High affinity oligonucleotide ligands to chorionic gonadotropin hormone and related glycoprotein hormones; U.S. Pat. No. 5,834,199, entitled Methods of identifying transition metal complexes that selectively cleave regulatory elements of mRNA and uses thereof; U.S. Pat. No. 5,834,184, entitled In vivo selection of RNA-binding peptides; U.S. Pat. No. 5,817,785, entitled Methods of producing nucleic acid ligands; U.S. Pat. No. 5,811,533, entitled High-affinity oligonucleotide ligands to vascular endothelial growth factor (VEGF); U.S. Pat. No. 5,804,390, entitled Use of nuclear magnetic resonance to identify ligands to target biomolecules; U.S. Pat. No. 5,795,721, entitled High affinity nucleic acid ligands of ICP4; U.S. Pat. No. 5,789,163, entitled Enzyme linked oligonucleotide assays (ELONAS); U.S. Pat. No. 5,789,160, entitled Parallel selex; U.S. Pat. No. 5,789,157, entitled Systematic evolution of ligands by exponential enrichment: tissue selex; U.S. Pat. No. 5,786,462, entitled High affinity ssDNA ligands of HIV-1 reverse transcriptase; U.S. Pat. No. 5,786,203, entitled Isolated nucleic acid encoding corticotropin-releasing factor.sub.2 receptors; U.S. Pat. No. 5,786,145, entitled Oligonucleotide competitors for binding of HIV RRE to REV protein and assays for screening inhibitors of this binding; U.S. Pat. No. 5,783,566, entitled Method for increasing or decreasing transfection efficiency; U.S. Pat. No. 5,780,610, entitled Reduction of nonspecific hybridization by using novel base-pairing schemes; U.S. Pat. No. 5,780,228, entitled High affinity nucleic acid ligands to lectins; U.S. Pat. No. 5,773,598, entitled Systematic evolution of ligands by exponential enrichment: chimeric selex; U.S. Pat. No. 5,770,434, entitled Soluble peptides having constrained, secondary conformation in solution and method of making same; U.S. Pat. No. 5,766,853, entitled Method for identification of high affinity nucleic acid ligands to selectins; U.S. Pat. No. 5,763,595, entitled Systematic evolution of ligands by exponential enrichment: Chemi-SELEX; U.S. Pat. No. 5,763,566, entitled Systematic evolution of ligands by exponential enrichment: tissue SELEX; U.S. Pat. No. 5,763,177, entitled Systematic evolution of ligands by exponential enrichment: photoselection of nucleic acid ligands and solution selex; U.S. Pat. No. 5,763,173, entitled Nucleic acid ligand inhibitors to DNA polymerases; U.S. Pat. No. 5,756,296, entitled Nucleotide-directed assembly of bimolecular and multimolecular drugs and devices; U.S. Pat. No. 5,756,291, entitled Aptamers specific for biomolecules and methods of making; U.S. Pat. No. 5,756,287, entitled High affinity HIV integrase inhibitors; U.S. Pat. No. 5,750,342, entitled Nucleic acid ligands of tissue target; U.S. Pat. No. 5,739,305, entitled Nucleotide-directed assembly of bimolecular and multimolecular drugs and devices; U.S. Pat. No. 5,734,034, entitled Nucleic acid ligand inhibitors of human neutrophil elastase; U.S. Pat. No. 5,733,732, entitled Methods for detecting primary adhalinopathy; U.S. Pat. No. 5,731,424, entitled High affinity TGF.beta. nucleic acid ligands and inhibitors; U.S. Pat. No. 5,731,144, entitled High affinity TGF.beta. nucleic acid ligands; U.S. Pat. No. 5,726,017, entitled High affinity HIV-1 gag nucleic acid ligands; U.S. Pat. No. 5,726,014, entitled Screening assay for the detection of DNA-binding molecules; U.S. Pat. No. 5,723,594, entitled High affinity PDGF nucleic acid ligands; U.S. Pat. No. 5,723,592, entitled Parallel selex; U.S. Pat. No. 5,723,289, entitled Parallel selex; U.S. Pat. No. 5,712,375, entitled Systematic evolution of ligands by exponential enrichment: tissue selex; U.S. Pat. No. 5,707,796, entitled Method for selecting nucleic acids on the basis of structure; U.S. Pat. No. 5,705,337, entitled Systematic evolution of ligands by exponential enrichment: chemi-SELEX; U.S. Pat. No. 5,698,442, entitled DNA encoding an 18 Kd CDK6 inhibiting protein; U.S. Pat. No. 5,698,426, entitled Surface expression libraries of heteromeric receptors; U.S. Pat. No. 5,698,401, entitled Use of nuclear magnetic resonance to identify ligands to target biomolecules; U.S. Pat. No. 5,693,502, entitled Nucleic acid ligand inhibitors to DNA polymerases; U.S. Pat. No. 5,688,935, entitled Nucleic acid ligands of tissue target; U.S. Pat. No. 5,688,670, entitled Self-modifying RNA molecules and methods of making; U.S. Pat. No. 5,686,592, entitled High-affinity oligonucleotide ligands to immunoglobulin E (IgE); U.S. Pat. No. 5,683,867, entitled Systematic evolution of ligands by exponential enrichment: blended SELEX; U.S. Pat. No. 5,681,702, entitled Reduction of nonspecific hybridization by using novel base-pairing schemes; U.S. Pat. No. 5,674,685, entitled High affinity PDGF nucleic acid ligands; U.S. Pat. No. 5,670,637, entitled Nucleic acid ligands; U.S. Pat. No. 5,668,265, entitled Bi-directional oligonucleotides that bind thrombin; U.S. Pat. No. 5,668,264, entitled High affinity PDGF nucleic acid ligands; U.S. Pat. No. 5,660,985, entitled High affinity nucleic acid ligands containing modified nucleotides; U.S. Pat. No. 5,660,855, entitled Lipid constructs for targeting to vascular smooth muscle tissue; U.S. Pat. No. 5,658,738, entitled Bi-directional oligonucleotides that bind thrombin; U.S. Pat. No. 5,656,739, entitled Nucleotide-directed assembly of bimolecular and multimolecular drugs and devices; U.S. Pat. No. 5,656,467, entitled Methods and materials for producing gene libraries; U.S. Pat. No. 5,654,151, entitled High affinity HIV Nucleocapsid nucleic acid ligands; U.S. Pat. No. 5,650,275, entitled Target detection method using spectroscopically detectable nucleic acid ligands; U.S. Pat. No. 5,648,214, entitled High-affinity oligonucleotide ligands to the tachykinin substance P; U.S. Pat. No. 5,641,629, entitled Spectroscopically detectable nucleic acid ligands; U.S. Pat. No. 5,639,868, entitled High-affinity RNA ligands for basic fibroblast growth factor; U.S. Pat. No. 5,639,428, entitled Method and apparatus for fully automated nucleic acid amplification, nucleic acid assay and immunoassay; U.S. Pat. No. 5,637,682, entitled High-affinity oligonucleotide ligands to the tachykinin substance P; U.S. Pat. No. 5,637,459, entitled Systematic evolution of ligands by exponential enrichment: chimeric selex; U.S. Pat. No. 5,635,615, entitled High affinity HIV nucleocapsid nucleic acid ligands; U.S. Pat. No. 5,631,156, entitled DNA encoding and 18 KD CDK6 inhibiting protein; U.S. Pat. No. 5,631,146, entitled DNA aptamers and catalysts that bind adenosine or adenosine-5′-phosphates and methods for isolation thereof; U.S. Pat. No. 5,629,407, entitled DNA encoding an 18 KD CDK6 inhibiting protein and antibodies thereto; U.S. Pat. No. 5,629,155, entitled High-affinity oligonucleotide ligands to immunoglobulin E (IgE); U.S. Pat. No. 5,622,828, entitled High-affinity oligonucleotide ligands to secretory phospholipase A2 (sPLA.sub.2); U.S. Pat. No. 5,621,082, entitled DNA encoding an 18 Kd CDK6 inhibiting protein; U.S. Pat. No. 5,599,917, entitled Inhibition of interferon-.gamma. with oligonucleotides; U.S. Pat. No. 5,597,696, entitled Covalent cyanine dye oligonucleotide conjugates; U.S. Pat. No. 5,587,468, entitled High affinity nucleic acid ligands to HIV integrase; U.S. Pat. No. 5,585,269, entitled Isolated DNA encoding c-mer protooncogene; U.S. Pat. No. 5,580,737, entitled High-affinity nucleic acid ligands that discriminate between theophylline and caffeine; U.S. Pat. No. 5,567,588, entitled Systematic evolution of ligands by exponential enrichment: Solution SELEX; U.S. Pat. No. 5,565,327, entitled Methods of diagnosing parasitic infections and of testing drug susceptibility of parasites; U.S. Pat. No. 5,527,894, entitled Ligands of HIV-1 tat protein; U.S. Pat. No. 5,512,462, entitled Methods and reagents for the polymerase chain reaction amplification of long DNA sequences; U.S. Pat. No. 5,503,978, entitled Method for identification of high affinity DNA ligands of HIV-1 reverse transcriptase; U.S. Pat. No. 5,472,841, entitled Methods for identifying nucleic acid ligands of human neutrophil elastase; and U.S. Pat. No. 5,459,015, entitled High-affinity RNA ligands of basic fibroblast growth factor.

VII. Delivery of Nucleic Acids

In one embodiment, nucleic acids or analogues are delivered that encode peptides, proteins or other biological modifiers.

Table 4 below lists a number of genes involved in the establishment, growth, invasion and metastasis of tumors and genes involved in the development of resistance to chemotherapeutic drugs that are particularly interesting as antisense targets. It should be understood that many of the genes listed in Table 4 are representatives of a larger gene family, the other members of which also constitute potentially important antisense targets, e.g., ADAMTS-1 is a member of the ADAMs gene family that encode cellular disintegrins and metalloproteinases, MMP-1 is a member of the matrix metalloproteinases (MMPs) gene family that encode zinc-dependent endoproteinases, etc.

TABLE 4

ABL1 COT GLI3 PAI2 ABL2 CREB1 GRO1 PCNA ABR CREBBP
GRO2 PDGFA ADAM11 CRK GRO3 PDGFB ADAMTS-1 CRKL HCK
PDGFRA AKT1 CSF1 HGF PDGFRB AKT2 CSF1R HKR3 PIM1
APC CSF2 HOX11 PLAT ARAF1 CSF2RA HOXA10 PLAU ARAF2
CSF2RB HOXB2 PLAUR AREG CSF2RY HPC1 PLG ARHA CSF3R
HSPA9 PMS1 ARHB D10S170 HRAS PMS2 ARHC DAP IFNB1
PPARA AT DAP3 IFNG PPARBP AXL DAPK1 IFNGR1 PPARG
BAD DBCCR1 IFNGR2 PTCH BAG1 DCC IRF4 PVTI BAI1
DDX6 JUN RAF1 BAK1 E2F1 JUNB RALA BAP1 E2F4 JUND
RALB BARD1 E4F1 KAI1 RARA BAX EGF KIT RARB BCL2
EGFR KRAS2 RARG BCL2A1 EIF3S2 LCK RASA1 BCL3
EIF356 LCN1 RB1 BCL5 EIF4E LCN2 RBBP6 BCL6
EIFE4EBP1 LCO REL BCNS ELE1 LCP1 RELA BCR ELK1
LCP2 REQ BCS ELK3 LPSA RET BL ELK4 LTA RMYC BLYM
EMP1 LTB ROS1 BMI1 EMS1 LTD RRAS BMYC EPHA1 LYN
SEA BRAF EPHA3 MAD SET BRCA1 ERBAL2 MADH4 SIS
BRCA2 ERBB2 MAF SKI BRCD1 ERBB3 MAFG SKIL CALCR
ERBB4 MAFK SMARCB1 CASP1 ERG MAP2K1 SPI1 CASP2
ERPL1 MAP2K4 SPINK1 CASP3 ERS1 MAP2K6 SRC CASP4
ESR2 MAP3K7 ST5 CASP5 ESRRA MAP3K8 SUPT3H CASP6
ESRRB MAP3KI4 SUPT5H CASP13 ESRRG MAPKAPK3 SUPT6H
CBL ETS1 M1S1 TAF2A CCNA1 ETS2 M4S1 TAF2H CCNA2
ETV3 M6P2 TAL1 CCNB1 ETV4 MPL TF CCNB2 ETV6 MAS1
THPO CCNC EVI1 MAX THRA CCND1 EWSR1 MCC THRB CCNP2
FAT MCF2 TIAM1 CCND3 FER MDM2 TIM CCNE1 FES MDR-1
TIMP-1 CCNE2 FGD1 MDR-2 TIMP-2 CCNF FGF1 MEL
TM4SF1 CCNG1 FGF2 MEN1 TNF CCNG2 FGF3 MET TP53
CCNH FGF4 MGR-2 TP53BP2 CCNK FGF5 MLH1 TP73 CCNT1
FGF6 MMP-1 VAV1 CCNT2 FGF7 MMP-2 VAV2 CDC23 FGF8
MMP-3 VDR CDC25A FGF9 MMP-9 VEGF CDC25C FGF10
MNAT1 VGF CDC2L1 FGF11 MOS VHL CDC2L2 FGF12 MPL
WNT1I CDC34 FGF13 MSH2 WNT2 CDH1 FGF14 MYB WNT5A
CDH5 FGF16 MYBL1 WT1 CDH7 FGF17 MYBL2 YES1 CDK2
FGF18 MYC CDK3 FGF19 MYCL1 CDK4 FGFR1 MYCN CDK5
FGFR2 NBL1 CDK6 FGFR3 NF1 CDK7 FGFR4 NF2 CDK8 FGR
NFKB2 CDK9 FKHL1 NKTR CDK10 FL11 NOS2A CDKL1 FLT1
NOS2B CDKL2 FMS NOS2C CDKN1A FPS NOS3 CDKN1B FOS
NOTCH4 CDKN1C FOSB NOV CDKN2A FOSL1 NRAS CDKN2B
FOSL2 NRG1 CDKN2C FYN NRG2 CDKN2D GADD45A NTRK1
CDKN3 GAK ODC1 CDL4 GLI PACE CHES1 GLI2 PAI1

It should be appreciated that in the above Table 4, an indicated gene means the gene and all currently known variants thereof, including the different mRNA transcripts that the gene and its variants can give rise to, and any further gene variants which may be elucidated. In general, however, such variants will have significant homology (sequence identify) to a sequence of Table 4 above, e.g., a variant will have at least about 70 percent homology (sequence identity) to a sequence of the above Table 4, more typically at least about 75, 80, 85, 90, 95, 97, 98 or 99 homology (sequence identity) to a sequence of the above Table 4. Homology of a variant can be determined by any of a number of standard techniques such as a BLAST program.

Sequences for the genes listed in Table 4 can be found in GenBank (http://www.ncbi.nlm.nih.gov/). The gene sequences may be genomic, cDNA or mRNA sequences. Preferred sequences are mammal genes containing the complete coding region and 5′ untranslated sequences. Particularly preferred are human cDNA sequences.

Tables 4 and 5 (Table 5 is attached to the end of this specification) n provide additional nonlimiting lists of nucleic acids, analogs and derivatvies that can be delivered according to the method described herein. It is understood that all or a portion of the above listed nucleic acids disclosed in Tables 4-5 or those listed above, or analogs or derivatives thereof can be coupled to the ligands of the transcobalamin receptor or intrinsic factor receptor. It is also understood that either the nucleic acids themselves or antisense molecules that are generated from the nucleic acid sequences from the above listed nucleic acids disclosed in Tables 3-6 can be conjugated to the ligands of the transcobalamin receptor or intrinsic factor receptor.

IX. Synthetic Methods

Various synthetic techniques are known for preparing the compounds of the present invention. For example, compounds wherein the residue of an antisense sequence is directly linked to the 6-position of a compound of formula I (i.e. in which X is L-T and L is a direct bond) can be prepared by reducing a corresponding Co (III) compound of formula I to form a nucleophilic Co (I) compound and treating this Co (I) compound with a residue of a antisense sequence (or a derivative thereof) comprising a suitable leaving group, such as a halide. Similarly, compounds wherein X is L-T and L is other than a direct bond can be prepared by preparing a nucleophilic Co (I) species as described herein above and reacting it with a linker comprising a suitable leaving group, such as a halide. Peptides and amino acids can be attached to the 6-position by reducing a corresponding Co (III) compound of formula I to form a nucleophilic Co (I) compound and treating the Co (I) compound with a suitable alkylating agent comprising an amino acid or peptide.

Coupling of L-T to the ribose moiety at K or G¹ may be accomplished by activating the natural OH at either K or G¹ with a suitable reagent such as succinic anhydride, to yield a reactive group such as a carboxylate. This technique is described in detail in Toraya, Bioinorg. Chem. 4:245-255, 1975.

Coupling of L-T to M can be accomplished using techniques described in detail in Jacobsen, Anal. Biochem. 113:164-171, 1981.

The residue of vitamin B₁₂ or its analog can be prepared by any suitable means known in the art. For example, a monocarboxylic acid or dicarboxylic acid of cobalamin can be prepared as disclosed in U.S. Pat. No. 5,739,313. These compounds can be prepared by the mild acid hydrolysis of cyanocobalamin, which has been shown to yield a mixture of mono-, a dicarboxylic acid and one tricarboxylic acid. These carboxylic acids are derived from the propionamide side chains designated b, d- and e-, as discussed hereinabove, which are more susceptible to hydrolysis than the amide groups on acetamide side chains a-, c- and g-. The b-, d- and e-monocarboxylic acids can be separated by column chromatography. L. Anton et al., J. Amer. Chem. Soc., 102, 2215 (1980). See, also, J B. Armitage et al., L Chem. Sot., 3349 (1953); K. Bernhauer, Biochem. Z., 344, 289 (1966); H. P. C. Hogenkamp et al., Biochemistry, 14, 3707 (1975); and L. Ellenbogen, in “Cobalamin,” Biochem. and Pathophysiol, B. Babior, ed., Wiley, N.Y. (1975) at chapter 5.

Additional compounds, intermediates and synthetic preparations thereof are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium III-Labeled DTPA-Adenosylcobalamin, Mayo Clinic Proc., 1999, 74:687-691.

(I) Synthesis of Antisense Sequence.

The nucleic acid conjugates used in accordance with this invention may be conveniently and routinely made through the well-known technique of solid phase synthesis. Equipment for such synthesis is sold by several vendors including Applied Biosystems. Any other means for such synthesis, either solution or solid phase, may also be employed. It is well known to use similar techniques to prepare oligonucleotides such as the phosphorothioates, 2′-alkoxy or 2′-alkoxyalkoxy derivatives, including 2′-O-methoxyethyl oligonucleotides (Martin, P., Helv. Chim. Acta, 1995, 78, 486-504), and morpholino peptide nucleic acids. It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other conjugated oligonucleotides.

“Peptide Nucleic Acids: Protocols and Applications” Editors: Peter E. Nielsen and Michael Egholm, May 1999 discloses PNA oligomer synthesis by BOC chemistry such as the Boc/Z strategy with both detailed manual and automated synthesis protocols. The automated protocol is designed to the ABI 433A peptide synthesiser. Common side reactions in PNA synthesis are described along with procedures for reducing their impact on PNA synthesis. Alternatively, PNA oligomers can be synthesized using Fmoc chemistry, such as with Fmoc/Bhoc protected monomers on a common DNA synthesizer, Expedite 8909, is described. The milder chemistry of this synthesis scheme provides for PNAs carrying sensitive reporter groups and the preparation of PNA-conjugates. Procedures for labeling, analysis and purification of PNA are also detailed.

Procedures for non-radioctive labeling of PNAs with biotin, fluorescein, rhodamine a.o., as well as ³²P-labeling of PNA-peptide chimeras containing the Kemptide sequence motif (H-Leu-Arg-Arg-Ala-Ser-Leu-Gly-NH₂) using protein kinase A (PKA) and g-³²P, are given both using on-resin solid phase coupling and solution phase post-modification are also well known. Furthermore, a method for.

The nuclear localization signal peptide, TAT (Tyr-Gly-Arg-Lys-Arg-Arg-Gln-Arg-Arg-Arg), can be synthesized as a peptide amide by any solution phase or solid-phase method known in the art. In a particular embodiment, the peptide amide is synthesized by solid phase methodology using a suitable resin, such as on Rink (4-2′,4′-dimethoxyphenyl-Fmoc-aminomethyl-phenoxy) co-polystyrene resin (Calbiochem-Novabiochem Corp., San Diego, Calif.). As a particular example, each protected-amino acid (for example with Fmoc) can be activated with an activating group, such as PyBop/HoBt/4-Methymorpholine and coupled to the resin-linked peptide chain in a suitable solvent, such as 1-methyl-2-pyrrolidinone (NMP), followed by deprotection of each protecting group. For example, if the amino acid is protected with Fmoc, the protecting groups can be removed with piperidine in a suitable solvent, such as NMP.

The peptide nucleic acid (PNA) can be sequentially added to the free amino group of the resin-bound TAT peptide, starting with the first base at the 3′-end of the PNA molecule. The synthesis of the PNA can be achieved using protected (2-aminoethyl)glycyl PNA monomers, such as Fmoc-N-(2-aminoethyl)glycyl PNA on a suitable synthesizer, such as the Expidite 8909 Nucleic Acid Synthesizer (Perspective Biosystems, Inc., Foster City, Calif.), according to cycle protocols developed by the manufacturer. The exocyclic amines of the bases adenine, guanine, and cytosine of each protected-PNA monomer can then be protected with the blocking group, such as benzhydryloxycarbonyl (Bhoc).

The protecting group of each PNA monomer is removed by any means known in the art. For example, if Fmoc protecting groups are used, they can be removed by treatment with 20% piperidine in a suitable solvent, such as dimethylformamide (DMF), followed by activation and coupling of the next PNA monomer (5 equiv.). In a particular embodiment of the invention, the activation and coupling is achieved with HATU, 2,6-lutidine and diisopropylethylamine (5 equiv.).

Finally, a linker group can be added to the PNA prior to linkage with the carrier molecule of the present invention. For example, addition of an AEEA [2(2-aminoethoxy)ethoxy]acid monomer can be added to the 5′-end of the synthesized PNA as a spacer group.

Alternatively, a method for the solid-support synthesis of PNA/DNA chimeras using monomethoxytrityl/acyl-protected monomeric building blocks can be used. The acid-labile monomethoxytrityl (Mmt) group can be employed for the temporary protection of the amino function of aminoethylglycine, while the exocyclic amino functions of the nucleobases are protected with ammonia-cleavable acyl protecting groups. This orthogonal protecting-group strategy is fully compatible with the standard phosphoramidite DNA synthesis method. The resulting PNA/DNA chimeras obey the Watson-Crick rules on binding to complementary DNA and RNA. Binding affinity of the PNA-DNA chimeras strongly depends on the PNA:DNA ratio. The PNA/DNA chimeras bind with higher affinity to RNA than to DNA, and the type of linking moiety between PNA and DNA could be adjusted to obtain optimal binding affinity. In addition to their binding properties, PNA-DNA chimeras can also assume biological functions, such as a primer function for DNA polymerases. Pure PNAs cannot induce RNase H cleavage of target RNA, which often supports the biological efficacy of antisense agents. In contrast, the DNA-PNA chimeras are able to stimulate cleavage of the target RNA by RNase H on formation of an RNA-chimera duplex.

(J) Coupling of Carrier to Antisense Sequence.

The carrier molecule, for example vitamin B₁₂ with a free carboxylate, can be added to the amino terminal groups of the antisense sequence, for example, a linker-PNA-TAT chimera, such as AEEA-PNA-TAT chimera, by activation of carrier's carboxylic acid, followed by coupling of the carrier to the antisense sequence. Such activation can be achieved using any means known in the art, but in particular can be achieved with PyBop/HoBt/4-Methymorpholine in DMF, and subsequent coupling of the mixture in DMF.

(K) Deprotection and Purification of the Carrier-Antisense Sequence Conjugate.

After coupling of the carrier, the conjugate can be deprotected, and in the case of solid phase synthesis, removed from the resin support. For example if the carrier-PNA-TAT chimera is on a rink-resin support, this can be achieved by treatment with a mixture of 90% TFA/5.0% water/2.5% ethanedithiol/2.5% thioanisole. The deprotected crude product can then be washed, separated, preferably by precipitation, and purified, preferably by reversed phase HPLC. The composition of the carrier-PNA-TAT product can then be analyzed by Electrospray Ionization (ESI) Mass Analysis.

(L) Compound of Formula I/Antisense Sequence Linkage

The invention provides a compound of formula I directly linked to one or more antisense sequence of the present invention, wherein X is CN, OH, CH₃, adenosyl or L-T, wherein T is preferably an antisense sequence (such as a Stabilized mimic); or a pharmaceutically acceptable salt thereof.

The residue of an antisense sequence of the present invention can be linked to the residue of a compound of formula I through an amide (e.g. —NRC(═O)— or —C(═O)NR—), ester (e.g. —OC(═O)— or —C(═O)O—), ether (e.g. —O—), amino (e.g. —NR—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C₁-C₆)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given residue of an antisense sequence (such as Stabilized mimic) using procedures that are known in the art.

The residue of the antisense sequence of the present invention can be directly linked to any synthetically feasible position on the residue of a compound of formula I. Suitable points of attachment include, for example, the b-carboxamide, the d-carboxamide and the e-carboxamide, as well as the 6-position and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amide, methyl-cobalamin-b-(4-aminobutyl)amide and adenosylcobalamin-b-(4-aminobutyl)amide) that are useful intermediates for the preparation of compounds of the present invention.

Compounds wherein the residue of an antisense sequence of the present invention is linked to the 6-position of a compound of formula I can be prepared by reducing a corresponding Co (III) compound of formula I to form a nucleophilic Co (I) compound and treating this Co (I) compound with a residue of an antisense sequence (or a derivative thereof) comprising a suitable leaving group, such as a halide (e.g. a chloride).

The invention also provides compounds having more than one residue of an antisense sequence(s) of the present invention directly linked to a compound of formula I. For example, the residue of an antisense sequence of the present invention can be directly linked to a residue of the b-carboxamide of the compound of formula I and a residue of another antisense sequence of the present invention can be directly linked to a residue of the d- or e-carboxamide of the compound of formula I. In addition, the residue of an antisense sequence of the present invention can be directly linked to the 6-position of the compound of formula I and a residue of another antisense sequence of the present invention can be directly linked, for example, to a residue of the b-, d- or e-carboxamide of the compound of formula I.

(M) Compound of Formula I/Linker/Antisense Sequence Linkage

In addition to being directly linked to the residue of a compound of formula I, the residue of an antisense sequence of the present invention can also be linked to the residue of a compound of formula I by a suitable linker. The structure of the linker is not crucial, provided the resulting compound of the invention has an effective therapeutic index as a drug and preferably can be orally administered. Suitable linkers are disclosed, for example, in U.S. Pat. No. 5,735,313; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; and references cited therein.

Suitable linkers include linkers that separate the residue of a compound of formula I and the residue of an antisense sequence of the present invention by about 5 angstroms to about 200 angstroms, inclusive, in length. Other suitable linkers include linkers that separate the residue of a compound of formula I and the residue of an antisense sequence of the present invention by about 5 angstroms to about 100 angstroms, inclusive, in length, as well as linkers that separate the residue of a compound of formula I and the residue of an antisense sequence of the present invention by about 5 angstroms to about 50 angstroms or by about 5 angstroms to about 25 angstroms, inclusive, in length.

The linker can be linked to any synthetically feasible position on the residue of a compound of formula I. Suitable points of attachment include, for example, a residue of the b-carboxamide, a residue of the d-carboxamide, a residue of the e-carboxamide, the 6-position (i.e. the position occupied by X in the compound of formula I), as well as a residue of the 5′-hydroxy group and a residue of the 3′ hydroxy group on the 5-membered sugar ring, although other points of attachment are possible. Based on the linkage that is desired, one skilled in the art can select suitably functionalized starting materials that can be derived from a compound of formula I and an antisense sequence of the present invention using procedures that are known in the art.

The linker can conveniently be linked to the residue of a compound of formula I or to the residue of an antisense sequence of the present invention through an amide (e.g. —NRC(═O)— or —C(═O)NR—), ester (e.g. —OC(═O)— or —C(═O)O—), ether (e.g. —O—), ketone (e.g. —C(═O)—) thioether (e.g. —S—), sulfinyl (e.g. —S(O)—), sulfonyl (e.g. —S(O)₂—), amino (e.g. —NR—) or a direct (e.g. C—C) linkage, wherein each R is independently H or (C₁-C₆)alkyl. The linkage can be formed from suitably functionalized starting materials using synthetic procedures that are known in the art. Based on the linkage that is desired, one skilled in the art can select suitably functional starting materials that can be derived from a residue of a compound of formula I, a residue of an antisense sequence of the present invention and from a given linker using procedures that are known in the art.

Specifically, the linker can be a divalent radical of the formula W-A-Q wherein A is (C₁-C₂₄)alkyl, (C₂-C₂₄) alkenyl, (C₂-C₂₄)alkynyl, (C₃-C₈)cycloalkyl or (C₆-C₁₀)aryl, wherein W and Q are each independently —NRC(═O)—, —C(═O)NR—, —OC(═O)—, —C(═O)O—, —O—, —S—, —S(O)₂—, —NR—, —C(═O)— or a direct bond (i.e. W and/or Q is absent); wherein each R is independently H or (C₁-C₆)alkyl.

Specifically, the linker can be a divalent radical of the formula W—(CH₂)ₙ-Q wherein, n is between about 1 and about 20, between about 1 and about 15, between about 2 and about 10, between about 2 and about 6 or between about 4 and about 6; wherein W and Q are each independently —NRC(═O)—, —C(═O)NR—, —OC(═O)—, —C(═O)O—, —O—, —S—, —S(O)—, —S(O)₂—, —C(═O)—, —NR— or a direct bond (i.e. W and/or Q is absent); wherein each R is independently H or (C₁-C₆)alkyl.

Specifically, W and Q can each independently be —NRC(═O)—, —C(═O)NR—, —OC(═O)—, —NR—, —C(═O)O—, —O— or a direct bond (i.e. W and/or Q is absent).

Specifically, the linker is a divalent radical, i.e. 1,ω-divalent radicals formed from a peptide or an amino acid. The peptide can comprise 2 to about 25 amino acids, 2 to about 15 amino acids or 2 to about 12 amino acids.

Specifically, the peptide can be poly-L-lysine (i.e. [—NHCH[(CH₂)₄NH₂]CO—]ₘ-Q, wherein Q is H, (C₁-C₁₄) alkyl or a suitable carboxy protecting group; and wherein m is about 2 to about 25. Specifically, the poly-L-lysine can contain about 5 to about 15 residues (i.e. m is between about 5 and about 15). More specifically, the poly-L-lysine can contain about 8 to about 11 residues (i.e. m is between about 8 and about 11).

Specifically, the peptide can be poly-L-glutamic acid, poly-L-aspartic acid, poly-L-histidine, poly-L-serine, poly-L-threonine, poly-L-tyrosine, poly-L-leucine, poly-L-lysine-L-phenylalanine or poly-L-lysine-L-tyrosine.

Specifically, the linker can be prepared from 1,6-diaminohexane H₂N(CH₂)₆NH₂, 1,5-diaminopentane H₂N(CH₂)₅NH₂, 1,4-diaminobutane H₂N(CH₂)₄NH₂ or 1,3-diaminopropane H₂N(CH₂)₃NH₂′

The invention also provides compounds having more than one antisense sequence of the present invention attached to a compound of formula I, each through a linker. For example, the residue of an antisense sequence of the present invention can conveniently be linked, through a linker, to a residue of the b-carboxamide of the compound of formula I and a residue of another antisense sequence of the present invention can conveniently be linked, through a linker, to a residue of the d- or e-carboxamide of the compound of formula I. In addition, the residue of an antisense sequence of the present invention can conveniently be linked, for example, through a linker, to the 6-position of the compound of formula I and a residue of another antisense sequence of the present invention can conveniently be linked, through a linker, to a residue of the b-, d- or e-carboxamide of the compound of formula I.

Compounds wherein the linker is linked to the 6-position of a compound of formula I can be prepared by preparing a nucleophilic Co (I) species as described herein above and reacting it with a linker comprising a suitable leaving group, such as a halide (e.g. a chloride).

The invention also provides compounds having more than one antisense sequence of the present invention attached to a compound of formula I, either directly or through a linker. For example, the residue of an antisense sequence of the present invention can conveniently be linked, either directly or through a linker, to a residue of the b-carboxamide of the compound of formula I and a residue of another antisense sequence of the present invention can conveniently be linked, either directly or through a linker, to a residue of the d- or e-carboxamide of the compound of formula I. In addition, the residue of an antisense sequence of the present invention can conveniently be linked, for example, either directly or through a linker, to the 6-position of the compound of formula I and a residue of another antisense sequence of the present invention can conveniently be linked, either directly or through a linker, to a residue of the b-, d- or e-carboxamide of the compound of formula I.

(N) Compound of Formula I/Detectable Radionuclide Linkage

In a particular embodiment of the invention, the conjugate also contains an imaging agent. Therefore, the invention provides compounds wherein a residue of compound of formula I is directly linked to a detectable radionuclide (e.g. non-metallic radionuclide). A detectable radionuclide (e.g. non-metallic radionuclide) can be linked directly to any synthetically feasible position on the residue of a compound of formula I. Suitable points of attachment include, for example, the b-carboxamide, the d-carboxamide and the e-carboxamide, as well as the 6-position and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amide, methylcobalamin-b-(4-aminobutyl)amide and adenosylcobalamin-b-(4-aminobutyl)amide) that are useful intermediates for the preparation of compounds of the present invention.

The invention also provides compounds having more than one detectable radionuclide (e.g. non-metallic radionuclides) directly linked to a compound of formula I. For example, the detectable radionuclide (e.g. non-metallic radionuclide) can be directly linked to a residue of the b-carboxamide of the compound of formula I and another detectable radionuclide (e.g. non-metallic radionuclide) can be directly linked to a residue of the d- or e-carboxamide of the compound of formula I. In addition, the detectable radionuclide (e.g. non-metallic radionuclide) can be directly linked to the 6-position of the compound of formula I and another detectable radionuclide (e.g. non-metallic radionuclide) can be directly linked, for example, to a residue of the b-, d- or e-carboxamide of the compound of formula I.

(O) Compound of Formula I/Linker/Detectable Radionuclide or Paramagnetic Metal Atom

When a detectable radionuclide (e.g. metallic radionuclide) or paramagnetic metal atom is linked to the residue of a compound of formula I by a suitable linker, the structure of the link is not crucial, provided it provides a compound of the invention which has an effective therapeutic and/or diagnostic index against the target cells and which will localize in or near the site of interest.

Suitable linkers include linkers that separate the residue of a compound of formula I and the detectable radionuclide by about 5 angstroms to about 200 angstroms, inclusive, in length. Other suitable linkers include linkers that separate the residue of a compound of formula I and the detectable radionuclide by about 5 angstroms to about 100 angstroms, as well as linkers that separate the residue of a compound of formula I and the detectable radionuclide by about 5 angstroms to about 50 angstroms, or by about 5 angstroms to about 25 angstroms. Suitable linkers are disclosed, for example, in U.S. Pat. No. 5,735,313.

The linkers can conveniently be linked to the residue of a compound of formula I through an amide (e.g. —NRC(═O)NR—), ester (e.g. —OC(═O)— or —C(═O)O—), thioether (e.g. —S—), sulfinyl (e.g. —S(O)—), Sulfonyl (e.g. —S(O)₂—) or a direct (e.g. C—C bond) linkage, wherein each R. is independently H or (C₁-C₁₄)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given linker using procedures that are known in the art.

The linker can be directly linked to any synthetically feasible position on the residue of a compound of formula I. Suitable points of attachment include, for example, the b-carboxamide, the d-carboxamide, ad the e-carboxamide, as well as the 6-position and the 5′-hydroxy and the 3′-hydroxy groups on the 5 membered sugar ring, although other points of attachment are possible. U.S. Pat. No. 5,739,313 discloses compound (e.g. cyano-cobalamin-b-(4-aminobytyl)amide, methylcobalamin-b-(4-aminobutyl)amide and adenosyl-cobalamin-b-(4-aminobutyl)amide) that are useful intermediates for the preparation of compounds of the present invention.

The invention also provides compounds having more than one linker attached to a compound of formula I. For example, the linker can be linked to a residue of the b-carboxamide of the compound of formula I and another linker can be directly linked to a residue of the d-carboxamide of the compound of formula I.

Specifically, the linker can comprise about 1 to about 20 detectable radionuclides. More specifically, the linker can comprise about 1 to 10 detectable radionuclides or about 1 to about 5 detectable radionuclides.

Specifically, the linker can be a divalent radical of the formula W-A wherein A is (C₁-C₆)alkyl, (C₂-C₆)alkenyl, (C₂-C₆)alkynyl, (C₃-C₈)cycloalkyl or (C₆-C₁₀)aryl, wherein W is —NRC(═O)—, —C(═O)NR—, —OC(═O)—, —O—, —S—, —S(O)_, S(O)₂—, —NR—, —C(═O)— or a direct bond, wherein each R is independently H or (C₁-C₆)alkyl; wherein A is linked to one or more non-metallic radionuclides.

Specifically, the linker can be an amino acid or a peptide. Specifically, the peptide can be poly-L-lysine, poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine or poly-L-lysine-L-tyrosine.

Specifically, the linker can be a chelating group capable of chelating one or more detectable radionuclides (e.g. metallic radionuclides). More specifically, the linker can be a detectable chelating group. Specifically, the chelating group can be DTPA.

The compounds disclosed herein can be prepared using procedures similar to those described in U.S. Pat. No. 5,739,313 or using procedures similar to those described herein. The residue of an antisense oligonucleotide (including Stabilized mimic) can be linked to the residue of a compound of formula I as described hereinabove. The detectable radionuclide can be linked to the residue of a compound of formula I as described hereinabove. Additional intermediates and synthetic procedures useful for preparing intermediates of the invention are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium III-Labeled DTPA-Adenosylcobalamin, Mayo Clinic Proc., 1999, 74:687-691; U.S. Application Ser. No. 60/129, 733 filed 16 Apr. 1999; U.S. Application Ser. No. 06/159, 874 filed 15 Oct. 1999; U.S. Application Ser. No. 60/159,753 filed 15 Oct. 1999; U.S. Application Ser. No. 60/159,873 filed 15 Oct. 1999; U.S. Pat. No. 5,739,313; U.S. Pat. No. 6,004,533; and references cited therein.

X. Therapeutic and Diagnostic Compositions and Administrations

Preferred modes of administration of the materials of the present invention to a mammalian host are parenteral, intravenous, intradermal, intra-articular, intra-synovial, intrathecal, intra-arterial, intracardiac, intramuscular, subcutaneous, intraorbital, intracapsular, intraspinal, intrasternal, topical, transdermal patch, via rectal, vaginal or urethral suppository, peritoneal, percutaneous, nasal spray, surgical implant, internal surgical paint, infusion pump or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

The TC- or IF-binding conjugates/imaging agents can, for example, be administered intravenously or intraperitoneally by infusion or injection. Solutions of the substance can be prepared in water, optionally mixed with a nontoxic surfactant. Dispersions can also be prepared in glycerol, liquid polyethylene glycols, triacetin and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the substance which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, normal saline, ethanol, a polyol (for example, glycerol, propylene glycol, liquid polyethylene glycols and the like), vegetable oils, nontoxic glyceryl esters and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, benzyl alcohol, sorbic acid, thimerosal and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the substance in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Injectable solutions are particularly advantageous for local administration of the therapeutic composition. In particular, parenchymal injection can be used to deliver the therapeutic composition directly to a tumorous growth. Intra-articular injection is a preferred alternative in cases of arthritis where the practitioner wishes to treat one or only a few (such as 2-6) joints. Additionally, the therapeutic compounds are injected directly into lesions (intra-lesion administration) in appropriate cases. Intradermal administration is an alternative for dermal lesions.

The therapeutic compound is optionally administered topically by the use of a transdermal therapeutic system (see, Barry, Dermatological Formulations, (1983) p. 181 and literature cited therein). Transdermal drug delivery (TDD) has several advantages over oral delivery. When compared to oral delivery, TDD avoids gastrointestinal drug metabolism, reduces first pass effects and provides a sustained release of drugs for up to seven days (Elias, et al. Percutaneous Absorption: Mechanisms-Methodology-Drug Delivery; Marcel Dekker, NY: 1, 1989). This method is especially useful with many therapeutic proteins that are susceptible to gastrointestinal degradation and exhibit poor gastrointestinal uptake. When compared to injections, TDD eliminates the associate pain and the possibility of infection. While such topical delivery systems have been designed largely for transdermal administration of low molecular weight drugs, by definition they are capable of percutaneous delivery. They can be readily adapted to administration of the therapeutic compounds of the invention by appropriate selection of the rate-controlling microporous membrane. Topical application can also be achieved by applying the compound of interest, in a cream, lotion, ointment or oil based carrier, directly to the skin. Typically, the concentration of therapeutic compound in a cream, lotion or oil is 1-2%.

For drug targeting to lung tissue, the therapeutic compound is formulated into a solution, suspension, aerosol or particulate dispersion appropriate for application to the pulmonary system. The therapeutic agent may be inhaled via nebulizer, inhalation capsule, inhalation aerosol, nasal solution, intratracheal as a solution via syringe or endotracheal tube as an aerosol or via as a nebulizer solution. Aerosols are prepared using an aqueous aerosol, liposomal preparation or solid particles containing the compound. A nonaqueous (e.g. fluorocarbon propellant) suspension could be used. Sonic nebulizers are preferred because they minimize exposing the therapeutic compound to shear, which can result in degradation of the compound.

Delivery of the cobalamin conjugates of the instant invention by the mucosal route also offers an attractive administration alternative. The prototype formulation for nasal solutions will contain the vitamin B₁₂ conjugate dissolved in a suitable aqueous or non-aqueous solvent such as propylene glycol, an antioxidant and aromatic oils as flavoring agents. The formulation may also contain suitable propellant(s).

For ophthalmic applications, the therapeutic compound is formulated into solutions, suspensions and ointments appropriate for use in the eye. For opthalmic formulations, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983).

Useful dosages of the compounds of formula I can be determined by comparing their in vitro activity and in vivo activity in animal models. Methods for the extrapolation of effective dosages in mice and other animals, to humans are known to the art; for example, see U.S. Pat. No. 4,938,949. The amount of the substance required for use in treatment will vary not only with the particular salt selected but also with the route of administration, the nature of the condition being treated and the age and condition of the patient and will be ultimately at the discretion of the attendant physician or clinician.

In general, however, a suitable dose for nuclear medicine (using a radioactive imaging agent) will be in the range of from about 0.1 μg/patient to about 1000 μg/patient, from about 0.5 to about 500 μg/patient or from 1 μg/patient to about 100 μg/patient.

A suitable dose for imaging medicine (using a paramagnetic imaging agent) will be in the range of from about 0.1 mg/patient to about 100 mg/patient, from about 0.5 to about 50 mg/patient or from 1 mg/patient to about 10 mg/patient.

For therapeutic applications, a suitable dose will be in the range of from about 0.05 picograms/kilogram to about 100 mg/kg, from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The substance is conveniently administered in unit dosage form; for example, containing 5 to 1000 mg, conveniently 10 to 750 mg, most conveniently, 50 to 500 mg of active ingredient per unit dosage form.

Ideally, the substance should be administered to achieve peak plasma concentrations of from about 0.05 to about 100 μM, preferably, about 1 to 50 μM, most preferably, about 2 to about 30 μM. This may be achieved, for example, by the intravenous injection of a 0.005 to 10% solution of the substance, optionally in saline or orally administered as a bolus containing about 0.5-250 mg of the substance. Desirable blood levels may be maintained by continuous infusion to provide about 0.01-5.0 mg/kg/hr or by intermittent infusions containing about 0.4-15 mg/kg of the substance.

The substance may conveniently be presented in a single dose or as divided doses administered at appropriate intervals, for example, as two, three, four or more sub-doses per day.

The cobalamin conjugates may be administered orally in combination with a pharmaceutically acceptable vehicle such as an inert diluent or an edible carrier. They may be enclosed in hard or soft shell gelatin capsules, may be compressed into tablets or may be incorporated directly with the food of the patient's diet. For oral therapeutic administration, the substance may be combined with one or more excipients and used in the form of ingestible tablets, buccal tablets, troches, capsules, elixirs, suspensions, syrups, wafers and the like. Such compositions and preparations should contain at least 0.1% of the substance. The percentage of the compositions and preparations may, of course, be varied and may conveniently be between about 2 to about 60% of the weight of a given unit dosage form. The amount of substance in such therapeutically useful compositions is such that an effective dosage level will be obtained.

Tablets, troches, pills, capsules and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the substance may be incorporated into sustained-release preparations and devices.

Sublingual tablets are designed to dissolve very rapidly. Examples of such formulations include ergotamine tartrate, isosorbide dinitrate, isoproterenol HCl. The formulation of these tablets contain, in addition to the drug, a limited number of soluble excipients, usually lactose and powdered sucrose, but occasionally dextrose and mannitol. The process of making sublingual tablets involves moistening the blended powder components with an alcohol-water solvent system containing approximately 60% alcohol and 40% water.

In addition to the cobalamin conjugate, the prototype formulation for sublingual tablets may contain a binder such as povidone or HPMC, diluents such as lactose, mannitol, starch or cellulose, a disintegrant such as pregelatinized or modified starch, lubricants such as magnesium stearate, stearic acid or hydrogenated vegetable oil, a sweetener such as saccharin or sucrose and suitable flavoring and coloring agents.

In one embodiment the invention provides surgical implant for localized delivery of an anti-proliferative agent comprising the nucleic acid conjugate of the present invention, and a biodegradable binder. The implant preferably is capable of releasing and delivering the nucleic acid conjugate to substantially all of an area of clear margin that results from a surgical lumpectomy, and is also preferably capable of releasing the nucleic acid conjugate at a substantially constant rate for at least one day.

The surgical implant can come in a variety of forms. In one embodiment the implant is a bolus, comprising a viscous and deformable material capable of being shaped and sized before implantation to complement a void created by a surgical lumpectomy, and sufficiently deformable upon implantation to contact substantially all of an area of clear margin.

The bolus will typically have a volume of at least about 1 cm³, and is often greater than 5, 10, 20 or 50 cm³ in volume. The implant can also comprise an outer layer and a core, wherein the outer later comprises the nucleic acid conjugate, and because it contacts the area of clear margin is able to release the nucleic acid conjugate directly to the adjacent tissue in the clear margin.

The surgical implant can also comprising a plurality of capsules that can be poured into the void created by a surgical lumpectomy. These capsules will contain the nucleic acid conjugate and a suitable binder. Because they are flowable, they can be poured into the void created by a surgical lumpectomy, and thereby contact substantially all of the area of clear margins.

Many suitable compositions for the implant are known and can be used in practicing the invention. Such compositions are described in, for example, Chasin et. al., Biodegradable Polymers as Drug Delivery Systems, Marcel Dekker Inc., NY, ISBN 0-8247-8344-1, the disclosure of which being incorporated herein by this reference. Preferable compositions are pharmaceutically acceptable, biodegradable, and meet the particular release profile characteristics that are required to achieve the administration regime involved.

The implant typically comprises a base composition which acts as a matrix to contain and hold the contents of the implant together. The base composition can, in turn, comprise one or more constituents. Examples of base compositions include polymers and copolymers of anhydrides, orthoester, lactic acid, glycolic acid, dioxonane, trimethylene carbonate, ε-caprolactone, phosphazene, and glyceryl monostearate.

In one embodiment the base composition for the matrix comprises a polyanhydride, which can be synthesized via the dehydration of diacid molecules by melt condensation. Degradation times can be adjusted from days to years according to the hydrophobicity of the monomer selected. The materials degrade primarily by surface erosion and possess excellent in vivo compatibility. In one embodiment the polyanhydride is formed from sebasic acid and hexadecandioic acid (poly(SA-HDA anhydride). Wafer-like implants using this base composition have been approved for use in brain cancer, as Giadel®, by Guilford Pharmaceuticals.

The implant optionally can comprise erosion and biodegradation enhancers that facilitate the erosion of the matrix, the dissolution of the core composition, or the uptake of the core composition via metabolic processes. Particularly suitable erosion and biodegradation enhancers are biodegradable in biological fluids, and biocompatible. Hydrophilic constituents are typical, because they are capable of enhancing the erosion of the implant in the presence of biological fluids. For example, K. Juni et al, Chem. Pharm. Bull., 33, 1609 (1985) disclose that the release rate of bleomycin from polylactic acid microspheres is greatly enhanced by incorporating fatty acid esters into the microspheres. Other exemplary hydrophilic constituents are described, for example, in Wade & Weller, Handbook of pharmaceutical Excipients (London: Pharmaceutical Press; Washington D.C.: American Pharmaceutical Ass'n 1995), and include the polyethylene glycols (“PEGs”), propylene glycol (“PG”), glycerin, and sorbitol.

Surfactants further enhance the erosion of the matrix and the release of the drug. Surfactants are generally capable of increasing the wettability and the solubility of the base composition in biological fluids, and thereby causing the disintegration and erosion of the implant. Surfactants can also help to break down the core composition matrix when, for example, the method of forming the dosage form has reduced the solubility of any of the constituents. Surfactants can also improve the uptake of the dosage forms into the bloodstream. Suitable surfactants include, for example, glyceryl based surfactants such as glyceryl monooleate and glyceryl monolaurate, polaxemers such as Pluronic F127, and polysorbates such as polyoxyethylene sorbitan monooleate (“Tween 80”).

The implant could also include components that retard the rate at which the implant erodes or biodegrades (erosion and/or biodegradation retardants). Hydrophobic constituents are a particularly suitable class of components for retarding the rate at which the outer layer biodegrades. Suitable hydrophobic constituents are described, for example, in the Handbook of Pharmaceutical Excipients, the disclosure from which being hereby incorporated by reference. Exemplary hydrophobic constituents include peanut oil, olive oil and castor oil.

Any proportions or types of constituents can be chosen that effectively achieve a desired release profile, and thereby carry out the prescribed administration regime. The most desirable base compositions generally release the drug substantially continuously, and biodegrade completely shortly after substantially all of the drug has been effectively released. The amount of drug included in the dosage forms is determined by the total amount of the drug to be administered, and the rate at which the drug is to be delivered. The total amount of the drug to be delivered is determined according to clinical requirements, and in keeping with the considerations that typically inform drug dosage determinations in other contexts. In one embodiment the implant comprises from zero to about 20 parts by weight erosion and/or biodegradation enhancers, from about 60 to about 100 parts by weight core base composition, and from about 1 to about 40 parts by weight of the nucleic acid conjugate of the present invention.

The surgical implant also can contain one or more other drugs having therapeutic efficacy in the intended applications, such as an antibiotic, an analgesic or an anesthetic.

The invention will now be illustrated by the following non-limiting Example.

### EXAMPLE

### Example 1

Preparation of Cyanocobalamin-b-(4-aminobutyl)amide

A mixture containing cyanocobalamin-b-carboxylic acid (1.0 g, 0.6 mmol), hydroxybenzotriazole (0.81 g, 6 mmol) and 1,4-diaminobutane dihydrochloride (4.8 g, 30 mmol) in 100 ml of water was adjusted to pH 7.8. 1-Ethyl-3-(3′-dimethylaminopropyl)carbodiimide (1.26 g, 6.6 mmol) was then added, the pH was adjusted to 6.4 and the reaction stirred at room temperature for 24 h. TLC on silica gel using n-butanol-acetic acid water (5:2:3) showed the reaction to be complete. Cyanocobalamin-b-(4-aminobutyl)amide was extracted into 92% aqueous phenol and the phenol layer was washed several times with equal volumes of water. To the phenol extract were added 3 volumes of diethylether and I volume of acetone. The desired cobalamin was removed from the organic phase by several extractions with water. The combined aqueous layers were extracted three times with diethylether to remove residual phenol, concentrated to approximately 200 ml in vacuo and crystallized from aqueous acetone. Yield 955 mg, 92%.

### Example 2

Proposed Preparation of Cyanocobalamin-b-(4-aminobutyl)amide-, Ciprofloxacin-, Levofloxacin-, Ofloxacin- and Sparfloxacin-Cobalamin Conjugates

A mixture containing cyanocobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 3

Preparation of Methylcobalamin-b-(4-aminobutyl)amide

Methylcobalamin-b-carboxylic acid (1.0 g, 0.6 mmol) was reacted with diaminobutane dihydrochloride as described above for the cyano derivative. The cobalamin was purified by extraction through phenol (see above) and the resulting aqueous solution was concentrated in vacuo. This solution was chromatographed on AG1-X2 200-400 mesh in the acetate form (20.times.2.5 cm) and the pass through collected. The pass through was concentrated to approximately 20 ml and the desired cobalamin crystallized from aqueous acetone. Yield 920 mg, 88%. Unreacted methylcobalamin-b-carboxylic acid was eluted with 1M acetic acid, concentrated and crystallized from aqueous acetone. Yield 60 mg, 6%.

### Example 4

Preparation of Adenosylcobalamin-b-(4-aminobutyl)amide

Adenosylcobalamin-b-carboxylic acid (500 mg, 0.3 mmol) was reacted with diaminobutane dihydrochloride (2.4 mg, 15 mmol) as described above. The cobalamin was purified by extraction through phenol (see above). The resulting aqueous solution was concentrated in vacuo and applied to AG-50×2, 200-400 mesh, in the hydrogen form (20.times.25 cm). The column was washed thoroughly with water to remove hydroxybenzotriazole and the desired cobalamin eluted with 1M ammonium hydroxide. After an additional extraction through phenol, adenosylcobalamin-b-(4-aminobutyl)amide was isolated as a glass. Yield 366 mg, 77%.

### Example 5

Preparation of Cyanocobalamin-b-(poly-L-lysine)amide

Two preparations of -poly-L-lysine hydrobromide, one containing approximately 8 residues and a second one containing about 11 residues were separately reacted with cyanocobalamin-1-carboxylic acid. To each polymer (500 mg) dissolved in 20 mL of water was added 150 mg (0.1 mmol) of cyanocobalamin-1-carboxylic acid, 338 mg (2.5 mmol) of hydroxybenzotriazole and 480 mg (2.5 mmol) of 1-ethyl-3(3-dimethyl-aminopropyl) carbodiimide. The pH was adjusted to 9 with 1M NaOH and the reaction mixtures were stirred at room temperature for 2-3 h. They were purified on G-10 sephadex: the sizing columns (3×40 cm) were eluted with water and 1.5 mL fractions collected. The fractions showing the presence of the cobalamin (OD at 550 mm) and the presence of polylysine (ninhydrin positive) were pooled and freeze-dried.

### Example 6

Synthesis of Peptide Nucleic Acid (PNA)-Nuclear Localization Peptide (TAT) Chimera

The nuclear localization signal peptide TAT (Tyr-Gly-Arg-Lys-Lys-Arg-Arg-Gln-Arg-Arg-Arg) is synthesized as a peptide amine by a solid-phase method on Rink (4-2′,4′-dimethozyphenyl-Fmoc-aminomethyl-phenoxy) co-polystyrene resin (0.1 mmole) with N′-Fmoc L-amino acids (Calbiochem-Novabiochem Corp., San Diego, Calif.). Ten equivalents (1.0 mmole) of each Fmoc-L-amino acid was activated with PyBop/HoBt/4-Methylmorpholine and coupled to the resin-linked peptide chain in 1-methyl-2-pyrrolidinone (NMP) for 2 h following deprotection of each N′-Fmoc protecting group with 20% piperidine in NMP for 30 minutes.

An anti-viral peptide nucleic acid (PNA) is sequentially added to the free amino group of the resin-bound TAT peptide, starting with the first base at the 3′-end of the PNA molecule. The synthesis of the PNA uses Fmoc-N-(2-aminoethyl) glycyl PNA monomers on an Expidite 8909 Nucleic Acid Synthesizer according to cycle protocols developed by the manufacturer (Perseptive Biosystems, Inc., Foster City, Calif.). The exocyclic amines of the bases adenine, guanine, and cytosine of each Fmoc-PNA monomer are protected with the blocking group benzhydryloxycarbonyl)Bhoc).

The Fmoc group of each PNA monomer is removed by treatment with 20% piperidine in dimethylformamide (DMF) for 15 min, followed by activation and coupling of the next PNA monomer (5 equivalents) with HATU (4.5 equiv.), 2,6-lutidine (7.5 equiv.) and diisopropylethylamine (5 equiv.) for 30 minutes. Addition of an AEEA [2(2-aminoethoxy) ethoxy]acetic acid monomer is added to the 5′-end of the synthesized PNA as a spacer group before linkage of the vitamin B₁₂ molecule.

### Example 7

Synthesis of Vitamin B₁₂ (B Carboxylate Form) to PNA-TAT Chimera

Vitamin B₁₂ (free carboxylate form) is added to the amino terminal groups of the AEEA-PNA-TAT chimera by activation of vitamin B₁₂'s carboxylic acid with PyBop/HoBt/4-Methymorpholine in DMF, and subsequent coupling of the mixture in DMF for 2 hours.

### Example 8

Deprotection and Purification of the Vitamin B₁₂-PNA-TAT Chimera

After coupling of the Vitamin B12, the vitamin B12-PNA-TAT chimera is deprotected and removed form the rink-resin support by treatment with a mixture of 90% TFA/5.0% water/2.5% ethanedithiol/2.5% thioanisole for 90 min at room temperature. The deprotected crude product is washed and separated by precipitation in 3×50 volumes of cold methyl t-butyl ether, and purified by reverse phase HPLC on Vydac C18 column (2.1)×25 cm) in 0.1% TFA/water with a 60 min gradient of 10%-89% acetonitrile in 0.1% TFA. The composition of the vitamin B₁₂-PNA-TAT product is analyzed by Electrospray Ionization (ESI) Mass Analysis on a PE SCIEX API 165 Biospectrometer (Applied Biosystems, Inc.)

The invention has been described with reference to various specific and preferred embodiments and techniques. It should be understood that many variations and modifications may be made while remaining within the spirit and scope of the invention.

TABLE 5

| Therapeutic | Exemplary | PCT/Patent | | | |
|---|---|---|---|---|---|
| Protein X | Identifier | Reference | Biological Activity | Exemplary Activity Assay | Preferred Indication Y |
| BMP-1 | GeneSeq Acession | WO8800205 | BMP1 belongs to the transforming growth | BMP-1 activity can be determined using the | Induction of Cartilage, |
| | P80618 | | factor-beta (TGFB) superfamily. Bone | following assays known in the art: Nat Genet. | Tissue and Bone Growth, |
| | | | morphogenic proteins induce cartilage and | 2001 Jan; 27(1): 84-8; Eur J Biochem 1996 Apr | and Diabetes |
| | | | bone formation, play important role in | 1; 237(1): 295-302; J Biol Chem, Vol. 274, | |
| | | | nephrogesis, and play an important role in the | Issue 16, 10897-10902, Apr. 16, 1999; and | |
| | | | development of many organs, including lung, | Hogan, B. L. M. (1996) Genes Dev. 10, 1580-1594. | |
| | | | heart, teeth, gut, skin, and particularly the | | |
| | | | kidney. | | |
| BMP-2 | GeneSeq | WO8800205 | BMP-2 belongs to the transforming growth | BMP-2 activity can be determined using the | Induction of Cartilage, |
| | Accession P80619 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, Issue | |
| | | | | 16, 10897-10902, Apr. 16, 1999; and Hogan, B. L. M. | |
| | | | | (1996) Genes Dev. 10, 1580-1594. | |
| BMP-2B | GeneSeq | U.S. Pat. No. 5631142 | BMP-2b belongs to the transforming growth | BMP-2b activity can be determined using the | Induction of Cartilage, |
| | Accession W24850 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; I Biol Cbcre, Vol. 274, Issue | |
| | | | | 16, 10897-10902, Apr. 16, 1999; and Hogan, B. L. M. | |
| | | | | (1996) Genes Dev. 10, 1580-1594. | |
| BMP-4 | GeneSeq | WO0020591 | BMP-4 belongs to the transforming growth | BMP-4 activity can be determined using the | Induction of Cartilage, |
| | Accession | | factor-beta (TGFB) superfamily. Bone | following assays known in the art: Nat Genet. | Tissue and Bone Growth, |
| | B02796 | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-5 | GeneSeq | WO0020591 | BMP-5 belongs to the transforming growth | BMP-5 activity can be determined using the | Induction of Cartilage, |
| | Accession | | factor-beta (TGFB) superfamily. Bone | following assays known in the art: Nat Genet. | Tissue and Bone Growth, |
| | B02797 | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-6 | GeneSeq | U.S. Pat. No. 5187076 | BMP-6 belongs to the transforming growth | BMP-6 activity can be determined using the | Induction of Cartilage, |
| | Accession | | factor-beta (TGFB) superfamily. Bone | following assays known in the art: Nat Genet. | Tissue and Bone Growth, |
| | R32904 | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| Osteogenic Protein- | GeneSeq | WO973462 | OP-1 belongs to the transforming growth | OP-1 activity can be determined using the | Induction of Cartilage, |
| 1; OP-1; BMP-7 | Accession | | factor-beta (TGFB) superfamily. Bone | following assays known in the art: Nat Genet. | Tissue and Bone Growth, |
| | W34783 | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| Osteogenic Protein-2 | GeneSeq Accession | WO9406399 | OP-2 belongs to the transforming growth | OP-2 activity can be determined using the | Induction of Cartilage, |
| | R57973 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M (1996) Genes Dev. 10, 1580-1594. | |
| GDP-1 | GeneSeq Accession | WO9406449 | Members of the TGF-beta family of proteins | The effect of GDF-1 on signaling can be | Developmental disorders, |
| | R60961 | | initiate cell signaling by binding to | assayed by treating Primary BAECs transferred | Induction of Cartilage, |
| | | | heteromeric receptor complexes of type I | with a construct called p3TP-Lux, containing a | Tissue and Bone Growth, |
| | | | (TbetaRI) and type II (TbetaRII) | TGF-beta responsive promoter fused to a | and Diabetes |
| | | | serine/threonine kinase receptors (reviewed | reporter gene, and measuring luciferase gene | |
| | | | by Massague, J. et al. (1994) Trends Cell | expression (Wrana et al., 1994, Nature 370: | |
| | | | Biol. 4: 172 178; Miyazono, K. et al. (1994) | 341-347). | |
| | | | Adv. Immunol. 55: 181-220). Activation of | | |
| | | | this heteromeric receptor complex occurs | | |
| | | | when TGF-beta binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg, R. A. (1995) PNA892: 1565-1569; | | |
| | | | Wrana, J. L. et al. (1994) Nature 370: 341 | | |
| | | | 347). | | |
| BMP-9 | GeneSeq Accession | WO9533830 | BMP-9 belongs to the transforming growth | BMP-9 activity can be determined using the | Induction of Cartilage, |
| | R86903 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-10 | GeneSeq Accession | WO9426893 | BMP-10 belongs to the transforming growth | BMP-10 activity can be determined using the | Induction of Cartilage, |
| | R66202 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-12 | GeneSeq Accession | WO9516035 | BMP-12 belongs to the transforming growth | BMP-12 activity can be determined using the | Induction of Cartilage, |
| | R78734 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-15 | GeneSeq Accession | W09636710 | BMP-15 belongs to the transforming growth | BMP-15 activity can be determined using the | Induction of Cartilage, |
| | W11261 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-17 | GeneSeq Accession | WO9929718 | BMP-17 belongs to the transforming growth | BMP-17 activity can be determined using the | Induction of Cartilage, |
| | Y17870 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| BMP-18 | GeneSeq | WO9929718 | BMP-18 belongs to the transforming growth | BMP-18 activity can be determined using the | Induction of Cartilage, |
| | Accession Y17871 | | factor-beta (TGFB) superfamily. Bone | following assays known in the art: Nat Genet. | Tissue and Bone Growth, |
| | | | morphogenic protein induces bone formation. | 2001 Jan; 27(1): 84-8; Eur J Biochem 1996 Apr | and Diabetes |
| | | | | 1; 237(1): 295-302; J Biol Chem, Vol. 274, | |
| | | | | Issue 16, 10897-10902, Apr. 16, 1999; and | |
| | | | | Hogan, B. L. M. (1996) Genes Dev. 10, 1580-1594. | |
| Inhibin alpha | GeneSeq | WO0020591 | The inhibin beta A subunit joins the alpha | Tumor suppressor activity of inhibin can be | Tumor suppression. |
| | Accession B02806 | | subunit to form a pituitary FSH secretion | determined using assays known in the art: | |
| | | | inhibitor. Inhibin has been shown to regulate | Matzuk et al., Nature 1992 Nov. 26: 360 | |
| | | | gonadal stromal cell proliferation negatively | (6402); 313-9. | |
| | | | and to have tumour-suppressor activity. In | | |
| | | | addition, serum levels of inhibin have been | | |
| | | | shown to reflect the size of granulosa-cell | | |
| | | | tumors and can therefore be used as a marker | | |
| | | | for primary as well as recurrent disease. | | |
| Inhibin beta | GeneSeq | WO0020591 | The inhibin beta A subunit joins the alpha | Tumor suppressor activity of inhibin can be | Tumor suppression. |
| | Accession | | subunit to form a pituitary FSH secretion | determined using assays known in the art: | |
| | H02808 | | inhibitor. Inhibin has been shown to regulate | Matzuk et al., Nature 1992 Nov. 26: 360 | |
| | | | gonadal stromal cell proliferation negatively | (6402); 313-9. | |
| | | | and to have tumour-suppressor activity. In | | |
| | | | addition, serum levels of inhibin have been | | |
| | | | shown to reflect the size of granulosa-cell | | |
| | | | tumors and can therefore be used as a marker | | |
| | | | for primary as well as recurrent disease. | | |
| Cerebus Protein | GeneSeq | WO9849296 | Cerebus is believed to be involved in the | BMP activity, in the presence of the antagonist | BMP Antagonist useful for |
| | Accession | | inhibition of BMP activity | Cerebus, can be determined using the following | Osteosarcoma, abnormal |
| | W86032 | | | assays known in the art: Nat Genet. 2001 Jan; | bone growth. |
| | | | | 27(1): 84-8; Eur J Biochem 1996 Apr 1; | |
| | | | | 237(1): 295-302; J Biol Chem, Vol. 274, Issue | |
| | | | | 16, 10897-10902, Apr. 16, 1999; and Hogan, B. L. M. | |
| | | | | (1996) Genes Dev. 10, 1580-1594. | |
| Soluble BMP | GeneSeq | WO9614579 | Soluble BMP receptor kinase protein-3 is | BMP activity, in the presence of the soluble | BMP Antagonist useful for |
| Receptor Kinase | Accession | | involved in thc binding of BMPs. Soluble | antagonist BMP receptor kinase protein-3, can | Osteosarcoma, abnormal |
| Protein-3 | R95227 | | BMP receptor kinase protein-3 is useful as an | be determined using the following assays | bone growth. |
| | | | antagonist for the inhibition of BMP activity. | known in the art: Nat Genet. 2001 Jan; | |
| | | | | 27(1): 84-8; Eur J Biochem 1996 Apr 1; | |
| | | | | 237(1): 295-302; J Biol Chem, Vol. 274, Issue | |
| | | | | 16, 10897-10902, Apr. 16, 1999; and Hogan, B. L. M. | |
| | | | | (1996) Genes Dev. 10, 1580-1594. | |
| BMP Processing | GeneSeq | WO9741250 | BMPs belong to the transforming growth | BMP activity, in the presence of the Furin, can | Bone formation or |
| Enzyme Furin | Accession | | factor-beta (TGFB) superfamily. Bone | be determined using the following assays | Regeneration Abnormalities |
| | W36099 | | morphogenic protein induces bone formation. | known in the art: Nat Genet. 2001 Jan; | |
| | | | | 27(1): 84-8; Eur J Biochem 1996 Apr 1; | |
| | | | | 237(1): 295-302; J Biol Chem, Vol. 274, Issue | |
| | | | | 16, 10897-10902, Apr. 16, 1999; and Hogan, B. L. M. | |
| | | | | (1996) Genes Dev. 10, 1580-1594. | |
| TGF-beta 1 | GeneSeq | WO9216228 | Members of the TGF-beta family of proteins | The effect of TGF betas on signaling can be | Useful for treating cancer |
| | Accession | | initiate cell signaling by binding to | assayed by treating Primary BAECs transfected | and to promote wound |
| | R29657 | | heteromeric receptor complexes of type I | with a construct called p3TP-Lux, containing a | healing. |
| | | | (TbetaRI) and type II (TbetaRII) | TGF-beta responsive promoter fused to a | |
| | | | serine/threonine kinase receptors (reviewed | reporter gene, and measuring luciferase gene | |
| | | | by Massague, J. et al. (1994) Trends Cell | expression (Wrana et al., 1994, Nature 370: | |
| | | | Biol. 4: 172 178; Miyazono, K. et al. (1994) | 341-347). | |
| | | | Adv. Immunol. 55: 181-220). Activation of | | |
| | | | this heteromeric receptor complex occurs | | |
| | | | when TGF-beta. binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg. R. A. (1995) PNA892: 1565-1569; | | |
| | | | Wrana, J. L. et al. (1994) Nature 370: 341. | | |
| TGF-beta 2 | GeneSeq | EP542679 | Members of the TGF-beta family of proteins | The effect of TGF betas on signaling can be | Useful for treating cancer |
| | Accession | | initiate cell signaling by binding to | assayed by treating Primary BAECs transfected | and to promote wound |
| | R39659 | | heteromeric receptor complexes of type I | with a construct called p3TP-Lux, containing a | healing. |
| | | | (TbetaRI) and type II (TbetaRII) | TGF-beta responsive promoter fused to a | |
| | | | serine/threonine kinase receptors (reviewed | reporter gene, and measuring luciferase gene | |
| | | | by Massague, J. et al. (1994) Trends Cell | expression (Wrana et al., 1994, Nature 370: | |
| | | | Biol. 4: 172 178; Miyazono, K. et al. (1994) | 341-347). | |
| | | | Adv. Immunol. 55: 181-220). Activation of | | |
| | | | this heteromeric receptor complex occurs | | |
| | | | when TGF-beta. binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg. R. A. (1995) PNA892: 1565-1569; | | |
| | | | Wrana, J. L. et al. (1994) Nature 370: 341. | | |
| ZTGF-beta 9 | GeneSeq | WO0015798 | Members of the TGF-beta family of proteins | The effect of TGF betas on signaling can be | Useful for treating cancer |
| | Accession | | initiate cell signaling by binding to | assayed by treating Primary BAECs transfected | and to promote wound |
| | Y70654 | | heteromeric receptor complexes of type I | with a construct called p3TP-Lux, containing a | healing. |
| | | | (TbetaRI) and type II (TbetaRII) | TGF-beta responsive promoter fused to a | |
| | | | serine/threonine kinase receptors (reviewed | reporter gene, and measuring luciferase gene | |
| | | | by Massague, J. et al. (1994) Trends Cell | expression (Wrana et al., 1994, Nature 370: | |
| | | | Biol. 4: 172 178; Miyazono, K. et al. (1994) | 341-347). | |
| | | | Adv. Immunol. 55: 181-220). Activation of | | |
| | | | this heteromeric receptor complex occurs | | |
| | | | when TGF-beta. binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg. R. A. (1995) PNA892: 1565-1569; | | |
| | | | Wrana, J. L. et al. (1994) Nature 370: 341. | | |
| Anti-TGF beta | | GB2305921 | Members of the TGF-beta family of proteins | The effect of TGF betas on signaling in the | Useful for control of fibrosis, |
| family antibodies | | | initiate cell signaling by binding to | presence of an anti-TGF beta antibody, can be | immune, and inflammatory |
| | | | heteromeric receptor complexes of type I | assayed by treating Primary BAECs transfected | disease. |
| | | | (TbetaRI) and type II (TbetaRII) | with a construct called p3TP-Lux, containing a | |
| | | | serine/threonine kinase receptors (reviewed | TGF-beta responsive promoter fused to a | |
| | | | by Massague, J. et al. (1994) Trends Cell | reporter gene, and measuring luciferase gene | |
| | | | Biol. 4: 172 178; Miyazono, K. et al. (1994) | expression (Wrana et al., 1994, Nature 370: | |
| | | | Adv. Immunol. 55: 181-220). Activation of | 341-347). | |
| | | | this heteromeric receptor complex occurs | | |
| | | | when TGF-beta. binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg. R. A. (1995) PNA892: 1565-1569; | | |
| | | | Wrana, J. L. et al. (1994) Nature 370: 341. | | |
| Latent TGF beta | GeneSeq | WO0012551 | Members of the TGF-beta family of proteins | The effect of TGF betas on signaling in the | Useful for inhibiting tissue or |
| binding protein II | Accession | | initiate cell signaling by binding to | presence of a TGF beta binding protein, can be | tumor growth. |
| | Y70552 | | heteromeric receptor complexes of type I | assayed by treating Primary BAECs transfected | |
| | | | (TbetaRI) and type II (TbetaRII) | with a construct called p3TP-Lux, containing a | |
| | | | serine/threonine kinase receptors (reviewed | TGF-beta responsive promoter fused to a | |
| | | | by Massague, J. et al. (1994) Trends Cell | reporter gene, and measuring luciferase gene | |
| | | | Biol. 4: 172 178; Miyazono, K. et al. (1994) | expression (Wrana et al., 1994, Nature 370: | |
| | | | Adv. Immunol. 55: 181-220). Activation of | 341-347). | |
| | | | this heteromeric receptor complex occurs | | |
| | | | when TGF-beta. binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg. R. A. (1995) PNA892: 1565-1569; | | |
| | | | Wrana, J. L. et al. (1994) Nature 370: 341. | | |
| MP52 | GeneSeq | WO9741250 | Members of the TGF-beta family of proteins | The effect of TGF betas on signaling can be | Bone formation or |
| | Accession | | initiate cell signaling by binding to | assayed by treating Primary BAECs transfected | Regeneration Abnormalities |
| | W36100 | | heteromeric receptor complexes of type I | with a construct called p3TP-Lux, containing a | |
| | | | (TbetaRI) and type II (TbetaRII) | TGF-beta responsive promoter fused to a | |
| | | | serine/threonine kinase receptors (reviewed | reporter gene, and measuring luciferase gene | |
| | | | by Massague, J. et al. (1994) Trends Cell | expression (Wrana et al., 1994, Nature 370: | |
| | | | Biol. 4: 172 178; Miyazono, K. et al. (1994) | 341-347). | |
| | | | Adv. Immunol. 55: 181-220). Activation of | | |
| | | | this heteromeric receptor complex occurs | | |
| | | | when TGF-beta. binds to TbetaRII, which | | |
| | | | then recruits and phosphorylates TbetaRI. | | |
| | | | Activated TbetaRI then propagates the signal | | |
| | | | to downstream targets (Chen, F. and | | |
| | | | Weinberg. R. A. (1995) PNA892: 1565-1569; | | |
| | | | Wrana, J. L. et al. (1994) Nature 370: 341. | | |
| b57 Protein | GeneSeq | WO9837195 | BMPs are involved in the induction of bone | BMP activity, in the presence of b57 protein, | BMP Antagonist useful for |
| | Accession | | formation. Specific antagonists are useful is | can be determined using the following assays | Osteosarcoma, abnormal |
| | W69293 | | preventing this activity from occurring. | known in the art: Nat Genet. 2001 Jan; | bone growth. |
| | | | | 27(1): 84-8; Eur J Biochem 1996 Apr 1; | |
| | | | | 237(1): 295-302; J Biol Chem, Vol. 274, Issue | |
| | | | | 16, 1089-10902, Apr. 16, 1999; and Hogan, | |
| | | | | B. L. M. (1996) Genes Deve. 10, 1580-1594. | |
| Resistin | GeneSeq | WO0064920 | This gene belongs to the family defined by | Ability of resistin to influence type II diabetes | Type II diabetes and |
| | Accession | | mouse FIZZI and FIZZ3/Resistin genes. The | can be determined using assays known in the | Syndrome X. |
| | W69293 | | characteristic feature of this family is the C- | art: Pontoglio et al., J Clin Invest 1998 | |
| | | | terminal stretch of 10 cys residues with | May 15; 101(10): 2215-22. | |
| | | | identical spacing. The mouse homolog of this | | |
| | | | protein is secreted by adipocytes, may be the | | |
| | | | hormone potantially linking obesity to type II | | |
| | | | diabetes. | | |
| Galectin-4 | GeneSeq | WO9703190 | Galectins are a family of carbohydrate-binding | Ability of Galectin-4 polypeptides to bind | Lactose intolerance. |
| | Accession | | proteins characterized by an affinity for beta- | lactose can be determined using assays known | |
| | W11841 | | galactoside containing glycoconjugates. | in the art: Wada, et al., J Biol Chem 1997 | |
| | | | | Feb 28; 272(9): 6078-86. | |
| APM-I; ACRP-30; | GeneSeq | W00026363 | ACPR30 gene is exclusively expressed in | Ability of ACRP30 polypeptides to influence | Obesity, Metabolic disorders, |
| Famoxin | Accession | | adipose tissue. ACRP30 is thought to increase | obesity and fat oxidation can be determined | Lipid Metabolism; Hormone |
| | Y71035 | | fatty acid oxidation by muscle tissue. | using assays known in the art: Fruebis et al., | Secretion. |
| | | | | Proc Nat'l Acad Sci USA 2001 Feb 13; | |
| | | | | 98(4): 2005-10. | |
| ACRP-30 | GeneSeq | WO0063376 | ACPR30 gene is exclusively expressed in | Ability of ACRP30 homologue polypeptides | Obesity, Metabolic disorders, |
| Homologue; | Accession | | adipose tissue. ACRP30 is thought to increase | to influence obesity and fat oxidation can be | Lipid Metabolism; Hormone |
| Complement | B30234 | | fatty acid oxidation by muscle tissue. | determined using assays known in the art: | Secretion. |
| Component Clq C | | | | Fruebis et al., Proc Nat'l Acad Sci USA 2001 | |
| | | | | Feb 13; 98(4): 2005-10. | |
| Calpain-10a | GeneSeq | WO0023603 | Calpain is believed to play a role in insulin | Ability of Calpain-10 to influence type II | Diabetes mellitus; Regulation |
| | Accession | | secretion and insulin activity, and therefore | diabetes can be determined using assays | of Insulin secretory response; |
| | Y79567 | | may be useful in the treatment of type II | known in the art: Pontoglio et al., J Clin Invest | Insulin mediated glucose |
| | | | diabetes. | 1998 May 15; 101(10): 2215-22. | transport disorders. |
| Calpain-10b | GeneSeq | WO0023603 | Calpain is believed to play a role in insulin | Ability of Calpain-10 to influence type II | Diabetes mellitus; Regulation |
| | Accession | | secretion and insulin activity, and therefore | diabetes can be determined using assays | of Insulin secretory response; |
| | Y79568 | | may be useful in the treatment of type II | known in the art: Pontoglio et al., J Clin Invest | Insulin mediated glucose |
| | | | diabetes. | 1998 May 15; 101(10): 2215-22. | transport disorders. |
| Calpain-10c | GeneSeq | WO0023603 | Calpain is believed to play a role in insulin | Ability of Calpain-10 to influence type II | Diabetes mellitus; Regulation |
| | Accession | | secretion and insulin activity, and therefore | diabetes can be determined using assays | of Insulin secretory response; |
| | Y79569 | | may be useful in the treatment of type II | known in the art: Pontoglio et al., J Clin Invest | Insulin mediated glucose |
| | | | diabetes. | 1998 May 15; 101(10): 2215-22. | transport disorders. |
| PDGF-D | GeneSeq | WO0027879 | Vascular Endothelial Growth Factor. | Proliferation assay using NR6R-3T3 cells | Wound Healing; |
| | Accession | | | (Rizzino 1988 Cancer Res. 48: 4266). | Atherosclermis. |
| | Y71130 | | | | |
| FasL | GeneSeq | WO9936079 | Activities associated with apoptosis and | Activity can be determined using Apoptosis | Apoptosis-related disorders; |
| | Accession | | immune system functions. | assays known in the art: Walczak et al. (1996) | Autoimmune disorders; Graft |
| | Y28594 | | | EMBOJ 16: 5386-5397. | v-Host disorders. |
| Chondro modulin- | GeneSeq | W00029579 | Chondromodulin proteins are cartilage | Ability of Chondromodulin-like protein to | Antianglogenic agent; |
| like protein | Accession | | proteins thought to confer resistance to | inhibit vascularization can be determined | Osteoblast proliferation |
| | Y71262 | | anglogeneis, and thus are useful as anti- | using assays known in the art: Hirakie et al., J | stimulator; prevents |
| | | | angiogenic agents that may have utility in | Biol Chem 1997 Dec 19; 272(51): 32419-26. | vascularization of cartilage |
| | | | combating cancer. | | tissue; Useful to treat cancer. |
| Patched | GeneSeq | U.S. Pat. No. 5837538 | Patched is a tumour-suppressor receptor for | Ability of soluble Patched to bind to and | Receptor for Hedgehog |
| | Accession | | Sonic hedgehog (shh), which is a protein that | inhibit the activities of shh can be determined | cellular proliferation |
| | W72969 | | controls developmental patterning and growth. | using assays known in the art: Stone et al., | signaling molecule. This |
| | | | | Nature 1996 Nov 14; 384(6605): 129-34. | receptor is useful as a means |
| | | | | | of preventing cellular |
| | | | | | proliferation via the shh |
| | | | | | signaling pathway, thus |
| | | | | | useful for cancers. |
| Patched-2 | GeneSeq | WO9953058 | Patched is a tumour-suppressor receptor for | Ability of soluble Patched to bind to and | Receptor for Hedgehog |
| | Accession | | Sonic hedgehog (shh), which is a protein that | inhibit the activities of shh can be determined | cellular proliferation |
| | Y43261 | | controls developmental patterning and growth. | using assays known in the art: Stone et al., | signaling molecule. This |
| | | | | Nature 1996 Nov 14; 384(6605): 129-34. | receptor is useful as a means |
| | | | | | of preventing cellular |
| | | | | | proliferation via the shh |
| | | | | | signaling pathway, thus |
| | | | | | useful for cancers. |
| Maspin; Protease | GeneSeq | WO9405804 | Maspin is a member of the serpin family of | The inhibitory effects of Maspin and other | Tumor suppressor which is |
| Inhibitor 5 | Accession | | serine protease inhibitors that is thought to | protease inhibitors can be assayed using | down-regulated in breast |
| | R50938 | | suppress tumor metastasis. | methods known in the art such as a labeled | cancers. The maspin protein |
| | | | | protease substrate, for example, Universal | has tumour suppressing and |
| | | | | Protease Substrate (casein, resorufin-labeled): | invasion suppressing activity. |
| | | | | Roche Molecular Biochemicals, Cat. No. | |
| | | | | 1080733. | |
| Endostatin | GeneSeq | WO0064946 | Endostatin is believed to inhibit effects of | The inhibitory effects of endostatin can be | Anti-angiogenic activity. |
| | Accession | | capillary endothelial cell proliferation. | assayed using assays disclosed by Cao et al. | Useful in the prevention |
| | B28399 | | | (1996) J. Biol. Chem. 271 29461-29467. | and/or treatment of cancers. |
| aFGF; FGF-1 | GeneSeq | EP298723 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | P94037 | | | Examples 23 and 39 disclosed herein. | epithelial cells and |
| | | | | | keratinocytes. Antagonists |
| | | | | | may be useful as anti-cancer |
| | | | | | agents. |
| bFGF; FGF-2 | GeneSeq | FR2642086 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | R06685 | | | Examples 23 and 39 disclosed herein. | epithelial cells and |
| | | | | | keratinocytes. Antagonists |
| | | | | | may be useful as anti-cancer |
| | | | | | agents. |
| FGF-3; INT-2 | GeneSeq | WO9503831 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | R07824 | | | Examples 23 and 39 disclosed herein. | epithelial cells and |
| | | | | | keratinocytes. Antagonists |
| | | | | | may be useful as anti-cancer |
| | | | | | agents. |
| FGF-4; HST-1; | GeneSeq | WO9503831 | Fibroblast Growth Factor | Proliferation assay using NR6R-3T3 cells | Promotion of growth and |
| HBGF-4 | Accession | | | (Rizzino 1988 Cancer Res. 48: 4266); | proliferation of cells, such as |
| | R07825 | | | Examples 23 and 39 disclosed herein. | epithelial cells and |
| | | | | | keratinocytes. Antagonists |
| | | | | | may be useful as anti-cancer |
| | | | | | agents. |
| FGF-5 | GeneSeq | WO9730155 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | W22600 | | | Examples 23 and 39 disclosed herein. | epithelial cells and |
| | | | | | keratinocytes. Antagonists |
| | | | | | may be useful as anti-cancer |
| | | | | | agents. |
| FGF-6; Heparin | GeneSeq | EP613946 | Fibroblast Growth Factor | Proliferation assay using NR6R-3T3 cells | Promotion of growth and |
| binding secreted | Accession | | | (Rizzino 1988 Cancer Res. 48: 4266); | proliferation of cells, such as |
| transforming factor-2 | R58555 | | | Examples 23 and 39 disclosed herein. | epithelial cells and |
| | | | | | keratinocytes. Antagonists |
| | | | | | may be useful as anti-cancer |
| | | | | | agents. |
| FGF-8 | GeneSeq | WO9524928 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | R80783 | | | Examples 23 and 39 disclosed herein. | epithelial cells and |
| | | | | | keratinocytes. Antagonists |
| | | | | | may be useful as anti-cancer |
| | | | | | agents. |
| FGF-9; Gila | GeneSeq | WO9503831 | Fibroblast Growth Factor | Proliferation assay using NR6R-3T3 cells | Promotion of growth and |
| activating factor | Accession | | | (Rizzino 1988 Cancer Res. 48: 4266); | proliferation of cells, such as |
| | R70822 | | | Examples 23 and 39 disclosed herein. | epithelial cells and |
| | | | | | keratinocytes. Antagonists |
| | | | | | may be useful as anti-cancer |
| | | | | | agents. |
| FGF-12; Fibroblast | GeneSeq | WO9635708 | Fibroblast Growth Factor | Proliferation assay using NR6R-3T3 cells | Promotion of growth and |
| growth factor | Accession | | | (Rizzino 1988 Cancer Res. 48: 4266); | proliferation of cells, such as |
| homologous factor-1 | W06309 | | | Examples 23 and 39 disclosed herein. | epithelial cells and |
| | | | | | keratinocytes. Antagonists |
| | | | | | may be useful as anti-cancer |
| | | | | | agents. |
| FGF-15 | GeneSeq | WO9927100 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | Y08582 | | | Examples 23 and 39 disclosed herein. | epithelial cells and |
| | | | | | keratinocytes. Antagonists |
| | | | | | may be useful as anti-cancer |
| | | | | | agents. |
| FGF-16 | GeneSeq | WO9918128 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | Y05474 | | | Examples 23 and 39 disclosed herein. | epithelial cells and |
| | | | | | keratinocytes. Antagonists |
| | | | | | may be useful as anti-cancer |
| | | | | | agents. |
| FGF-18 | GeneSeq | WO9927100 | Fibroblast Growth Factor | Proliferation assay using NR6R-3T3 cells | Promotion of growth and |
| | Accession | | | (Rizzino 1988 Cancer Res. 48: 4266); | proliferation of cells, such as |
| | Y08590 | | | Examples 23 and 39 disclosed herein. | epithelial cells and |
| | | | | | keratinocytes. Antagonists |
| | | | | | may be useful as anti-cancer |
| | | | | | agents. |
| fit-3 ligand | GeneSeq | EP627487 | Stem Cell Progenitor | Chemokine activities can be determined using | Promotion of immune cell |
| | Accession | | | assays known in the art: Methods in Molecular | growth and/or differentiation. |
| | R67541 | | | Biology, 2000, vol. 138: Chemokine | |
| | | | | Protocols. Edited by: A. E. I. Proudfoot, T. N. C. Wells, | |
| | | | | and C. A. Power. © Humana Press Inc., | |
| | | | | Totowa, NJ. | |
| VEGF-110 | GeneSeq | WO0013702 | Promotes the growth and/or proliferation of | VEGF activity can be determined using assays | Promotion of growth and |
| | Accession | | endothelial cells. | known in the art, such as those disclosed in | proliferation of cells, such as |
| | Y69417 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGB-121 | GeneSeq | WO0071713 | Promotes the growth and/or proliferation of | VEGF activity can be determined using assays | Promotion of growth and |
| | Accession | | endothelial cells. | known in the art, such as those disclosed in | proliferation of cells, such as |
| | B50432 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-138 | GeneSeq | WO9940197 | Promotes the growth and/or proliferation of | VEGF activity can be determined using assays | Promotion of growth and |
| | Accession | | endothelial cells. | known in the art, such as those disclosed in | proliferation of cells, such as |
| | Y43483 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-145 | GeneSeq | WO0013702 | Promotes the growth and/or proliferation of | VEGF activity can be determined using assays | Promotion of growth and |
| | Accession | | endothelial cells. | known in the art, such as those disclosed in | proliferation of cells, such as |
| | Y69413 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-162 | GeneSeq | W09940197 | Promotes the growth and/or proliferation of | VEGF activity can be determined using assays | Promotion of growth and |
| | Accession | | endothelial cells. | known in the art, such as those disclosed in | proliferation of cells, such as |
| | Y43484 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-165 | GeneSeq | WO0013702 | Promotes the growth and/or proliferation of | VEGF activity can be determined using assays | Promotion of growth and |
| | Accession | | endothelial cells. | known in the art, such as those disclosed in | proliferation of cells, such as |
| | Y69414 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-182 | GeneSeq | W09940197 | Promotes the growth and/or proliferation of | VEGF activity can be determined using assays | Promotion of growth and |
| | Accession | | endothelial cells. | known in the art, such as those disclosed in | proliferation of cells, such as |
| | Y43483 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-189 | GeneSeq | WO0013702 | Promotes the growth and/or proliferation of | VEGF activity can be determined using assays | Promotion of growth and |
| | Accession | | endothelial cells. | known in the art, such as those disclosed in | proliferation of cells, such as |
| | Y69415 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-206 | GeneSeq | W00013702 | Promotes the growth and/or proliferation of | VEGF activity can be determined using assays | Promotion of growth and |
| | Accession | | endothelial cells. | known in the art, such as those disclosed in | proliferation of cells, such as |
| | Y69416 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-D | GeneSeq | WO9807832 | Promotes the growth and/or proliferation of | VEGF activity can be determined using assays | Promotion of growth and |
| | Accession | | endothelial cells. | known in the art, such as those disclosed in | proliferation of cells, such as |
| | W53240 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF-E; VEGF-X | GeneSeq | W09947677 | Promotes the growth and/or proliferation of | VEGF activity can be determined using assays | Promotion of growth and |
| | Accession | | endothelial cells. | known in the art, such as those disclosed in | proliferation of cells, such as |
| | Y33679 | | | International Publication No. WO0045835, for | vascular endothelial cells. |
| | | | | example. | Antagonists may be useful as |
| | | | | | anti-angiogenic agents, and |
| | | | | | may be applicable for cancer. |
| VEGF Receptor; | GeneSeq | WO9831794 | Receptor for VEGF polypeptides | VEGF activity, in the presence of flk-1 | VEGF Receptor. Fusion |
| KDR; flk-1 | Accession | | | polypeptides, can be determined using assays | protein with the extracellular |
| | W69679 | | | known in the art, such as those disclosed in | domain is useful as an anti- |
| | | | | International Publication No. WO0045835, for | angiogenic agent. |
| | | | | example. | Antagonists may be useful in |
| | | | | | the promotion of |
| | | | | | angiogenesis. |
| Soluble VEGF | GeneSeq | U.S. Pat. No. 5712380 | Receptor for VEGF polypeptides | VEGF activity, in the presence of VEGF | VEGF Receptor. Fusion |
| Receptor | Accession | | | Receptor polypeptides, can be determined | protein with the extracellular |
| | W47037 | | | using assays known in the art, such as those | domain is useful as an anti- |
| | | | | disclosed in International Publication No. | angiogenic agent. |
| | | | | WO0045835, for example. | Antagonists may be useful in |
| | | | | | the promotion of |
| | | | | | angiogenesis. |
| flt-1 | GeneSeq | WO0021560 | Receptor for VEGF polypeptides | VEGF activity, in the presence of flt-1 | VEGF Receptor. Fusion |
| | Accession | | | polypeptides, can be determined using assays | protein with the extracellular |
| | Y70751 | | | known in the art, such as those disclosed in | domain is useful as an anti- |
| | | | | International Publication No. WO0045835, for | angiogenic agent. |
| | | | | example. | Antagonists may be useful in |
| | | | | | the promotion of |
| | | | | | angiogenesis. |
| VEGF R-3; flt-4 | GeneSeq | WO0058511 | Receptor for VEGF polypeptides | VEGF activity, in the presence of flt-4 | VEGF Receptor. Fusion |
| | Accession | | | polypeptides, can be determined using assays | protein with the extracellular |
| | B29047 | | | known in the art, such as those disclosed in | domain is useful as an anti- |
| | | | | International Publication No. WO0045835, for | angiogenic agent. |
| | | | | example. | Antagonists may be useful in |
| | | | | | the promotion of |
| | | | | | angiogenesis. |
| Neuropilin-1 | GeneSeq | WO9929858 | Vascular Endothelial Growth Factor | VEGF activity can be determined using assays | Promotion of growth and |
| | Accession | | | known in the art, such as those disclosed in | proliferation of cells, such as |
| | Y06319 | | | International Publication No. WO0045835, for | vascular endothelial cells. |
| | | | | example. | Antagonists may be useful as |
| | | | | | anti-angiogenic agents, and |
| | | | | | may be applicable for cancer. |
| Neuropilin-2 | GeneSeq | WO9929858 | Vascular Endothelial Growth Factor | VEGF activity can be determined using assays | Promotion of growth and |
| | Accession | | | known in the art, such as those disclosed in | proliferation of cells, such as |
| | Y03618 | | | International Publication No. WO0045835, for | vascular endothelial cells. |
| | | | | example. | Antagonists may be useful as |
| | | | | | anti-angiogenic agents, and |
| | | | | | may be applicable for cancer. |
| Human fast twitch | GeneSeq | W09730085 | Troponins are contractile proteins that are | Ability of soluble Troponins to inhibit | Anti-angiogenesis |
| skeletal muscle | Accession | | thought to inhibit angiogencsis. High levels | anglogenesis can be determined using assays | |
| troponin C | W22597 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| Human fast twitch | GeneSeq | W09730085 | Troponins are contractile proteins that are | Ability of soluble Troponins to inhibit | Anti-angiogenesis |
| skeletal muscle | Accession | | thought to inhibit angiogencsis. High levels | anglogenesis can be determined using assays | |
| troponin I | W18054 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| Human fast twitch | GeneSeq | W09730085 | Troponins are contractile proteins that are | Ability of soluble Troponins to inhibit | Anti-angiogenesis |
| skeletal muscle | Accession | | thought to inhibit angiogencsis. High levels | anglogenesis can be determined using assays | |
| troponin T | W22599 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| fragment. | GeneSeq | W09719955 | Troponins are contractile proteins that are | Ability of soluble Troponins to inhibit | Anti-angiogenesis |
| myofibrillar protein | Accession | | thought to inhibit angiogencsis. High levels | anglogenesis can be determined using assays | |
| troponin I | W18053 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| myofibrillar protein | GeneSeq | W09719955 | Troponins are contractile proteins that are | Ability of soluble Troponins to inhibit | Anti-angiogenesis |
| troponin I | Accession | | thought to inhibit angiogencsis. High levels | anglogenesis can be determined using assays | |
| | W18054 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| Troponin peptides | GeneSeq | WO9933874 | Troponins are contractile proteins that are | Ability of soluble Troponins to inhibit | Anti-angiogenesis |
| | Accessions | | thought to inhibit angiogencsis. High levels | anglogenesis can be determined using assays | |
| | Y29581, Y29582, | | may contribute to the difficulty encountered in | known in the art:. Proc Natl Acad Sci USA | |
| | Y29583, Y29584, | | revascularizing the ischemic myocardium after | 1999 Mar 16; 96(6): 2645-50. | |
| | Y29585, and Y29586 | | cardiovascular injury. | | |
| Human fast twitch | GeneSeq | WO0054770 | Troponins are contractile proteins that are | Ability of soluble Troponins to inhibit | Anti-angiogenesis |
| skeletal muscle | Accession | | thought to inhibit angiogencsis. High levels | anglogenesis can be determined using assays | |
| Troponin subunit C | B00134 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| Human fast twitch | GeneSeq | WO0054770 | Troponins are contractile proteins that are | Ability of soluble Troponins to inhibit | Anti-angiogenesis |
| skeletal muscle | Accession | | thought to inhibit angiogencsis. High levels | anglogenesis can be determined using assays | |
| Troponin subunit I | B00135 | | may contribute to the difficulty encountered in | known in the art:. Proc Natl Acad Sci USA | |
| Protein | | | revascularizing the ischemic myocardium after | 1999 Mar 16; 96(6): 2645-50. | |
| | | | cardiovascular injury. | | |
| Human fast twitch | GeneSeq | WO0054770 | Troponins are contractile proteins that are | Ability of soluble Troponins to inhibit | Anti-angiogenesis |
| skeletal muscle | Accession | | thought to inhibit angiogencsis. High levels | anglogenesis can be determined using assays | |
| Troponin subunit T | B00136 | | may contribute to the difficulty encountered in | known in the art:. Proc Natl Acad Sci USA | |
| | | | revascularizing the ischemic myocardium after | 1999 Mar 16; 96(6): 2645-50. | |
| | | | cardiovascular injury. | | |
| Activator Inbibitor- | GeneSeq | WO9013648 | PAIs are believed to play a role in cancer, and | Methods that measure plasminogen activator | Anti-angiogenesis; blood- |
| 1; PAI-1 | Accession | | cardiovascular disease and blood-clotting | inhibitor (PAI) activity are known in the art, | clotting disorders. |
| | R08411 | | disorders. | for example, assay the ability of PAI to inhibit | |
| | | | | tissue plasminogen activator (tPA) or | |
| | | | | urokinase (uPA): J Biochem Biophys Methods | |
| | | | | 2000 Sep 11; 45(2): 127-40, Breast Cancer Res | |
| | | | | Treat 1996; 41(2): 141-6. Methods that | |
| | | | | measure anti-angiogenesis activity are known | |
| | | | | in the art, for example, Proc Natl Acad Sci USA | |
| | | | | 1999 Mar 16; 96(6): 2645-50. | |
| Plasminogen | GeneSeq | DE3722673 | PAIs are believed to play a role in cancer, and | Methods that measure plasminogen activator | Anti-angiogenesis; blood- |
| Activator Inhibitor- | Accession | | cardiovascular disease and blood-clotting | inhibitor (PAI) activity are known in the art, | clotting disorders. |
| 2; PAI-2 | P94160 | | disorders. | for example, assay the ability of PAI to inhibit | |
| | | | | tissue plasminogen activator (tPA) or | |
| | | | | urokinase (uPA): J Biochem Biophys Methods | |
| | | | | 2000 Sep 11; 45(2): 127-40, Breast Cancer Res | |
| | | | | Treat 1996; 41(2): 141-6. Methods that | |
| | | | | measure anti-angiogenesis activity are known | |
| | | | | in the art, for example, Proc Natl Acad Sci USA | |
| | | | | 1999 Mar 16; 96(6): 2645-50. | |
| Activator Inhibitor- | GeneSeq | WO9102057 | PAIs are believed to play a role in cancer, and | Methods that measure plasminogen activator | Anti-angiogenesis; blood- |
| 2; PAI-2 | Accession | | cardiovascular disease and blood-clotting | inhibitor (PAI) activity are known in the art, | clotting disorders. |
| | R10921 | | disorders. | for example, assay the ability of PAI to inhibit | |
| | | | | tissue plasminogen activator (tPA) or | |
| | | | | urokinase (uPA): J Biochem Biophys Methods | |
| | | | | 2000 Sep 11; 45(2): 127-40, Breast Cancer Res | |
| | | | | Treat 1996; 41(2): 141-6. Methods that | |
| | | | | measure anti-angiogenesis activity are known | |
| | | | | in the art, for example, Proc Natl Acad Sci USA | |
| | | | | 1999 Mar 16; 96(6): 2645-50. | |
| Human PAI-1 | GeneSeq | WO9105048 | PAIs are believed to play a role in cancer, and | Methods that measure plasminogen activator | Anti-angiogenesis; blood- |
| mutants | Accessions | | cardiovascular disease and blood-clotting | inhibitor (PAI) activity are known in the art, | clotting disorders. |
| | R11755, R11756, | | disorders. | for example, assay the ability of PAI to inhibit | |
| | R11757, R11758, | | | tissue plasminogen activator (tPA) or | |
| | R11759, R11760, | | | urokinase (uPA): J Biochem Biophys Methods | |
| | R11761, R11762 and | | | 2000 Sep 11; 45(2): 127-40, Breast Cancer Res | |
| | R11763 | | | Treat 1996; 41(2): 141-6. Methods that | |
| | | | | measure anti-angiogenesis activity are known | |
| | | | | in the art, for example, Proc Natl Acad Sci USA | |
| | | | | 1999 Mar 16; 96(6): 2645-50. | |
| CXCR3; CXC | GeneSeq | WO0018431 | Chemokines are a family of related small, | Chemokine activities can be determined using | Soluble CXCR3 polypeptides |
| | Accession | | secreted proteins involved in biological | assays known in the art: Methods in Molecular | may be useful for inhibiting |
| | Y79372 | | processes ranging from hematopoiesis, | Biology, 2000, vol. 138: Chemokine | chemokine activities and viral |
| | | | angiogenesis, and leukocyte trafficking. | Protocols. Edited by: A. E. I. Proudfoot, T. N. C. Wells, | infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| Modified Rantes | GeneSeq | WO9737005 | Chemokines are a family of related small, | Chemokine activities can be determined using | Immune disorders. |
| | Accession | | secreted proteins involved in biological | assays known in the art: Methods in Molecular | |
| | W38129 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| RANTES | GeneSeq | EP905240 | Chemokines are a family of related small, | Chemokine activities can be determined using | Immune disorders. |
| | Accession | | secreted proteins involved in biological | assays known in the art: Methods in Molecular | |
| | Y05299 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| MCI-1a | GeneSeq | WO9509232 | Chemokines are a family of related small, | Chemokine activities can be determined using | Immune disorders. |
| | Accession | | secreted proteins involved in biological | assays known in the art: Methods in Molecular | |
| | R73914 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| MCP-1b | GeneSeq | WO9929728 | Chemokines are a family of related small, | Chemokine activities can be determined using | Immune disorders. |
| | Accession | | secreted proteins involved in biological | assays known in the art: Methods in Molecular | |
| | Y26176 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| MCP-1 receptor | GeneSeq | WO9519436 | Chemokines are a family of related small, | Chemokine activities can be determined using | Soluble MCP-1 Receptor |
| | Accession | | secreted proteins involved in biological | assays known in the art: Methods in Molecular | polypeptides may be useful |
| | R79165 | | processes ranging from hematopoiesis, | Biology, 2000, vol. 138: Chemokine | for inhibiting chemokine |
| | | | angiogenesis, and leukocyte trafficking. | Protocols. Edited by: A. E. I. Proudfoot, T. N. C. Wells, | activities and viral infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| MCP-3 | GeneSeq | W09509232 | Chemokines are a family of related small, | Chemokine activities can be determined using | Immune disorders. |
| | Accession | | secreted proteins involved in biological | assays known in the art: Methods in Molecular | |
| | R73915 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| MCP-4 receptor | GeneSeq | W09809171 | Chemokines are a family of related small, | Chemokine activities can be determined using | Soluble MCP-4 Receptor |
| | Accession | | secreted proteins involved in biological | assays known in the art: Methods in Molecular | polypeptides may be useful |
| | W56689 | | processes ranging from hematopoiesis, | Biology, 2000, vol. 138: Chemokine | for inhibiting chemokine |
| | | | angiogenesis, and leukocyte trafficking. | Protocols. Edited by: A. E. I. Proudfoot, T. N. C. Wells, | activities and viral infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| RANTES receptor | GeneSeq | U.S. Pat. No. 5652133 | Chemokines are a family of related small, | Chemokine activities can be determined using | Soluble RANTES Receptor |
| | Accession | | secreted proteins involved in biological | assays known in the art: Methods in Molecular | polypeptides may be useful |
| | W29588 | | processes ranging from hematopoiesis, | Biology, 2000, vol. 138: Chemokine | for inhibiting chemokine |
| | | | angiogenesis, and leukocyte trafficking. | Protocols. Edited by: A. E. I. Proudfoot, T. N. C. Wells, | activities and viral infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| CCR5 variant | GeneSeq | WO9854317 | Chemokines are a family of related small, | Chemokine activities can be determined using | Soluble CCR5 polypeptides |
| | Accession | | secreted proteins involved in biological | assays known in the art: Methods in Molecular | may be useful for inhibiting |
| | W88238 | | processes ranging from hematopoiesis, | Biology, 2000, vol. 138: Chemokine | chemokine activities and viral |
| | | | angiogenesis, and leukocyte trafficking. | Protocols. Edited by: A. E. I. Proudfoot, T. N. C. Wells, | infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| CCR7 | GeneSeq | U.S. Pat. No. 6153441 | Chemokines are a family of related small, | Chemokine activities can be determined using | Soluble CCR7 polypeptides |
| | Accession | | secreted proteins involved in biological | assays known in the art: Methods in Molecular | may be useful for inhibiting |
| | B50859 | | processes ranging from hematopoiesis, | Biology, 2000, vol. 138: Chemokine | chemokine activities and viral |
| | | | angiogenesis, and leukocyte trafficking. | Protocols. Edited by: A. E. I. Proudfoot, T. N. C. Wells, | infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| CXC3 | GeneSeq | WO9727299 | Chemokines are a family of related small, | Chemokine activities can be determined using | Immune disorders. |
| | Accession | | secreted proteins involved in biological | assays known in the art: Methods in Molecular | |
| | W23345 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| Eotaxin | GeneSeq | WO9700960 | Chemokines are a family of related small, | Chemokine activities can be determined using | Immune disorders. |
| | Accession | | secreted proteins involved in biological | assays known in the art: Methods in Molecular | |
| | W10099 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| Neurotactin | GeneSeq | U.S. Pat. No. 6013257 | Neurotactin may play a role in chemotactic | Chemotactic leukocyte migration assays are | Immune disorders. |
| | Accessions | WO9742224 | leukocyte migration and brain inflammation | known in the art, for example: J. Immunol. | |
| | Y77537, W34307, | | processes. | Methods 33, ((1980)); Nature 1997 Jun 5; | |
| | Y53259, and, | | | 387(6633): 611-7. | |
| | Y77539 | | | | |
| Human CKbeta-9 | GeneSeq | U.S. Pat. No. 6153441 | Chemokines are a family of related small, | Chemokine activities can be determined using | Immune disorders. |
| | Accession | | secreted proteins involved in biological | assays known in the art: Methods in Molecular | |
| | B50860 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| Lymphotactin | GeneSeq | WO0073320 | Chemokines are a family of related small, | Chemokine activities can be determined using | Immune disorders. |
| | Accession | | secreted proteins involved in biological | assays known in the art: Methods in Molecular | |
| | B50052 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power, © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G. | | |
| MIP-3 alpha | GeneSeq | WO9801557 | Chemokines are a family of related small, | Chemokine activities can be determined using | Immune disorders. |
| | Accession | | secreted proteins involved in biological | assays known in the art: Methods in Molecular | |
| | W44398 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G. | | |
| MIP-3 beta | GeneSeq | WO9801557 | Chemokines are a family of related small, | Chemokine activities can be determined using | Immune disorders. |
| | Accession | | secreted proteins involved in biological | assays known in the art: Methods in Molecular | |
| | W44399 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G. | | |
| MIP-Gamma | GeneSeq | WO9504158 | Chemokines are a family of related small, | Chemokine activities can be determined using | Immune disorders. |
| | Accession | | secreted proteins involved in biological | assays known in the art: Methods in Molecular | |
| | R70798 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G. | | |
| Stem Cell | GeneSeq | WO9104274 | Chemokines are a family of related small, | Chemokine activities can be determined using | Hematopoietic growth factors. |
| Inhibitory | Accession | | secreted proteins involved in biological | assays known in the art: Methods in Molecular | |
| Factor | R11553 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G. | | |
| thrombopoietin | GeneSeq | WO9521920 | Thrombopoietin is involved in the regulation | Thrombopoietin (TPO) can be assayed to | Hematopoietic growth factors. |
| | Accession | | of the growth and differentiation of | determine regulation of growth and | |
| | R79905 | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| c-kit ligand; | GeneSeq | EP992579 and | C-kit ligan is thought to stimulate the | Chemokine activities can be determined using | Hematopoietic growth factors. |
| SCF; Mast cell | Accession | EP676470 | proliferation of mast cells, and is able to | assays known in the art: Methods in Molecular | |
| growth factor; | Y53284, R83978 | | augment the proliferation of both myeloid and | Biology, 2000, vol. 138: Chemokine | |
| MGF; | and R83977 | | lymphoid hematopoietic progenitors in bone | Protocols. Edited by: A. E. I. Proudfoot, T. N. C. Wells, | |
| Fibrosarcoma- | | | marrow culture. C-kit ligand is also though to | and C. A. Power. © Humana Press Inc., | |
| derived stem | | | act synergistically with other cytokines. | Totowa, NJ. | |
| cell factor | | | | | |
| Platelet derived | GeneSeq Accession | WO0066736 | Vascular Endothelial Growth Factor | VEGF activity can be determined using assays | Promotion of growth and |
| growth factor | B48653 | | | known in the art, such as those disclosed in | proliferation of cells, such as |
| | | | | International Publication No. WO0045835, for | vascular endothelial cells. |
| | | | | example. | Antagonists may be useful as |
| | | | | | anti-angiogenic agents, and |
| | | | | | may be applicable for cancer. |
| Melanoma | GeneSeq | WO9503328 | Melanoma inhibiting protein has melanoma- | Tumor suppressor activity of melanoma | Cancer; melanoma |
| inhibiting protein | Accession R69811 | | inhibiting activity and can be used to treat | inhibiting protein can be determined using | |
| | | | cancer (melanoma, glioblastoma, | assays known in the art: Matzuk et al., Nature | |
| | | | neuroblastoma, small cell lung cancer, | 1992 Nov 26; 360(6402): 313-9. | |
| | | | neuroectodermal tumors) or as an | | |
| | | | immunosuppressant (it inhibits IL-2 or | | |
| | | | phytohaemagglutinin induced proliferation of | | |
| | | | peripheral blood lymphocytes. | | |
| Glioma-derived | GeneSeq | EP399816 | Vascular Endothelial Growth Factor | VEGF activity can be determined using assays | Promotion of growth and |
| growth factor | Accession R08120 | | | known in the art, such as those disclosed in | proliferation of cells, such as |
| | | | | International Publication No. WO0045835, for | vascular endothelial cells. |
| | | | | example. | Antagonists may be useful as |
| | | | | | anti-angiogenic agents, and |
| | | | | | may be applicable for cancer. |
| Platelet derived | GeneSeq | EP682110 | Vascular Endothelial Growth Factor | VEGF activity can be determined using assays | Promotion of growth and |
| growth factor | Accession R84759 | | | known in the art, such as those disclosed in | proliferation of cells, such as |
| precursor A | | | | International Publication No. WO0045835, for | vascular endothelial cells. |
| | | | | example. | Antagonists may be useful as |
| | | | | | anti-angiogenic agents, and |
| | | | | | may be applicable for cancer. |
| Platelet derived | GeneSeq | EP682110 | Vascular Endothelial Growth Factor | VEGF activity can be determined using assays | Promotion of growth and |
| growth factor | Accession R84760 | | | known in the art, such as those disclosed in | proliferation of cells, such as |
| precursor B | | | | International Publication No. WO0045835, for | vascular endothelial cells. |
| | | | | example. | Antagonists may be useful as |
| | | | | | anti-angiogenic agents, and |
| | | | | | may be applicable for cancer. |
| Platelet derived | GeneSeq | EP282317 | Vascular Endothelial Growth Factor | VEGF activity can be determined using assays | Promotion of growth and |
| growth factor Bv-sis | Accession P80595 | | | known in the art, such as those disclosed in | proliferation of cells, such as |
| | and P80596 | | | International Publication No. WO0045835, for | vascular endothelial cells. |
| | | | | example. | Antagonists may be useful as |
| | | | | | anti-angiogenic agents, and |
| | | | | | may be applicable for cancer. |
| Placental Growth | GeneSeq Accessions | WO9206194 | Vascular Endothelial Growth Factor | VEGF activity can be determined using assays | Promotion of growth and |
| Factor | R23059 and R23060 | | | known in the art, such as those disclosed in | proliferation of cells, such as |
| | | | | International Publication No. WO0045835, for | vascular endothelial cells. |
| | | | | example. | Antagonists may be useful as |
| | | | | | anti-angiogenic agents, and |
| | | | | | may be applicable for cancer. |
| Placental Growth | GeneSeq Accession | DE19748734 | Vascular Endothelial Growth Factor | VEGF activity can be determined using assays | Promotion of growth and |
| Factor-2 | Y08289 | | | known in the art, such as those disclosed in | proliferation of cells, such as |
| | | | | International Publication No. WO0045835, for | vascular endothelial cells. |
| | | | | example. | Antagonists may be useful as |
| | | | | | anti-angiogenic agents, and |
| | | | | | may be applicable for cancer. |
| Thrombopoietin | GeneSeq Accession | WO0000612 | Thrombopoietin is involved in the regulation | Thrombopoietin (TPO) can be assayed to | Thrombocytopenia, cancer. |
| derivative1 | Y77244 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Thrombopoietin | GeneSeq Accession | WO0000612 | Thrombopoietin is involved in the regulation | Thrombopoietin (TPO) can be assayed to | Thrombocytopenia, cancer. |
| derivative2 | Y77255 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Thrombopoietin | GeneSeq Accession | WO0000612 | Thrombopoietin is involved in the regulation | Thrombopoietin (TPO) can be assayed to | Thrombocytopenia, cancer. |
| derivative3 | Y77262 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Thrombopoietin | GeneSeq Accession | WO0000612 | Thrombopoietin is involved in the regulation | Thrombopoietin (TPO) can be assayed to | Thrombocytopenia, cancer. |
| derivative4 | Y77267 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Thrombopoietin | GeneSeq Accession | WO0000612 | Thrombopoietin is involved in the regulation | Thrombopoietin (TPO) can be assayed to | Thrombocytopenia, cancer. |
| derivative5 | Y77246 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Thrombopoietin | GeneSeq Accession | WO0000612 | Thrombopoietin is involved in the regulation | Thrombopoietin (TPO) can be assayed to | Thrombocytopenia, cancer. |
| derivative6 | Y77253 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Thrombopoietin | GeneSeq Accession | WO0000612 | Thrombopoietin is involved in the regulation | Thrombopoietin (TPO) can be assayed to | Thrombocytopenia, cancer. |
| derivative7 | Y77256 | | of the growth and differentiation of | determine regulation of growth and | |
| | | | megakaryocytes and preceptors thereof. | differentiation of megakaryocytes. Mol Cell | |
| | | | | Biol 2001 Apr; 21(8): 2659-70; Exp Hematol | |
| | | | | 2001 Jan; 29(1): 51-8 and within. | |
| Fractalkine | GeneSeq Accession | U.S. Pat. No. 6043086 | Fractalkine is believed to play a role in | Fractalkine activity can be determined using | Immune disorders. |
| | Y53255 | | chemotactic leukocyte migration and | Chemotactic leukocyte migration assays | |
| | | | neurological disorders. | known in the art, for example: J. Immunol. | |
| | | | | Methods 33, ((1980)); Nature 1997 Jun | |
| | | | | 5; 387(6633): 611-7. | |
| CXC3 | GeneSeq | WO9757599 | Chemokines are a family of related small, | Chemokine activities can be determined using | Immune disorders. |
| | Accession | | secreted proteins involved in biological | assays known in the art: Methods in Molecular | |
| | W23345 | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| CCR7 | GeneSeq | U.S. Pat. No. 6153441 | Chemokines are a family of related small, | Chemokine activities can be determined using | Soluble CCR7 polypeptides |
| | Accession B50859 | | secreted proteins involved in biological | assays known in the art: Methods in Molecular | may be useful for inhibiting |
| | | | processes ranging from hematopoiesis, | Biology, 2000, vol. 138: Chemokine | chemokine activities and viral |
| | | | angiogenesis, and leukocyte trafficking. | Protocols. Edited by: A. E. I. Proudfoot, T. N. C. Wells, | infection. |
| | | | Members of this family are involved in a | and C. A. Power. © Humana Press Inc., | |
| | | | similarly diverse range of pathologies | Totowa, NJ. | |
| | | | including inflammation, allergy, tissue | | |
| | | | rejection, viral infection, and tumor biology. | | |
| | | | The chemokines exert their effects by acting | | |
| | | | on a family of seven transmembrane G-protein | | |
| | | | coupled receptors. Over 40 human | | |
| | | | chemokines have been described, which bind | | |
| | | | to ˜17 receptors thus far identified. | | |
| Nerve Growth | GeneSeq | EP414151 | Nerve Growth Factor | Proliferation assay using NR6R-3T3 cells | Neurological disorders, |
| Factor-beta | Accession R11474 | | | (Rizzino 1988 Cancer Res. 48: 4266) | cancer |
| Nerve Growth | GeneSeq | EP859056 | Nerve Growth Factor | Proliferation assay using NR6R 3T3 cells | Neurological disorders, |
| Factor-beta2 | Accession | | | (Rizzino 1988 Cancer Res. 48: 4266 | cancer |
| | W69725 | | | | |
| Neurotrophin-3 | GeneSeq | WO9821234 | Neurotrophins regulate neuronal cell | Trk tyrosine kinase activation assays | Neurological disorders, |
| | Accession | | survival and synaptic plasticity. | known in the art can be used to assay for | cancer |
| | W8889 | | | neurotrophin activity, for example, Proc | |
| | | | | Natl Acad Sci USA 2001 Mar | |
| | | | | 13; 98(6): 3555-3560. | |
| Neurotrophin-3 | GeneSeq | WO9325684 | Neurotrophins regulate neuronal cell | Trk tyrosine kinase activation assays | Neurological disorders, |
| | Accession R47100 | | survival and synaptic plasticity. | known in the art can be used to assay for | cancer |
| | | | | neurotrophin activity, for example, Proc | |
| | | | | Natl Acad Sci USA 2001 Mar | |
| | | | | 13; 98(6): 3555-3560. | |
| Neurotrophin-4a | GeneSeq | WO9325684 | Neurotrophins regulate neuronal cell | Trk tyrosine kinase activation assays | Neurological disorders, |
| | Accession R47101 | | survival and synaptic plasticity. | known in the art can be used to assay for | cancer |
| | | | | neurotrophin activity, for example, Proc | |
| | | | | Natl Acad Sci USA 2001 Mar | |
| | | | | 13; 98(6): 3555-3560. 13; 98(6): 3555-3560 | |
| Neurotrophin-4b | GeneSeq | WO9325684 | Neurotrophins regulate neuronal cell | Trk tyrosine kinase activation assays | Neurological disorders, |
| | Accession R47102 | | survival and synaptic plasticity. | known in the art can be used to assay for | cancer |
| | | | tyrosine kinases. | neurotrophin activity, for example, Proc | |
| | | | | Natl Acad Sci USA 2001 Mar | |
| | | | | 13; 98(6): 3555-3560. | |
| Neurotrophin-4c | GeneSeq | WO9325684 | Neurotrophins regulate neuronal cell | Trk tyrosine kinase activation assays | Neurological disorders, |
| | Accession R47103 | | survival and synaptic plasticity. | known in the art can be used to assay for | cancer |
| | | | tyrosine kinases. | neurotrophin activity, for example, Proc | |
| | | | | Natl Acad Sci USA 2001 Mar | |
| | | | | 13; 98(6): 3555-3560. | |
| Neurotrophin-4d | GeneSeq | WO9325684 | Neurotrophins regulate neuronal cell | Trk tyrosine kinase activation assays | Neurological disorders, |
| | Accession R47102 | | survival and synaptic plasticity. | known in the art can be used to assay for | cancer |
| | | | tyrosine kinases. | neurotrophin activity, for example, Proc | |
| | | | | Natl Acad Sci USA 2001 Mar | |
| | | | | 13; 98(6): 3555-3560. | |
| Platelet-Derived | GeneSeq | U.S. Pat. No. 5219739 | Vascular Endothelial Growth Factor | VEGF activity can be determined using | Promotion of growth and |
| Growth Factor | Accession R38918 | | | assays known in the art, such as those | proliferation of cells, such |
| A chain | | | | disclosed in International Publication No. | as vascular endothelial |
| | | | | W00045835, for example. | cells. Hematopoietic and |
| | | | | | immune disorders. |
| | | | | | Antagonists may be useful |
| | | | | | as anti-angiogenic agents, |
| | | | | | and may be applicable for |
| | | | | | cancer |
| Platelet-Derived | GeneSeq | U.S. Pat. No. 5219739 | Vascular Endothelial Growth Factor | VEGF activity can be determined using | Promotion of growth and |
| Growth Factor | Accession R38919 | | | assays known in the art, such as those | proliferation of cells, such |
| B chain | | | | disclosed in International Publication No. | as vascular endothelial |
| | | | | W00045835, for example. | cells. Hematopoietic and |
| | | | | | immune disorders. |
| | | | | | Antagonists may be useful |
| | | | | | as anti-angiogenic agents, |
| | | | | | and may be applicable for |
| | | | | | cancer |
| Stromal Derived | GeneSeq | WO9948528 | Stromal Growth Factor | Proliferation assay using NR6R-3T3 cells | Hematopoietic, immune |
| Factor-1 alpha | Accession | | | (Rizzino 1988 Cancer Res. 48: 4266) | disorders, cancer |
| | Y39995 | | | | |
| Stromal Derived | GeneSeq | CA2117953 | Stromal Growth Factor | Proliferation assay using NR6R-3T3 cells | Hematopoietic, immune |
| Factor-1 beta | Accession | | | (Rizzino 1988 Cancer Res. 48: 4266) | disorders, cancer |
| | R75420 | | | | |
| Tarc | GeneSeq | WO9711969 | Chemotactic for T lymphocytes. May | Chemotactic leukocyte migration assays | Antiinflammatory. |
| | Accession | | play a role in T-cell development. | are known in the art, for example: J. | Immune disorders, cancer |
| | W14917 | | Thought to bind CCR8 and CCR4 | Immunol. Methods 33 ((1980)) | |
| Prolactin | GeneSeq | WO9521625 | Prolactin is involved in immune cell | Immune coil proliferation and suppression | Reproductive system |
| | Accession R78691 | | proliferation and apoptosis. | of apoptosis by prolactin can be assayed by | disorders, cancer. |
| | | | | methods well-known in the art, for | |
| | | | | example, Buckley, AR and Buckley DJ, | |
| | | | | Ann NY Acad Sci 2000; 917: 522-33, and | |
| | | | | within. | |
| Prolactin2 | GeneSeq | U.S. Pat. No. 5955346 | Prolactin is involved in immune cell | Immune coil proliferation and suppression | Reproductive system |
| | Accession | | proliferation and apoptosis. | of apoptosis by prolactin can be assayed by | disorders, cancer. |
| | Y31764 | | | methods well-known in the art, for | |
| | | | | example, Buckley, AR and Buckley DJ, | |
| | | | | Ann NY Acad Sci 2000; 917: 522-33, and | |
| | | | | within. | |
| Follicle | GeneSeq | EP974359 | FSH stimulates secretion of interleukin-1 | FSH activities can be determined using | Reproductive system |
| stimulating | Accession | | by cells isolated from women in the | assays known in the art; J Gend Specif | disorders, cancer. |
| hormone Alpha | Y54160 | | follicular phase | Med 1999 Nov-Dec; 2(6): 30-4; Mol Cell | |
| subunit | | | | Endocrinol. 1997 Nov 15; 134(2): 109-18. | |
| Follicle | GeneSeq | EP974359 | FSH stimulates secretion of interleukin-1 | FSH activities can be determined using | Reproductive system |
| stimulating | Accession | | by cells isolated from women in the | assays known in the art; J Gend Specif | disorders, cancer. |
| hormone Beta | Y54161 | | follicular phase | Med 1999 Nov-Dec; 2(6): 30-4; Mol Cell | |
| subunit | | | | Endocrinol. 1997 Nov 15; 134(2): 109-18. | |
| Substance P | GeneSeq | WO0054053 | Substance P is associated with | Immuneregulation and bone marrow, cell | diabetes mellitus, |
| (tachykinin) | Accession | | immunoregulation. | proliferation by substance P can be | hypertension, cancer |
| | B23027 | | | assayed by methods well-known in the art, | |
| | | | | for example, Lai et al. Proc Natl Acad Sci | |
| | | | | USA 2001 Mar 27; 98(7): 3970-5; Jallat- | |
| | | | | Daloz et al. Allergy Asthma Proc 2001 | |
| | | | | Jan-Feb; 22(1): 17-23; Kahler et al. Exp | |
| | | | | Lung Res 2001 Jan-Feb; 27(1): 25-46; and | |
| | | | | Adamus MA and Dabrowski ZJ. J Cell | |
| | | | | Biochem 2001; 81(3)499-506. | |
| Ocytocin | GeneSeq | WO0053755 | Oxytocin is involved in the induction of | Oxytocin and prostaglandin E(2) release | inflammatory disorders |
| (Neurophysin I) | Accession | | prostaglandin (E2) release as well as an | and Ocytocin (Ca2+) increase can be | immunologic disorders, |
| | B24085 and | | increased amount of calcium release by | assayed by methods well-known in the art, | cancer |
| | B24086 | | smooth muscle cells. | for example, Pavan et al., AM J Obset | |
| | | | | Gynecol 2000 Jul; 183(1): 76-82 and Holda | |
| | | | | et al., Cell Calcium 1996 Jul; 20(1): 43 51. | |
| Vasopressin | GeneSeq | WO0053755 | Vasopressinis believed to have a direct | Vasopressin activity can be determined | inflammatory disorders |
| (Neurophysin II) | Accession | | antidiuretic action on the kidney, and it is | using assays known in the art, for example, | immunologic disorders, |
| | B24085 and | | thought to cause vasoconstriction of the | Endocr Regul 1996 Mar; 30(1): 13-17. | cancer |
| | B24086 | | peripheral vessels. | | |
| IL-1 | GeneSeq | EP165654 | Interleukins are a group of | Interleukin activity can be determined | inflammatory disorders, |
| | Accession | | multifunctional cytokines synthesized by | using assays known in the art: Matthews et | immunologic disorders, |
| | P60326 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarclio (1989) | |
| | | | and lymphocytes), chemotaxis of | Cytokine 1, 14-20. | |
| | | | neutrophils and T lymphocytes, and/or | | |
| | | | inhibition of interferons. | | |
| IL-1 mature | GeneSeq | EP456332 | Interleukins are a group of | Interleukin activity can be determined | inflammatory disorders, |
| | Accession | | multifunctional cytokines synthesized by | using assays known in the art: Matthews et | immunologic disorders, |
| | R14855 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarclio (1989) | |
| | | | and lymphocytes), chemotaxis of | Cytokine 1, 14-20. | |
| | | | neutrophils and T lymphocytes, and/or | | |
| | | | inhibition of interferons. | | |
| IL-1 beta | GeneSeq | WO9922763 | Interleukins are a group of | Interleukin activity can be determined | inflammatory disorders, |
| | Accession | | multifunctional cytokines synthesized by | using assays known in the art: Matthews et | immunologic disorders, |
| | Y08322 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarclio (1989) | |
| | | | and lymphocytes), chemotaxis of | Cytokine 1, 14-20. | |
| | | | neutrophils and T lymphocytes, and/or | | |
| | | | inhibition of interferons. | | |
| IL-3 variants | GeneSeq | WO8806161 | Interleukins are a group of | Interleukin activity can be determined | inflammatory disorders, |
| | Accession | | multifunctional cytokines synthesized by | using assays known in the art: Matthews et | immunologic disorders, |
| | P80382, P80383, | | lymphocytes, monocytes, and | al., in Lymphokines and Interferens: A | cancer |
| | P80384, and | | macrophages. Known functions include | Practical Approach, Clemens et al., eds, | |
| | P80381 | | stimulating proliferation of immune cells | IRL Press, Washington, D.C. 1987, pp. | |
| | | | (e.g., T helper cells, B cells, eosinophils, | 221-225; and Kitamura et al (1989) J Cell | |
| | | | and lymphocytes), chemotaxis of | Physiol. 140 323-334. | |
| | | | neutrophils and T lymphocytes, and/or | | |
| | | | inhibition of interferons. | | |
| IL-4 | GeneSeq | WO8702990 | Interleukins are a group of | Interleukin activity can be determined | inflammatory disorders, |
| | Accession | | multifunctional cytokines synthesized by | using assays known in the art: Matthews et | immunologic disorders, |
| | P70615 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Siegel & Mostowski (1990) J | |
| | | | and lymphocytes), chemotaxis of | Immunol Methods 132, 287-295. | |
| | | | neutrophils and T lymphocytes, and/or | | |
| | | | inhibition of interferons. | | |
| IL-4 muteins | GeneSeq | WO9747744 | Interleukins are a group of | Interleukin activity can be determined | inflammatory disorders, |
| | Accession | | multifunctional cytokines synthesized by | using assays known in the art: Matthews et | immunologic disorders, |
| | W52151 | | lymphocytes, monocytes, and | al., in Lymphokines and Interferens: A | cancer |
| | W52152 | | macrophages. Known functions include | Practical Approach, Clemens et al., eds, | |
| | W52153 | | stimulating proliferation of immune cells | IRL Press, Washington, D.C. 1987, pp. | |
| | W52154 | | (e.g., T helper cells, B cells, eosinophils, | 221-225; and Siegel & Mostowski (1990) J | |
| | W52155 | | and lymphocytes), chemotaxis of | Immunol Methods 132, 287-295. | |
| | W52156 | | neutrophils and T lymphocytes, and/or | | |
| | W52157 | | inhibition of interferons. | | |
| | W52158 | | | | |
| | W52159 | | | | |
| | W52160 | | | | |
| | W52161 | | | | |
| | W52162 | | | | |
| | W52163 | | | | |
| | W52164 | | | | |
| | and W52165 | | | | |
| IL-1 alpha | GeneSeq | EP324447 | Interleukins are a group of | Interleukin activity can be determined | inflammatory disorders, |
| | Accession | | multifunctional cytokines synthesized by | using assays known in the art: Matthews et | immunologic disorders, |
| | P90108 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| IL-3 variants | GeneSeq | WO9307171 | Interleukins are a group of | Interleukin activity can be determined | inflammatory disorders, |
| | Accession | | multifunctional cytokines synthesized by | using assays known in the art: Matthews et | immunologic disorders, |
| | R38561, R38562, | | lymphocytes, monocytes, and | al., in Lymphokines and Interferens: A | cancer |
| | R38563, R38564, | | macrophages. Known functions include | Practical Approach, Clemens et al., eds, | |
| | R38565, R38566, | | stimulating proliferation of immune cells | IRL Press, Washington, D.C. 1987, pp. | |
| | R38567, R38568, | | (e.g., T helper cells, B cells, eosinophils, | 221-225; and Aarden et al (1987) Eur. J. | |
| | R38569, R38570, | | and lymphocytes), chemotaxis of | Immunol 17, 1411-16. | |
| | R38571, and | | neutrophils and T lymphocytes, and/or | | |
| | R38572 | | inhibition of interferons. | | |
| IL-6 | GeneSeq | WO9402512 | Interleukins are a group of | Interleukin activity can be determined | inflammatory disorders, |
| | Accession | | multifunctional cytokines synthesized by | using assays known in the art: Matthews et | immunologic disorders, |
| | R45717 and | | lymphocytes, monocytes, and | al., in Lymphokines and Interferens: A | cancer |
| | R45718 | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Aarden et al (1987) Eur. J. | |
| | | | and lymphocytes), chemotaxis of | Immunol 17, 1411-16. | |
| | | | neutrophils and T lymphocytes, and/or | | |
| | | | inhibition of interferons. | | |
| IL-13 | GeneSeq | WO9404680 | Interleukins are a group of | Interleukin activity can be determined | inflammatory disorders, |
| | Accession | | multifunctional cytokines synthesized by | using assays known in the art: Matthews et | immunologic disorders, |
| | R48624 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Boutelier et al (1995) J. | |
| | | | and lymphocytes), chemotaxis of | Immunol. Methods 181, 29. | |
| | | | neutrophils and T lymphocytes, and/or | | |
| | | | inhibition of interferons. | | |
| IL-4 mutein | GeneSeq | DE4137333 | Interleukins are a group of | Interleukin activity can be determined | inflammatory disorders, |
| | Accession | | multifunctional cytokines synthesized by | using assays known in the art: Matthews et | immunologic disorders, |
| | R47182 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Siegel & Mostowski (1990) J | |
| | | | and lymphocytes), chemotaxis of | Immunol Methods 132, 287-295. | |
| | | | neutrophils and T lymphocytes, and/or | | |
| | | | inhibition of interferons. | | |
| IL-4 mutein | GeneSeq | DE4137333 | Interleukins are a group of | Interleukin activity can be determined | inflammatory disorders, |
| Y124X | Accession | | multifunctional cytokines synthesized by | using assays known in the art: Matthews et | immunologic disorders, |
| | R47183 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Siegel & Mostowski (1990) J | |
| | | | and lymphocytes), chemotaxis of | Immunol Methods 132, 287-295. | |
| | | | neutrophils and T lymphocytes, and/or | | |
| | | | inhibition of interferons. | | |
| IL-4 mutein | GeneSeq | DE4137333 | Interleukins are a group of | Interleukin activity can be determined | inflammatory disorders, |
| Y124G | Accession | | multifunctional cytokines synthesized by | using assays known in the art: Matthews et | immunologic disorders, |
| | R47184 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Siegel & Mostowski (1990) J | |
| | | | and lymphocytes), chemotaxis of | Immunol Methods 132, 287-295. | |
| | | | neutrophils and T lymphocytes, and/or | | |
| | | | inhibition of interferons. | | |
| Human | GeneSeq | WO9317698 | Interleukins are a group of | Interleukin activity can be determined | inflammatory disorders, |
| Interleukin-10 | Accession | | multifunctional cytokines synthesized by | using assays known in the art: Matthews et | immunologic disorders, |
| (precursor) | R41664 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Thompson-Snipes et al | |
| | | | and lymphocytes), chemotaxis of | (1991) J. Exp. Med. 173, 507-510. | |
| | | | neutrophils and T lymphocytes, and/or | | |
| | | | inhibition of interferons. | | |
| Human | GeneSeq | WO9318783-A | Interleukins are a group of | Interleukin activity can be determined | inflammatory disorders, |
| Interleukin-10 | Accession | | multifunctional cytokines synthesized by | using assays known in the art: Matthews et | immunologic disorders, |
| | R42642 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Thompson-Snipes et al | |
| | | | and lymphocytes), chemotaxis of | (1991) J. Exp. Med. 173, 507-510. | |
| | | | neutrophils and T lymphocytes, and/or | | |
| | | | inhibition of interferons. | | |
| Human | GeneSeq | EP569042 | Interleukins are a group of | Interleukin activity can be determined | inflammatory disorders, |
| interleukin-1 | Accession | | multifunctional cytokines synthesized by | using assays known in the art: Matthews et | immunologic disorders, |
| beta precursor. | R42447 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarello | |
| | | | and lymphocytes), chemotaxis of | (1989) Cytokine 1, 14-20. | |
| | | | neutrophils and T lymphocytes, and/or | | |
| | | | inhibition of interferons. | | |
| Interleukin- | GeneSeq | EP578278 | Interleukins are a group of | Interleukin activity can be determined | inflammatory disorders, |
| 1alpha | Accession | | multifunctional cytokines synthesized by | using assays known in the art: Matthews et | immunologic disorders, |
| | R45364 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| Human | GeneSeq | JP04063595 | Interleukins are a group of | Interleukin activity can be determined | inflammatory disorders, |
| interleukin-3 | Accession | | multifunctional cytokines synthesized by | using assays known in the art: Matthews et | immunologic disorders, |
| variant | R22814 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Kitamura et al (1989) J Cell | |
| | | | and lymphocytes), chemotaxis of | Physiol. 140 323-334. | |
| | | | neutrophils and T lymphocytes, and/or | | |
| | | | inhibition of interferons. | | |
| IL-1i fragments | GeneSeq | EP541920 | Interleukins are a group of | Interleukin activity can be determined | inflammatory disorders, |
| | Accession | | multifunctional cytokines synthesized by | using assays known in the art: Matthews et | immunologic disorders, |
| | R35484 and | | lymphocytes, monocytes, and | al., in Lymphokines and Interferens: A | cancer |
| | R35485 | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarclio (1989) | |
| | | | and lymphocytes), chemotaxis of | Cytokine 1, 14-20. | |
| | | | neutrophils and T lymphocytes, and/or | | |
| | | | inhibition of interferons. | | |
| IL-1 inhibitor | GeneSeq | EPS541920 | Interleukins are a group of | Interleukin activity can be determined | inflammatory disorders, |
| (IL-li) | Accession | | multifunctional cytokines synthesized by | using assays known in the art: Matthews et | immunologic disorders, |
| | R35486 and | | lymphocytes, monocytes, and | al., in Lymphokines and Interferens: A | cancer |
| | R35484 | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarclio (1989) | |
| | | | and lymphocytes), chemotaxis of | Cytokine 1, 14-20. | |
| | | | neutrophils and T lymphocytes, and/or | | |
| | | | inhibition of interferons. | | |
| ICE 22 kD | GeneSeq | EP533350 | Interleukins are a group of | Interleukin activity can be determined | inflammatory disorders, |
| subunit. | Accession | | multifunctional cytokines synthesized by | using assays known in the art: Matthews et | immunologic disorders, |
| | R33780 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| ICE 20 kD | GeneSeq | EP533350 | Interleukins are a group of | Interleukin activity can be determined | inflammatory disorders, |
| subunit. | Accession | | multifunctional cytokines synthesized by | using assays known in the art: Matthews et | immunologic disorders, |
| | R33781 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| ICE 10 kD | GeneSeq | EP533350 | Interleukins are a group of | Interleukin activity can be determined | inflammatory disorders, |
| subunit | Accession | | multifunctional cytokines synthesized by | using assays known in the art: Matthews et | immunologic disorders, |
| | R33782 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| Human | GeneSeq | WO9317698 | Interleukins are a group of | Interleukin activity can be determined | inflammatory disorders, |
| Interleukin-10 | Accession | | multifunctional cytokines synthesized by | using assays known in the art: Matthews et | immunologic disorders, |
| (precursor) | R41664 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Thompson-Snipes et al | |
| | | | and lymphocytes), chemotaxis of | (1991) J. Exp. Med. 173, 507-510. | |
| | | | neutrophils and T lymphocytes, and/or | | |
| | | | inhibition of interferons. | | |
| Human | GeneSeq | WO9318783 | Interleukins are a group of | Interleukin activity can be determined | inflammatory disorders, |
| Interleukin-10 | Accession | | multifunctional cytokines synthesized by | using assays known in the art: Matthews et | immunologic disorders, |
| | R42642 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Thompson-Snipes et al | |
| | | | and lymphocytes), chemotaxis of | (1991) J. Exp. Med. 173, 507-510. | |
| | | | neutrophils and T lymphocytes, and/or | | |
| | | | inhibition of interferons. | | |
| Human | GeneSeq | EP569042 | Interleukins are a group of | Interleukin activity can be determined | inflammatory disorders, |
| Interleukin-1 | Accession | | multifunctional cytokines synthesized by | using assays known in the art: Matthews et | immunologic disorders, |
| beta precursor | R42447 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Kitamura et al (1989) J Cell | |
| | | | and lymphocytes), chemotaxis of | Physiol. 140 323-334. | |
| | | | neutrophils and T lymphocytes, and/or | | |
| | | | inhibition of interferons. | | |
| Human | GeneSeq | WO9403492 | Interleukins are a group of | Interleukin activity can be determined | inflammatory disorders, |
| interleukin-6 | Accession | | multifunctional cytokines synthesized by | using assays known in the art: Matthews et | immunologic disorders, |
| | R49041 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Aarden et al (1987) Eur. J. | |
| | | | and lymphocytes), chemotaxis of | Immunol 17, 1411-16. | |
| | | | neutrophils and T lymphocytes, and/or | | |
| | | | inhibition of interferons. | | |
| Mutant | GeneSeq | WO9411402 | Interleukins are a group of | Interleukin activity can be determined | inflammatory disorders, |
| Interleukin 6 | Accession | | multifunctional cytokines synthesized by | using assays known in the art: Matthews et | immunologic disorders, |
| S176R | R54990 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Aarden et al (1987) Eur. J. | |
| | | | and lymphocytes), chemotaxis of | Immunol 17, 1411-16. | |
| | | | neutrophils and T lymphocytes, and/or | | |
| | | | inhibition of interferons. | | |
| Interleukin 6 | GeneSeq | JP06145063 | Interleukins are a group of | Interleukin activity can be determined | inflammatory disorders, |
| | Accession | | multifunctional cytokines synthesized by | using assays known in the art: Matthews et | immunologic disorders, |
| | R55256 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Aarden et al (1987) Eur. J. | |
| | | | and lymphocytes), chemotaxis of | Immunol 17, 1411-16. | |
| | | | neutrophils and T lymphocytes, and/or | | |
| | | | inhibition of interferons. | | |
| Interleukin 8 | GeneSeq | JP06100595 | Interleukins are a group of | Interleukin activity can be determined | Soluble IL-8 receptor |
| (IL-8) receptor | Accession | | multifunctional cytokines synthesized by | using assays known in the art: Matthews et | polypeptides may be useful |
| | R53932 | | lymphocytes, monocytes, and | al., in Lymphokines and Interferens: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Holmes et al (1991) Science | |
| | | | and lymphocytes), chemotaxis of | 253, 1278-80. | |
| | | | neutrophils and T lymphocytes, and/or | | |
| | | | inhibition of interferons. | | |
| Human | GeneSeq | U.S. Pat. No. 5328988 | Interleukins are a group of | Interleukin activity can be determined | inflammatory disorders, |
| interleukin-7 | Accession | | multifunctional cytokines synthesized by | using assays known in the art: Matthews et | immunologic disorders, |
| | R59919 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Park et al (1990) J. Exp. | |
| | | | and lymphocytes), chemotaxis of | Med. 171, 1073-79. | |
| | | | neutrophils and T lymphocytes, and/or | | |
| | | | inhibition of interferons. | | |
| IL-3 containing | GeneSeq | WO9521254 | Interleukins are a group of | Interleukin activity can be determined | inflammatory disorders, |
| fusion protein. | Accession | | multifunctional cytokines synthesized by | using assays known in the art: Matthews et | immunologic disorders, |
| | R79342 and | | lymphocytes, monocytes, and | al., in Lymphokines and Interferens: A | cancer |
| | R79344 | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Kitamura et al (1989) J Cell | |
| | | | and lymphocytes), chemotaxis of | Physiol. 140 323-334. | |
| | | | neutrophils and T lymphocytes, and/or | | |
| | | | inhibition of interferons. | | |
| IL-3 mutant | GeneSeq | ZA9402636 | Interleukins are a group of | Interleukin activity can be determined | inflammatory disorders, |
| proteins | Accession | | multifunctional cytokines synthesized by | using assays known in the art: Matthews et | immunologic disorders, |
| | R79254, R79255, | | lymphocytes, monocytes, and | al., in Lymphokines and Interferens: A | cancer |
| | R79256, R79257, | | macrophages. Known functions include | Practical Approach, Clemens et al., eds, | |
| | R79258, R79259, | | stimulating proliferation of immune cells | IRL Press, Washington, D.C. 1987, pp. | |
| | R79260, R79261, | | (e.g., T helper cells, B cells, eosinophils, | 221-225; and Giri et al (1994) EMBO J. 13 | |
| | R79262, R79263, | | and lymphocytes), chemotaxis of | 2822-2830. | |
| | R79264, R79265, | | neutrophils and T lymphocytes, and/or | | |
| | R79266, R79267, | | inhibition of interferons. | | |
| | R79268, R79269, | | | | |
| | R79270, R79271, | | | | |
| | R79272, R79273, | | | | |
| | R79274, R79275, | | | | |
| | R79276, R79277, | | | | |
| | R79278, R79279, | | | | |
| | R79280, R79281, | | | | |
| | R79282, R79283, | | | | |
| | R79284, and | | | | |
| | R79285 | | | | |
| IL-12 p40 | GeneSeq | AU9466072 | Interleukins are a group of | Interleukin activity can be determined | inflammatory disorders, |
| subunit. | Accession | | multifunctional cytokines synthesized by | using assays known in the art: Matthews et | immunologic disorders, |
| | R63018 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| AGF | GeneSeq | WO9429344 | Interleukins are a group of | Interleukin activity can be determined | inflammatory disorders, |
| | Accession | | multifunctional cytokines synthesized by | using assays known in the art: Matthews et | immunologic disorders, |
| | R64240 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| Human | GeneSeq | WO9519786 | Interleukins are a group of | Interleukin activity can be determined | inflammatory disorders, |
| interlaukin-12 40 kD | Accession | | multifunctional cytokines synthesized by | using assays known in the art: Matthews et | immunologic disorders, |
| subunit | R79187 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Hori et al (1987), Blood 70, | |
| | | | and lymphocytes), chemotaxis of | 1069-1078. | |
| | | | neutrophils and T lymphocytes, and/or | | |
| | | | inhibition of interferons. | | |
| Human | GeneSeq | WO9530695 | Interleukins are a group of | Interleukin activity can be determined | Soluble IL-8 receptor |
| interleukin-15 | Accession | | multifunctional cytokines synthesized by | using assays known in the art: Matthews et | polypeptides may be useful |
| receptor from | R90843 | | lymphocytes, monocytes, and | al., in Lymphokines and Interferens: A | for inhibiting interleukin |
| clone P1 | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Giri et al (1994) EMBO J. 13 | |
| | | | and lymphocytes), chemotaxis of | 2822-2830. | |
| | | | neutrophils and T lymphocytes, and/or | | |
| | | | inhibition of interferons. | | |
| Human | GeneSeq | WO9604306 | Interleukins are a group of | Interleukin activity can be determined | inflammatory disorders, |
| interleukin-7 | Accession | | multifunctional cytokines synthesized by | using assays known in the art: Matthews et | immunologic disorders, |
| | R92796 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Park et al (1990) J. Exp. | |
| | | | and lymphocytes), chemotaxis of | Med. 171, 1073-79. | |
| | | | neutrophils and T lymphocytes, and/or | | |
| | | | inhibition of interferons. | | |
| interleukin-9 | GeneSeq | WO9604306 | Interleukins are a group of | Interleukin activity can be determined | inflammatory disorders, |
| | Accession | | multifunctional cytokines synthesized by | using assays known in the art: Matthews et | immunologic disorders, |
| | R92797 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| interleukin-3 | GeneSeq | WO604306 | Interleukins are a group of | Interleukin activity can be determined | inflammatory disorders, |
| | Accession | | multifunctional cytokines synthesized by | using assays known in the art: Matthews et | immunologic disorders, |
| | R92801 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Kitamura et al (1989) J Cell | |
| | | | and lymphocytes), chemotaxis of | Physiol. 140 323-334. | |
| | | | neutrophils and T lymphocytes, and/or | | |
| | | | inhibition of interferons. | | |
| Human | GeneSeq | WO9604306 | Interleukins are a group of | Interleukin activity can be determined | inflammatory disorders, |
| interleukin-5 | Accession | | multifunctional cytokines synthesized by | using assays known in the art: Matthews et | immunologic disorders, |
| | R92802 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Kitamura et al (1989) J Cell | |
| | | | and lymphocytes), chemotaxis of | Physiol. 140 323-334. | |
| | | | neutrophils and T lymphocytes, and/or | | |
| | | | inhibition of interferons. | | |
| Recombinant | GeneSeq | DE19617202 | Interleukins are a group of | Interleukin activity can be determined | inflammatory disorders, |
| interleukin-16 | Accession | | multifunctional cytokines synthesized by | using assays known in the art: Matthews et | immunologic disorders, |
| | W33373 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Lim et al (1996) J. Immunol. | |
| | | | and lymphocytes), chemotaxis of | 156, 2566-70. | |
| | | | neutrophils and T lymphocytes, and/or | | |
| | | | inhibition of interferons. | | |
| Human IL-16 | GeneSeq | DE19617202 | Interleukins are a group of | Interleukin activity can be determined | inflammatory disorders, |
| protein | Accession | | multifunctional cytokines synthesized by | using assays known in the art: Matthews et | immunologic disorders, |
| | W33234 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Lim et al (1996) J. Immunol. | |
| | | | and lymphocytes), chemotaxis of | 156, 2566-70. | |
| | | | neutrophils and T lymphocytes, and/or | | |
| | | | inhibition of interferons. | | |
| Thrl 17 human | GeneSeq | WO9708321 | Interleukins are a group of | Interleukin activity can be determined | inflammatory disorders, |
| interleukin 9 | Accession | | multifunctional cytokines synthesized by | using assays known in the art: Matthews et | immunologic disorders, |
| | W27521 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| Metl 17 human | GeneSeq | WO9708321 | Interleukins are a group of | Interleukin activity can be determined | inflammatory disorders, |
| interleukin 9 | Accession | | multifunctional cytokines synthesized by | using assays known in the art: Matthews et | immunologic disorders, |
| | W27522 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| Human | GeneSeq | EP86-4585 | Interleukins are a group of | Interleukin activity can be determined | inflammatory disorders, |
| intracellular IL-1 | Accession | | multifunctional cytokines synthesized by | using assays known in the art: Matthews et | immunologic disorders, |
| receptor | W77158 | | lymphocytes, monocytes, and | al., in Lymphokines and Interferens: A | cancer |
| antagonist. | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| Human | GeneSeq | EP864585 | Interleukins are a group of | Interleukin activity can be determined | inflammatory disorders, |
| interleukin-18 | Accession | | multifunctional cytokines synthesized by | using assays known in the art: Matthews et | immunologic disorders, |
| protein (IL-18) | W77158 | | lymphocytes, monocytes, and | al., in Lymphokines and Interferens. A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and USHIO et al (1996) J. | |
| | | | and lymphocytes), chemotaxis of | Immunol. 156, 4274-79. | |
| | | | neutrophils and T lymphocytes, and/or | | |
| | | | inhibition of interferons. | | |
| Human | GeneSeq | EP861663 | Interleukins are a group of | Interleukin activity can be determined | inflammatory disorders, |
| interleukin-18 | Accession | | multifunctional cytokines synthesized by | using assays known in the art: Matthews et | immunologic disorders, |
| | W77077 | | lymphocytes, monocytes, and | al., in Lymphokines and Interferens: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and USHIO et al (1996) J. | |
| | | | and lymphocytes), chemotaxis of | Immunol. 156, 4274-79. | |
| | | | neutrophils and T lymphocytes, and/or | | |
| | | | inhibition of interferons. | | |
| Human | GeneSeq | EP861663 | Interleukins are a group of | Interleukin activity can be determined | inflammatory disorders, |
| interleukin 18 | Accessions | | multifunctional cytokines synthesized by | using assays known in the art: Matthews | immunologic disorders, |
| derivatives | W77083, | | lymphocytes, monocytes, and | et al., in Lymphokines and Interferons. A | cancer |
| | W77084, | | macrophages. Known functions include | Practical Approach, Clemens et al., eds, | |
| | W77085, | | stimulating proliferation of immune cells | IRL Press, Washington, D.C. 1987, pp. | |
| | W77086, | | (e.g., T helper cells, B cells, eosinophils, | 221-225; and Ushio et al (1996) J. | |
| | W77087, | | and lymphocytes), chemotaxis of | Immunol, 156, 4274-79. | |
| | W77088, and | | neutrophils and T lymphocytes, and/or | | |
| | W77089 | | inhibition of interferons. | | |
| Interleukin-9 | GeneSeq | WO9827997 | Interleukins are a group of | Interleukin activity can be determined | inflammatory disorders, |
| (IL-9) mature | Accession | | multifunctional cytokines synthesized by | using assays known in the art: Matthews | immunologic disorders, |
| protein (Thr117 | W68158 | | lymphocytes, monocytes, and | et al., in Lymphokines and Interferons: A | cancer |
| version). | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| IL-9 mature | GenSeq Accession | WO9827997 | Interleukins are a group of | Interleukin activity can be determined | inflammatory disorders, |
| protein variant | W68157 | | multifunctional cytokines synthesized by | using assays known in the art: Matthews | immunologic disorders, |
| (Met117 version) | | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| Human IL-9 | GeneSeq | WO9824904 | Interleukins are a group of | Interleukin activity can be determined | inflammatory disorders, |
| receptor protein | Accession | | multifunctional cytokines synthesized by | using assays known in the art: Matthews | immunologic disorders, |
| variant #3. | W64058 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| Human IL-9 | GenSeq Accession | WO9824904 | Interleukins are a group of | Interleukin activity can be determined | Soluble IL-9 receptor |
| receptor protein | W64060 | | multifunctional cytokines synthesized by | using assays known in the art: Matthews | polypeptides may be useful |
| variant fragment | | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| Human IL-9 | GeneSeq | WO9824904 | Interleukins are a group of | Interleukin activity can be determined | Soluble IL-9 receptor |
| receptor protein | Accession | | multifunctional cytokines synthesized by | using assays known in the art: Matthews | polypeptides may be useful |
| variant #3. | W64061 | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Yang et al (1989) Blood 74, | |
| | | | and lymphocytes), chemotaxis of | 1880-84. | |
| | | | neutrophils and T lymphocytes, and/or | | |
| | | | inhibition of interferons. | | |
| Human | GeneSeq | WO9817689 | Interleukins are a group of | Interleukin activity can be determined | inflammatory disorders, |
| Interleukin-12 | Accession | | multifunctional cytokines synthesized by | using assays known in the art: Matthews | immunologic disorders, |
| p40 protein | W51311 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Hori et al (1987), Blood 70, | |
| | | | and lymphocytes), chemotaxis of | 1069-1078. | |
| | | | neutrophils and T lymphocytes, and/or | | |
| | | | inhibition of interferons. | | |
| Human | GeneSeq | WO9817689 | Interleukins are a group of | Interleukin activity can be determined | inflammatory disorders, |
| Interleukin-12 | Accession | | multifunctional cytokines synthesized by | using assays known in the art: Matthews | immunologic disorders, |
| p35 protein | W51312 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Hori et al (1987), Blood 70, | |
| | | | and lymphocytes), chemotaxis of | 1069-1078. | |
| | | | neutrophils and T lymphocytes, and/or | | |
| | | | inhibition of interferons. | | |
| Human protein | GeneSeq | DE19649233- | Interleukins are a group of | Interleukin activity can be determined | inflammatory disorders, |
| with IL-16 | Accession | | multifunctional cytokines synthesized by | using assays known in the art: Matthews | immunologic disorders, |
| activity | W63753 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Lim et al (1996) J. Immunol. | |
| | | | and lymphocytes), chemotaxis of | 156, 2566-70. | |
| | | | neutrophils and T lymphocytes, and/or | | |
| | | | inhibition of interferons. | | |
| Human protein | GeneSeq | DE19649233- | Interleukins are a group of | Interleukin activity can be determined | inflammatory disorders, |
| with IL-16 | Accession | | multifunctional cytokines synthesized by | using assays known in the art: Matthews | immunologic disorders, |
| activity | W59425 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Lim et al (1996) J. Immunol. | |
| | | | and lymphocytes), chemotaxis of | 156, 2566-70. | |
| | | | neutrophils and T lymphocytes, and/or | | |
| | | | inhibition of interferons. | | |
| Human | GeneSeq | U.S. Pat. No. 5747024 | Interleukins are a group of | Interleukin activity can be determined | inflammatory disorders, |
| interleukin-15 | Accession | | multifunctional cytokines synthesized by | using assays known in the art: Matthews | immunologic disorders, |
| | W53878 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Giri et al (1994) EMBO J. 13 | |
| | | | and lymphocytes), chemotaxis of | 2822-2830. | |
| | | | neutrophils and T lymphocytes, and/or | | |
| | | | inhibition of interferons. | | |
| Human wild-type | GeneSeq | WO9747744 | Interleukins are a group of | Interleukin activity can be determined | inflammatory disorders, |
| interleukin-4 | Accession | | multifunctional cytokines synthesized by | using assays known in the art: Matthews | immunologic disorders, |
| (hIL-4) protein | W52149 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Siegel & Mostowski (1990) J | |
| | | | and lymphocytes), chemotaxis of | Immunol Methods 132, 287-295. | |
| | | | neutrophils and T lymphocytes, and/or | | |
| | | | inhibition of interferons. | | |
| interleukin-4 | GeneSeq | WO9747744 | Interleukins are a group of | Interleukin activity can be determined | inflammatory disorders, |
| muteins | Accessions | | multifunctional cytokines synthesized by | using assays known in the art: Matthews | immunologic disorders, |
| | W52150, | | lymphocytes, monocytes, and | et al., in Lymphokines and Interferons: A | cancer |
| | W52151, | | macrophages. Known functions include | Practical Approach, Clemens et al., eds, | |
| | W52153, | | stimulating proliferation of immune cells | IRL Press, Washington, D.C. 1987, pp. | |
| | W52154, | | (e.g., T helper cells, B cells, eosinophils, | 221-225; and Siegel & Mostowski (1990) J | |
| | W52155, | | and lymphocytes), chemotaxis of | Immunol Methods 132, 287-295. | |
| | W52156, | | neutrophils and T lymphocytes, and/or | | |
| | W52157, | | inhibition of interferons. | | |
| | W52158, | | | | |
| | W52159, | | | | |
| | W52160, | | | | |
| | W52161, | | | | |
| | W52162, | | | | |
| | W52163, | | | | |
| | W52164, | | | | |
| | W52165, | | | | |
| | W52166, and | | | | |
| | W52167 | | | | |
| Human | GeneSeq | WO9935268 | Interleukins are a group of | Interleukin activity can be determined | inflammatory disorders, |
| interleukin 1 delta | Accession | | multifunctional cytokines synthesized by | using assays known in the art: Matthews | immunologic disorders, |
| | Y28408 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| Human | GeneSeq | WO9935268 | Interleukins are a group of | Interleukin activity can be determined | inflammatory disorders, |
| interleukin-I | Accession | | multifunctional cytokines synthesized by | using assays known in the art: Matthews | immunologic disorders, |
| receptor | Y24395 | | lymphocytes, monocytes, and | et al., in Lymphokines and Interferons: A | cancer |
| antagonist beta | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| Human EDIRF II | GeneSeq | WO9932632 | Interleukins are a group of | Interleukin activity can be determined | inflammatory disorders, |
| protein sequence | Accession | | multifunctional cytokines synthesized by | using assays known in the art: Matthews | immunologic disorders, |
| | Y22199 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| Human EDIRF I | GeneSeq | WO9932632 | Interleukins are a group of | Interleukin activity can be determined | inflammatory disorders, |
| protein sequence | Accession | | multifunctional cytokines synthesized by | using assays known in the art: Matthews | immunologic disorders, |
| | Y22197 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| Human IL-1RD10 | GeneSeq | WO9919480 | Interleukins are a group of | Interleukin activity can be determined | Soluble IL-1RD10 receptor |
| protein sequence | Accession | | multifunctional cytokines synthesized by | using assays known in the art: Matthews | polypeptides may be useful |
| | Y14131 | | lymphocytes, monocytes, and | et al., in Lymphokines and Interferons: A | for inhibiting interleukin |
| | | | macrophages. Known functions include | Practical Approach, Clemens et al., eds, | activites. |
| | | | stimulating proliferation of immune cells | IRL Press, Washington, D.C. 1987, pp. | |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| Human IL-1RD9 | GeneSeq | WO9919480 | Interleukins are a group of | Interleukin activity can be determined | Soluble IL-1RD10 receptor |
| | Accession | | multifunctional cytokines synthesized by | using assays known in the art: Matthews | polypeptides may be useful |
| | Y14122 | | lymphocytes, monocytes, and | et al., in Lymphokines and Interferons: A | for inhibiting interleukin |
| | | | macrophages. Known functions include | Practical Approach, Clemens et al., eds, | activites. |
| | | | stimulating proliferation of immune cells | IRL Press, Washington, D.C. 1987, pp. | |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| Human DNAX | GeneSeq | WO9919491 | Interleukins are a group of | Interleukin activity can be determined | inflammatory disorders, |
| interleukin-40 | Accession | | multifunctional cytokines synthesized by | using assays known in the art: Matthews | immunologic disorders, |
| | Y09196 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| (DIL-40) | GeneSeq | WO9919491 | Interleukins are a group of | Interleukin activity can be determined | inflammatory disorders, |
| alternative | Accession | | multifunctional cytokines synthesized by | using assays known in the art: Matthews | immunologic disorders, |
| sequence | Y09197 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| IL-11 | GeneSeq | WO9405318 | Interleukins are a group of | Interleukin activity can be determined | inflammatory disorders, |
| | Accession | | multifunctional cytokines synthesized by | using assays known in the art: Matthews | immunologic disorders, |
| | R50176 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Lu et al (1994) J immunol. | |
| | | | and lymphocytes), chemotaxis of | Methods 173, 19. | |
| | | | neutrophils and T lymphocytes, and/or | | |
| | | | inhibition of interferons. | | |
| Human | GeneSeq | EP566410 | Interleukins are a group of | Interleukin activity can be determined | inflammatory disorders, |
| adipogenesis | Accession | | multifunctional cytokines synthesized by | using assays known in the art: Matthews | immunologic disorders, |
| inhibitory factor | R43260 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| IL-11 | GeneSeq | JP08127539 | Interleukins are a group of | Interleukin activity can be determined | inflammatory disorders, |
| | Accession | | multifunctional cytokines synthesized by | using assays known in the art: Matthews | immunologic disorders, |
| | W02202 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Lu et al (1994) J immunol. | |
| | | | and lymphocytes), chemotaxis of | Methods 173, 19. | |
| | | | neutrophils and T lymphocytes, and/or | | |
| | | | inhibition of interferons. | | |
| IL-14 | GeneSeq | WO9416074 | Interleukins are a group of | Interleukin activity can be determined | inflammatory disorders, |
| | Accession | | multifunctional cytokines synthesized by | using assays known in the art: Matthews | immunologic disorders, |
| | R55800 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Ambrus et al (1993) PNAS | |
| | | | and lymphocytes), chemotaxis of | 90, 63330-34. | |
| | | | neutrophils and T lymphocytes, and/or | | |
| | | | inhibition of interferons. | | |
| IL-17 receptor | GeneSeq | U.S. Pat. No. 6072033 | Interleukins are a group of | Interleukin activity can be determined | Soluble IL-17 receptor |
| | Accession | | multifunctional cytokines synthesized by | using assays known in the art: Matthews | polypeptides may be useful |
| | B03807 | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Yao et al (1995) J. Immunol. | |
| | | | and lymphocytes), chemotaxis of | 155, 5483-86. | |
| | | | neutrophils and T lymphocytes, and/or | | |
| | | | inhibition of interferons. | | |
| IL-17 | GeneSeq | WO9518826 | Interleukins are a group of | Interleukin activity can be determined | inflammatory disorders, |
| | Accession | | multifunctional cytokines synthesized by | using assays known in the art: Matthews | immunologic disorders, |
| | R76573 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Yao et al (1995) J. Immunol. | |
| | | | and lymphocytes), chemotaxis of | 155, 5483-86. | |
| | | | neutrophils and T lymphocytes, and/or | | |
| | | | inhibition of interferons. | | |
| CTLA-8 | GeneSeq | WO9704097 | Interleukins are a group of | Interleukin activity can be determined | inflammatory disorders, |
| | Accession | | multifunctional cytokines synthesized by | using assays known in the art: Matthews | immunologic disorders, |
| | W13651 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225. | |
| | | | and lymphocytes), chemotaxis of | | |
| | | | neutrophils and T lymphocytes, and/or | | |
| | | | inhibition of interferons. | | |
| IL-19 | GeneSeq | WO9808870 | Interleukins are a group of | Interleukin activity can be determined | inflammatory disorders, |
| | Accession | | multifunctional cytokines synthesized by | using assays known in the art: Matthews | immunologic disorders, |
| | W37935 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Gallagher et al (2000) Genes | |
| | | | and lymphocytes), chemotaxis of | Immun. 1, 442-50. | |
| | | | neutrophils and T lymphocytes, and/or | | |
| | | | inhibition of interferons. | | |
| IL-21 (TIF) | GeneSeq | WO0024758 | Interleukins are a group of | Interleukin activity can be determined | inflammatory disorders, |
| | Accession | | multifunctional cytokines synthesized by | using assays known in the art: Matthews | immunologic disorders, |
| | Y92879 | | lymphocytes, monocytes, and | et al., in Lymphokines and Interferons: A | cancer |
| | | | macrophages. Known functions include | Practical Approach, Clemens et al., eds, | |
| | | | stimulating proliferation of immune cells | IRL Press, Washington, D.C. 1987, pp. | |
| | | | (e.g., T helper cells, B cells, eosinophils, | 221-225; and Parrish-Novak et al (2000) | |
| | | | and lymphocytes), chemotaxis of | Nature 408, 57-63. | |
| | | | neutrophils and T lymphocytes, and/or | | |
| | | | inhibition of interferons. | | |
| IL-8 receptor | GeneSeq | WO9306229 | Interleukins are a group of | Interleukin activity can be determined | Soluble IL-8 receptor |
| | Accession | | multifunctional cytokines synthesized by | using assays known in the art: Matthews | polypeptides may be useful |
| | R33420 | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Holmes et al (1991) Science | |
| | | | and lymphocytes), chemotaxis of | 253, 1278-80.. | |
| | | | neutrophils and T lymphocytes, and/or | | |
| | | | inhibition of interferons. | | |
| Human type II | GeneSeq | U.S. Pat. No. 5464937 | Interleukins are a group of | Interleukin activity can be determined | Soluble type II interleukin- |
| interleukin-1 | Accession | | multifunctional cytokines synthesized by | using assays known in the art: Matthews | 1 receptor polypeptides |
| receptor | R85480 | | lymphocytes, monocytes, and | et al., in Lymphokines and Interferons: A | may be useful for |
| | | | macrophages. Known functions include | Practical Approach, Clemens et al., eds, | inhibiting interleukin |
| | | | stimulating proliferation of immune cells | IRL Press, Washington, D.C. 1987, pp. | activities. |
| | | | (e.g., T helper cells, B cells, eosinophils, | 221-225; and Orencole & Dinarello (1989) | |
| | | | and lymphocytes), chemotaxis of | Cytokine 1, 14-20. | |
| | | | neutrophils and T lymphocytes, and/or | | |
| | | | inhibition of interferons. | | |
| Human | GeneSeq | EP638644 | Interleukins are a group of | Interleukin activity can be determined | Soluble IL-12 receptor |
| interleukin-12 | Accession | | multifunctional cytokines synthesized by | using assays known in the art: Matthews | polypeptides may be useful |
| receptor | R69632 | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Hori et al (1987), Blood 70, | |
| | | | and lymphocytes), chemotaxis of | 1069-1078. | |
| | | | neutrophils and T lymphocytes, and/or | | |
| | | | inhibition of interferons. | | |
| Interleukin 8 | GeneSeq | U.S. Pat. No. 5440021 | Interleukins are a group of | Interleukin activity can be determined | Soluble IL-8 receptor B |
| receptor B | Accession | | multifunctional cytokines synthesized by | using assays known in the art: Matthews | polypeptides may be useful |
| | R80758 | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Holmes et al (1991) Science | |
| | | | and lymphocytes), chemotaxis of | 253, 1278-80. | |
| | | | neutrophils and T lymphocytes, and/or | | |
| | | | inhibition of interferons. | | |
| Human IL-8 | GeneSeq | JP08103276 | Interleukins are a group of | Interleukin activity can be determined | Soluble IL-8 receptor A |
| receptor protein | Accession B09989 | | multifunctional cytokines synthesized by | using assays known in the art: Matthews | polypeptides may be useful |
| hIL8RA | | | lymphocytes, monocytes, and | et al., in Lymphokines and Interferons: A | for inhibiting interleukin |
| | | | macrophages. Known functions include | Practical Approach, Clemens et al., eds, | activities. |
| | | | stimulating proliferation of immune cells | IRL Press, Washington, D.C. 1987, pp. | |
| | | | (e.g., T helper cells, B cells, eosinophils, | 221-225; and Holmes et al (1991) Science | |
| | | | and lymphocytes), chemotaxis of | 253, 1278-80. | |
| | | | neutrophils and T lymphocytes, and/or | | |
| | | | inhibition of interferons. | | |
| Human IL-8 | GeneSeq Accession | JP08103276 | Interleukins are a group of multifunctional | Interleukin activity can be determined using | Soluble IL-8 receptor |
| receptor protein | B09990 | | cytokines synthesized by lymphocytes, | asays known in the art: Matthews et al., in | polypeptides may be useful |
| hIL8R | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Holmes et al (1991) Science 253, 1278-80. | |
| | | | neutrophils and T lymphocytes, and/or | | |
| | | | inhibition of interferons. | | |
| Interleukin-2 | GeneSeq Accession | WO9621732- | Interleukins are a group of multifunctional | Interleukin activity can be determined using | Soluble IL-2 receptor |
| receptor associated | R97569 | | cytokines synthesized by lymphocytes, | assays known in the art: Matthews et al., in | polypeptides may be useful |
| protein p43 | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Gillis et al (1978) J. Immunol. 120, 2027. | |
| | | | neutrophils and T lymphocytes, and/or | | |
| | | | inhibition of interferons. | | |
| Human interleukin- | GeneSeq Accession | WO9629408 | Interleukins are a group of multifunctional | Interleukin activity can be determined using | Soluble IL-17 receptor |
| 17 receptor | W04185 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Yao | |
| | | | eosinophils, and lymphocytes), chemotaxis of | et al (1995) J. Immunol. 155, 5483-86. | |
| | | | neutrophils and T lymphocytes, and/or | | |
| | | | inhibition of interferons. | | |
| Human interleukin- | GeneSeq Accession | WO9619574 | Interleukins are a group of multifunctional | Interleukin activity can be determined using | Soluble IL-11 receptor |
| 11 receptor | R99090 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Lu | |
| | | | eosinophils, and lymphocytes), chemotaxis of | et al (1994) J immunol. Methods 173, 19. | |
| | | | neutrophils and T lymphocytes, and/or | | |
| | | | inhibition of interferons. | | |
| Human interleukin- | GeneSeq Accession | WO9623067 | Interleukins are a group of multifunctional | Interleukin activity can be determined using | Inflammatory disorders, |
| 1 receptor accessory | W01911 | | cytokines synthesized by lymphocytes, | assays known in the art: Matthews et al., in | immunologic disorders, |
| protein | | | monocytes, and macrophages. Known | Lymphokines and Interferons: A Practical | cancer |
| | | | functions include stimulating proliferation of | Approach, Clemens et al., eds, IRL Press, | |
| | | | immune cells (e.g., T helper cells, B cells, | Washington, D.C. 1987, pp. 221-225; and | |
| | | | eosinophils, and lymphocytes), chemotaxis of | Orencole & Dinarello (1989) Cytokine 1, 14-20. | |
| | | | neutrophils and T lymphocytes, and/or | | |
| | | | inhibition of interferons. | | |
| AGF Protein | GeneSeq Accession | U.S. Pat. No. 5488032 | Interleukins are a group of multifunctional | Interleukin activity can be determined using | Inflammatory disorders, |
| | R92749 | | cytokines synthesized by lymphocytes, | assays known in the art: Matthews et al., in | immunologic disorders, |
| | | | monocytes, and macrophages. Known | Lymphokines and Interferons: A Practical | cancer |
| | | | functions include stimulating proliferation of | Approach, Clemens et al., eds, IRL Press, | |
| | | | immune cells (e.g., T helper cells, B cells, | Washington, D.C. 1987, pp. 221-225. | |
| | | | eosinophils, and lymphocytes), chemotaxis of | | |
| | | | neutrophils and T lymphocytes, and/or | | |
| | | | inhibition of interferons. | | |
| Human interleukin- | GeneSeq Accession | W09607739 | Interleukins are a group of multifunctional | Interleukin activity can be determined using | Soluble IL-type-3 receptor |
| 1 type-3 receptor | R91064 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities |
| | | | immune cells (e.g., T helper cells, B cells, | Washington, D.C. 1987, pp. 221-225; and | |
| | | | eosinophils, and lymphocytes), chemotaxis of | Orencole & Dinarello (1989) Cytokine 1, 14-20. | |
| | | | neutrophils and T lymphocytes, and/or | | |
| | | | inhibition of interferons. | | |
| Human interleukin- | GeneSeq Accession | WO9720926 | Interleukins are a group of multifunctional | Interleukin activity can be determined using | Soluble IL-13 beta receptor |
| 13 beta receptor | W24972 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Boutelier et al (1995) J. Immunol. Methods | |
| | | | neutrophils and T lymphocytes, and/or | 181, 29. | |
| | | | inhibition of interferons. | | |
| Human interleukin- | GeneSeq Accession | WO9720926 | Interleukins are a group of multifunctional | Interleukin activity can be determined using | Soluble IL-13 alpha receptor |
| 13 alpha receptor | W24973 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Boutelier et al (1995) J. Immunol. Methods | |
| | | | neutrophils and T lymphocytes, and/or | 181, 29. | |
| | | | inhibition of interferons. | | |
| Human interleukin- | GeneSeq Accession | U.S. Pat. No. 5599905 | Interleukins are a group of multifunctional | Interleukin activity can be determined using | Soluble IL-4 receptor |
| 4 receptor | W13499 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Siegel & Mostowski (1990) J Immunol | |
| | | | neutrophils and T lymphocytes, and/or | Methods 132, 287-295. | |
| | | | inhibition of interferons. | | |
| Human interleukin- | GeneSeq Accession | EP759466 | Interleukins are a group of multifunctional | Interleukin activity can be determined using | Soluble IL-12 beta-2 receptor |
| 12 beta-2 receptor | W12771 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Hori | |
| | | | eosinophils, and lymphocytes), chemotaxis of | et al (1987), Blood 70, 1069-1078. | |
| | | | neutrophils and T lymphocytes, and/or | | |
| | | | inhibition of interferons. | | |
| Human interleukin- | GeneSeq Accession | EP759466 | Interleukins are a group of multifunctional | Interleukin activity can be determined using | Soluble IL-12 beta-1 receptor |
| 12 beta-1 receptor. | W12772 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Hori | |
| | | | eosinophils, and lymphocytes), chemotaxis of | et al (1987), Blood 70, 1069-1078. | |
| | | | neutrophils and T lymphocytes, and/or | | |
| | | | inhibition of interferons. | | |
| Human IL-9 | GeneSeq | WO9824904 | Interleukins are a group of multifunctional | Interleukin activity can be determined using | Soluble IL-9 receptor |
| receptor protein | Accessions | | cytokines synthesized by lymphocytes, | assays known in the art: Matthews et al., in | polypeptides may be useful |
| | W64055, W64056, | | monocytes, and macrophages. Known | Lymphokines and Interferons: A Practical | for inhibiting interleukin |
| | and W64057 | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Yang et al (1989), Blood 74, 1880-84.. | |
| | | | neutrophils and T lymphocytes, and/or | | |
| | | | inhibition of interferons. | | |
| IL-10 receptor | GeneSeq Accession | U.S. Pat. No. 5716804 | Interleukins are a group of multifunctional | Interleukin activity can be determined using | Soluble IL-10 receptor |
| | W41804 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Thompson-Snipes et al (1991) J. Exp. Med. | |
| | | | neutrophils and T lymphocytes, and/or | 173, 507-510. | |
| | | | inhibition of interferons. | | |
| Human IL-6 | GeneSeq Accession | JP11196867 | Interleukins are a group of multifunctional | Interleukin activity can be determined using | Soluble IL-6 receptor |
| receptor | Y30938 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Aarden et al (1987) Eur. J. Immunol 17, 1411-16. | |
| | | | neutrophils and T lymphocytes, and/or | | |
| | | | inhibition of interferons. | | |
| Il-17 receptor | GeneSeq Accession | U.S. Pat. No. 6096305 | Interleukins are a group of multifunctional | Interleukin activity can be determined using | Soluble IL-17 receptor |
| | Y97181 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Yao | |
| | | | eosinophils, and lymphocytes), chemotaxis of | et al (1995) J. Immunol. 155, 5483-86. | |
| | | | neutrophils and T lymphocytes, and/or | | |
| | | | inhibition of interferons. | | |
| Il-17 receptor | GeneSeq Accession | U.S. Pat. No. 6100235 | Interleukins are a group of multifunctional | Interleukin activity can be determined using | Soluble IL-17 receptor |
| | Y97131 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Yao | |
| | | | eosinophils, and lymphocytes), chemotaxis of | et al (1995) J. Immunol. 155, 5483-86. | |
| | | | neutrophils and T lymphocytes, and/or | | |
| | | | inhibition of interferons. | | |
| Human | GeneSeq Accession | EP509826 | Interleukins are a group of multifunctional | Interleukin activity can be determined using | Soluble IL-3 receptor |
| interleukin-3 | R25300 | | cytokines synthesized by lymphocytes, | assays known in the art: Matthews et al., in | polypeptides may be useful |
| receptor | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Kitamura et al (1989) J Cell Physiol. 140 323-334. | |
| | | | neutrophils and T lymphocytes, and/or | | |
| | | | inhibition of interferons. | | |
| Human GM-CSF | GeneSeq Accession | WO9102063 | Interleukins are a group of multifunctional | Interleukin activity can be determined using | Soluble GM-CSF receptor |
| receptor | R10919 | | cytokines synthesized by lymphocytes, | assays known in the art: Matthews et al., in | polypeptides may be useful |
| | | | monocytes, and macrophages. Known | Lymphokines and Interferons. A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225. | |
| | | | eosinophils, and lymphocytes), chemotaxis of | | |
| | | | neutrophils and T lymphocytes, and/or | | |
| | | | inhibition of interferons. | | |
| Human IL-5 | GeneSeq Accession | EP492214 | Interleukins are a group of multifunctional | Interleukin activity can be determined using | Soluble IL-5 receptor alpha |
| receptor alpha chain | R25064 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Kitamura et al (1989) J Cell Physiol. 140, 323-334. | |
| | | | neutrophils and T lymphocytes, and/or | | |
| | | | inhibition of interferons. | | |
| Il-5 receptor | GeneSeq Accession | WO9847923 | Interleukins are a group of multifunctional | Interleukin activity can be determined using | Soluble IL-5 receptor |
| | W82842 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Kitamura et al (1989) J Cell Physiol. 140, 323-334. | |
| | | | neutrophils and T lymphocytes, and/or | | |
| | | | inhibition of interferons. | | |
| Il-6 receptor | GeneSeq Accession | JP05091892 | Interleukins are a group of multifunctional | Interleukin activity can be determined using | Soluble IL-6 receptor |
| | R37215 | | cytokines synthesized by lymphocytes, | assays known in the art: Matthews et al., in | polypeptides may be useful |
| | | | monocytes, and macrophages. Known | Lymphokines and Interferons A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and | |
| | | | eosinophils, and lymphocytes), chemotaxis of | Aarden et al (1987) Eur. J. Immunol 17, 1411-16. | |
| | | | neutrophils and T lymphocytes, and/or | | |
| | | | inhibition of interferons. | | |
| Human B cell | GeneSeq Accession | AU8928720 | Interleukins are a group of multifunctional | Interleukin activity can be determined using | Soluble B cell stimulating |
| stimulating factor-2 | P90525 | | cytokines synthesized by lymphocytes, | assays known in the art: Matthews et al., in | factor-2 receptor |
| receptor | | | monocytes, and macrophages. Known | Lymphokines and Interferons: A Practical | polypeptides may be useful |
| | | | functions include stimulating proliferation of | Approach, Clemens et al., eds, IRL Press, | for inhibiting interleukin |
| | | | immune cells (e.g., T helper cells, B cells, | Washington, D.C. 1987, pp. 221-225. | activities. |
| | | | eosinophils, and lymphocytes), chemotaxis of | | |
| | | | neutrophils and T lymphocytes, and/or | | |
| | | | inhibition of interferons. | | |
| IL-7 receptor clone | GeneSeq Accession | EP403114 | Interleukins are a group of multifunctional | Interleukin activity can be determined using | Soluble IL-7 receptor |
| | R08330 | | cytokines synthesized by lymphocytes, | assays known in the art: Matthews et al., in | polypeptides may be useful |
| | | | monocytes, and macrophages. Known | Lymphokines and Interferons: A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Park | |
| | | | eosinophils, and lymphocytes), chemotaxis of | et al (1990) J. Exp. Med. 171, 1073-79. | |
| | | | neutrophils and T lymphocytes, and/or | | |
| | | | inhibition of interferons. | | |
| EPO receptor; | GeneSeq Accession | WO9008822 | EPO Receptor is involved in the proliferation | EPO Receptor activity can be determined | Inflammatory disorders, |
| EPOR | R06512 | | and differentiation of erythroblasts. | using assays known in the art, such as, J Biol | immunologic disorders, |
| | | | | Chem 2001 Mar 23; 276(12: 8995-9002; JAK2 | cancer, erythroblast |
| | | | | protein tyrosine kinase activity: Blood 1994 | proliferation and |
| | | | | Sep 1; 84(5): 1501-7 and Mol Cell Biol. 1994 | differentiation |
| | | | | Oct; 14(10: 6506-14. | |
| IL-15 receptor | GeneSeq Accession | WO9530695 | Interleukins are a group of multifunctional | Interleukin activity can be determined using | Soluble IL-15 receptor |
| | R90843 | | cytokines synthesized by lymphocytes, | assays known in the art: Matthews et al., in | polypeptides may be useful |
| | | | monocytes, and macrophages. Known | Lymphokines and Interferons. A Practical | for inhibiting interleukin |
| | | | functions include stimulating proliferation of | Approach, Clemens et al., eds, IRL Press, | activities. |
| | | | immune cells (e.g., T helper cells, B cells, | Washington, D.C. 1987, pp. 221-225; and Giri | |
| | | | eosinophils, and lymphocytes), chemotaxis of | et al (1994) EMBO J. 13 2822-2830. | |
| | | | neutrophils and T lymphocytes, and/or | | |
| | | | inhibition of interferons. | | |
| CD137; 4-1BB | GeneSeq Accession | WO9507984 | Activities associated with apoptosis, NF-kB | Apoptosis activity, NF-kB activation, and B | Soluble 4-1BB receptor |
| Receptor Protein | R70977 | | activation, and co-stimulation of immune | and T cell co-stimulation can be determined | polypeptides may be useful |
| | | | cells such as T and B cells. | using assays known in the art: Moore et al., | for inhibiting apoptosis, NF- |
| | | | | 1999, Science, 285(5425): 260-3; Song HY et | kB activation, and/or co- |
| | | | | al., 1997 Proc Natl Acad Sd USA | stimulation of immune cells |
| | | | | 94(18): 9792-6; Epsevik and Nissen-Meyer, | such as B and T cells. |
| | | | | 1986, J. Immunol. Methods. | |
| BCMA | GeneSeq Accession | WO0068378 | Activities associated with apoptosis, NF-kB | Apoptosis activity, NF-kB activation, and B | Soluble BCMA receptor |
| | Y71979 | | activation, and co-stimulation of immune | and T cell co-stimulation can be determined | polypeptides may be useful |
| | | | cells such as T and B cells. | using assays known in the art: Moore et al., | for inhibiting apoptosis, NF- |
| | | | | 1999, Science, 285(5425): 260-3; Song HY et | kB activation, and/or co- |
| | | | | al., 1997 Proc Natl Acad Sci USA | stimulation of immune cells |
| | | | | 94(18): 9792-6; Epsevik and Nissen-Meyer, | such as B and T cells. |
| | | | | 1986, J. Immunol. Methods. | |
| CD27 | GeneSeq Accession | WO9201049 | Activities associated with apoptosis, NF-kB | Apoptosis activity, NF-kB activation, and B | Soluble CD27 polypeptides |
| | R20814 | | activation, and co-stimulation of immune | and T cell co-stimulation can be determined | may be useful for inhibiting |
| | | | cells such as T and B cells. | using assays known in the art: Moore et al., | apoptosis, NF-kB activation, |
| | | | | 1999, Science, 285(5425): 260-3; Song HY et | and/or co-stimulation of |
| | | | | al., 1997 Proc Natl Acad Sci USA | immune cells such as B and |
| | | | | 94(18): 9792-6; Epsevik and Nissen-Meyer, | T cells. |
| | | | | 1986, J. Immunol. Methods. | |
| CD30 | GeneSeq Accession | DE4200043 | Activities associated with apoptosis, NF-kB | Apoptosis activity, NF-kB activation, and B | Soluble CD30 polypeptides |
| | R35478 | | activation, and co-stimulation of immune | and T cell co-stimulation can be determined | may be useful for inhibiting |
| | | | cells such as T and B cells. | using assays known in the art: Moore et al., | apoptosis, NF-kB activation, |
| | | | | 1999, Science, 285(5425): 260-3; Song HY et | and/or co-stimulation of |
| | | | | al., 1997 Proc Natl Acad Sci USA | immune cells such as B and |
| | | | | 94(18): 9792-6; Epsevik and Nissen-Meyer, | T cells. |
| | | | | 1986, J. Immunol. Methods. | |
| CD40 | GeneSeq | WO9945944 | Activities associated with apoptosis, NF-kB | Apoptosis activity, NF-kB activation, and B | Soluble CD40 polypeptides |
| | Accession | | activation, and co-stimulation of immune | and T cell co-stimulation can be determined | may be useful for inhibiting |
| | Y33499 | | cells such as T and B cells. | using assays known in the art: Moore et al., | apoptosis, NF-kB activation, |
| | | | | 1999, Science 285(5425): 260-3; Song HY et | and/or co-stimulation of |
| | | | | al., 1997 Proc Natl Acad Sci USA | immune cells such as B and |
| | | | | 94(18): 9792-6; Epsevik and Nissen-Meyer, | T cells. |
| | | | | 1986, J. Immunol. Methods. | |
| EDAR | Genbank | | Activities associated with apoptosis, NF-kB | Apoptosis activity, NF-kB activation, and B | Immune Disorders, |
| | Accession | | activation, and co-stimulation of immune | and T cell co-stimulation can be determined | Lymphomas, X-linked |
| | AAD50077 | | cells such as T and B cells. | using assays known in the art: Moore et al., | hypohidrotic ectodermal |
| | | | | 1999, Science, 285(5425): 260-3; Song HY et | dysplasia |
| | | | | al., 1997 Proc Natl Acad Sci USA | |
| | | | | 94(18): 9792-6; Epsevik and Nissen-Meyer, | |
| | | | | 1986, J. Immunol. Methods. | |
| OX40; ACT-4 | GeneSeq | WO9512673 | Activities associated with apoptosis, NF-kB | Apoptosis activity, NF-kB activation, and B | Immune Disorders, |
| | Accession R74737 | | activation, and co-stimulation of immune | and T cell co-stimulation can be determined | Lymphomas, T cell disorders |
| | | | cells such as T and B cells. | using assays known in the art: Moore et al., | |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| TACI | GeneSeq | WO9839361 | Activities associated with apoptosis, NF-kB | Apoptosis activity, NF-kB activation, and B | Soluble TACI receptor |
| | Accession | | activation, and co-stimulation of immune | and T cell co-stimulation can be determined | polypeptides may be useful |
| | W75783 | | cells such as T and B cells. | using assays known in the art: Moore et al., | for inhibiting apoptosis, |
| | | | | 1999, Science, 285(5425): 260-3; Song HY et | NF-kB activation, and/or co- |
| | | | | al., 1997 Proc Natl Acad Sci USA | stimulation of immune cells |
| | | | | 94(18): 9792-6; Epsevik and Nissen-Meyer, | such as B and T cells. |
| | | | | 1986, J. Immunol. Methods. | |
| TNF-R | GeneSeq | AU9058976 | Activities associates with apoptosis, NF-kB | Apoptosis activity, NF-kB activation, and B and | Soluble TNF-R receptor |
| | Accession R10986 | | activation, and co-stimulation of immune | T cell co-stimulation can be determined using | polypeptides may be useful |
| | | | cells such as T and B cells. | assays known in the art: Moore et al., 1999, | for inhibiting apoptosis, |
| | | | | Science, 285(5425): 260-3; Song HY et al., | NF-kB activation, and/or co- |
| | | | | 1997 Proc Natl Acad Sci USA 94(18): 9792-6; | stimulation of immune cells |
| | | | | Epsevik and Nissen-Meyer, 1986, J. Immunol. | such as B and T cells. |
| | | | | Methods. | |
| TNF-RII; TNF | GeneSeq | EP418014 | Activities associated with apoptosis, NF-kB | Apoptosis activity, NF-kB activation, and B | Soluble TNFR-II receptor |
| p75 receptor; | Accession R11141 | | activation, and co-stimulation of immune | and T cell co-stimulation can be determined | polypeptides may be useful |
| Death Receptor | | | cells such as T and B cells. | using assays known in the art: Moore et al., | for inhibiting apoptosis, |
| | | | | 1999, Science, 285(5425): 260-3; Song HY et | NF-kB activation, and/or co- |
| | | | | al., 1997 Proc Natl Acad Sci USA | stimulation of immune cells. |
| | | | | 94(18)9792-6; Epsevik and Nissen-Meyer, | such as B and T cells. |
| | | | | 1986, J. Immunol. Methods. | |
| hAPO-4; TROY | GeneSeq | WO9911791 | Activities associated with apoptosis, NF-kB | Apoptosis activity, NF-kB activation, and B | Immune Disorders, Cancers |
| | Accession | | activation, and co-stimulation of immune | and T cell co-stimulation can be determined | |
| | W93581 | | cells such as T and B cells. | using assays known in the art: Moore et al., | |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| TNF-alpha | GeneSeq | EP205038 | Activities associated with apoptosis, NF-kB | Apoptosis activity, NF-kB activation, and B | Inflammatory disorders, |
| precursor | Accession P60074 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| Human TNF- | GeneSeq | EP619372 | Activities associated with apoptosis, NF-kB | Apoptosis activity, NF-kB activation, and B | Inflammatory disorders, |
| alpha | Accession R62463 | | activation, and co-stimulation of immune | and T cell co-stimulation can be determined | immunologic disorders, |
| | | | cells such as T and B cells | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| Human TNF- | GeneSeq | EP563714 | Activities associated with apoptosis, NF-kB | Apoptosis activity, NF-kB activation, and B | Inflammatory disorders, |
| alpha | Accession R42679 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| Human TNF- | GeneSeq | WO0064479 | Activities associated with apoptosis, NF-kB | Apoptosis activity, NF-kB activation, and B | Inflammatory disorders, |
| beta (LT-alpha) | Accession B37799 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| LT-alpha | GeneSeq | EP250000 | Activities associated with apoptosis, NF-kB | Apoptosis activity, NF-kB activation, and B | Inflammatory disorders, |
| | Accession P70107 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| LT-beta | GeneSeq | WO9413808 | Activities associated with apoptosis, NF-kB | Apoptosis activity, NF-kB activation, and B | Inflammatory disorders, |
| | Accession R56869 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18)9792-6; Epsevik and Nissen-Meyer, | |
| | | | | 1986, J. Immunol. Methods. | |
| OPGL | GeneSeq | WO9846751 | Activities associated with apoptosis, NF-kB | Apoptosis activity, NF-kB activation, and B | Inflammatory disorders, |
| | Accession | | activation, and co-stimulation of immune | and T cell co-stimulation can be determined | immunologic disorders, |
| | W83195 | | cells such as T and B cells. | using assays known in the art: Moore et al., | cancer, loss of bone mass |
| | | | | 1999, Science, 285(5425): 260-3; Song HY et | |
| | | | | al., 1997 Proc Natl Acad Sci USA | |
| | | | | 94(18)9792-6; Epsevik and Nissen-Meyer, | |
| | | | | 1986, J. Immunol. Methods. | |
| FasL | GeneSeq | WO9903999 | Activities associated with apoptosis, NF-kB | Apoptosis activity, NF-kB activation, and B | Inflammatory disorders, |
| | Accession | | activation, and co-stimulation of immune | and T cell co-stimulation can be determined | immunologic disorders, |
| | W98071 | | cells such as T and B cells. | using assays known in the art: Moore, et al., | cancer |
| | | | | 1999, Science, 285(5425): 260-3; Song HY et | |
| | | | | al., 1997 Proc Natl Acad Sci USA | |
| | | | | 94(18)9792-6; Epsevik and Nissen-Meyer, | |
| | | | | 1986, J. Immunol. Methods. | |
| FasL | GeneSeq | WO9903998 | Activities associated with apoptosis, NF-kB | Apoptosis activity, NF-kB activation, and B | Inflammatory disorders, |
| | Accession | | activation, and co-stimulation of immune | and T cell co-stimulation can be determined | imunologic disorders, cancer |
| | W95041 | | cells such as T and B cells. | using assays known in the art: Moore et al., | |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| CD27L | GeneSeq | WO9405691 | Activities associated with apoptosis, NF-kB | Apoptosis activity, NF-kB activation, and B | Inflammatory disorders, |
| | Accession R50121 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| CD30 ligand | GeneSeq | WO9324135 | Activities associated with apoptosis, NF-kB | Apoptosis activity, NF-kB activation, and B | Inflammatory disorders, |
| | Accession R45007 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| CD40L | GeneSeq | WO9529935 | Activities associated with apoptosis, NF-kB | Apoptosis activity, NF-kB activation, and B | Inflammatory disorders, |
| | Accession R85486 | | activation, and co-stimulation of immune | and T cell co-stimulation can be determined | immunologic disorders, |
| | | | cells such as T and B cells. | using assays known in the art: Moore, et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| 4-1BB ligand | GeneSeq | U.S. Pat. No. 5674704 | Activities associated with apoptosis, NF-kB | Apoptosis activity, NF-kB activation, and B | Inflammatory disorders, |
| | Accession | | activation, and co-stimulation of immune | and T cell co-stimulation can be determined | immunologic disorders, |
| | W26657 | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| FAS Ligand | GeneSeq | WO0058465 | Activities associated with apoptosis, NF-kB | Apoptosis activity, NF-kB activation, and B | Soluble DcR3 polypeptides |
| Inhibitory | Accession B19335 | | activation, and co-stimulation of immune | and T cell co-stimulation can be determined | may be useful for inhibiting |
| Protein (DcR3) | | | cells such as T and B cells. | using assays known in the art: Moore et al., | apoptosis, NF-kB activation, |
| | | | | 1999, Science, 285(5425): 260-3; Song HY et | and/or co-stimulation of |
| | | | | al., 1997 Proc Natl Acad Sci USA | immune cells such as B and |
| | | | | 94(18): 9792-6; Epsevik and Nissen-Meyer, | T cells. |
| | | | | 1986, J. Immunol. Methods | |
| OX40L | GeneSeq | WO9521915 | Activities associated with apoptosis, NF-kB | Apoptosis activity, NF-kB activation, and B | Inflammatory disorders, |
| | Accession R79903 | | activation, and co-stimulation of immune | and T cell co-stimulation can be determined | immunologic disorders, |
| | | | cells such as T and B cells. | using assays known in the art: Moore et al., | cancer |
| | | | | 1999, Science, 285(5425): 260-3; Song HY et | |
| | | | | al., 1997 Proc Natl Acad Sci USA | |
| | | | | 94(18): 9792-6; Epsevik and Nissen-Meyer, | |
| | | | | 1986, J. Immunol. Methods. | |
| Protease | GeneSeq | WO9106561 | Peptides that inhibit the function/binding of | HIV protease activities are known in the art: | HIV, inflammatory disorders, |
| inhibitor | Accessions | | HIV | HIV protease assays: EP0387231. One can | immunologic disorders, |
| peptides | R12435, R12436, | | | modify the assay to look for inhibition using | cancer, viral infections |
| | R12437, R12438, | | | any of the disclosed protease inhibitor | |
| | R12439, R12440, | | | polypeptides. | |
| | and R1244 | | | | |
| Retroviral protease | GeneSeq | EP387231 | Peptides that inhibit the function/binding of | HIV protease activities are known in the art: | HIV, inflammatory disorders, |
| inhibitors | Accessions | | HIV | HIV protease assays: EP0387231. One can | immunologic disorders, |
| | R06660, R06661, | | | modify the assay to look for inhibition using | cancer, viral infections |
| | R06662, R06663, | | | any of the disclosed protease inhibitor | |
| | R06664, R06665, | | | polypeptides. | |
| | R06666, R06667, | | | | |
| | R06668, R06669, | | | | |
| | R06670, R06671, | | | | |
| | R06672, R06673, | | | | |
| | R06674, R06675, | | | | |
| | and R06676 | | | | |
| HIV protease | GeneSeq | WO9301828 | Peptides that inhibit the function/binding of | HIV protease activities are known in the art: | HIV, inflammatory disorders, |
| inhibiting | Accessions | | HIV | HIV protease assays: EP0387231. One can | immunologic disorders, |
| peptides | R59293, R59294, | | | modify the assay to look for inhibition using | cancer, viral infections |
| | R59295, R59296, | | | any of the disclosed protease inhibitor | |
| | R59297, | | | polypeptides. | |
| | R59298, R59299, | | | | |
| | R592300, R59301, | | | | |
| | R59302, R59301, | | | | |
| | R59302, R59303, | | | | |
| | R59304, R59305, | | | | |
| | R59306, R59307, | | | | |
| | R59308, R59309, | | | | |
| | R59310, R59311, | | | | |
| | R59312, R59313, | | | | |
| | R59314, R59315, | | | | |
| | R59316, R59317 | | | | |
| | R59318, R59319, | | | | |
| | R59320, R59321, | | | | |
| | R59322, R59323, | | | | |
| | R59324, R59325, | | | | |
| | R59326, R59327, | | | | |
| | R59328, R59329, | | | | |
| | R59330, R59331, | | | | |
| | R59332, R59333, | | | | |
| | R59334, R59335, | | | | |
| | R59336, R59337, | | | | |
| | R59338, R59339, | | | | |
| | R59340, R59341, | | | | |
| | R59342, R59343, | | | | |
| | R59344, R59345, | | | | |
| | R59346, R59347, | | | | |
| | R59348, R59349, | | | | |
| | and R59350 | | | | |
| HIV-1 protease | GeneSeq | DE4412174 | Peptides that inhibit the function/binding of | HIV protease activities are known in the art: | HIV, inflammatory disorders, |
| hinibitors | Accessions | | HIV | HIV protease assays: EP0387231. One can | immunologic disorders, |
| | R86326, R86327, | | | modify the assay to look for inhibition using | cancer, viral infections |
| | R86328, R86329, | | | any of the disclosed protease inhibitor | |
| | R86330, R86331, | | | polypeptides. | |
| | R86332, R86333, | | | | |
| | R86334, R86335, | | | | |
| | R86336, R86337, | | | | |
| | R86338, R86339, | | | | |
| | R86340, R86341, | | | | |
| | R86342, R86343, | | | | |
| | R86344, R86345, | | | | |
| | R86346, R86347, | | | | |
| | R86348, R86349, | | | | |
| | R86350, R86351, | | | | |
| | R86352, R86353, | | | | |
| | R86354, R86355, | | | | |
| | R86356, R86357, | | | | |
| | R86358, R86359, | | | | |
| | R86360, R86361, | | | | |
| | R86362, R86363, | | | | |
| | R86364, R86365, | | | | |
| | R86366, R86367, | | | | |
| | R86368, R86369, | | | | |
| | R86370, and R86371 | | | | |
| HIV Inhibitor | GeneSeq | WO9959615 | Peptides that inhibit the function/binding of | HIV protease activities are known in the art: | HIV, inflammatory disorders, |
| Peptide | Accession | | HIV | HIV protease assays: EP0387231. One can | immunologic disorders, |
| | Y89687 | | | modify the assay to look for inhibition using | cancer, viral infections |
| | | | | any of the disclosed protease inhibitor | |
| | | | | polypeptides. | |
| HIV Inhibitor | GenSeq Accession | WO9948513 | Peptides that inhibit the function/binding of | HIV Protease activities are known in the art; | HIV, inflammatory disorders, |
| Peptide | Y31955 | | HIV | HIV protease assays: EP0387231. One can | immunologic disorders, |
| | | | | modify the assay to look for inhibition using | cancer, viral infections. |
| | | | | any of the disclosed protease inhibitor | |
| | | | | polypeptides. | |
| HIV Inhibitor | www.sciencexpress.org; | | Peptides that inhibit the function/binding of | HIV protease activities are known in the art: | HIV, inflammatory disorders, |
| Peptide | Published | | HIV | HIV protease assays: EP0387231. One can | immunologic disorders, |
| | online 12 Jan. | | | modify the assay to look for inhibition using | cancer, viral infections |
| | 2001; | | | any of the disclosed protease inhibitor | |
| | 10.1126/science.1057453 | | | polypeptides. | |
| Human monocyte | GeneSeq Accession | WO9509232 | Chemokines are a family of small, secreted | Chemokine activities can be determined using | Immune disorders, |
| chemoattractant | R73915 | | proteins involved in biological processes | assays known in the art: Methods in | particularly useful for |
| factor hMCP-3 | | | ranging from hematopoiesis, angiogenesis, | Molecular Biology, 2000, vol. 138: | treating bacterial and/or viral |
| | | | and leukocyte trafficking. Members of this | Chemokine Protocols, Edited by: A. E. I. Proudfoot, | menigitis |
| | | | family are involved in a similarly diverse | T. N. C. Wells, and C. A. Power. © | |
| | | | range of pathologies including inflammation, | Humana Press Inc., Totowa, NJ | |
| | | | allergy, tissue rejection, viral infection, and | | |
| | | | tumor biology. The chemokines exert their | | |
| | | | effects by acting on a family of seven | | |
| | | | transmembrane G-protein-coupled receptors. | | |
| | | | Over 40 human chemokines have been | | |
| | | | described, which bind to - 17 receptors thus | | |
| | | | far identified. | | |
| Human monocyte | GeneSeq Accession | WO9509232 | Chemokines are a family of related small, | Chemokine activities can be determined using | Immune disorders, |
| chemoattractant | R73914 | | secreted proteins involved in biological | assays known in the art: Methods in | particularly useful for |
| factor hMCP-1 | | | processes ranging from hematopoiesis, | Molecular Biology, 2000, vol. 138: | treating bacterial and/or viral |
| | | | angiogenesis, and leukocyte trafficking. | Chemokine Protocols. Edited by: A. E. I. Proudfoot, | menigitis |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified. | | |
| Human gro-beta | GeneSeq | WO9429341 | Chemokines are a family of small, secreted | Chemokine activities can be determined using | Immune disorders, |
| chemokine | Accessions R66699 | | proteins involved in biological processes | assays known in the art: Methods in | inflammatory disorders, |
| | and W17671 | | ranging from hematopoiesis, angiogenesis, | Molecular Biology, 2000, vol. 138: | blood-related disorders, stem |
| | | | and leukocyte trafficking. Members of this | Chemokine Protocols. Edited by: A. E. I. Proudfoot, | cell transplantation, cancer |
| | | | family are involved in a similarly diverse | T. N. C. Wells, and C. A. Power. © | |
| | | | range of pathologies including inflammation, | Humana Press Inc., Totowa, NJ | |
| | | | allergy, tissue rejection, viral infection, and | | |
| | | | tumor biology. The chemokines exert their | | |
| | | | effects by acting on a family of seven | | |
| | | | transmembrane G-protein-coupled receptors. | | |
| | | | Over 40 human chemokines have been | | |
| | | | described, which bind to - 17 receptors thus | | |
| | | | far identified. | | |
| Human gro-gamma | GeneSeq | WO9429341 | Chemokines are a family of related small, | Chemokine activities can be determined using | Immune disorders, |
| chemokine | Accessions R66700 | | secreted proteins involved in biological | assays known in the art: Methods in | inflammatory disorders, |
| | and W17672 | | processes ranging from hematopoiesis, | Molecular Biology, 2000, vol. 138: | blood-related disorders, stem |
| | | | angiogenesis, and leukocyte trafficking. | Chemokine Protocols. Edited by: A. E. I. Proudfoot, | cell transplantation, cancer |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Human gro-alpha | GeneSeq | WO9429341 | Chemokines are a family of related small, | Chemokine activities can be determined using | Immune disorders, |
| chemokine | Accessions R66698 | | secreted proteins involved in biological | assays known in the art: Methods in | inflammatory disorders, |
| | and W18024 | | processes ranging from hematopoiesis, | Molecular Biology, 2000, vol. 138: | blood-related disorders, stem |
| | | | angiogenesis, and leukocyte trafficking. | Chemokine Protocols. Edited by: A. E. I. Proudfoot, | cell transplantation, cancer |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Human eosinophil- | GeneSeq Accession | WO9632481 | Chemokines are a family of related small, | Chemokine activities can be determined using | Immune disorders, |
| expressed | W05186 | | secreted proteins involved in biological | assays known in the art: Methods in | particularly treatment of |
| chemokine (EEC) | | | processes ranging from hematopoiesis, | Molecular Biology, 2000, vol. 138: | eosinophilia, inflammation, |
| | | | angiogenesis, and leukocyte trafficking. | Chemokine Protocols. Edited by: A. E. I. Proudfoot, | allergies, asthma, leukaemia |
| | | | Members of this family are involved in a | T. N. C. Wells, and C. A. Power. © | and lymphoma |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Chemokine-like | GeneSeq | WO9613587 | Chemokines are a family of related small, | Chemokine activities can be determined using | Cancer and blood-related |
| protein PF4-414 | Accessions R92318 | | secreted proteins involved in biological | assays known in the art: Methods in | disorders, particularly |
| Full-Length and | and R99809 | | processes ranging from hematopoiesis, | Molecular Biology, 2000, vol. 138: | myelosuppression |
| Mature | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Chemokine-like | GeneSeq Accession | WO9613587 | Chemokines are a family of related small, | Chemokine activities can be determined using | Cancer and blood-related |
| protein IL-8M3 | R99812 | | secreted proteins involved in biological | assays known in the art: Methods in | disorders, particularly |
| | | | processes ranging from hematopoiesis, | Molecular Biology, 2000, vol. 138: | myelosuppression |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ; and Holmes | |
| | | | including inflammation, allergy, tissue | et al (1991) Science 253, 1278-80. | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Human interleukin- | GeneSeq Accession | WO9613587 | Chemokines are a family of related small, | Chemokine activities can be determined using | Cancer and blood-related |
| 8 (IL-8) | R99814 | | secreted proteins involved in biological | assays known in the art: Methods in | disorders, particularly |
| | | | processes ranging from hematopoiesis, | Molecular Biology, 2000, vol. 138: | myelosuppression |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ; and Holmes | |
| | | | including inflammation, allergy, tissue | et al (1991) Science 253, 1278-80. | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Chemokine-like | GeneSeq | WO9613587 | Chemokines are a family of related small, | Chemokine activities can be determined using | Cancer and blood-related |
| protein IL-8M1 | Accessions R99815 | | secreted proteins involved in biological | assays known in the art: Methods in | disorders, particularly |
| Full-Length and | and R99803 | | processes ranging from hematopoiesis, | Molecular Biology, 2000, vol. 138: | myelosuppression |
| Mature | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ; and Holmes | |
| | | | including inflammation, allergy, tissue | et al (1991) Science 253, 1278-80. | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to - 17 receptors thus far identified | | |
| Chemokine-like | GeneSeq | WO9613587 | Chemokines are a family of related small, | Chemokine activities can be determined using | Cancer and blood-related |
| protein IL —8M8 | Accessions R99816 | | secreted proteins involved in biological | assasys known in the art: Methods in | disorders, particularly |
| Full-Length and | and R99805 | | processes ranging from hematopoiesis, | Molecular Biology, 2000, vol. 138: | myelosuppression. |
| Mature | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ; and Holmes | |
| | | | including inflammation, allergy, tissue | et al (1991) Science 253, 1278-80. | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine-like | GeneSeq | WO9613587 | Chemokines are a family of related small, | Chemokine activities can be determined using | Cancer and blood-related |
| protein IL-8M8 | Accessions R99817 | | secreted proteins involved in biological | assasys known in the art: Methods in Molecular | disorders, particularly |
| Full-Length and | and R99806 | | processes ranging from hematopoiesis, | Biology, 2000, vol. 138: Chemokine Protocols. | myelosuppression. |
| Mature | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ; | |
| | | | similarly diverse range of pathologies | and Holmes et at (1991) Science 253, 1278-80. | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified | | |
| Chemokine-like | GeneSeq Accessions | WO9613587 | Chemokines are a family of related small, | Chemokine activities can be determined using | Cancer and blood-related |
| protein IL-8M8 | R99818 and R99804 | | secreted proteins involved in biological | assasys known in the art: Methods in Molecular | disorders, particularly |
| Full-Length and | | | processes ranging from hematopoiesis, | Biology, 2000, vol. 138: Chemokine Protocols. | myelosuppression. |
| Mature | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ; | |
| | | | similarly diverse range of pathologies | and Holmes et al (1991) Science 253, 1278-80. | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine-like | GeneSeq Accessions | WO9613587 | Chemokines are a family of related small, | Chemokine activities can be determined using | Cancer and blood-related |
| protein IL-8M8 | R99819 and R99807 | | secreted proteins involved in biological | assasys known in the art: Methods in Molecular | disorders, particularly |
| Full-Length and | | | processes ranging from hematopoiesis, | Biology, 2000, vol. 138: Chemokine Protocols. | myelosuppression. |
| Mature | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine-like | GeneSeq Accessions | WO9613587 | Chemokines are a family of related small, | Chemokine activities can be determined using | Cancer and blood-related |
| protein IL-8M8 | R99822 and R9807 | | secreted proteins involved in biological | assasys known in the art: Methods in Molecular | disorders, particularly |
| Full-Length and | | | processes ranging from hematopoiesis, | Biology, 2000, vol. 138: Chemokine Protocols. | myelosuppression. |
| Mature | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human foetal spleen | GeneSeq Accession | WO9622374 | Chemokines are a family of related small, | Chemokine activities can be determined using | Immune disorders |
| expressed | R98499 | | secreted proteins involved in biological | assasys known in the art: Methods in Molecular | |
| chemokine, FSEC | | | processes ranging from hematopoiesis, | Biology, 2000, vol. 138: Chemokine Protocols. | |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Liver expressed | GeneSeq Accession | WO9616979 | Chemokines are a family of related small, | Chemokine activities can be determined using | Inflammation of the liver |
| chemokine- | R95689 | | secreted proteins involved in biological | assasys known in the art: Methods in Molecular | |
| 1(LVEC-1) | | | processes ranging from hematopoiesis, | Biology, 2000, vol. 138: Chemokine Protocols. | |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Liver expressed | GeneSeq Accession | WO9616979 | Chemokines are a family of related small, | Chemokine activities can be determined using | Inflammation of the liver |
| chemokine- | R95690 | | secreted proteins involved in biological | assasys known in the art: Methods in Molecular | |
| 2(LVEC-2) | | | processes ranging from hematopoiesis, | Biology, 2000, vol. 138: Chemokine Protocols. | |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Pituitary expressed | GeneSeq Accession | WO9616979 | Chemokines are a family of related small, | Chemokine activities can be determined using | Inflammation, particularly of |
| chemokine (PGEC) | R95691 | | secreted proteins involved in biological | assasys known in the art: Methods in Molecular | the liver |
| | | | processes ranging from hematopoiesis, | Biology, 2000, vol. 138: Chemokine Protocols. | |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Adenoid-expressed | GeneSeq Accession | WO9617868 | Chemokines are a family of related small, | Chemokine activities can be determined using | Inflammation, angiogenesis, |
| chemokine (ADEC) | R97664 | | secreted proteins involved in biological | assasys known in the art: Methods in Molecular | tumorigenesis, |
| | | | processes ranging from hematopoiesis, | Biology, 2000, vol. 138: Chemokine Protocols. | musculoskeletal disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO9741230 | Chemokines are a family of related small, | Chemokine activities can be determined using | Immune disorders, cell |
| chemokineCC-2 | Accession | | secreted proteins involved in biological | assays known in the art: Methods in | migration, proliferation, and |
| | W38170 | | processes ranging from hematopoiesis, | Molecular Biology, 2000, vol. 138; | differentiation disorders |
| | | | angiogenesis, and leukocyte trafficking. | Chemokine protocols. Edited by: A. E. I. Proudfoot, | |
| | | | Members of this family are involved in a | T. N. C. Wells, and C. A. Power. © | |
| | | | similarly diverse range of pathologies | Humana Press Inc. Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane | | |
| | | | G-protein-coupled receptors. Over 40 human | | |
| | | | chemokines have been described, which bind | | |
| | | | to ˜17 receptors thus far identified. | | |
| Human | GeneSeq | WO9741230 | Chemokines are a family of related small, | Chemokine activities can be determined using | Immune disorders, cell |
| chemokine | Accession | | secreted proteins involved in biological | assays known in the art: Methods in molecular | migration, proliferation, and |
| HCC-1 | W38171 | | processes ranging from hematopiesis, | Biology 2000, vol. 138: Chemokine Protocols. | differentiation disorders |
| | | | anglogenesis and leukocyte trafficking. | Edited by A. E. I. Proudfoot. T. N. C. Wells and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane | | |
| | | | G-protein-coupled receptors. Over 40 human | | |
| | | | chemokines have been described, which bind | | |
| | | | to ˜17 receptors thus far identified. | | |
| Human | GeneSeq | WO9741230 | Chemokines are a family of related small, | Chemokine activities can be determined using | Immune disorders, cell |
| chemokine CC-3 | Accession | | secreted proteins involved in biological | assays known in the art: Methods in molecular | migration, proliferation and |
| | W38172 | | processes ranging from hemotopoiesis, | Biology, 2000, vol. 138: Chemokine Protocols, | differentiation disorders |
| | | | anglogenesis, and leukocyte trafficking. | Edited by A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Novel | GeneSeq | WO9739126 | Chemokines are a family of related small, | Chemokine activities can be determined using | Immune disorders, vascular |
| betachemokine | Accession | | secreted proteins involved in biological | assays known in the art: Methods in molecular | disorders, cancer |
| designated PTEC | W27271 | | processes ranging from hemotopoiesis, | Biology, 2000, vol. 138: Chemokine Protocols, | |
| | | | anglogenesis, and leukocyte trafficking. | Edited by A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human CX3C | GeneSeq | WO9727299 | Chemokines are a family of related small, | Chemokine activities can be determined using | Immune disorders, |
| 111 amino acid | Accession | | secreted proteins involved in biological | assays known in the art: Methods in molecular | inflammatory diseases, |
| chemokine | W23344 | | processes ranging from hemotopoiesis, | Biology, 2000, vol. 138: Chemokine Protocols, | abnormal proliferation, |
| | | | anglogenesis, and leukocyte trafficking. | Edited by A. E. I. Proudfoot, T. N. C. Wells, and | regeneration, degeneration, |
| | | | Members of this family are involved in a | C. A. Power © Humana Press Inc., Totowa, NJ | and atrophy |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human CCF18 | GeneSeq | WO9721812 | Chemokines are a family of related small, | Chemokine activities can be determined using | Abnormal physiology and |
| chemokine | Accession | | secreted proteins involved in biological | assays known in the art: Methods in molecular | development disorders, can |
| | W25942 | | processes ranging from hemotopoiesis, | Biology, 2000, vol. 138: Chemokine Protocols, | also be used as an anti-viral |
| | | | anglogenesis, and leukocyte trafficking. | Edited by A. E. I. Proudfoot, T. N. C. Wells, and | agent |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human beta- | GeneSeq | WO9725427 | Chemokines are a family of related small, | Chemokine activities can be determined using | Chemotaxis, blood-related |
| chemokine | Accession | | secreted proteins involved in biological | assays known in the art: Methods in molecular | disorders, viral infection, |
| H1305 (MCP-2) | W26655 | | processes ranging from hemotopoiesis, | Biology, 2000, vol. 138: Chemokine Protocols, | HIV, wound healing, cancer |
| | | | anglogenesis, and leukocyte trafficking. | Edited by A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO9712914 | Chemokines are a family of related small, | Chemokine activities can be determined using | Inflammatory and immune |
| eosinocyte CC | Accession | | secreted proteins involved in biological | assays known in the art: Methods in molecular | disorders |
| type chemokine | W14990 | | processes ranging from hemotopoiesis, | Biology, 2000, vol. 138: Chemokine Protocols, | |
| eotaxin | | | anglogenesis, and leukocyte trafficking. | Edited by A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human thymus | GeneSeq | WO9711969 | Chemokines are a family of related small, | Chemokine activities can be determined using | Inflammatory and immune |
| and activation | Accession | | secreted proteins involved in biological | assays known in the art: Methods in molecular | disorders |
| regulated | W14018 | | processes ranging from hemotopoiesis, | Biology, 2000, vol. 138: Chemokine Protocols, | |
| cytokine | | | anglogenesis, and leukocyte trafficking. | Edited by A. E. I. Proudfoot, T. N. C. Wells, and | |
| (TARC) | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO9712041 | Chemokines are a family of related small, | Chemokine activities can be determined using | Cancer, would healing, |
| chemokine beta- | Accession | | secreted proteins involved in biological | assays known in the art: Methods in molecular | immune disorders |
| 8 short forms | W16315 | | processes ranging from hemotopoiesis, | Biology, 2000, vol. 138: Chemokine Protocols, | |
| | | | anglogenesis, and leukocyte trafficking. | Edited by A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Microphage derived | GeneSeq Accession | WO9640923 | Chemokines are a family of related small, | Chemokine activities can be determined using | Inflammatory diseases, |
| chemokine, MDC | W20058 | | secreted proteins involved in biological | assays known in the art: Methods in Molecular | wound healin, |
| | | | processes ranging from hermaatopoiesis, | Biology, 2000, vol. 138: Chemokine | angiogenesis |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO9844117 | Chemokines are a family of related small, | Chemokine activities can be determined using | Inflammatory and |
| ZSIG-35 | W30565 | | secreted proteins involved in biological | assays known in the art: Methods in Molecular | immune diseases |
| | | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Primate CC | GeneSeq | WO98328658 | Chemokines are a family of related small, | Chemokine activities can be determined using | Immune and |
| chemokine | Accesssion W69990 | | secreted proteins involved in biological | assays known in the art: Methods in Molecular | inflammatory disorders, |
| “ILINCK” | | | processes ranging from hematopoiesis, | Biology, 2000, vol. 138: Chemokine | abnormal proliferation, |
| | | | angiogenesis, and leukocyte trafficking. | Protocols. Edited by: A. E. I. Proudfoot, T. N. C. Wells, | regeneration, generation |
| | | | | and C. A. Power. © Humana Press Inc., | and atrophy disorders |
| | | | | Totowa, NJ | |
| Primate CXC | GeneSeq Accession | WO9832858 | Chemokines are a family of related small, | Chemokine activities can be determined using | Immune and |
| chemokine “IBICK” | W69989 | | secreted proteins involved in biological | assays known in the art: Methods in Molecular | inflammatory disorders, |
| | | | processes ranging from hematopoiesis, | Biology, 2000, vol. 138: Chemokine | abnormal proliferation, |
| | | | angiogenesis, and leukocyte trafficking. | Protocols. Editd by: A. E. I. Proudfoot, T. N. C. Wells, | regeneration, generation |
| | | | Members of this family are involved in a | and C. A. Power. © Humana Press Inc., | and atrophy disorders |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human CC-type | GeneSeq Accession | WO9831809 | Chemokines are a family of related small, | Chemokine activities can be determined using | Immune, inflammatory, |
| chemokine protein | W69163 | | secreted proteins involved in biological | assays known in the art: Methods in Molecular | and infectious disorders, |
| designated SLC | | | processes ranging from hematopoiesis, | Biology, 2000, vol. 138: Chemokine | cancer |
| (secondary | | | angiogenesis, and leukocyte trafficking. | Protocols. Edited by: A. E. I. Proudfoot, T. N. C. Wells, | |
| lymphoid | | | Members of this family are involved in a | and C. A. Power. © Humana Press Inc., | |
| chemokine) | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human CC | GeneSeq Accession | WO9826071 | Chemokines are a family of related small, | Chemokine activities can be determined using | Cancer and infectious |
| chemokine ELC | W62542 | | secreted proteins involved in biological | assays known in the art: Methods in Molecular | diseases, particularly |
| protein | | | processes ranging from hematopoiesis, | Biology, 2000, vol. 138: Chemokine | herpes virus |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human DVic-1 | GeneSeq | Wo9823750 | Chemokines are a family of related small, | Chemokine activities can be determined using | Abnormal proliferation, |
| C-C chemokine | Accession | | secreted proteins involved in biological | assays known in the art: Methods in Molecular | regeneration, |
| | W60649 | | processes ranging from hematopoiesis, | Biology, 2000, vol. 138: Chemokine | degeneration, and atrophy |
| | | | angiogenesis, and leukocyte trafficking. | Protocols. Edited by: A. E. I. Proudfoot, T. N. C. Wells, | disorders, including |
| | | | Members of this family are involved in a | and C. A. Power. © Humana Press Inc., | cancer |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human C-C | GeneSeq | WO9823750 | Chemokines are a family of related small, | Chemokine activities can be determined using | Immune disorders, cell |
| chemokine | Accession | | secreted proteins involved in biological | assays known in the art: Methods in Molecular | proliferation disorders, |
| DGWCC | W60650 | | processes ranging from hematophoiesis, | Biology, 2000, vol. 138: Chemokine | cancer |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identifed. | | |
| Human STCP-1 | GeneSeq | WO9824907 | Chemokines are a family of related small, | Chemokine activities can be determined using | Immune disorders, |
| | Accession | | secreted proteins involved in biological | assays known in the art: Methods in Molecular | particularly T cell related |
| | W62783 | | processes ranging from hematopoiesis, | Biology, 2000, vol. 138: Chemokine | disorders, viral infection, |
| | | | angiogenesis, and leukocyte trafficking. | Protocols. Edited by: A. E. I. Proudfoot, T. N. C. Wells, | and inflammation, |
| | | | Members of this family are involved in a | and C. A. Power. © Humana Press Inc., | especially joint |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Exodua protein | GeneSeq | WO9821330 | Chamokines are a family of related small, | Chemokine activities can be determined using | Immune and |
| | Accession | | secreted proteins involved in biological | assays known in the art: Methods in Molecular | inflammatory disorders, |
| | W61279 | | processes ranging from hematopoiesis, | Biology, 2000, vol. 138: Chemokine Protocols. | angiogenesis, cancer, and |
| | | | angiogenesis, and leukocyte trafficking. | Edited by: A. E. I. Proudfoot, T. N. C. Wells, and | proliferation disorders, |
| | | | Members of this family are involved in a | C. A. Power. © Humana Press Inc., Totowa, | particularly |
| | | | similarly diverse range of pathologies | NJ | myeloproliferative |
| | | | including inflammation, allergy, tissue | | diseases |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO9814581 | Chemokines are a family of related small, | Chemokine activities can be determined using | Cancer and degenerative |
| Chr19Kine | Acession | | secreted proteins involved in biological | assays known in the art: Methods in | disorders |
| protein | W50887 | | processes ranging from hematopoiesis, | Molecular Biology, 2000, vol. 138: | |
| | | | angiogenesis, and leukocyte trafficking. | Chemokine Protocols, Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human T cell | GeneSeq | U.S. Pat. No. 5780268 | Chemokines area family of related small, | Chemokine activities can be determined using | Immune, inflammatory, and |
| mixed | Accession | | secreted proteins involved in biological | assays known in the art: Mehtods of Molecular | infectious disorders, cancer |
| lymphocyte | W58703 | | processes ranging from hematopoiesis, | Biology, 2000, vol. 138: Chemokine Protocols. | |
| reaction | | | angiogenesis, and leukocyte trafficking. | Edited by: A. E. I. Proudfoot, T. N. C. Wells, and | |
| expressed | | | Members of this family are involved in a | C. A. Power © Humana Press Inc., Totowa, NJ | |
| chemokine | | | similarly diverse range of pathologies | | |
| (TMEC) | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human 6CKine | GeneSeq | W09814581 | Chemokines area family of related small, | Chemokine activities can be determined using | Cancer and degenerative |
| protein | Accession | | secreted proteins involved in biological | assays known in the art: Mehtods of Molecular | disorders |
| | W50885 | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| human liver and | GeneSeq | WO9817800 | Chemokines area family of related small, | Chemokine activities can be determined using | Immune, inflammatory, and |
| activation | Accession | | secreted proteins involved in biological | assays known in the art: Mehtods of Molecular | infectious disorders, cancer |
| regulated | W57475 | | processes ranging from hematopoiesis, | Biology, 2000, vol. 138: Chemokine Protocols. | |
| chemokine | | | angiogenesis, and leukocyte trafficking. | Edited by: A. E. I. Proudfoot, T. N. C. Wells, and | |
| (LARC) | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| RANTES | GeneSeq | WO9744462 | Chemokines area family of related small, | Chemokine activities can be determined using | Infectious diseases, |
| peptide | Accession | | secreted proteins involved in biological | assays known in the art: Mehtods of Molecular | particularly HIV |
| | W29538 | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| RANTES 8-68 | GeneSeq | WO9744462 | Chemokines area family of related small, | Chemokine activities can be determined using | Infectious diseases, |
| | Accession | | secreted proteins involved in biological | assays known in the art: Mehtods of Molecular | particularly HIV |
| | W29529 | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| RANTES 9-68 | GeneSeq | WO9744462 | Chemokines area family of related small, | Chemokine activities can be determined using | Infectious diseases, |
| | Accession | | secreted proteins involved in biological | assays known in the art: Mehtods of Molecular | particularly HIV |
| | W29528 | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO9811226 | Chemokines area family of related small, | Chemokine activities can be determined using | Abnormal proliferation, |
| chemokine | Accession | | secreted proteins involved in biological | assays known in the art: Mehtods of Molecular | regeneration, degeneration or |
| protein 331D5 | W59433 | | processes ranging from hematopoiesis, | Biology, 2000, vol. 138: Chemokine Protocols. | atrophy, including cancer |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO9811226 | Chemokines area family of related small, | Chemokine activities can be determined using | Abnormal proliferation, |
| chemokine | Accession | | secreted proteins involved in biological | assays known in the art: Mehtods of Molecular | regeneration, degeneration or |
| protein 61164 | W59430 | | processes ranging from hematopoiesis, | Biology, 2000, vol. 138: Chemokine Protocols. | atrophy, including cancer |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9809171 | Chemokines area family of related small, | Chemokine activities can be determined using | Immune, Inflammatory, and |
| MCP-4 | Accession | | secreted proteins involved in biological | assays known in the art: Mehtods of Molecular | infectious diseases |
| | W56690 | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human stromal cell- | GeneSeq Accession | FR2751658 | Chemokines are a family of related small, | Chemokine activities can be determined using | HIV infections |
| derived chemokine, | W50766 | | secreted proteins involved in biological | assays known in the art: Methods in | |
| SDF-1 | | | processes ranging from hematopoiesis, | Molecular Biology, 2000, vol. 138: | |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Thymus expressed | GeneSeq Accession | WO9801557 | Chemokines are a family of related small, | Chemokine activities can be determined using | Immune and inflammatory |
| chemokine (TECK) | W44397 | | secreted proteins involved in biological | assays known in the art: Methods in Molecular | disorders |
| | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO9801557 | Chemokines are a family of related small, | Chemokine activities can be determined using | Immune and inflammatory |
| MIP-3alpha | W44398 | | secreted proteins involved in biological | assays known in the art: Methods in Molecular | disorders |
| | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO9801557 | Chemokines are a family of related small, | Chemokine activities can be determined using | Immune and inflammatory |
| MIP-3beta | W44399 | | secreted proteins involved in biological | assays known in the art: Methods in Molecular | disorders |
| | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human monocyte | GeneSeq Accession | WO9802459 | Chemokines are a family of related small, | Chemokine activities can be determined using | Immune disorders, |
| chemotactic | W42072 | | secreted proteins involved in biological | assays known in the art: Methods in Molecular | respiratory disorders, cancer |
| proprotein (MCPP) | | | processes ranging from hematopoiesis, | Biology, 2000, vol. 138: Chemokine Protocols. | |
| sequence | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Macrophage- | GeneSeq Accessions | U.S. Pat. No. 5688927/ | Chemokines are a family of related small, | Chemokine activities can be determined using | Immune, and inflammatory |
| derived chemokine | W40811 and Y24414 | U.S. Pat. No. 5932703 | secreted proteins involved in biological | assays known in the art: Methods in Molecular | disorders, cancer |
| (MDC) | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Macrophage derived | GeneSeq Accession | U.S. Pat. No. 5932703 | Chemokines are a family of related small, | Chemokine activities can be determined using | Immune and inflammatory |
| chemokine analogue | Y24416 | | secreted proteins involved in biological | assays known in the art: Methods in Molecular | disorders |
| MDC-eyfy | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Macrophage derived | GeneSeq Accession | U.S. Pat. No. 5932703 | Chemokines are a family of related small, | Chemokine activities can be determined using | Immune and inflammatory |
| chemokine analogue | Y24413 | | secreted proteins involved in biological | assays known in the art: Methods in Molecular | disorders |
| MDC (n + 1) | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Macrophage derived | GeneSeq Accession | U.S. Pat. No. 5932703 | Chemokines are a family of related small, | Chemokine activities can be determined using | Immune and inflammatory |
| chemokine analogue | Y24415 | | secreted proteins involved in biological | assays known in the art: Methods in Molecular | disorders |
| MDC-y1 | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human type CC | GeneSeq Accession | JP11243960 | Chemokines are a family of related small, | Chemokine activities can be determined using | Allergic diseases and HIV |
| chemokine eotaxin | Y43178 | | secreted proteins involved in biological | assays known in the art: Methods in Molecular | infection |
| 3 protein sequence | | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human MCP-3 and | GeneSeq Acession | WO9946392 | Chemokines are a family of related small, | Chemokine activities can be determined using | Cancer and immune |
| human Muc-1 core | Y29893 | | secreted proteins involved in biological | assays known in the art: Methods in Molecular | disorders, particularly HIV |
| epitope (VNT) | | | processes ranging from hematopoiesis, | Biology, 2000, vol. 138: Chemokine | infection |
| fusion protein | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarily diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human IP-10 and | GeneSeq | WO9946392 | Chemokines are a family of related small, | Chemokine activities can be determined using | Cancer and immune |
| human Muc-1 core | Accession Y29894 | | secreted proteins involved in biological | assays known in the art: Methods in Molecular | disorders, particularly HIV |
| epitope (VNT) | | | processes ranging from hematopoiesis, | Biology, 2000, vol. 138: Chemokine Protocols. | infection |
| fusion protein | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human IP-10 and | GeneSeq | W09946392 | Chemokines are a family of related small, | Chemokine activities can be determined using | Cancer and immune |
| HIV-1 gp 120 | Accession Y29897 | | secreted proteins involved in biological | assays known in the art: Methods in Molecular | disorders, particularly HIV |
| hypervariable | | | processes ranging from hematopoiesis, | Biology, 2000, vol. 138: Chemokine Protocols. | infection |
| region fusion | | | angiogenesis, and leukocyte trafficking. | Edited by: A. E. I. Proudfoot, T. N. C. Wells, and | |
| protein | | | Members of this family are involved in a | C. A. Power. © Humana Press Inc., Totowa, NJ | |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human mammary | GeneSeq | WO9936540 | Chemokines are a family of related small, | Chemokine activities can be determined using | Breast disease, including |
| associated | Accessions | | secreted proteins involved in biological | assays known in the art: Methods in Molecular | cancer |
| chemokine | Y29092 and Y29093 | | processes ranging from hematopoiesis, | Biology, 2000, vol. 138: Chemokine Protocols. | |
| (MACK) protein | | | angiogenesis, and leukocyte trafficking. | Edited by: A. E. I. Proudfoot, T. N. C. Wells, and | |
| Full-Length and | | | Members of this family are involved in a | C. A. Power. © Humana Press Inc., Totowa, NJ | |
| Mature | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Tim-1 protein | GeneSeq | WO9933990 | Chemokines are a family of related small, | Chemokine activities can be determined using | Inflammation due to stimuli |
| | Accession | | secreted proteins involved in biological | assays known in the art: Methods in Molecular | such as heart attacks and |
| | Y28290 | | processes ranging from hematopoiesis, | Biology, 2000, vol. 138: Chemokine Protocols. | stroke, infection, physical |
| | | | angiogenesis, and leukocyte trafficking. | Edited by: A. E. I. Proudfoot, T. N. C. Wells, and | trauma, UV or ionizing |
| | | | Members of this family are involved in a | C. A. Power. © Humana Press Inc., Totowa, NJ | radiation, burns, frostbite or |
| | | | similarily diverse range of pathologies | | corrosive chemicals |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human Lkn-1 Full- | GeneSeq Accessions | WO9928473 and | Chemokines are a family of related small, | Chemokine activities can be determined using | HIV infection and cancer, |
| Length and Mature | Y17280, Y17274, | WO9928472 | secreted proteins involved in biological | assays known in the art: Methods in Molecular | particularly leukemia |
| protein | Y17281, and Y17275 | | processes ranging from hematopoiesis, | Biology, 2000, vol. 138: Chemokine Protocols. | |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| N-terminal modified | GeneSeq Accession | WO9920759 | Chemokines are a family of related small, | Chemokine activities can be determined using | Inhibit or stimulate |
| chemokine met- | Y05818 | | secreted proteins involved in biological | assays known in the art: Methods in Molecular | angiogenesis, inhibit the |
| hSDF-1 alpha | | | processes ranging from hematopoiesis, | Biology, 2000, vol. 138: Chemokine Protocols. | binding of HIV |
| | | | angiogenesis, and leukocyte trafficking. | Edited by: A. E. I. Proudfoot, T. N. C. Wells, and | |
| | | | Members of this family are involved in a | C. A. Power. © Humana Press Inc., Totowa, NJ | |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| N-terminal modified | GeneSeq Accession | WO9920759 | Chemokines are a family of related small, | Chemokine activities can be determined using | Inhibit or stimulate |
| chemokine met- | Y05819 | | secreted proteins involved in biological | assays known in the art: Methods in Molecular | angiogenesis, inhibit the |
| hSDF-1 beta | | | processes ranging from hematopoiesis, | Biology, 2000, vol. 138: Chemokine Protocols. | binding of HIV, |
| | | | angiogenesis, and leukocyte trafficking. | Edited by: A. E. I. Proudfoot, T. N. C. Wells, and | antiinflammatory; |
| | | | Members of this family are involved in a | C. A. Power. © Humana Press Inc., Totowa, NJ | immunosuppressant |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| N-terminal modified | GeneSeq Accession | WO9920759 | Chemokines are a family of related small, | Chemokine activities can be determined using | Inhibit or stimulate |
| chemokine | Y05820 | | secreted proteins involved in biological | assays known in the art: Methods in Molecular | angiogenesis, inhibit the |
| GroHEK/hSDF- | | | processes ranging from hematopoiesis, | Biology, 2000, vol. 138: Chemokine Protocols. | binding of HIV, |
| 1alpha | | | angiogenesis, and leukocyte trafficking. | Edited by: A. E. I. Proudfoot, T. N. C. Wells, and | antiinflammatory; |
| | | | Members of this family are involved in a | C. A. Power. © Humana Press Inc., Totowa, NJ | immunosuppressant |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| N-terminal modified | GeneSeq Accession | WO9920759 | Chemokines are a family of related small, | Chemokine activities can be determined using | Inhibit or stimulate |
| chemokine | Y05821 | | secreted proteins involved in biological | assays known in the art: Methods in Molecular | angiogenesis, inhibit the |
| GroHEK/hSDF- | | | processes ranging from hematopoiesis, | Biology, 2000, vol. 138: Chemokine Protocols. | binding of HIV, |
| 1beta. | | | angiogenesis, and leukocyte trafficking. | Edited by: A. E. I. Proudfoot, T. N. C. Wells, and | antiinflammatory; |
| | | | Members of this family are involved in a | C. A. Power. © Humana Press Inc., Totowa, NJ | immunosuppressant |
| | | | similarily diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Increase or enhance an |
| Eotaxin | Accession | | secreted proteins involved in biological | assays known in the art: Methods in | inflammatory response, an |
| | Y14230 | | processes ranging from hematopoiesis, | Molecular Bilogy, 2000, vol. 138: Chemokine | immune response |
| | | | agiogenesis, and leukocye trafficking. | Protocols. Edited by: A. E. I. Proudfoot, | orhaematopoietic cell- |
| | | | Members of this family are involved in a | T. N. C. Wells, and C. A. Power. © Humana | associated activity; treat a |
| | | | similarly diverse range of pathologies | Press Inc., Totowa, NJ | vascular indication; Cancer; |
| | | | including inflammation, allergy, tissue | | enhance wound healing, to |
| | | | rejection, viralk infection, and tumor biology. | | prevent or treat asthma, |
| | | | The chemokines exert their effects by acting | | organ transplant rejction, |
| | | | on a family of seven transmembrane G- | | rheumatoid arthritis or |
| | | | protein-coupled receptors. Over 40 human | | allergy |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hMCP1a | Accession | | secreted proteins involved in biological | assays known in the art: Methods in | disorders, Wound healing, |
| | Y14225 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ | response, |
| | | | including inflammation, allergy, tissue | | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hMCP1b | Accession | | secreted proteins involved in biological | assays known in the art: Methods in | disorders, Wound healing, |
| | Y14226 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ | response, |
| | | | including inflammation, allergy, tissue | | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hSDF1b | Accession | | secreted proteins involved in biological | assays known in the art: Methods in | disorders, Wound healing, |
| | Y14228 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ | response, |
| | | | including inflammation, allergy, tissue | | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hIL-8 | Accession | | secreted proteins involved in biological | assays known in the art: Methods in | disorders, Wound healing, |
| | Y14229 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ; and Holmes et al | response, |
| | | | including inflammation, allergy, tissue | (1991) Science 253, 1278-80. | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hMCP1 | Accession | | secreted proteins involved in biological | assays known in the art: Methods in | disorders, Wound healing, |
| | Y14222 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ | response, |
| | | | including inflammation, allergy, tissue | | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hMCP2 | Accession | | secreted proteins involved in biological | assays known in the art: Methods in | disorders, Wound healing, |
| | Y14223 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ | response, |
| | | | including inflammation, allergy, tissue | | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Chemokine | GeneSeq | WO9912968 | Chemokines are a family of related small, | Chemokine activities can be determined using | Immune disorders, Vascular |
| hMCP3 | Accession | | secreted proteins involved in biological | assays known in the art: Methods in | disorders, Wound healing, |
| | Y14224 | | processes ranging from hematopoiesis, | Molecular Bilogy, 2000, vol. 138: Chemokine | cancer, prevent organ |
| | | | agiogenesis, and leukocye trafficking. | Protocols. Edited by: A. E. I. Proudfoot, | transplant rejection, Increase |
| | | | Members of this family are involved in a | T. N. C. Wells, and C. A. Power. © Humana | or enhance an inflammatory |
| | | | similarly diverse range of pathologies | Press Inc., Totowa, NJ | response, |
| | | | including inflammation, allergy, tissue | | |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| C-C chemokine, | GeneSeq | EP905240 | Chemokines are a family of related small, | Chemokine activities can be determined using | Inflammatory, Immune and |
| MCP2 | Accession | | secreted proteins involved in biological | assays known in the art: Methods in | infectious diseases; |
| | Y05300 | | processes ranging from hematopoiesis, | Molecular Bilogy, 2000, vol. 138: Chemokine | pulmonary diseases and skin |
| | | | agiogenesis, and leukocye trafficking. | Protocols. Edited by: A. E. I. Proudfoot, | disorders; tumours, and |
| | | | Members of this family are involved in a | T. N. C. Wells, and C. A. Power. © Humana | angiogenesis-and |
| | | | similarly diverse range of pathologies | Press Inc., Totowa, NJ | haematopoiesis-related |
| | | | including inflammation, allergy, tissue | | diseases |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Wild type | GeneSeq | EP906954 | Chemokines are a family of related small, | Chemokine activities can be determined using | Inflammatory, Immune and |
| monocyte | Accession | | secreted proteins involved in biological | assays known in the art: Methods in | infectious diseases; |
| chemotactic | Y07233 | | processes ranging from hematopoiesis, | Molecular Bilogy, 2000, vol. 138: Chemokine | pulmonary diseases and skin |
| protein 2 | | | agiogenesis, and leukocye trafficking. | Protocols. Edited by: A. E. I. Proudfoot, | disorders; tumours, and |
| | | | Members of this family are involved in a | T. N. C. Wells, and C. A. Power. © Humana | angiogenesis-and |
| | | | similarly diverse range of pathologies | Press Inc., Totowa, NJ | haematopoiesis-related |
| | | | including inflammation, allergy, tissue | | diseases |
| | | | rejection, viralk infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Truncated | GeneSeq | EP906954 | Chemokines area family of related small, | Chemokines activities can be determined using | Inflammatory, immune and |
| monocyte | Accession | | secreted proteins involved in biological | assays known in the art: Methods in Molecular | infectious diseases; pulmonry |
| chemotactic | Y07234 | | processes ranging from hematopoiesis, | Biology, 2000, vol. 138: Chemokine Protocols. | diseases and skin disorders; |
| protein 2 (6-76) | | | angiogenesis, and leukocyte trafficking. | Edited by: A. E. I. Proudfoot, T. N. C. Wells, and | tumours, and angiogenesis- |
| | | | Members of this family are involved in a | C. A. Power, Humana Press Inc., Totowa, NJ | and haematopoiesis-related |
| | | | similarly diverse range of pathologies | | diseases |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Truncated | GeneSeq | EP905241; | Chemokines area family of related small, | Chemokines activities can be determined using | Inflammatory, immune and |
| RANTES | Accessions | EP906954 | secreted proteins involved in biological | assays known in the art: Methods in Molecular | infectious diseases; pulmonry |
| protein (3-68) | Y07236 and | | processes ranging from hematopoiesis, | Biology, 2000, vol. 138: Chemokine Protocols. | diseases and skin disorders; |
| | Y07232 | | angiogenesis, and leukocyte trafficking. | Edited by: A. E. I. Proudfoot, T. N. C. Wells, and | tumours, and angiogenesis- |
| | | | Members of this family are involved in a | C. A. Power, Humana Press Inc., Totowa, NJ | and haematopoiesis-related |
| | | | similarly diverse range of pathologies | | diseases |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Wild type | GeneSeq | EP905241 | Chemokines area family of related small, | Chemokines activities can be determined using | Inflammatory, immune and |
| monocyte | Accession | | secreted proteins involved in biological | assays known in the art: Methods in Molecular | infectious diseases; pulmonry |
| chemotactic | Y07237 | | processes ranging from hematopoiesis, | Biology, 2000, vol. 138: Chemokine Protocols. | diseases and skin disorders; |
| protein 2 | | | angiogenesis, and leukocyte trafficking. | Edited by: A. E. I. Proudfoot, T. N. C. Wells, and | tumours, and angiogenesis- |
| | | | Members of this family are involved in a | C. A. Power, Humana Press Inc., Totowa, NJ | and haematopoiesis-related |
| | | | similarly diverse range of pathologies | | diseases |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Truncated | GeneSeq | EP905241 | Chemokines area family of related small, | Chemokines activities can be determined using | Inflammatory, immune and |
| monocyte | Accession | | secreted proteins involved in biological | assays known in the art: Methods in Molecular | infectious diseases; pulmonry |
| chemotactic | Y07238 | | processes ranging from hematopoiesis, | Biology, 2000, vol. 138: Chemokine Protocols. | diseases and skin disorders; |
| protein 2 (6-76) | | | angiogenesis, and leukocyte trafficking. | Edited by: A. E. I. Proudfoot, T. N. C. Wells, and | tumours, and angiogenesis- |
| | | | Members of this family are involved in a | C. A. Power, Humana Press Inc., Totowa, NJ | and haematopoiesis-related |
| | | | similarly diverse range of pathologies | | diseases |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| A partial | GeneSeq | EP897980 | Chemokines area family of related small, | Chemokines activities can be determined using | Soluble CXCR4B receptor |
| CXCR4B | Accession | | secreted proteins involved in biological | assays known in the art: Methods in Molecular | polypeptides may be useful for |
| protein | W97363 | | processes ranging from hematopoiesis, | Biology, 2000, vol. 138: Chemokine Protocols. | inhibiting chemokine activities |
| | | | angiogenesis, and leukocyte trafficking. | Edited by: A. E. I. Proudfoot, T. N. C. Wells, and | and viral infection. |
| | | | Members of this family are involved in a | C. A. Power, Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Interferon | GeneSeq | U.S. Pat. No. 5871723 | Chemokines area family of related small, | Chemokines activities can be determined using | Angiogenesis, Cancer, |
| gamma- | Accession | | secreted proteins involved in biological | assays known in the art: Methods in Molecular | Inflammatory and Immune |
| inducible | W96709 | | processes ranging from hematopoiesis, | Biology, 2000, vol. 138: Chemokine Protocols. | disorders, Cardio-Vascular |
| protein (IP-10) | | | angiogenesis, and leukocyte trafficking. | Edited by: A. E. I. Proudfoot, T. N. C. Wells, and | discorders, Musco-skeletal |
| | | | Members of this family are involved in a | C. A. Power, Humana Press Inc., Totowa, NJ | disorders |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| A monokine | GeneSeq | U.S. Pat. No. 5871723 | Chemokines area family of related small, | Chemokines activities can be determined using | Angiogenesis, Cancer, |
| induced by | Accession | | secreted proteins involved in biological | assays known in the art: Methods in Molecular | Inflammatory and Immune |
| gamma- | W96710 | | processes ranging from hematopoiesis, | Biology, 2000, vol. 138: Chemokine Protocols. | disorders, Cardio-Vascular |
| interferon | | | angiogenesis, and leukocyte trafficking. | Edited by: A. E. I. Proudfoot, T. N. C. Wells, and | discorders, Musco-skeletal |
| (MIG) | | | Members of this family are involved in a | C. A. Power, Humana Press Inc., Totowa, NJ | disorders |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Interleukin-8 | GeneSeq | U.S. Pat. No. 5871723 | Chemokines area family of related small, | Chemokines activities can be determined using | Angiogenesis, Cancer, |
| (IL-8) protein. | Accession | | secreted proteins involved in biological | assays known in the art: Methods in Molecular | Inflammatory and Immune |
| | W96711 | | processes ranging from hematopoiesis, | Biology, 2000, vol. 138: Chemokine Protocols. | disorders, Cardio-Vascular |
| | | | angiogenesis, and leukocyte trafficking. | Edited by: A. E. I. Proudfoot, T. N. C. Wells, and | discorders, Musco-skeletal |
| | | | Members of this family are involved in a | C. A. Power, Humana Press Inc., Totowa, NJ; | disorders |
| | | | similarly diverse range of pathologies | and Holmes et al (1991) Science 253, 1278-80. | |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Epithelial | GeneSeq | U.S. Pat. No. 5871723 | Chemokines area family of related small, | Chemokines activities can be determined using | Angiogenesis, Cancer, |
| neutrophil | Accession | | secreted proteins involved in biological | assays known in the art: Methods in Molecular | Inflammatory and Immune |
| activating | W96712 | | processes ranging from hematopoiesis, | Biology, 2000, vol. 138: Chemokine Protocols. | disorders, Cardio-Vascular |
| protein-78 | | | angiogenesis, and leukocyte trafficking. | Edited by: A. E. I. Proudfoot, T. N. C. Wells, and | discorders, Musco-skeletal |
| (ENA-78) | | | Members of this family are involved in a | C. A. Power, Humana Press Inc., Totowa, NJ | disorders |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Growth related | GeneSeq | U.S. Pat. No. 5871723 | Chemokines area family of related small, | Chemokines activities can be determined using | Angiogenesis, Cancer, |
| oncogene-alpha | Accession | | secreted proteins involved in biological | assays known in the art: Methods in Molecular | Inflammatory and Immune |
| (GRO-alpha). | W96713 | | processes ranging from hematopoiesis, | Biology, 2000, vol. 138: Chemokine Protocols. | disorders, Cardio-Vascular |
| | | | angiogenesis, and leukocyte trafficking. | Edited by: A. E. I. Proudfoot, T. N. C. Wells, and | discorders, Musco-skeletal |
| | | | Members of this family are involved in a | C. A. Power, Humana Press Inc., Totowa, NJ | disorders |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a fmaily of seven transmembrane G- | | |
| | | | protein-coupled receptors. Over 40 human | | |
| | | | chemokines have been described, which bind | | |
| | | | to ˜17 receptors thus far identified. | | |
| Growth related | GeneSeq Accession | U.S. Pat. No. 5871723 | Chemokines are a family of related small, | Chemokine activities can be determined using | Angiogenesis, Cancer, |
| oncogene-beta | W96714 | | secreted proteins involved in biological | assays known in the art: Methods in | Inflammatory and Immune |
| (GRO-beta). | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified | | |
| Growth related | GeneSeq Accession | U.S. Pat. No. 5871723 | Chemokines are a family of related small, | Chemokine activities can be determined using | Angiogenesis, Cancer, |
| oncogene-gamma | W96715 | | secreted proteins involved in biological | assays known in the art: Methods in | Inflammatory and Immune |
| (GRO-gamma) | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| A platelet basic | GeneSeq Accession | U.S. Pat. No. 5871723 | Chemokines are a family of related small, | Chemokine activities can be determined using | Angiogenesis, Cancer, |
| protein (PBP) | W96716 | | secreted proteins involved in biological | assays known in the art: Methods in | Inflammatory and Immune |
| | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Connective tissue | GeneSeqAccession | U.S. Pat. No. 5871723 | Chemokines are a family of related small, | Chemokine activities can be determined using | Angiogenesis, Cancer, |
| activating protein- | S96717 | | secreted proteins involved in biological | assays known in the art: Methods in | Inflammatory and Immune |
| III (CTAP-III) | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Beta- | GeneSeq Accession | U.S. Pat. No. 5871723 | Chemokines are a family of related small, | Chemokine activities can be determined using | Angiogenesis, Cancer, |
| thromboglobulin | W96718 | | secreted proteins involved in biological | assays known in the art: Methods in | Inflammatory and Immune |
| protein (beta-TG) | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Neutrophil | GeneSeq Accession | U.S. Pat. No. 5871723 | Chemokines are a family of related small, | Chemokine activities can be determined using | Angiogenesis, Cancer, |
| activating peptide-2 | W96719 | | secreted proteins involved in biological | assays known in the art: Methods in | Inflammatory and Immune |
| (NAP-2) | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Granulocyte | GeneSeq Accession | U.S. Pat. No. 5871723 | Chemokines are a family of related small, | Chemokine activities can be determined using | Angiogenesis, Cancer, |
| chemotactic protein- | W96720 | | secreted proteins involved in biological | assays known in the art: Methods in | Inflammatory and Immune |
| 2 (GCP-2) | | | processes ranging from hematopoiesis, | Molecular Biology, 2000, vol. 138: | disorders, Cardio-Vascular |
| | | | angiogenesis, and leukocyte trafficking. | Chemokine Protocols. Edited by: A. E. I. Proudfoot, | disorders, Musco-skeletal |
| | | | Members of this family are involved in a | T. N. C. Wells, and C. A. Power, © | disorders |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | EP887409 | Chemokines are a family of related small, | Chemokine activities can be determined using | Immune disorders, viral, |
| MIG-beta protein | W90124 | | secreted proteins involved in biological | assays known in the art: Methods in | parasitic, fungal or bacterial |
| | | | processes ranging from hematopoiesis, | Molecular Biology, 2000, vol. 138: | infections, Cancer; |
| | | | angiogenesis, and leukocyte trafficking. | Chemokine Protocols. Edited by: A. E. I. Proudfoot, | autoimmune diseases or |
| | | | Members of this family are involved in a | T. N. C. Wells, and C. A. Power, © | transplant rejection |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human ZCHEMO-8 | GeneSeq Accession | WO9854326 | Chemokines are a family of related small, | Chemokine activities can be determined using | Immune disorders, cancer, |
| | W82716 | | secreted proteins involved in biological | assays known in the art: Methods in | myelopoietic disorders, |
| | | | processes ranging from hematopoiesis, | Molecular Biology, 2000, vol. 138: | autoimmune disorders and |
| | | | angiogenesis, and leukocyte trafficking. | Chemokine Protocols. Edited by: A. E. I. Proudfoot, | immunodeficiencies, |
| | | | Members of this family are involved in a | T. N. C. Wells, and C. A. Power, © | Inflammatory and infectious |
| | | | similarly diverse range of pathologies | Humana Press Inc., Totowa, NJ | diseases, Vascular disorders, |
| | | | including inflammation, allergy, tissue | | wound healing |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human Act-2 | GeneSeq Accession | WO9854326 | Chemokines are a family of related small, | Chemokine activities can be determined using | Immune disorders, cancer, |
| protein | W82717 | | secreted proteins involved in biological | assays known in the art: Methods in | myelopoietic disorders, |
| | | | processes ranging from hematopoiesis, | Molecular Biology, 2000, vol. 138: | autoimmune disorders and |
| | | | angiogenesis, and leukocyte trafficking. | Chemokine Protocols. Edited by: A. E. I. Proudfoot, | immunodeficiencies, |
| | | | Members of this family are involved in a | T. N. C. Wells, and C. A. Power, © | Inflammatory and infectious |
| | | | similarly diverse range of pathologies | Humana Press Inc., Totowa, NJ | diseases, Vascular disorders, |
| | | | including inflammation, allergy, tissue | | wound healing |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human SISD | GeneSeq | WO9854326 | Chemokines are a family of related small, | Chemokine activities can be determined using | Immune disorders, cancer, |
| protein | Acession | | secreted proteins involved in biological | assays known in the art: Methods in | myelopoietic disorders, |
| | W82720 | | processes ranging from hematopoiesis, | Molecular Biology, 2000, vol. 138: | autoimmune disorders and |
| | | | angiogenesis, and leukocyte trafficking. | Chemokine Protocols, Edited by: A. E. I. Proudfoot, | immunodeficiencies, |
| | | | Members of this family are involved in a | T. N. C. Wells, and C. A. Power. © | Inflammatory and infectious |
| | | | similarly diverse range of pathologies | Humana Press Inc., Totowa, NJ | diseases, Vascular disorders, |
| | | | including inflammation, allergy, tissue | | wound healing |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human M110 | GeneSeq | WO9854326 | Chemokines area family of related small, | Chemokine activities can be determined using | Immune disorders, cancer, |
| protein | Accession | | secreted proteins involved in biological | assays known in the art: Mehtods of Molecular | myelopoietic disorders, |
| | W82721 | | processes ranging from hematopoiesis, | Biology, 2000, vol. 138: Chemokine Protocols. | autoimmune disorders and |
| | | | angiogenesis, and leukocyte trafficking. | Edited by: A. E. I. Proudfoot, T. N. C. Wells, and | immunodeficiencies, |
| | | | Members of this family are involved in a | C. A. Power © Humana Press Inc., Totowa, NJ | Inflammatory and infectious |
| | | | similarly diverse range of pathologies | | diseases, Vascular disorders, |
| | | | including inflammation, allergy, tissue | | wound healing |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human M11A | GeneSeq | W09854326 | Chemokines area family of related small, | Chemokine activities can be determined using | Immune disorders, cancer, |
| protein | Accession | | secreted proteins involved in biological | assays known in the art: Mehtods of Molecular | myelopoietic disorders, |
| | W82722 | | processes ranging from hematopoiesis, | Biology, 2000, vol. 138: Chemokine Protocols. | autoimmune disorders and |
| | | | angiogenesis, and leukocyte trafficking. | Edited by: A. E. I. Proudfoot, T. N. C. Wells, and | imunodeficiencies, |
| | | | Members of this family are involved in a | C. A. Power © Humana Press Inc., Totowa, NJ | Inflammatory and infectious |
| | | | similarly diverse range of pathologies | | diseases, Vascular disorders, |
| | | | including inflammation, allergy, tissue | | wound healing |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human CCC3 | GeneSeq | WO9854326 | Chemokines area family of related small, | Chemokine activities can be determined using | Immune disorders, cancer, |
| protein | Accession | | secreted proteins involved in biological | assays known in the art: Mehtods of Molecular | myelopoietic disorders, |
| | W82723 | | processes ranging from hematopoiesis, | Biology, 2000, vol. 138: Chemokine Protocols. | autoimmune disorders and |
| | | | angiogenesis, and leukocyte trafficking. | Edited by: A. E. I. Proudfoot, T. N. C. Wells, and | immunodeficiencies, |
| | | | Members of this family are involved in a | C. A. Power © Humana Press Inc., Totowa, NJ | Inflammatory and infectious |
| | | | similarly diverse range of pathologies | | diseases, Vascular disorders, |
| | | | including inflammation, allergy, tissue | | wound healing |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| A human L105 | GeneSeq | WO9856818 | Chemokines area family of related small, | Chemokine activities can be determined using | Cancer, wound healing |
| chemokine | Accession | | secreted proteins involved in biological | assays known in the art: Mehtods of Molecular | |
| designated | W87588 | | processes ranging from hematopoiesis, | Biology, 2000, vol. 138: Chemokine Protocols. | |
| huL105_3. | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| A human L105 | GeneSeq | WO9856818 | Chemokines area family of related small, | Chemokine activities can be determined using | Cancer, wound healing |
| chemokine | Accession | | secreted proteins involved in biological | assays known in the art: Mehtods of Molecular | |
| designated | W87589 | | processes ranging from hematopoiesis, | Biology, 2000, vol. 138: Chemokine Protocols. | |
| huL105_7. | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human mature | GeneSeq | WO9848828 | Chemokines area family of related small, | Chemokine activities can be determined using | Infectious diseases, sepsis |
| gro-alpha | Accession | | secreted proteins involved in biological | assays known in the art: Mehtods of Molecular | |
| polypeptide | W81498 | | processes ranging from hematopoiesis, | Biology, 2000, vol. 138: Chemokine Protocols. | |
| used to treat | | | angiogenesis, and leukocyte trafficking. | Edited by: A. E. I. Proudfoot, T. N. C. Wells, and | |
| sepsis | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human mature | GeneSeq | WO9848828 | Chemokines area family of related small, | Chemokine activities can be determined using | Infectious diseases, sepsis |
| gro-gamma | Accession | | secreted proteins involved in biological | assays known in the art: Mehtods of Molecular | |
| polypeptide | W81500 | | processes ranging from hematopoiesis, | Biology, 2000, vol. 138: Chemokine Protocols. | |
| used to treat | | | angiogenesis, and leukocyte trafficking. | Edited by: A. E. I. Proudfoot, T. N. C. Wells, and | |
| sepsis | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human thymus | GeneSeq | WO0053635 | Chemokines area family of related small, | Chemokine activities can be determined using | Inflammatory disorders, |
| expressed | Accessions | | secreted proteins involved in biological | assays known in the art: Mehtods of Molecular | cancer, Immune and vascular |
| chemokine | B19607 and | | processes ranging from hematopoiesis, | Biology, 2000, vol. 138: Chemokine Protocols. | disorders |
| TECK and | B19608 | | angiogenesis, and leukocyte trafficking. | Edited by: A. E. I. Proudfoot, T. N. C. Wells, and | |
| TECK variant | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human | GeneSeq | WO0042071 | Chemokines area family of related small, | Chemokine activities can be determined using | Autoimmune disorders, |
| chemokine | Accession B15791 | | secreted proteins involved in biological | assays known in the art: Mehtods of Molecular | Immune, Vascular and |
| SDF1alpha | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot, T. N. C. Wells, and | |
| | | | Members of this family are involved in a | C. A. Power © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| GROalpha | B15793 | | secreted proteins involved in biological | assasys known in the art: Methods in | Immune, Vascular and |
| | | | processes ranging from hematopoiesis, | Molecular Biology, 2000, vol. 138: | Inflammatory diorders |
| | | | angiogenesis, and leukocyte trafficking. | Chemokine Protocols. Edited by: A. E. I. Proudfoot; | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| eotaxin | B15794 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| MIG | B15803 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| PF4 | B15804 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| I-309 | B15805 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| HCC-1 | B15806 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| C10 | B15807 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| CCR-2 | B15808 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| ENA-78 | B15809 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| GRObeta | B15810 | | secreted proteins involved in biological | assasys known in the art: Methods in Molecular | Immune, Vascular and |
| | | | processes ranging from hematopoiesis, | Biology, 2000, vol. 138: Chemokine Protocols. | Inflammatory disorders |
| | | | angiogenesis, and leukocyte trafficking. | Edited by: A. E. I. Proudfoot; T. N. C. Wells, and | |
| | | | Members of this family are involved in a | C. A. Power. © Humana Press Inc., Totowa, NJ | |
| | | | similarly diverse range of pathologies | | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| IP-10 | B15811 | | secreted proteins involved in biological | assays known in the art: Methods in | Immune, Vascular and |
| | | | processes ranging from hematopoiesis, | Molecular Biology, 2000, vol. 138: | Inflammatory disorders |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| SDF1beta | B15812 | | secreted proteins involved in biological | assays known in the art: Methods in | Immune, Vascular and |
| | | | processes ranging from hematopoiesis, | Molecular Biology, 2000, vol. 138: | Inflammatory disorders |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| GRO alpha | B15813 | | secreted proteins involved in biological | assays known in the art: Methods in | Immune, Vascular and |
| | | | processes ranging from hematopoiesis, | Molecular Biology, 2000, vol. 138: | Inflammatory disorders |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0042071 | Chemokines are a family of related small, | Chemokine activities can be determined using | Autoimmune disorders, |
| MIP1beta | B15831 | | secreted proteins involved in biological | assays known in the art: Methods in | Immune, Vascular and |
| | | | processes ranging from hematopoiesis, | Molecular Biology, 2000, vol. 138: | Inflammatory disorders |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| A human C-C | GeneSeq Accession | U.S. Pat. No. 6096300 | Chemokines are a family of related small, | Chemokine activities can be determined using | Cancer |
| chemokine | B07939 | | secreted proteins involved in biological | assays known in the art: Methods in | |
| designated exodus | | | processes ranging from hematopoiesis, | Molecular Biology, 2000, vol. 138: | |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | U.S. Pat. No. 6084071 | Chemokines are a family of related small, | Chemokine activities can be determined using | Chemotaxis, Gene Therapy, |
| L105_7 | Y96922 | | secreted proteins involved in biological | assays known in the art: Methods in | Wound healing |
| | | | processes ranging from hematopoiesis, | Molecular Biology, 2000, vol. 138: | |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | U.S. Pat. No. 6084071 | Chemokines are a family of related small, | Chemokine activities can be determined using | Chemotaxis, Gene Therapy, |
| L105_3 | Y96923 | | secreted proteins involved in biological | assays known in the art: Methods in | Wound healing |
| | | | processes ranging from hematopoiesis, | Molecular Biology, 2000, vol. 138: | |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human secondary | GeneSeq Accession | WO0038706 | Chemokines are a family of related small, | Chemokine activities can be determined using | Cancer, Vascular and |
| lymphoid | B01434 | | secreted proteins involved in biological | assays known in the art: Methods in | Immune disorders |
| chemokine (SLC) | | | processes ranging from hematopoiesis, | Molecular Biology, 2000, vol. 138: | |
| | | | angiogenesis, and leukocyte trafficking. | Chemokine Protocols. Edited by: A. E. I. Proudfoot, | |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human non-ELR | GeneSeq Accession | WO0029439 | Chemokines are a family of related small, | Chemokine activities can be determined using | Immune and Inflammatory |
| CXC chemokine | Y96310 | | secreted proteins involved in biological | assays known in the art: Methods in | disorders, Cancer, |
| H174 | | | processes ranging from hematopoiesis, | Molecular Biology, 2000, vol. 138: | Haemostatic and |
| | | | angiogenesis, and leukocyte trafficking. | Chemokine Protocols. Edited by: A. E. I. Proudfoot, | thrombolytic activity |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human non-ELR | GeneSeq Accession | WO0029439 | Chemokines are a family of related small, | Chemokine activities can be determined using | Immune and Inflammatory |
| CXC chemokine | Y96311 | | secreted proteins involved in biological | assays known in the art: Methods in | disorders, Cancer, |
| IP10 | | | processes ranging from hematopoiesis, | Molecular Biology, 2000, vol. 138: | haemostatic and thrombolytic |
| | | | angiogenesis, and leukocyte trafficking. | Chemokine Protocols. Edited by: A. E. I. Proudfoot, | activity |
| | | | Members of this family are involved in a | T. N. C. Wells, and C. A. Power. © | |
| | | | similarly diverse range of pathologies | Humana Press Inc., Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human non-ELR | GeneSeq Accession | WO0029439 | Chemokines are a family of related small, | Chemokine activities can be determined using | Immune and Inflammatory |
| CXC chemokine | Y96313 | | secreted proteins involved in biological | assays known in the art: Methods in Molecular | disorders, Cancer, |
| Mig | | | processes ranging from hematopoiesis, | Biology, 2000, vol. 138: Chemokine | haemostatic and thrombolytic |
| | | | angiogenesis, and leukocyte trafficking. | Protocols. Edited by: A. E. I. Proudfoot, T. N. C. Wells, | activity |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0028035 | Chemokines are a family of related small, | Chemokine activities can be determined using | Cancer, wound healing, |
| Ckbeta-7 | Y96280 | | secreted proteins involved in biological | assays known in the art: Methods in Molecular | inflammatory and |
| | | | processes ranging from hematopoiesis, | Biology, 2000, vol. 138: Chemokine | immunoregulatory disorders |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0028035 | Chemokines are a family of related small, | Chemokine activities can be determined using | Cancer, wound healing, |
| MIP-1alpha | Y96281 | | secreted proteins involved in biological | assays known in the art: Methods in Molecular | inflammatory and |
| | | | processes ranging from hematopoiesis, | Biology, 2000, vol. 138: Chemokine | immunoregulatory disorders |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human mature | GenSeq Accession | WO0028035 | Chemokines are a family of related small, | Chemokine activities can be determined using | Cancer, wound healing, |
| chemokine Ckbeta- | Y96282 | | secreted proteins involved in biological | assays known in the art: Methods in Molecular | inflammatory and |
| 7 (optionally | | | processes ranging from hematopoiesis, | Biology, 2000, vol. 138: Chemokine | immunoregulatory disorders |
| truncated) | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human chemokine | GeneSeq Accession | WO0018431 | Chemokines are a family of related small, | Chemokine activities can be determined using | Soluble CXCR3 polypeptides |
| receptor CXCR3 | Y79372 | | secreted proteins involved in biological | assays known in the art: Methods in Molecular | may be useful for inhibiting |
| | | | processes ranging from hematopoiesis, | Biology, 2000, vol. 138: Chemokine | chemokine activities and |
| | | | angiogenesis, and leukocyte trafficking. | Protocols. Edited by: A. E. I. Proudfoot, T. N. C. Wells, | viral infection. |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human neurotactin | GeneSeq Accession | U.S. Pat. No. 6043086 | Chemokines are a family of related small, | Chemokine activities can be determined using | Neurological disorders, |
| chemokine like | Y53259 | | secreted proteins involved in biological | assays known in the art: Methods in Molecular | Immune and respiratory |
| domain | | | processes ranging from hematopoiesis, | Biology, 2000, vol. 138: Chemokine | disorders |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified. | | |
| Human CC type | GeneSeq Accession | JP11302298 | Chemokines are a family of related small, | Chemokine activities can be determined using | Cancer and infectious |
| chemokine | Y57771 | | secreted proteins involved in biological | assays known in the art: Methods in Molecular | diseases |
| interleukin C | | | processes ranging from hematopoiesis, | Biology, 2000, vol. 138: Chemokine | |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified | | |
| Human CKbeta-9 | GeneSeq Accession | U.S. Pat. No. 6153441 | Chemokines are a family of related small, | Chemokine activities can be determined using | Cancer, Auto-immune and |
| | B50860 | | secreted proteins involved in biological | assays known in the art: Methods in Molecular | inflammatory disorders, |
| | | | processes ranging from hematopoiesis, | Biology, 2000, vol. 138: Chemokine | Cardiovascular disorders |
| | | | angiogenesis, and leukocyte trafficking. | Protocols. Edited by: A. E. I. Proudfoot, T. N. C. Wells, | |
| | | | Members of this family are involved in a | and C. A. Power. © Humana Press Inc., | |
| | | | similarly diverse range of pathologies | Totowa, NJ | |
| | | | including inflammation, allergy, tissue | | |
| | | | rejection, viral infection, and tumor biology. | | |
| | | | The chemokines exert their effects by acting | | |
| | | | on a family of seven transmembrane G- | | |
| | | | protein-coupled receptors. Over 40 human | | |
| | | | chemokines have been described, which bind | | |
| | | | to ˜17 receptors thus far identified | | |
| Preproapolipoprotein | GeneSeq Accession | WO9637608 | Apoa-1 participates in the reverse transport of | Lipid binding activity can be determined using | Useful for cardiovascular |
| “paris” variant | W08602 | | cholesterol from tissues to the liver for | assays known in the art, such as, for example, | disorders, cholesterol |
| | | | excretion by promoting cholesterol efflux | the Cholesterol Efflux Assays of Takahaski et | disorders, and |
| | | | from tissues and by acting as a cofactor for | al., P.N.A.S., Vol. 96, Issue 20, 11358-11363, | Hyperlipidaemia |
| | | | the lecithin cholesterol acyltransferase (lcat). | Sep. 28, 1999. | |
| Preproapolipoprotein | | 5,721,114 | Apoa-1 participates in the reverse transport of | Lipid binding activity can be determined using | Useful for cardiovascular |
| “milano” variant | | | cholesterol from tissues to the liver for | assays known in the art, such as, for example, | disorders, cholesterol |
| | | | excretion by promoting cholesterol efflux | the Cholesterol Efflux Assays of Takahaski et | disorders, and |
| | | | from tissues and by acting as a cofactor for | al., P.N.A.S., Vol. 96, Issue 20, 11358-11363, | Hyperlipidaemia |
| | | | the lecithin cholesterol acyltransferase (lcat). | Sep. 28, 1999. | |
| Glycodelin-A; | GeneSeq Accession | WO9628169 | Naturally produced female contraceptive that | Glycodelin-A activity can be determined using | Naturally derived |
| Progesterone- | W00289 | | is removed rapidly from the body following | the hemizona assay as described in Oehninger, S., | contraceptive useful for the |
| associated | | | 2-3 days production. Uses include | Coddington, C. C., Hodgen, G. D., and | prevention of pregnancy. |
| endometrial protein | | | contraception | Seppala, M (1995) Fertil. Steril. 63, 377-383. | |
| NOGO-A | Genbank Accession | | NOGO polypeptides are potent inhibitors of | Inhibition of Neurite outgrowth. Antagonists | NOGO-A polypeptide |
| | CAB99248 | | neurite growth. | to NOGO polypeptides may promote the | antagonists are useful for the |
| | | | | outgrowth of neurites, thus inducing | promotion of neural growth, |
| | | | | regeneration of neurons. | which could be useful in the |
| | | | | | treatment of neural disorders |
| | | | | | and dysfunction due to |
| | | | | | degenerative diseases or |
| | | | | | trauma; useful in the |
| | | | | | treatment of neoplastic |
| | | | | | diseases of the CNS; induce |
| | | | | | regeneration of neurons or to |
| | | | | | promote the structural |
| | | | | | plasticity of the CNS. |
| NOGO-B | Genbank Accession | | NOGO polypeptides are potent inhibitors of | Inhibition of Neurite outgrowth. Antagonists | NOGO-B polypeptide |
| | CAB99249 | | neurite growth. | to NOGO polypeptides may promote the | antagonists are useful for the |
| | | | | outgrowth of neurites, thus inducing | promotion of neural growth, |
| | | | | regeneration of neurons. | which could be useful in the |
| | | | | | treatment of neural disorders |
| | | | | | and dysfunction due to |
| | | | | | degenerative diseases or |
| | | | | | trauma; useful in the |
| | | | | | treatment of neoplastic |
| | | | | | diseases of the CNS; induce |
| | | | | | regeneration of neurons or to |
| | | | | | promote the structural |
| | | | | | plasticity of the CNS. |
| NOGO-C | Genbank Accession | | NOGO polypeptides are potent inhibitors of | Inhibition of Neurite outgrowth. Antagonists | NOGO-C polypeptide |
| | CAB99250 | | neurite growth. | to NOGO polypeptides may promote the | antagonists are useful for the |
| | | | | outgrowth of neurites, thus inducing | promotion of neural growth, |
| | | | | regeneration of neurons. | which could be useful in the |
| | | | | | treatment of neural disorders |
| | | | | | and dysfunction due to |
| | | | | | degenerative diseases or |
| | | | | | trauma; useful in the |
| | | | | | treatment of neoplastic |
| | | | | | diseases of the CNS; induce |
| | | | | | regeneration of neurons or to |
| | | | | | promote the structural |
| | | | | | plasticity of the CNS. |
| NOGO-66 Receptor | Genbank Accession | | NOGO polypeptides are potent inhibitors of | Inhibition of Neurite outgrowth by mediating | NOGO-66 receptor |
| | AAG53612 | | neurite growth, and are thought to mediate | the biological effects of NOGO polypeptides. | polypeptides are useful for |
| | | | their effects through the NOGO-66 Receptor. | Soluble NOGO-66 receptor polypeptides may | the promotion of neural |
| | | | | promote the outgrowth of neurites, thus | growth, which could be |
| | | | | inducing regeneration of neurons. | useful in the treatment of |
| | | | | | neural disorders and |
| | | | | | dysfunction due to |
| | | | | | degenerative diseases or |
| | | | | | trauma; useful in the |
| | | | | | treatment of neoplastic |
| | | | | | diseases of the CNS; induce |
| | | | | | regeneration of neurons or to |
| | | | | | promote the structural |
| | | | | | plasticity of the CNS. |
| Antibodies specific | | U.S. Pat. No. 5416197 | These antibodies are useful for the promotion | Collapsin activity, which is thought to inhibit | Useful for the promotion of |
| for collapsin | | | of neurite outgrowth | the outgrowth of neurites, can be assayed in | neural growth, which could |
| | | | | the presence of antibodies specific for | be useful in the treatment of |
| | | | | collapsing using assays known in the art, such | neural disorders and |
| | | | | as, for example, the collapse assay disclosed | dysfunction due to |
| | | | | by Luo et al., Cell 1993 Oct 22; 75(2): 217-27 | degenerative diseases or |
| | | | | | trauma. |
| Humanized Anti- | | WO9845331 | These agents have anti-inflammatory and | VEGF activity can be determined using assays | Promotion of growth and |
| VEGF Antibodies, | | | anti-cancer applications | known in the art, such as those disclosed in | proliferation of cells, such as |
| and fragments | | | | International Publication No. WO0045835, for | vascular endothelial cells. |
| thereof | | | | example. | Antagonists may be useful as |
| | | | | | anti-angiogenic agents, and |
| | | | | | may be applicable for cancer |
| Humanized Anti- | | WO0029584 | These agents have anti-inflammatory and | VEGF activity can be determined using assays | Promotion of growth and |
| VEGF Antibodies, | | | anti-cancer applications | known in the art, such as those disclosed in | proliferation of cells, such as |
| and fragments | | | | International Publication No. WO0045835, for | vascular endothelial cells. |
| thereof | | | | example. | Antagonists may be useful as |
| | | | | | anti-angiogenic agents, and |
| | | | | | may be applicable for cancer |
| Membrane bound | GeneSeq. Accession | WO9963088 | Cancer, Immune Disorders | These proteins can be used for linking | Activities can be determined |
| proteins | Y66631-Y66765 | | | bioactive molecules to cells and for | using assay known in the art, |
| | | | | modulating biological activities of cells, using | suchas, for example, the |
| | | | | the polypeptides for specific targeting. The | assays disclosed in |
| | | | | polypeptide targeting can be used to kill the | International Publication No. |
| | | | | target cells, e.g. for the treatment of cancers. | WO0121658. |
| | | | | These proteins are useful for the treatment of | |
| | | | | immune system disorders. | |
| Secreted and | GenSeq Accession | WO0053756 | Cancer, Immune Disorders | These proteins can be used for linking | Activities can be determined |
| Transmembrane | B44241-B44334 | | | bioactive molecules to cells and for | using assay known in the art, |
| polypeptides | | | | modulating biological activities of cells, using | suchas, for example, the |
| | | | | the polypeptides for specific targeting. The | assays disclosed in |
| | | | | polypeptide targeting can be used to kill the | International Publication No. |
| | | | | target cells, e.g. for the treatment of cancers. | WO0121658 |
| | | | | These proteins are useful for the treatment of | |
| | | | | immune system disorders. | |
| Secreted and | GeneSeq Accession | WO9946281 | Cancer, Immune Disorders | These proteins can be used for linking | Activities can be determined |
| Transmembrane | Y41685-Y41774 | | | bioactive molecules to cells and for | using assay known in the art, |
| polypeptides | | | | modulating biological activities of cells, using | suchas, for example, the |
| | | | | the polypeptides for specific targeting. The | assays disclosed in |
| | | | | polypeptide targeting can be used to kill the | International Publication No. |
| | | | | target cells, e.g. for the treatment of cancers. | WO0121658 |
| | | | | These proteins are useful for the treatment of | |
| | | | | immune system disorders. | |

We claim:

1. A compound comprising an optionally stabilized nucleic acid or nucleic acid analogue, which encodes for a peptide, protein or other biological modifier, conjugated directly or via a linker to a ligand for the transcobalamin receptor or intrinsic factor receptor.

2. A compound comprising an optionally stabilized aptamer conjugated directly or via a linker to a ligand for the transcobalamin receptor or intrinsic factor receptor

3. A compound comprising an optionally stabilized antisense sequence conjugated directly or via a linker to a ligand for the transcobalamin receptor or intrinsic factor receptor.

4. A compound comprising an optionally stabilized antisense mimic conjugated directly or via a linker to a ligand for the transcobalamin receptor or intrinsic factor receptor.

5. The compound of claim 4, wherein the mimic is selected from the group consisting of that nucleic acids, analogs and derivatives thereof PNA, MNA, LNA, PCO and ENA

6. The compound of claim 5, wherein the mimic is PNA.

7. The compound of claim 5, wherein the mimic is MNA.

8. The compound of claim 5, wherein the mimic is LNA.

9. The compound of claim 5, wherein the mimic is PCO.

10. The compound of claim 5, wherein the mimic is ENA.

11. A compound of the formula: 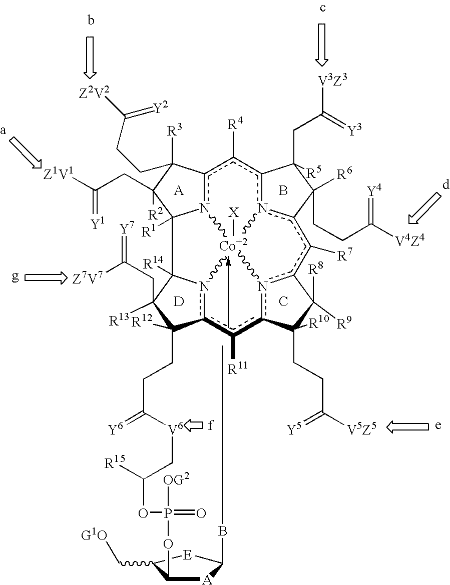
or its enantiomer, diastereomer, salt or prodrug thereof, wherein:
(xvi) X is hydrogen, cyano, amino, amido, hydroxyl, adenosyl L-T, alkyl, alkenyl, alkynyl, cylcoalkyl, aryl, aralkyl, heterocycle, heteroaryl or alkylheteroaryl;
(xvii) B is a divalent heterocycle wherein the radical positions can be within the ring or a substituent to the ring such that at least one radical is on a heteroatom to form a dative bond with cobalt, optionally substituted by L-T;
(xviii) A is O, S, NJ¹, CR¹⁰⁰R¹⁰¹ or C(R¹⁰⁰)V⁸Z⁸;
(xix) E is O or S;
(xx) G¹ and G² are independently hydrogen, alkyl, acyl, silyl, phosphate, or L-T;
(xxi) Y¹, Y², Y³ Y⁴, Y⁵, Y⁶ and Y⁷ independently are O, S or NJ²;
(xxii) V¹, V², V³, V⁴, V⁵, V⁶, V⁷ and V⁸ independently are O, S or NJ³; CR¹⁰²R¹⁰³, or a direct bond;
(xxiii) Z¹, Z², Z³, Z⁴, Z¹, Z⁷ and Z⁸ independently are R¹⁰⁴ or L-T;
(xxiv) each L is independently a direct bond or the residue of a multivalent moiety that does not significantly impair the ability of the compound to bind transcobalamin II;
(xxv) each T is independently a nucleic acid sequence useful in antisense technology, a peptide nucleic acid or morpholino nucleic acidan optionally stabilized (i) nucleic acid or nucleic acid analogue which can encode for a peptide, protein or other biological modifier; (ii) a “nonsense” sequence, sometimes also referred to as an aptamer; (iii) an antisense sequence or (iv) an antisense mimic, including but not limited to PNA, MNA, LNA, PCO or ENA;
(xxvi) at least one of Z¹, Z², Z³, Z⁴, Z⁵, Z⁷, Z⁸, A, B, G¹, and G² comprises an a nucleic acid sequence useful in antisense technology, a peptide nucleic acid or morpholino nucleic acid;
(xxvii) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xxviii) R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(xxix) R¹³ and R¹⁴ optionally can come together to form a pi bond; and
(xxx) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

12. The compound of claims 1-11 wherein the ligand is vitamin B₁₂ B₁₂ or a derivative thereof including selected from 5′-adenosylcobalamin, methylcobalamin, hydroxycobalamin or cyanocobalamin

13. The compound of claim 11, wherein T is conjugated through at least one of Z¹, Z², Z³ Z⁴ or Z⁵.

14. The compound of claim 13, wherein T is conjugated though the “b” carboxamide of vitamin B₁₂ (“Z²”).

15. A pharmaceutical composition comprising a compound of claims 1-14.

16. A method of gene therapy comprising:
injecting a host cell or organism with a nucleic acid conjugated to a ligand for a cell surface receptor selected from the group consisting of transcobalamin II receptor and intrinsic factor receptor, wherein the nucleic acid.

17. The method of claim 16, wherein the nucleic acid is operably linked to a promoter which directs the expression of the protein encoded by the nucleic acid.

18. A method for the delivery in vivo or in vitro of an optionally stabilized nucleic acid or nucleic acid analogue, which encodes for a peptide, protein or other biological modifier, comprising conjugating it directly or via a linker to a ligand for the transcobalamin receptor or intrinsic factor receptor.

19. A method for the delivery in vivo or in vitro of an optionally stabilized aptamer comprising conjugating it directly or via a linker to a ligand for the transcobalamin receptor or intrinsic factor receptor

20. A method for the delivery in vivo or in vitro of an optionally stabilized antisense sequence comprising conjugating it directly or via a linker to a ligand for the transcobalamin receptor or intrinsic factor receptor.

21. A method for the delivery in vivo or in vitro of an optionally stabilized antisense mimic comprising conjugating it directly or via a linker to a ligand for the transcobalamin receptor or intrinsic factor receptor.

22. The method of claim 21, wherein the mimic is selected from the group consisting of that nucleic acids, analogs and derivatives thereof PNA, MNA, LNA, PCO and ENA

23. The method of claim 22, wherein the mimic is PNA.

24. The method of claim 22, wherein the mimic is MNA.

25. The method of claim 22, wherein the mimic is LNA.

26. The method of claim 22, wherein the mimic is PCO.

27. The method of claim 22, wherein the mimic is ENA.
